(19) United States
(12) Patent Application Publication
(10) Pub. No.: US 2025/0057962 A1
(43) Pub. Date: Feb. 20, 2025

(54) PROCESSABLE COMPOSITIONS AND USE FOR THE SAME

(71) Applicant: Ripple Therapeutics Corporation, Toronto (CA)

(72) Inventors: Ian Charles PARRAG, Mississauga (CA); Matthew Alexander John STATHAM, Milton (CA); Kyle Giovanni BATTISTON, Toronto (CA); Wendy Alison NAIMARK, Toronto (CA); Jonathan DAY, Toronto (CA); Emily BALDWIN, Toronto (CA)

(21) Appl. No.: 18/706,880

(22) PCT Filed: Nov. 2, 2022
(86) PCT No.: PCT/IB2022/000649

§ 371 Date: May 2, 2024

Related U.S. Application Data
(60) Provisional application No. 63/275,281, filed on Nov. 3, 2021.
(60) Provisional application No. 63/275,283, filed on Nov. 3, 2021.

(51) Int. Cl. A61K 47/55; A61K 9/00; A61K 47/54

(52) U.S. Cl. CPC A61K 47/55; A61K 9/0051; A61K 47/554

(57) ABSTRACT

Provided herein are compounds having the formula D1-L-D2, wherein D1 is a processable group, L is a linker, and D2 is a drug. Also described herein are pharmaceutical compositions comprising said compounds and methods for the treatment of ocular diseases or disorders including glaucoma, ocular hypertension, ocular inflammation, diabetic macular edema, posterior inflammation, anterior inflammation, macular degeneration, post-cataract surgery and retinal vein occlusion.

### CROSS-REFERENCE

This application claims the benefit of U.S. Provisional Application No. 63/275,281 filed Nov. 3, 2021, and U.S. Provisional Application No. 63/275,283 filed Nov. 3, 2021, which are each hereby incorporated by reference in their entirety herein.

### BACKGROUND OF THE INVENTION

The global controlled release drug delivery market size in 2019 was estimated at over $40 B. While there are some controlled delivery systems for delivering an active pharmaceutical ingredient (API) to a treatment site, the systems generally contain excipients, such as polymer carriers, that can create technical and/or regulatory challenges, such as, for example, burst release kinetics, low drug loading, and adverse inflammatory responses. Further, such excipients can be inactive components, and such systems containing such inactive components may cause adverse effects and/or require removal. Providing a therapeutic consisting entirely or almost entirely of API could address such technical and regulatory challenges.

### SUMMARY OF THE INVENTION

Provided in some embodiments herein are compounds comprising a first radical (D1) and a second radical (D2) (e.g., having the formula: D1-L-D2). In certain instances, D1 is a processable group (also referred to herein as a processable radical), L is a linker, and D2 is a drug (also referred herein as a drug radical). In some embodiments, L is a hydrolyzable linker, such that when the compound of formula D1-L-D2 is (e.g., ophthalmically) administered (or when present in or otherwise exposed to an aqueous environment, such as a buffering solution, tears, serum, or the like), D1 and D2 are released (e.g., in their free, non-radical form). In certain instances, the (e.g., covalent) joining of a group D1 to an active agent D2 (e.g., non-processable active agent) through a linker L (e.g., D1-L-D2), provides a compound comprising an otherwise non-processable drug (e.g., D2-L-D2 (e.g., D2-D2)) in a processable form. In certain instances, a drug (such as a therapeutically active agent (e.g., prostaglandin) provided herein) is joined with a processable group (such as a steroid or other radical of a formula described herein, such as Formula (I) or Formula (ID)). In certain instances, a drug (such as a prostaglandin provided herein) is joined with a processable group (such as a steroid or other radical of a formula described herein, such as Formula (I) or Formula (ID)). In certain instances, the processable group D1 may or may not itself be processable when in free form, but when combined with D2 (e.g., through a linker L) provides a solid (e.g., at a physiological temperature) that is processable (e.g., at a temperature above a physiological temperature).

Provided in certain instances herein is a platform for providing compounds and implants (e.g., with high drug content, low excipient content (e.g., that would otherwise need to be removed), and other benefits, such as described herein) that provide long-lasting release of therapeutics (e.g., a therapeutically active agent (e.g., prostaglandin or steroid) provided herein) in biological and therapeutic applications, such as in ocular (e.g., implant) administration and/or via implantation. Provided in certain instances herein is a platform for providing compounds and implants (e.g., with high drug content, low excipient content (e.g., that would otherwise need to be removed), and other benefits, such as described herein) that provide long-lasting release of therapeutics (e.g., prostaglandins and/or steroids) in biological and therapeutic applications such as via implantation and/or in ocular (e.g., implant) administration.

In some instances, compounds provided herein (e.g., joining a non-processable drug (e.g., a prostaglandin radical) to a processable drug (e.g., a steroid) radical, such as through a (e.g., hydrolyzable) linker) are processable into forms (e.g., implants, coatings, or other bodies), such as that are capable of being administered to (e.g., an eye of) an individual in need thereof. In some instances, such compounds are processable without the need for additional excipients or materials (e.g., controlled release polymers, matrices, or other components). In certain instances, no or low amounts of additional excipients or materials facilitate high levels of drug delivery, while limiting impact of drug delivery (e.g., a small implant can have high quantities of drug).

In certain instances, such compounds (or implants comprising such compounds) are administered to (e.g., implanted into) an individual, such that sustained and/or otherwise controlled (e.g., local) delivery of the drug is achieved. In some instances, delivery of the compounds (e.g., in the form of an implant, coating, etc.) facilitate delivery of a drug component for an extended period of time, such as for weeks, months, or more. In certain instances, compounds, formulations, and implants provided herein facilitate the long term delivery of drugs to an individual in need thereof, such as without the need for frequent dosing. For example, as discussed herein, ocular drugs, such as prostaglandins, are often formulated and administered as eye drops, such as with daily administration. In some instances, rigid compliance to frequent administration is required to maintain (e.g., optimal) therapeutic efficacy. With the compounds provided herein, however, long term delivery of such drugs can be achieved from weeks, months, or more, with infrequent administration (e.g., once a year, twice a year, or the like).

In some embodiments, the group D2 is an active agent or drug. In some embodiments, the group D1 is also an active agent or drug. In some embodiments, D1 and D2 are both effective in the treatment of a single indication, such that administration of a compound herein provides a combination therapeutic effect. In some instances, D1 provides a therapeutic benefit (e.g., in its free form) and D2 treats or prevents an effect (e.g., a side-effect) of D1 (in its free form). In some embodiments, D1 is an inactive agent (e.g., an agent that (in its free form) does not provide a (e.g., significant, measurable, and/or direct) therapeutic effect and/or benefit (e.g., to an individual (e.g., in need thereof)) and D2 is an active agent (e.g., a therapeutically active agent) (or drug). In some embodiments, the therapeutically active agent is released (e.g., at a controlled rate (e.g., over an extended period of time)) from a composition provided herein (e.g., an article, implant, or coating). In some embodiments, such as wherein the compound is formulated as or with an implant, the inactive agent acts as a carrier for providing a therapeutically active agent to a (e.g., treatment site of) an individual (e.g., in need thereof) (e.g., for an extended period of time) and the therapeutic agent provides a therapeutically beneficial effect for an extended period of time.

In some embodiments, such as in therapies for the treatment of glaucoma, the anti-inflammatory effect of the steroid and the ocular pressure lowering effect of the prostaglandin both provide therapeutic effect. In some embodiments, such as wherein the compound is formulated as or with an implant, D1 is an anti-inflammatory (e.g., steroid) and reduces or minimizes an inflammatory response to the implant. In some instances, D1 (e.g., a steroid radical) reduces or minimizes inflammation (e.g., in its free form) and D2 (e.g., a prostaglandin radical) treats or prevents an effect (e.g., a side-effect) of D1 (in its free form). For example, in some embodiments, D1 is a steroid and D2 is a prostaglandin.

Provided in some embodiments herein are compounds, such as described herein, (e.g., pharmaceutical) compositions comprising compounds described herein, and methods of making and using compounds provided herein. In some embodiments, methods of using the compounds provided herein include methods of treating disorders in individuals in need thereof, such as disorders treatable by a drug D2 (e.g., in its free form). In some embodiments, methods of treatment provided herein comprise methods of treating ocular disorders, such as glaucoma. It is to be understood that disclosures of methods provided herein explicitly include disclosures of pharmaceutical compositions comprising (e.g., an effective amount) of a compound provided herein for such uses.

In some instances, provided herein is a compound that delivers a therapeutically effective amount of (e.g., a free form of) a therapeutic agent (e.g., steroid and/or a prostaglandin), such as a therapeutically active agent (e.g., steroid or prostaglandin) described herein.

Provided in some embodiments herein is a compound comprising a first radical and a second radical, the first radical has a structure represented by Formula (I′):

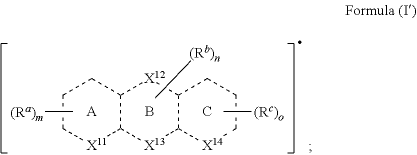

wherein:
   [symbol] is a single bond or a double bond;
   each R^{a}, R^{b}, and R^{c} are independently selected the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted;
      or any one of R^{a}, R^{b}, or R^{c} are taken together with another of R^{a}, R^{b}, or R^{c} to form a substituted or an unsubstituted cycloalkyl or heterocycloalkyl;
   X¹¹, X¹¹, X¹³, and X¹⁴ are each independently selected from the group consisting of a bond and Q_{y}, wherein each Q is independently selected from the group consisting of —O—, —NR—, —S(R)ₓ—, and —C(R)₂—;
      each x is independently 0-5;
      each y is independently 1-3;
      each z is independently 1 or 2;
      each R is independently selected from the group consisting of H, halogen, alkyl, heteroalkyl, cycloalkyl, heterocycloalkyl, alkoxy, amino, hydroxy, and thiol, or is taken together with another R to form an oxo; and
   each of m, n, and o are independently 0-6;
the second radical being a therapeutically active agent (or drug) (e.g., in its free form) and the first radical (e.g., a radical of a carrier) being different than the second radical;
the first radical and the second radical being attached to a linker (e.g., that links the first radical and the second radical);
wherein, either the first radical, the second radical, or both the first radical and the second radical is not a steroid,
or a pharmaceutically-acceptable salt or solvate thereof.

In some embodiments, the linker (e.g., L) is not a bond.

Provided in some embodiments herein is a compound having a structure represented by Formula (I):

D1-L-D2 Formula (I);
wherein:
   D1 is a radical of a carrier (e.g., a radical of an inactive agent (e.g., a non-medicinal agent (e.g., an agent that (in its free form) does not provide a (e.g., significant, measurable, and/or direct) therapeutic effect and/or benefit (e.g., to an individual (e.g., in need thereof)));
   D2 is a radical of an active agent (e.g., a therapeutically active agent) (or drug);
   L is -(Q¹-M-Q²)-;
   Q¹ and Q² are each independently absent or (C═X¹)X²;
      X¹ is O or S;
      X² is O, S, or NR¹;
         R¹ is hydrogen or C₁-C₆ alkyl; and
   M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl),
   wherein, either the first radical, the second radical, or both the first radical and the second radical is not a steroid,
or a pharmaceutically-acceptable salt or solvate thereof.

In some embodiments, the first radical or D1 is a radical of a natural compound (e.g., naturally occurring in nature or in the body).

In some embodiments, the first radical or D1 comprises a three-ring core structure (e.g., having a structure represented by Formula (I′)), wherein the three-ring core structure is a processable carrier group). In some embodiments, D1 (the carrier agent) (e.g., in its free form) is a therapeutically active agent (or drug). In some embodiments, D1 (the carrier agent) (e.g., in its free form) is not a therapeutically active agent (or drug).

In some embodiments, the first radical or D1 has a structure represented by Formula (I′).

In some embodiments, the first radical or D1 is a radical of a steroid (e.g., an anti-inflammatory steroid (e.g., dexamethasone, hydrocortisone, or triamcinolone), an angiostatic steroid (e.g., anecortave), or a benign steroid (e.g., cholesterol, cholic acid, or deoxycholic acid)).

In some embodiments, the first radical or D1 is a radical of any compound provided in Table 1 or Table 2.

In some embodiments, the first radical or D1 is a radical of a compound selected from the group consisting of ursolic acid, amyrin, isoarborinol, boswellic acid, chamaecydin, cucurbalsaminol A, cycloartenol, lanosterol, dichapetalin, oleanolic acid, lepidolide, panaxatriol, riboflavin (vitamin B2), santonic acid, tetrahydrocannabiorcol, plicadin, annonamine, boldine, pukatiene, and (−)-stepholidine.

In some embodiments, the first radical, the second radical, D1, or D2 is a radical of: an angiotensin-converting-enzyme (ACE) inhibitor (e.g., enalapril, captopril, cilazapril, fosinopril, lisinopril, moexipril, perindopril, quinapril, ramipril, trandolapril, zofenopril, or the like), an immunosuppressant (e.g., everolimus, tacrolimus, or the like), an angiotensin 11 receptor blocker (e.g., candesartan, eprosartan, irbesartan, losartan, olmesartan, telmisartan, valsartan, azilsartan, or the like), an atypical antipsychotic (e.g., paliperidone or the like), a human immunodeficiency virus (HIV) integrase inhibitor (e.g., dolutegravir or the like), a tyrosine kinase inhibitor (e.g., axitinib or the like), a beta-3-adrenergic agonist (e.g., mirabegron or the like), a 1,3-benzoxazole (e.g., tafamidis or the like), a statin (rosuvastatin, simvastatin, or the like), a vasopressin receptor antagonist (e.g., tolvaptan or the like), an antineoplastic (e.g., belzutifan, dichapetalin, or the like), a dopamine agonist (e.g., rotigotine or the like), an acetyl cholinesterase inhibitor (e.g., donepezil, galantamine, or the like), an anti-epileptic (e.g., valproic acid or the like), an opioid agonist (e.g., oxycodone, hydrocodone, or the like), an opioid antagonist (e.g., naltrexone, naloxone, nalmefene, or the like), an antimuscarinic (e.g., fesoterodine, tolterodine, or the like), an anticholinergic (e.g., darifenacin or the like), a vasodilator (e.g., treprostinil or the like), an anti-arrhythmic (e.g., propafenone or the like), an nonsteroidal anti-inflammatory drug (NSAID) (e.g., naproxen, bromfenac, celecoxib, indomethacin, or the like), an antidepressant (e.g., paroxetine, bupropion, noritriptyline, or the like), a vitamin (e.g., a vitamin D₃ (e.g., calcifediol or the like)), an antihistamine (e.g., cetirizine, desloratadine, or the like), a triterpenoid topoisomerase inhibitor (e.g., betulinic acid or the like), a triterpenoid (e.g., acetoxolone or the like), a mineralocorticoid (e.g., fludrocortisone or the like), nicotinic acetylcholine receptor agonist (e.g., varenicline or the like), a fatty acid synthase (FAS) inhibitor (e.g., bicycol or the like), a carbonic anhydrase inhibitor (e.g., dorzolamide or the like), an alpha-adrenergic agonist (e.g., brimonidine), a sympathomimetic (e.g., epinephrine), a Rho-associated kinase (ROCK) inhibitor (e.g., fasudil, hydroxyfasudil, ripasudil, netarsudil, belumosudil, verosudil, or thiazovivin), a prostaglandin (e.g., latanoprost, latanoprost acid, travoprost, travoprost acid, tafluprost, tafluprost acid, bimatoprost, or bimatoprost acid), or any combination thereof.

In some embodiments, the second radical or D2 is a radical of: an angiotensin-converting-enzyme (ACE) inhibitor (e.g., enalapril, captopril, cilazapril, fosinopril, lisinopril, moexipril, perindopril, quinapril, ramipril, trandolapril, zofenopril, or the like), an immunosuppressant (e.g., everolimus, tacrolimus, or the like), an angiotensin II receptor blocker (e.g., candesartan, eprosartan, irbesartan, losartan, olmesartan, telmisartan, valsartan, azilsartan, or the like), an atypical antipsychotic (e.g., paliperidone or the like), a human immunodeficiency virus (HIV) integrase inhibitor (e.g., dolutegravir or the like), a tyrosine kinase inhibitor (e.g., axitinib or the like), a beta-3-adrenergic agonist (e.g., mirabegron or the like), a 1,3-benzoxazole (e.g., tafamidis or the like), a statin (rosuvastatin, simvastatin, or the like), a vasopressin receptor antagonist (e.g., tolvaptan or the like), an antineoplastic (e.g., belzutifan, dichapetalin, or the like), a dopamine agonist (e.g., rotigotine or the like), an acetyl cholinesterase inhibitor (e.g., donepezil, galantamine, or the like), an anti-epileptic (e.g., valproic acid or the like), an opioid agonist (e.g., oxycodone, hydrocodone, or the like), an opioid antagonist (e.g., naltrexone, naloxone, nalmefene, or the like), an antimuscarinic (e.g., fesoterodine, tolterodine, or the like), an anticholinergic (e.g., darifenacin or the like), a vasodilator (e.g., treprostinil or the like), an anti-arrhythmic (e.g., propafenone or the like), an nonsteroidal anti-inflammatory drug (NSAID) (e.g., naproxen, bromfenac, celecoxib, indomethacin, or the like), an antidepressant (e.g., paroxetine, bupropion, noritriptyline, or the like), a vitamin (e.g., a vitamin D₃ (e.g., calcifediol or the like)), an antihistamine (e.g., cetirizine, desloratadine, or the like), a triterpenoid topoisomerase inhibitor (e.g., betulinic acid or the like), a triterpenoid (e.g., acetoxolone or the like), a mineralocorticoid (e.g., fludrocortisone or the like), nicotinic acetylcholine receptoragonist (e.g., varenicline or the like), a fatty acid synthase (FAS) inhibitor (e.g., bicycol or the like), a carbonic anhydrase inhibitor (e.g., dorzolamide or the like), an alpha-adrenergic agonist (e.g., brimonidine), a sympathomimetic (e.g., epinephrine), a Rho-associated kinase (ROCK) inhibitor (e.g., fasudil, hydroxyfasudil, ripasudil, netarsudil, belumosudil, verosudil, or thiazovivin), a prostaglandin (e.g., latanoprost, latanoprost acid, travoprost, travoprost acid, tafluprost, tafluprost acid, bimatoprost, or bimatoprost acid), or any combination thereof.

In some embodiments, the second radical or D2 is a radical of: a nonsteroidal anti-inflammatory drug (NSAID) (e.g., naproxen, bromfenac, celecoxib, indomethacin, or the like), a tyrosine kinase inhibitor (e.g., axitinib or the like), a statin (e.g., rosuvastatin, simvastatin, or the like), an antineoplastic (e.g., belzutifan, dichapetalin, fluorouracil, or the like), or any combination thereof.

In some embodiments, the second radical or D2 is a radical of a steroid (e.g., an anti-inflammatory steroid (e.g., dexamethasone, hydrocortisone, or triamcinolone) or an angiostatic steroid (e.g., anecortave)).

In some embodiments, the second radical or D2 is a prostaglandin radical.

In some embodiments, the second radical or D2 is a radical of a prostaglandin (e.g., latanoprost, latanoprost acid, travoprost, travoprost acid, tafluprost, tafluprost acid, bimatoprost, or bimatoprost acid).

In some embodiments, the second radical or D2 has a structure represented by Formula (I′).

In some embodiments, the second radical or D2 is a radical of any therapeutic agent (or drug) provided herein (e.g., any therapeutic agent (or drug) provided in Table 2).

In some embodiments, the first radical or D1 and the second radical or D2 comprises a three-ring core structure (e.g., having a structure represented by Formula (I′))).

In some embodiments, the first radical or D1 and the second radical or D2 has a structure represented by Formula (I′).

In some embodiments, the linker (e.g., L) is or comprises (e.g., a diradical (e.g., a molecular species (e.g., an organic compound) with two electrons occupying degenerate molecular orbitals) of) one or more linker groups, each linker group being independently selected from any linker or linker group provided herein (e.g., any linker or linker group provided in Table 3).

In some embodiments, the first radical or D1 is a radical of any carrier provided herein (e.g., any carrier provided in Table 1 or Table 2), the second radical or D2 is a radical of any therapeutic agent (or drug) provided herein (e.g., any therapeutic agent (or drug) provided in Table 2), and the linker (e.g., L) is or comprises (e.g., a diradical (e.g., a molecular species (e.g., an organic compound) with two electrons occupying degenerate molecular orbitals) of) one or more linker groups, each linker group being independently selected from any linker or linker group provided herein (e.g., any linker or linker group provided in Table 3).

Provided in some embodiments herein is a compound comprising a structure of Formula (IE):

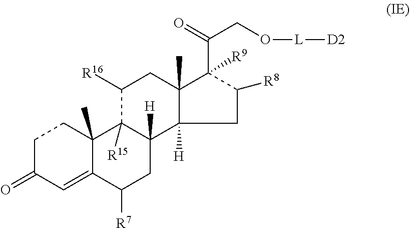

wherein:
   [symbol] is a single bond or a double bond;
   R⁷ is hydrogen or halogen;
   R⁸ is hydrogen or C₁-C₄ alkyl;
   R⁹ is absent, hydrogen, or hydroxyl;
   R¹¹ is absent, hydrogen, or halogen;
   R¹⁶ is hydrogen or hydroxyl;
   D2 is a radical of an active agent (e.g., a therapeutically active agent) (or drug);
   L is -(Q¹-M-Q²)-;
   Q¹ and Q² are each independently absent or (C═X¹)X²;
      X¹ is O or S;
      X² is O, S, or NR¹;
         R¹ is hydrogen or C₁-C₆ alkyl; and
   M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl);
   wherein D2 is not a steroid;
   or a pharmaceutically acceptable salt or solvate thereof.

In some embodiments, R⁷ is hydrogen.

In some embodiments, R⁸ is methyl. In some embodiments, R⁸ is hydrogen.

In some embodiments, R⁹ is hydroxyl.

In some embodiments, R¹⁵ is fluoro. In some embodiments, R¹⁵ is absent. In some embodiments, R¹⁵ is hydrogen.

In some embodiments, R¹⁶ is hydroxyl. In some embodiments, R¹⁶ is hydrogen.

In some embodiments, wherein the compound has a structure of Formula (IE-i):

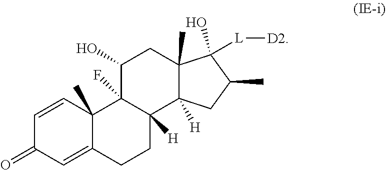

In some embodiments, the compound has a structure of Formula (IE-ii):

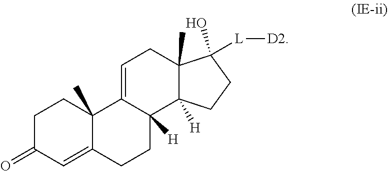

In some embodiments, the compound has a structure of Formula (IE-iii):

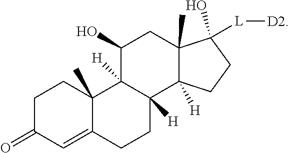

Provided in some embodiments is a compound comprising a first radical and a second radical, the first radical comprising a structure of Formula (IF):

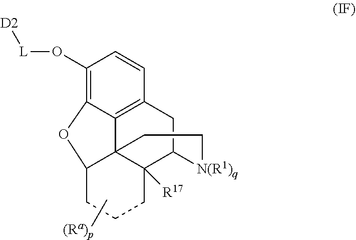

wherein:
   [symbol] is a single bond or a double bond;
   each R^{a} is independently selected the group consisting of hydrogen, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted;
      or a first R¹ is taken together with another R¹ to form oxo, a substituted or an unsubstituted cycloalkyl or heterocycloalkyl;
   each R¹ is independently H, alkyl, heteroalkyl, cycloalkyl, or heterocycloalkyl, wherein the alkyl, heteroalkyl, cycloalkyl, or heterocycloalkyl are optionally substituted;
   R¹⁷ is H or —OH;
   p is 0-8; and
   q is 1 or 2;
   D2 is a radical of an active agent (e.g., a therapeutically active agent) (or drug);
   L is -(Q¹-M-Q²)-;
   Q¹ and Q² are each independently absent or (C═X¹)X²;
      X¹ is O or S;
      X² is O, S, or NR¹;
         R¹ is hydrogen or C₁-C₆ alkyl; and
   M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl);
   wherein D2 is not an opioid;
   or a pharmaceutically acceptable salt or solvate thereof.

In some embodiments, R¹ is substituted alkyl.

In some embodiments, R¹⁷ is hydroxyl.

In some embodiments, R^{a} is hydrogen.

In some embodiments, Q¹ and Q² are each independently absent, C═O, C=S, (C═O)O, (C═O)S, (C═O)N, or (C═S)S. In some embodiments, Q¹ and Q² are each independently absent, C═O, or (C═O)O.

In some embodiments, M is substituted or unsubstituted alkyl (e.g., C₁-C₆ alkyl), substituted or unsubstituted heteroalkyl (e.g., C₁-C₆ heteroalkyl), or substituted or unsubstituted aryl. In some embodiments, M is substituted or unsubstituted alkyl (e.g., C₁-C₆ alkyl (e.g., alkyl-carbocyclyl-alkyl)) or substituted or unsubstituted heteroalkyl (e.g., C₁-C₆ heteroalkyl).

In some embodiments, L is —(CH₂CH₂O)—, —(CH₂CH₂O)₂—(CH₂CH₂O)₃—, -methyl-cyclohexyl-methyl-, —CH₂—, —CH₂CH₂—, —CH₂CH₂CH₂—, —CH₂CH₂CH₂CH₂—, —CH₂CH₂CH₂CH₂CH₂—, or —CH₂CH₂CH₂CH₂CH₂CH₂—.

In some embodiments, the compound does not have the structure:

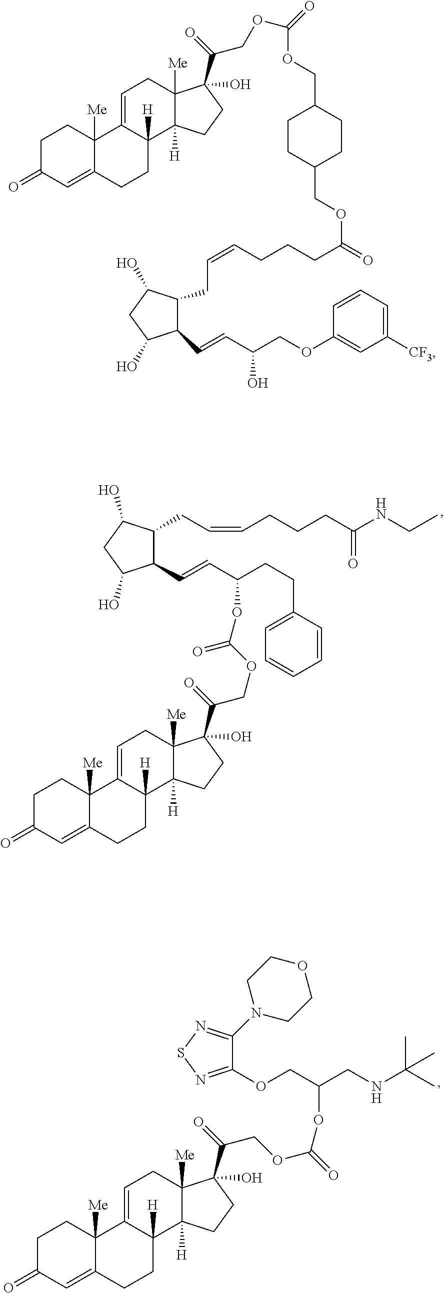

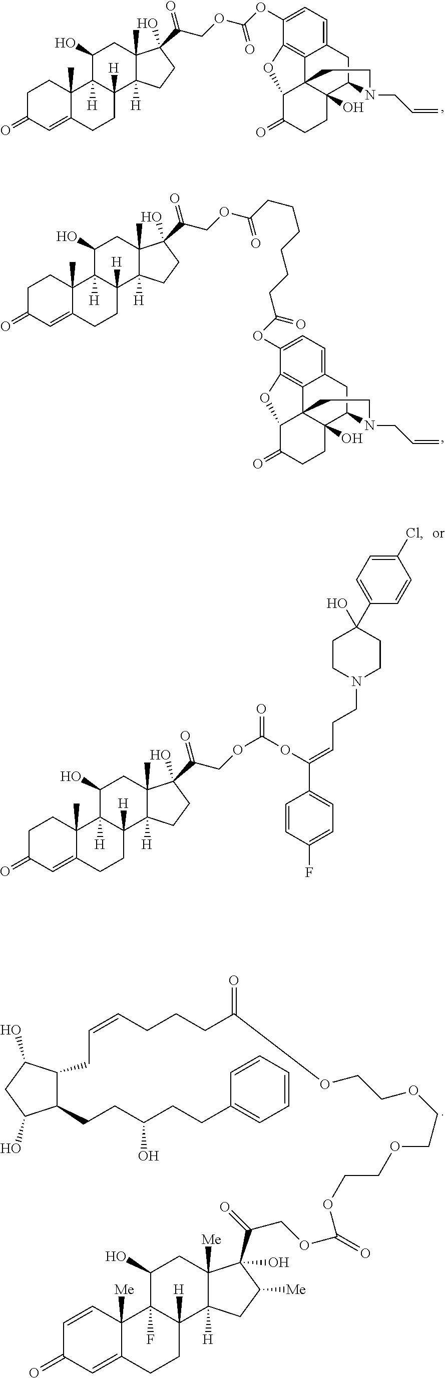

In some embodiments, the compound is:

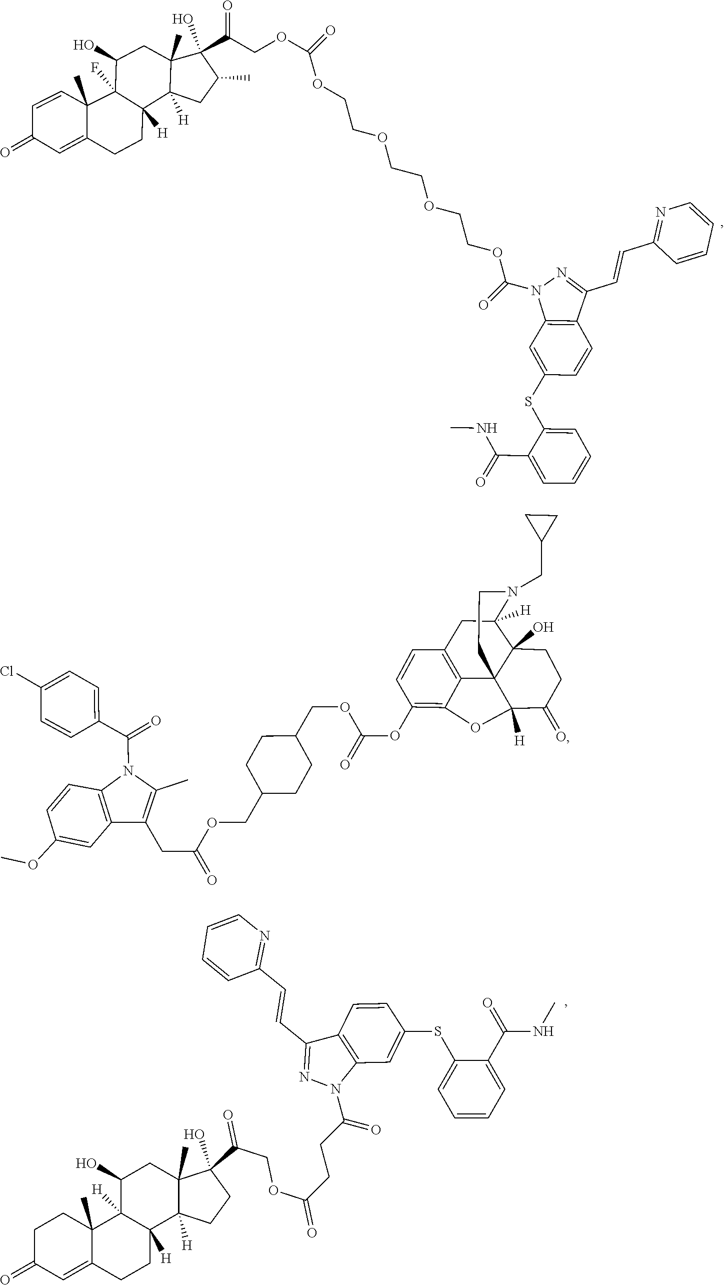

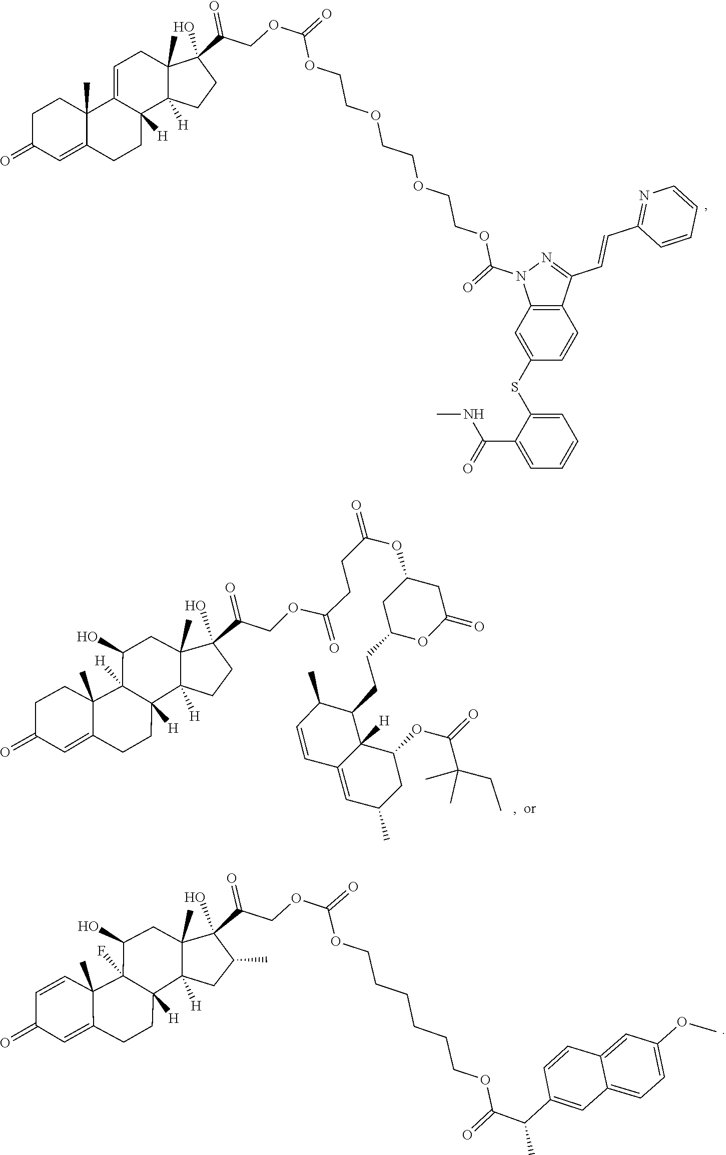 [SMILES: CNC(=O)c1ccccc1Sc1ccc2c(/C=C/c3ccccn3)nn(C(=O)OCCOCCOCCOC(=O)OCC(=O)[C@@]3(O)CC[C@H]4[C@@H]5CCC6=CC(=O)CC[C@]6(C)C5=CC[C@@]43C)c2c1 ; CCC(C)(C)C(=O)O[C@@H]1C[C@H](C)C=C2C=C[C@@H](C)[C@@H](CC[C@@H]3C[C@H](OC(=O)CCC(=O)OCC(=O)[C@@]4(O)CC[C@H]5[C@@H]6CCC7=CC(=O)CC[C@]7(C)[C@H]6[C@@H](O)C[C@@]54C)CC(=O)O3)[C@@H]21 ; COc1ccc2cc([C@H](C)C(=O)OCCCCCCOC(=O)OCC(=O)[C@@]3(O)[C@H](C)C[C@H]4[C@@H]5CCC6=CC(=O)C=C[C@]6(C)[C@@]5(F)[C@@H](O)C[C@@]43C)ccc2c1]

Prostaglandins (prostaglandin analogs) are a front-line medication in the treatment of glaucoma and can be used in the treatment of other ocular disorders. In some instances, prostaglandins are useful for lowering intraocular pressure (IOP), a major risk factor in glaucoma. Typically, prostaglandins are ophthalmically formulated and delivered in the form of eye drops. To provide efficacy, however, frequent ophthalmic administration of prostaglandins is often required. For example, latanoprost given once a day has been reported to have a mean IOP lowering of about 35%.

Provided in some embodiments herein is a compound having a structure represented by Formula (ID):

D1-L-D2 Formula (ID);
wherein:
   D1 is a steroid radical;
   D2 is a prostaglandin radical;
   L is -(Q¹-M-Q²)-;
   Q¹ and Q² are each independently absent or (C═X¹)X²;
      X¹ is O or S;
      X² is O, S, or NR¹;
         R¹ is hydrogen or C₁-C₆ alkyl; and
   M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl),
or a pharmaceutically-acceptable salt or solvate thereof.

Provided in some embodiments herein is a compound having a structure represented by Formula (IV):

D1-L²-D2 Formula (IV);
wherein:
   D1 is a steroid radical;
   D2 is a substituted prostaglandin radical; and
   L² is a linker,
or a pharmaceutically-acceptable salt or solvate thereof.

In some embodiments, D1 is an angiostatic steroid (e.g., anecortave).

In some embodiments, D1 is a benign steroid (e.g., cholesterol).

In some embodiments, D1 is a corticosteroid (e.g., glucocorticoid or mineralcorticoid), a sex steroid, a neurosteroid, an aminosteroid, or a secosteroid.

In some embodiments, D1 is anecortave (e.g., anecortave desacetate).

Provided in some embodiments herein is a compound having a structure represented by Formula (II):

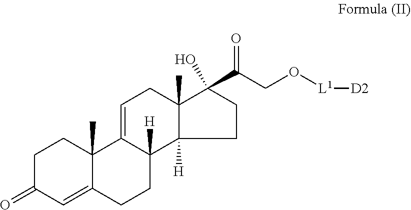

or a pharmaceutically acceptable salt or solvate thereof,
wherein,
   D2 is a prostaglandin radical;
   L¹ is -(Q¹-M-Q²)-;
   Q¹ and Q² are each independently absent or (C═X¹)X²;
      X¹ is O or S;
      X² is O, S, or NR¹;
         R¹ is hydrogen or C₁-C₆ alkyl; and
   M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl).

In some embodiments, D2 is selected from the group consisting of latanoprost, latanoprost acid, travoprost, travoprost acid, tafluprost, tafluprost acid, bimatoprost, bimatoprost acid, sepetaprost, sepetaprost acid, 7-[(1R,2R,3R,5S)-3,5-dihydroxy-2-[(3R)-3-hydroxy-5-phenylpentyl]cyclopentyl]-4-(3-thioxo-3H-1,2-dithiol-5-yl)phenyl ester, 5Z-heptenoic acid, latanoprostene bunod, and (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl 6-(nitrooxy)hexanoate, or a fragment or radical of any of the foregoing.

In some embodiments, D2 is selected from the group consisting of substituted latanoprost, substituted latanoprost acid, substituted travoprost, substituted travoprost acid, substituted tafluprost, substituted tafluprost acid, substituted bimatoprost, substituted bimatoprost acid, substituted sepetaprost, substituted sepetaprost acid, substituted 7-[(1R,2R,3R,5S)-3,5-dihydroxy-2-[(3R)-3-hydroxy-5-phenylpentyl]cyclopentyl]-4-(3-thioxo-3H-1,2-dithiol-5-yl)phenyl ester, 5Z-heptenoic acid, substituted latanoprostene bunod, and substituted (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl 6-(nitrooxy)hexanoate, or a fragment or radical of any of the foregoing.

In some embodiments, D2 is substituted latanoprost (e.g., latanoprost substituted with substituted alkyl (e.g., alkyl substituted with one or more alkyl substituent, each alkyl substituent being independently selected from the group consisting of oxo and aryl)), substituted bimatoprost (e.g., bimatoprost substituted with substituted alkyl (e.g., alkyl substituted with one or more alkyl substituent, each alkyl substituent being independently selected from the group consisting of oxo and aryl)), substituted travoprost (e.g., travoprost substituted with substituted alkyl (e.g., alkyl substituted with one or more alkyl substituent, each alkyl substituent being independently selected from the group consisting of oxo and aryl)), or an acid or radical thereof.

In some embodiments, D2 is substituted C1-latanoprost (radical), substituted C15-latanoprost (radical), substituted C1-bimatoprost (radical), substituted C15-bimatoprost (radical), substituted C1-travoprost (radical), or substituted C15-travoprost (radical) (e.g., the carbon atom (e.g., C1 or C15) indicating which carbon atom the hydroxy radical (e.g., R⁶, R^{6′}, or R¹¹) is attached)).

In some embodiments, D2 is C1-bimatoprost (radical) substituted at C15 with substituted alkyl (e.g., alkyl substituted with one or more alkyl substituent, each alkyl substituent being independently selected from the group consisting of oxo and aryl). In some embodiments, D2 is C1-bimatoprost (radical) substituted at C15 with alkyl substituted with oxo. In some embodiments, D2 is C1-bimatoprost (radical) substituted at C15 with alkyl substituted with oxo and aryl. In some embodiments, D2 is C1-bimatoprost (radical) substituted at C9 and C11 with substituted alkyl (e.g., alkyl substituted with oxo), the C9 and C11 being taken together with the substituted alkyl (e.g., to form a substituted heterocyclyl). In some embodiments, D2 is C1-bimatoprost (radical) substituted at C9 and C11 with alkyl substituted with oxo, the C9 and C11 being taken together with the alkyl substituted with oxo (e.g., to form a substituted heterocyclyl).

In some embodiments, D2 is C1-latanoprost (radical) substituted at C15 with substituted alkyl (e.g., alkyl substituted with one or more alkyl substituent, each alkyl substituent being independently selected from the group consisting of oxo and aryl). In some embodiments, D2 is C1-latanoprost (radical) substituted at C15 with alkyl substituted with oxo. In some embodiments, D2 is C1-latanoprost (radical) substituted at C15 with alkyl substituted with oxo and aryl. In some embodiments, D2 is C1-latanoprost (radical) substituted at C9 and C11 with substituted alkyl (e.g., alkyl substituted with oxo), the C9 and C11 being taken together with the substituted alkyl (e.g., to form a substituted heterocyclyl). In some embodiments, D2 is C1-latanoprost (radical) substituted at C9 and C11 with alkyl substituted with oxo, the C9 and C11 being taken together with the alkyl substituted with oxo (e.g., to form a substituted heterocyclyl).

In some embodiments, D2 is a radical represented by a structure of Formula (III):

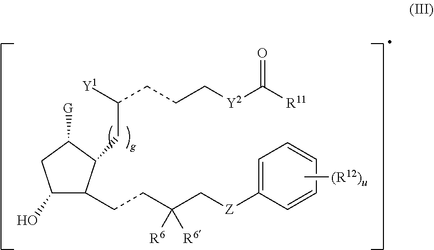

wherein:
   each [symbol] is independently a single bond or a double bond;
   G is OH;
   Y¹ is hydrogen;
      or G is taken together with Y¹ to form —O—CH₂—;
   Y² is a bond or alkylene (e.g., —CH₂—);
   g is 1 or 2;
   Z is —O— or alkylene (e.g., —CH₂—);
   R⁶ and R^{6′} are each independently hydrogen, halogen, alkyl, —OR¹⁴, or R⁶ and R^{6′} are taken together to form an oxo (e.g., R⁶ and R^{6′} are each independently hydrogen, halogen, or OH);
      R¹⁴ is hydrogen, unsubstituted or substituted alkyl (e.g., substituted or unsubstituted C₁-C₆ alkyl (e.g., alkyl substituted with NO₂ or alkyl substituted with oxo and ONO₂)), or a point of attachment to the linker (e.g., L, L1, or L²);
   R¹¹ is —OR¹³ or —NR^{13a}R^{13b}.
      R¹³, R^{13a}, and R^{13b} are each independently selected from the group consisting of hydrogen, unsubstituted or substituted alkyl (e.g., substituted or unsubstituted C₁-C₆ alkyl (e.g., alkyl substituted with NO₂ or alkyl substituted with oxo and ONO₂)), substituted or unsubstituted aryl (e.g., aryl substituted with (cyclic) heteroalkyl (e.g., aryl substituted with 3H-1,2-dithiole-3-thione)), or a point of attachment to the linker (e.g., L, L¹, or L²);
   each R¹² is independently halogen (e.g., fluoro or chloro) or alkyl (e.g., haloalkyl (e.g., CF₃)); and
   u is 0-5.

In some embodiments, G is OH.

In some embodiments, Y¹ is hydrogen.

In some embodiments, Y² is alkylene (e.g., —CH₂—).

In some embodiments, g is 1.

In some embodiments, G is OH, Y¹ is hydrogen, Y² is —CH₂—, and g is 1.

In some embodiments, R¹³ and R¹⁴ are each independently selected from the group consisting of hydrogen, unsubstituted or substituted alkyl (e.g., substituted or unsubstituted C₁-C₆ alkyl (e.g., alkyl substituted with NO₂ or alkyl substituted with oxo and ONO₂)), substituted or unsubstituted aryl (e.g., aryl substituted with (cyclic) heteroalkyl (e.g., aryl substituted with 3H-1,2-dithiole-3-thione)), or a point of attachment to the linker (e.g., L, L¹, or L²).

In some embodiments, R¹⁴, R¹³, R^{13a}, and R^{13b} are each independently selected from the group consisting of hydrogen, substituted or unsubstituted C₁-C₆ alkyl, or a point of attachment to the linker (e.g., L, L¹, or L²).

In some embodiments, D2 is a radical represented by a structure of Formula (III-A):

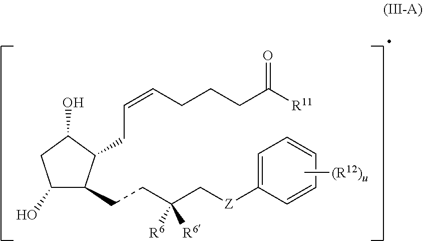

wherein:
   [symbol] a single bond or a double bond;
   Z is —O— or —CH₂—;
   R⁶ and R^{6′} are each independently hydrogen, halogen, or —OR¹⁴;
      R¹⁴ is hydrogen or a point of attachment to the linker (e.g., L, L¹, or L²);
   R¹¹ is —OR¹³ or —NR^{13a}R^{13b};
      R¹³, R^{13a}, and R^{13b} are each independently hydrogen, C₁-C₃ alkyl, or a point of attachment to the linker (e.g., L, L¹, or L²);
   each R¹² is independently halogen (e.g., fluoro or chloro) or alkyl (e.g., haloalkyl (e.g., CF₃)); and
   u is 0-5.

In some embodiments, G together with Y¹ forms —O—CH₂—, and g is 2, and D2 is a radical represented by a structure of formula (III-B):

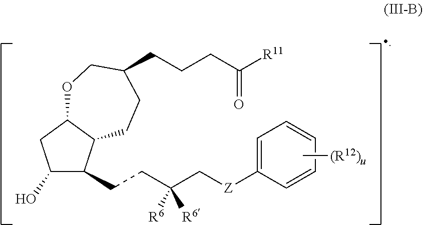

In some embodiments, Z is —O—. In some embodiments, Z is —CH₂—.

In some embodiments, R¹² is CF₃ and u is 1. In some embodiments, R¹² is F and u is 2.

In some embodiments, u is 0. In some embodiments, u is 1. In some embodiments, u is 2.

In some embodiments, R⁶ and R^{6′} are each independently fluoro. In some embodiments, R⁶ is OH and R^{6′} is hydrogen.

In some embodiments, R¹¹ is OH or NH(C₁-C₃ alkyl). In some embodiments, R¹¹ is —NHCH₂CH₃. In some embodiments, R¹ is OH.

In some embodiments, R⁶ is —OR¹⁴ and R¹⁴ is a point of attachment to the linker (e.g., L, L¹, or L²), or R¹¹ is —OR¹³ and R¹³ is a point of attachment to the linker (e.g., L, L¹, or L²). In some embodiments, R⁶ is —OR¹⁴ and R¹⁴ is a point of attachment to the linker (e.g., L, L¹, or L²). In some embodiments, R¹¹ is —OR¹³ and R¹³ is a point of attachment to the linker (e.g., L, L¹, or L²).

In some embodiments, R¹¹ is —OR¹³, R³ is a point of attachment to the linker (e.g., L, L¹, or L²), R⁶ is OH, and R^{6′} is hydrogen.

In some embodiments, R¹¹ is OH, R⁶ is —OR¹⁴, R¹⁴ is a point of attachment to the linker (e.g., L, L¹, or L²), and R^{6′} is hydrogen.

In some embodiments, D2 is latanoprost, bimatoprost, travoprost, or an acid or radical thereof.

In some embodiments, D2 is C1-latanoprost (radical), C15-latanoprost (radical), C1-bimatoprost (radical), C15-bimatoprost (radical), C1-travoprost (radical), or C15-travoprost (radical) (e.g., the carbon atom (e.g., C1 or C15) indicating which carbon atom the hydroxy radical (e.g., R⁶, R^{6′}, or R¹¹) is attached)).

In some embodiments, Q¹ and Q² are each independently absent, C═O, C=S, (C═O)O, (C═O)S, (C═O)N, or (C═S)S. In some embodiments, Q¹ and Q² are each independently C═O or (C═O)O. In some embodiments, Q¹ and Q² are each independently is C═O. In some embodiments, Q¹ and Q² are C═O. In some embodiments, Q¹ is C═O and Q² is absent. In some embodiments, Q¹ is absent and Q² is C═O. In some embodiments, Q¹ and Q² are (C═O)O. In some embodiments, Q¹ is (C═O)O and Q² is absent. In some embodiments, Q¹ is absent and Q² is (C═O)O. In some embodiments, Q¹ is C═O and Q² is (C═O)S. In some embodiments, Q¹ is (C═O)S and Q² is C═O. In some embodiments, Q¹ is C=O and Q² is (C═S)S. In some embodiments, Q¹ is (C═S)S and Q² is C═O. In some embodiments, Q¹ is C=O and Q² is (C═O)N. In some embodiments, Q¹ is (C═O)N and Q² is C═O.

In some embodiments, M is substituted or unsubstituted alkyl (e.g., C₁-C₆ alkyl), substituted or unsubstituted heteroalkyl (e.g., C₁-C₆ heteroalkyl), or substituted or unsubstituted aryl.

In some embodiments, M is substituted or unsubstituted alkyl (e.g., C₁-C₆ alkyl).

In some embodiments, M is substituted alkyl (e.g., C₁-C₆ alkyl), the alkyl being substituted with one or more substituent, each substituent being independently selected from the group consisting of oxo, halo, alkyl, and heteroalkyl (e.g., —NHCOCH₃). In some embodiments, M is alkyl (e.g., C₁-C₆ alkyl) substituted with oxo. In some embodiments, M is alkyl (e.g., C₁-C₆ alkyl) substituted with one or more —NHCOCH₃. In some embodiments, M is —CH(NHCOCH₃)CH₂—.

In some embodiments, M is unsubstituted alkyl (e.g., C₁-C₆ alkyl). In some embodiments, M is —(CH₂)ₘ—, m being 1-10. In some embodiments, M is —CH₂—, —CH₂CH₂—, or —CH₂CH₂CH₂—.

In some embodiments, M is substituted or unsubstituted heteroalkyl (e.g., C₁-C₆ heteroalkyl). In some embodiments, M is unsubstituted heteroalkyl (e.g., C₁-C₆ heteroalkyl). In some embodiments, M is —SCH₂CH₂—.

In some embodiments, M is substituted or unsubstituted aryl. In some embodiments, M is unsubstituted aryl (e.g., unsubstituted phenyl).

In some embodiments, M is alkyl (e.g., methyl) substituted with oxo and Q¹ and Q² are absent.

In some embodiments, Q¹ and Q² are (C═O)O; and M is —CH₂CH₂CH₂—.

In some embodiments, Q¹ and Q² are C═O; and M is —CH₂CH₂— or phenyl.

In some embodiments, Q¹ is C═O and Q² is absent; and M is —CH₂—, —CH₂CH₂—, —CHCH₃—, or —CH(NHCOCH₃)CH₂—.

In some embodiments, Q¹ is C═O and Q² is absent; and M is —SCH₂CH₂—.

In some embodiments, Q¹ is C═O and Q² is S(C═O); and M is —CH₂CH₂—.

In some embodiments, Q¹ is C═O and Q² is S(C═S); and M is —CH₂CH₂—.

In some embodiments, Q¹ is C═O and Q² is N(C═O); and M is —CH₂CH₂—.

In some embodiments, the linker (e.g., L, L¹, or L²) is C═O, —C═OCH₂CH₂C=O—, —C=OphenylC═O—, —C═OCH₂CH₂—, —(C═O)OCH₂CH₂CH₂O(C═O)—, —C═OCH(NHCOCH₃)CH₂—, —C═OCH₂O(C═O)—, —(C═O)OCH₂C═O—, —C═OCH(CH₃)O(C═O)—, —(C═O)OCH(CH₃)C═O—, —C═OCH₂CH₂S—, —C═OCH₂CH₂SC═O—, —C═OCH₂CH₂SC═S—, or —C═OCH₂CH₂NHC═O—. In some embodiments, the linker (e.g., L, L¹, or L²) is —C═OCH₂CH₂C=O—, —C═OphenylC═O—, —C═OCH₂CH₂—, —(C═O)OCH₂CH₂CH₂O(C═O)—, —C═OCH(NHCOCH₃)CH₂—, —C═OCH₂O(C═O)—, —(C═O)OCH₂C=O—, —C═OCH(CH₃)O(C═O)—, —(C═O)OCH(CH₃)C═O—, —C═OCH₂CH₂S—, —C═OCH₂CH₂SC═O—, —C═OCH₂CH₂SC═S—, or —C═OCH₂CH₂NHC=O—. In some embodiments, the linker (e.g., L, L¹, or L²) is C═O. In some embodiments, the linker (e.g., L, L¹, or L²) is a bond.

In some embodiments, L² is a bond.

In some embodiments, the compound does not have the structure:

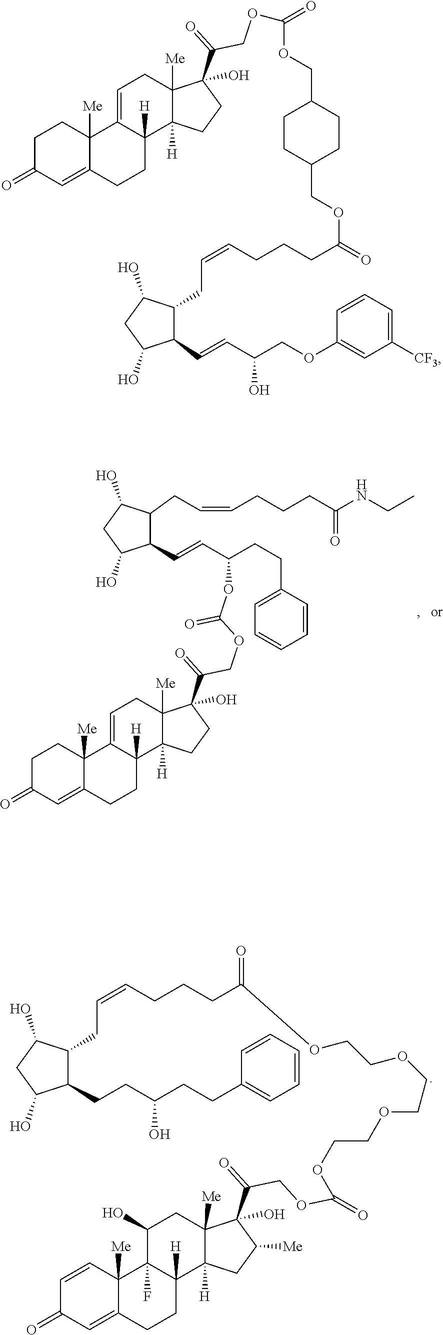

In some embodiments, the compound is represented by the structure:

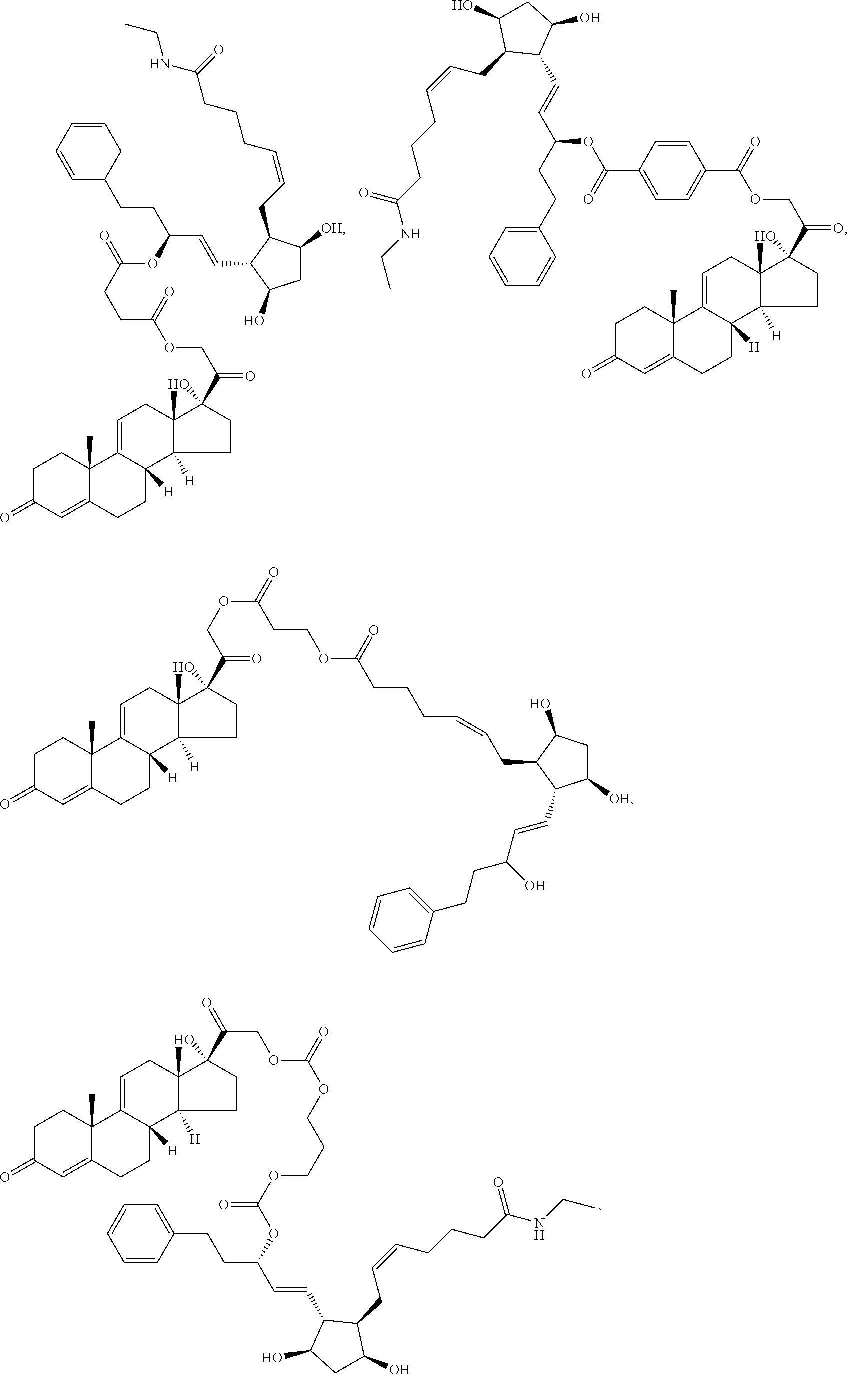

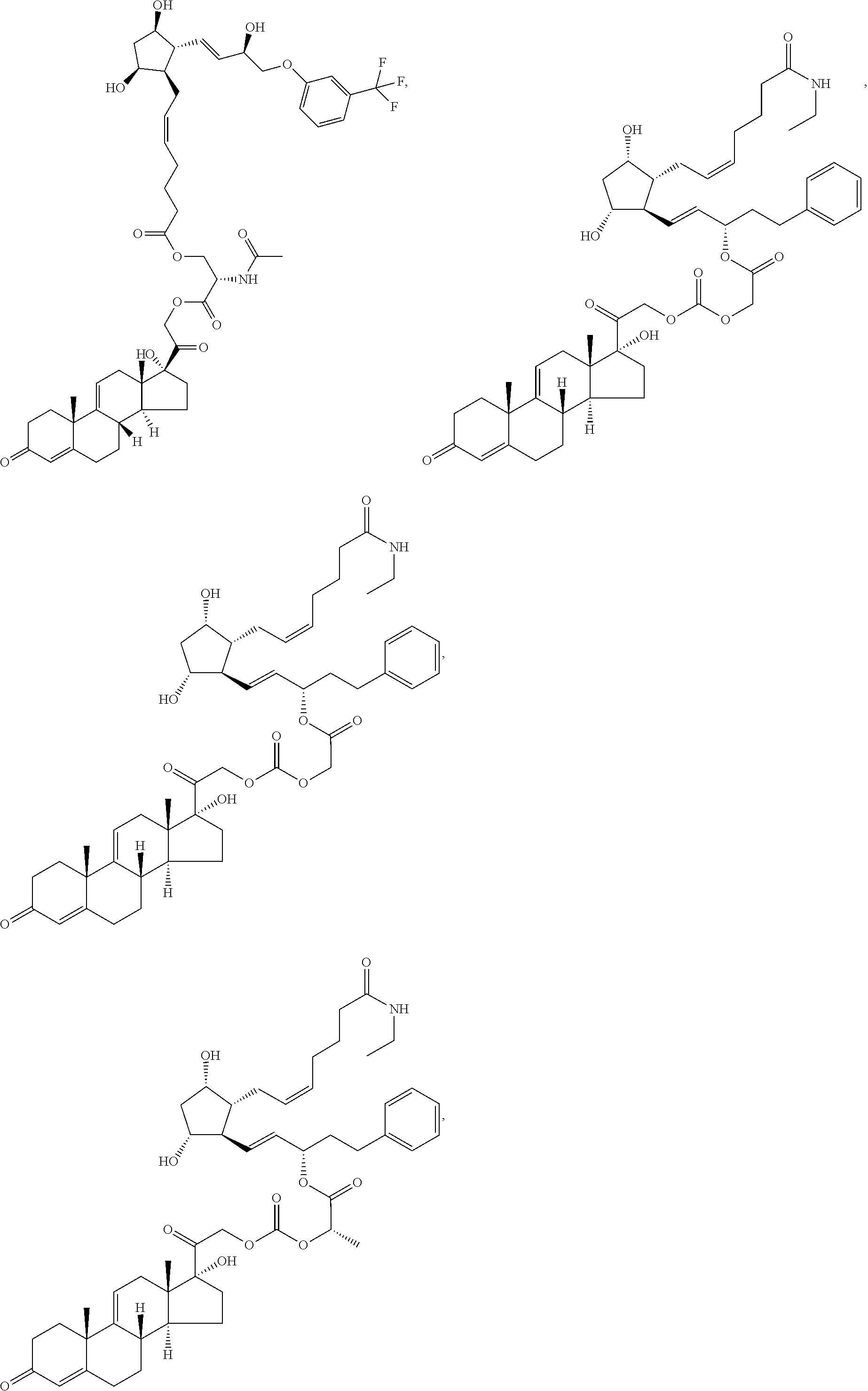

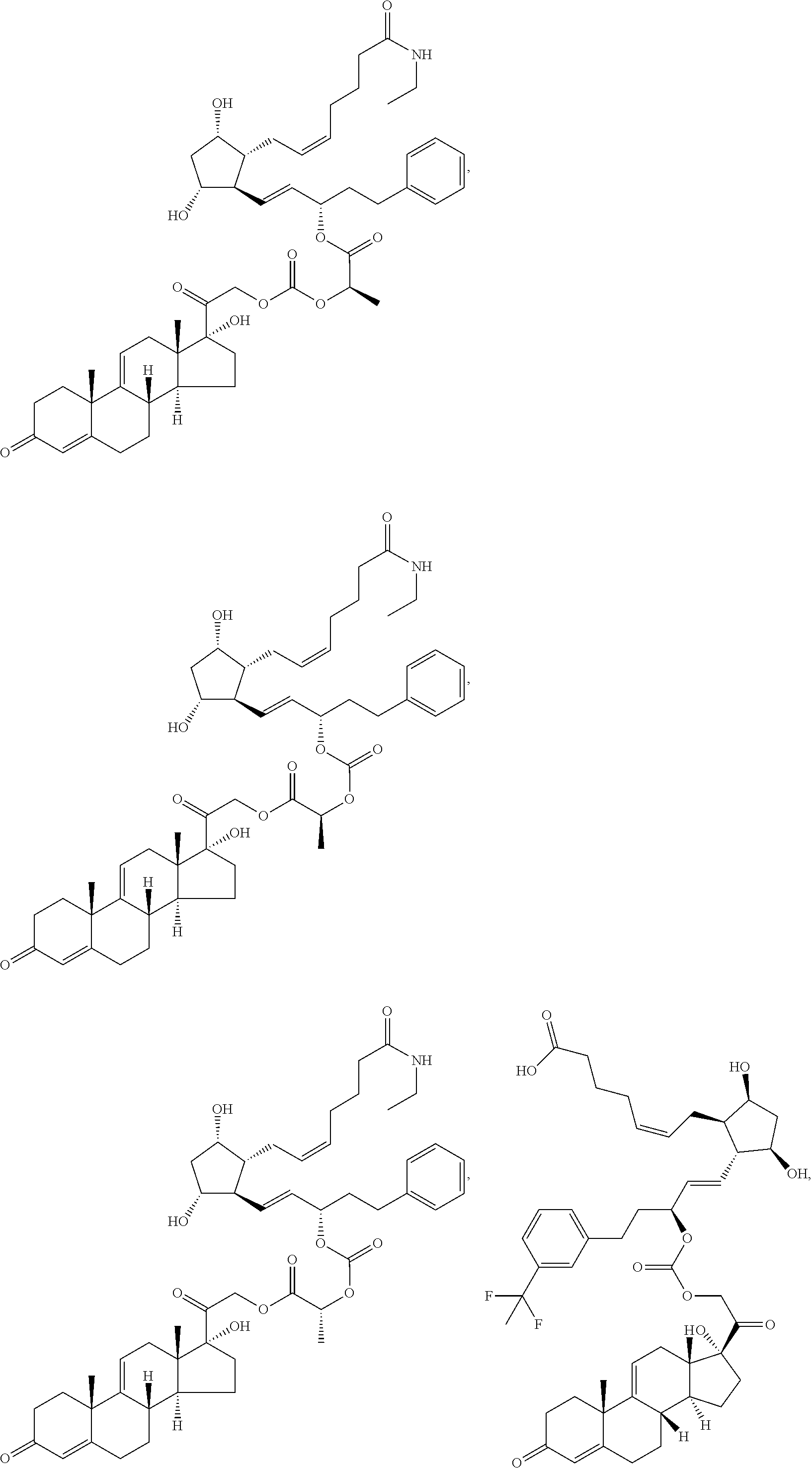

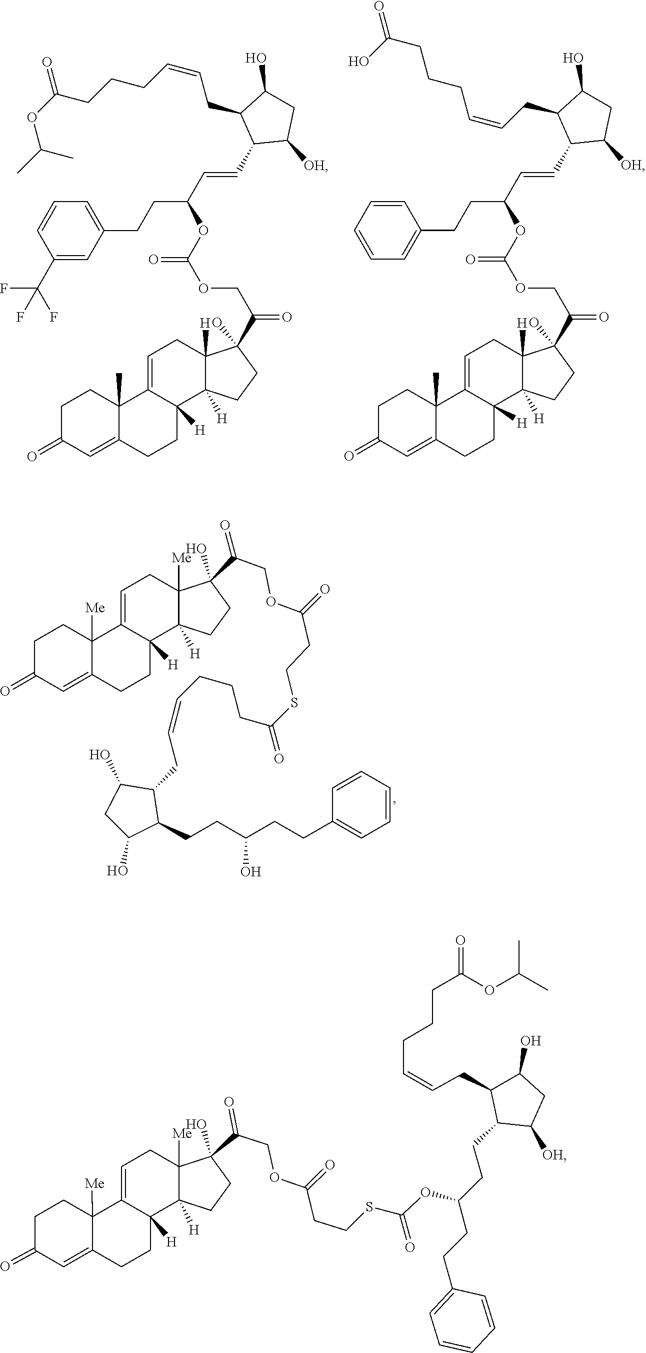

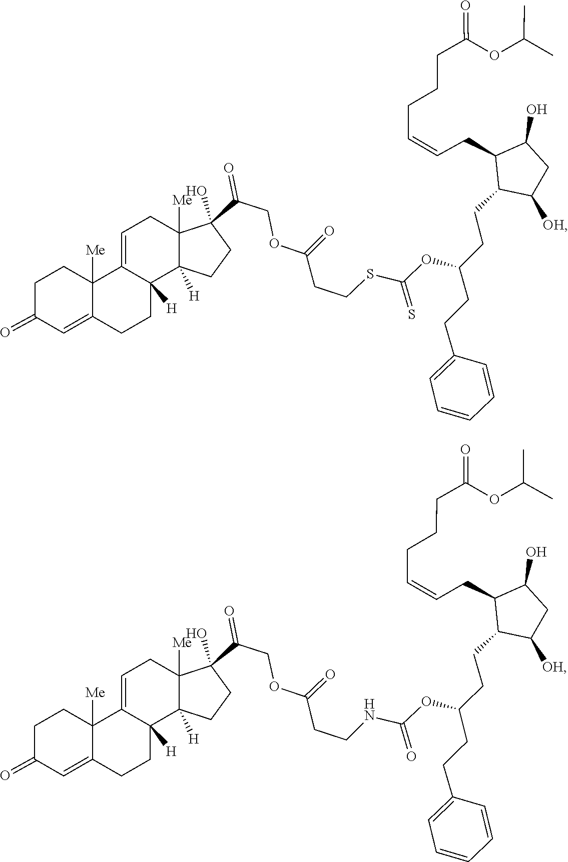

or a pharmaceutically acceptable salt or solvate thereof.

Provided in some embodiments herein is a compound represented by the structure:

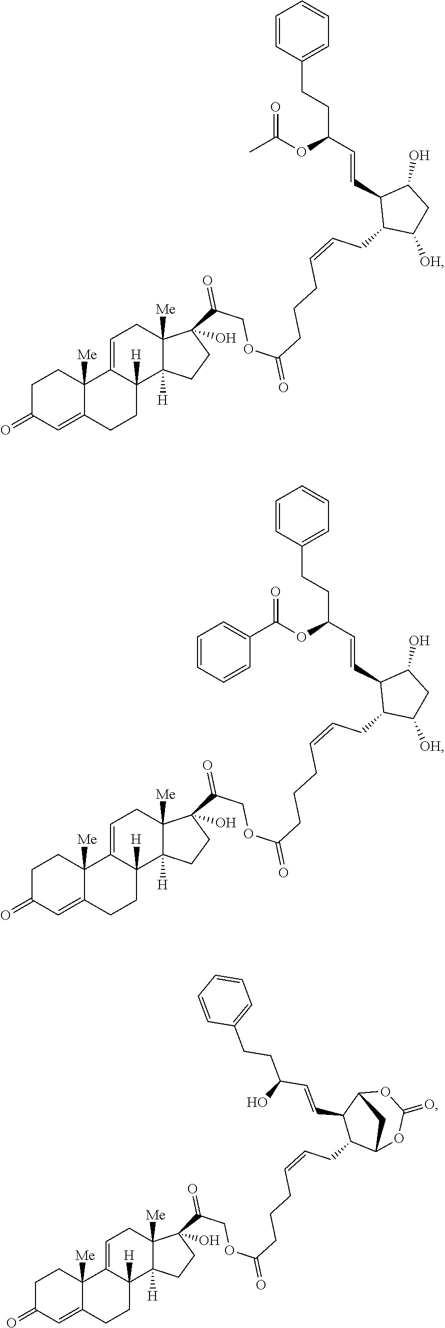

or a pharmaceutically acceptable salt or solvate thereof.

Provided in some embodiments herein is a pharmaceutical implant or article comprising a compound of any one of the preceding claims, or a pharmaceutically-acceptable salt thereof.

In some embodiments, the pharmaceutical implant or article of any one of the preceding claims, wherein the implant or article comprises at least 50 wt. % (at least 60 wt. %, at least 70 wt. %, at least 80 wt. %, at least 90 wt. %, at least 95 wt. %, at least 98 wt. %, or the like) of the compound or pharmaceutically acceptable salt thereof. In some embodiments, the implant or article comprises at least 70 wt. % of the compound or pharmaceutically acceptable salt thereof. In some embodiments, the implant or article comprises at least 90 wt. % (e.g., about 90 wt. % or more, about 95 wt. % or more, or about 99 wt. % or more) of the compound or pharmaceutically acceptable salt thereof.

In some embodiments, the implant or article undergoes surface erosion to release the compound, the first radical (in its free form), D1 (in its free form), the second radical (in its free form), and/or D2 (in its free form). In some embodiments, D1 and D2 are released (in their free form) from the pharmaceutical implant or article at near zero-order in a buffered solution or in vivo. In some embodiments, D1 and D2 are released from the pharmaceutical implant or article (in their free form) at 37° C. in 100% bovine serum or at 37° C. in phosphate buffered saline (PBS) at a rate such that t₁₀ is greater than or equal to 1/10 of t₅₀. In some embodiments, D1 and D2 are released from the pharmaceutical implant or article (in their free form) at 37° C. in 1% fetal bovine serum (FBS) in phosphate buffered saline (PBS) at a rate such that t₁₀ is greater than or equal to 1/10 of t₅₀.

In some embodiments, the implant or article undergoes surface erosion to release the compound, the steroid radical (in its free form), and/or the prostaglandin radical (in its free form). In some embodiments, the steroid radical and the prostaglandin radical are released (in their free form) from the pharmaceutical implant or article at near zero-order in a buffered solution or in vivo. In some embodiments, the steroid radical and the prostaglandin radical are released from the pharmaceutical implant or article (in their free form) at 37° C. in 100% bovine serum or at 37° C. in phosphate buffered saline (PBS) at a rate such that t₁₀ is greater than or equal to 1/10 of t₅₀. In some embodiments, the steroid radical and the prostaglandin radical are released from the pharmaceutical implant or article (in their free form) at 37° C. in 1% fetal bovine serum (FBS) in phosphate buffered saline (PBS) at a rate such that t₁₀ is greater than or equal to 1/10 of t₅₀.

Provided in some embodiments herein is a pharmaceutical composition comprising any compound provided herein, or a pharmaceutically-acceptable salt thereof, and at least one pharmaceutically-acceptable excipient.

In some embodiments, the implant, article, or composition is in a form suitable for ophthalmic administration, subcutaneous administration, intramuscular, or intraspinal administration. In some embodiments, the ophthalmic administration is intraocular, intracameral, intravitreal, suprachoroidal, punctal, retrobulbar, or subconjunctival.

In some embodiments, the implant, article, or composition is in a form suitable for ophthalmic administration, subcutaneous administration, intramuscular, or intraspinal administration.

In some embodiments, the implant, article, or composition is in a form suitable for ophthalmic administration. In some embodiments, the ophthalmic administration is intraocular, intracameral, intravitreal, suprachoroidal, punctal, retrobulbar, or subconjunctival.

Provided herein is a method of treating a medical indication or abnormality in an individual in need thereof, the method comprising administering to the individual a compound, pharmaceutically acceptable salt, implant, article, coating, or composition of any compound described herein.

Provided herein is a method of treating an inflammatory disease or disorder in an individual in need thereof, the method comprising administering to the individual any compound, pharmaceutically acceptable salt, implant, article, or composition described herein.

Provided herein is a method of treating a post-operative condition in an individual in need thereof, the method comprising administering to the individual any compound, pharmaceutically acceptable salt, implant, article, or composition described herein.

Provided herein is a method of treating an ophthalmic disease or disorder in an individual in need thereof, the method comprising administering to the individual any compound, pharmaceutically acceptable salt, implant, article, or composition described herein. In some embodiments, the ophthalmic disease or disorder is glaucoma. In some embodiments, the ophthalmic disease or disorder is ocular hypertension. In some embodiments, the ophthalmic disease or disorder is selected from the group consisting of ocular inflammation, diabetic macular edema, posterior inflammation, anterior inflammation, macular degeneration (e.g., wet macular degeneration (AMD) or dry AMD), post-cataract surgery, and retinal vein occlusion.

In some embodiments, the article or implant is at least partially biodegradable. In some embodiments, the article or implant is non-biodegradable. In some embodiments, removal of the article or implant is not required (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)). In some embodiments, the article or implant is not removed (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A-1D shows the chemical structure for a prodrug ester and an active pharmaceutical ingredient for a prostaglandin (e.g., travoprost (FIG. 1A) and travoprost acid (FIG. 1B), respectively) and a steroid (e.g., anecortave acetate (FIG. 1C) and anecortave desacetate (FIG. 1D), respectively) exemplified herein.

FIG. 2A-2D shows the chemical structure (FIG. 2A) and the heat processed pellet (FIG. 2B) of a steroid-prostaglandin heterodimer (travoprost-anecortave, Compound 1) exemplified herein. FIG. 2C shows the drug release profile for Compound 1 (pellet) in fetal bovine serum (FBS) over 15 days. FIG. 2D represents the 15-day progression of the surface erosion drug release profile for the pellet of Compound 1 in FBS.

FIG. 3A-3D shows the chemical structure (FIG. 3A) and the heat processed pellet (FIG. 3B) of a steroid-prostaglandin heterodimer (travoprost-dexamethasone, Compound 2) exemplified herein. FIG. 3C shows the drug release profile for Compound 2 (pellet) in phosphate-buffered saline (PBS) over 30 days. FIG. 3D represents the 30-day progression of the (e.g., surface erosion) drug release profile and swelling profile for the pellet of Compound 2 in PBS.

FIGS. 4A & 4B shows the chemical structure for a prodrug ester and an active pharmaceutical ingredient for a prostaglandin (e.g., latanoprost (FIG. 4A) and latanoprost acid (FIG. 4B), respectively) exemplified herein.

FIG. 5A-5D shows the chemical structure (FIG. 5A) and the heat processed pellet (FIG. 5B) of a steroid-prostaglandin heterodimer (latanoprost-dexamethasone, Compound 3) exemplified herein. FIG. 5C shows the drug release profile for Compound 3 (pellet) in fetal bovine serum (FBS) over 30 days. FIG. 5D represents the 30-day progression of the (e.g., surface erosion) drug release profile and swelling profile for the pellet of Compound 3 in FBS.

FIG. 6A-6C shows the chemical structure (FIG. 6A) of a steroid-prostaglandin heterodimer (latanoprost-estrone, Compound 4) exemplified herein. FIG. 6B shows the drug release profile for Compound 4 (coating on polymeric substrate) in fetal bovine serum (FBS) over 8 days. FIG. 6C represents the 7-day progression of the surface coating drug release of Compound 4 in FBS.

FIG. 7A-7D shows the chemical structure (FIG. 7A) and the heat processed pellet (FIG. 7B) of a steroid-prostaglandin heterodimer (bimatoprost-anecortave, Compound 5) exemplified herein. FIG. 7C shows the drug release profile for Compound 5 (pellet) in fetal bovine serum (FBS) over 25 days. FIG. 7D represents the 28-day progression of the (e.g., surface erosion) drug release profile for the pellet of Compound 5 in FBS.

FIG. 8A-8D shows the chemical structure (FIG. 8A) and the heat processed pellet (FIG. 8B) of a steroid-prostaglandin heterodimer (latanoprost-anecortave, Compound 6) exemplified herein. FIG. 8C shows the drug release profile for each of latanoprost acid (●) and anecortave desacetate (◯) (pellet) in fetal bovine serum (FBS) over 30 days. FIG. 8D represents the 28-day progression of the (e.g., surface erosion) drug release profile for the pellet of Compound 6 in FBS.

FIG. 9A-9D shows the chemical structure (FIG. 9A) and the heat processed pellet (FIG. 9B) of a steroid-prostaglandin heterodimer (tafluprost-anecortave, Compound 7) exemplified herein. FIG. 9C shows the drug release profile for Compound 7 (pellet) in fetal bovine serum (FBS) over 30 days. FIG. 9D represents the 28-day progression of the (e.g., surface erosion) drug release profile for the pellet of Compound 7 in FBS.

FIG. 10A-10C shows an extruded rod of a steroid-prostaglandin heterodimer (bimatoprost-anecortave, Compound 5) exemplified herein (FIG. 10A). FIG. 10B shows the drug release profile of Compound 5 (extruded rod) in fetal bovine serum (FBS) over 30 days. FIG. 10C represents the 30-day progression of the (e.g., surface erosion) drug release profile for the extruded rod of Compound 5 in FBS.

FIGS. 11A & 11B shows an extruded rod of a steroid-prostaglandin heterodimer (travoprost-anecortave, Compound 1) exemplified herein (FIG. 11A). FIG. 11B shows the drug release profile of Compound 1 (extruded rod) in fetal bovine serum (FBS) over 30 days.

FIG. 12A-12C shows an extruded rod of a steroid-prostaglandin heterodimer (latanoprost-anecortave, Compound 6) exemplified herein (FIG. 12A). FIG. 12B shows the drug release profile of Compound 6 (extruded rod) in fetal bovine serum (FBS) over 70 days. FIG. 12C represents the 70-day progression of the (e.g., surface erosion) drug release profile for the extruded rod of Compound 6 in FBS.

FIG. 13A-13C shows an extruded rod of a steroid-prostaglandin heterodimer (tafluprost-anecortave, Compound 7) exemplified herein (FIG. 13A). FIG. 13B shows the drug release profile of Compound 7 (extruded rod) in fetal bovine serum (FBS) over 100 days. FIG. 13C represents the 100-day progression of the (e.g., surface erosion) drug release profile for the extruded rod of Compound 7 in FBS.

FIG. 14 shows an extruded rod of a steroid-prostaglandin heterodimer (bimatoprost-anecortave, Compound 5) exemplified herein in a rabbit eye.

FIG. 15A-15C shows the chemical structure (FIG. 15A) and the heat processed pellet (FIG. 15B) of a steroid-prostaglandin heterodimer (travoprost-cyclohexanedimethanol-anecortave, Compound 8) exemplified herein. FIG. 15C shows the drug release profile for Compound 8 (pellet) in fetal bovine serum (FBS) over 8 days.

FIG. 16 shows the chemical structure of a steroid-prostaglandin heterodimer (travoprost-naltrexone, Compound 9) exemplified herein.

FIG. 17 shows the chemical structure of a steroid-β-blocker heterodimer (timolol-deoxycholic acid, Compound 10) exemplified herein.

FIG. 18 shows purity of Compound 5 before (pre-sterilization) or post sterilization of Compound 22 in ethylene oxide, or by gamma-radiation or E-beam.

FIGS. 19A & 19B shows the chemical structure (FIG. 19A) and the heat processed pellet (FIG. 19B) of a steroid-prostaglandin heterodimer (Bimatoprost (C₁₅)-anecortave, Compound 11) exemplified herein.

FIGS. 20A & 20B shows the chemical structure (FIG. 20A) and the heat processed pellet (FIG. 20B) of a steroid-prostaglandin heterodimer (Bim-C15-Succ-Anec, Compound 31) provided herein.

FIGS. 21A & 21B shows the chemical structure (FIG. 21A) and the heat processed pellet (FIG. 21B) of a steroid-prostaglandin heterodimer (Bim-C15-Tere-Anec, Compound 32) provided herein.

FIGS. 22A & 22B shows the chemical structure (FIG. 22A) and the heat processed pellet (FIG. 22B) of a steroid-prostaglandin heterodimer (BimOEt-C15-Anec, Compound 33) provided herein.

FIGS. 23A & 23B shows the chemical structure (FIG. 23A) and the heat processed pellet (FIG. 23B) of a steroid-prostaglandin heterodimer (Bim(acid)-C1-Anec-C15-Acetate, Compound 48) provided herein.

FIG. 24 shows the chemical structure a steroid-prostaglandin heterodimer (Bim(acid)-C1-Anec-C15-Benzoyl, Compound 52) provided herein.

FIG. 25A-D shows the chemical structure (FIG. 25A) and the heat processed pellet (FIG. 25B-D) of a steroid-kinase inhibitor heterodimer (anecortave-TEG-axitinib, Compound 57) exemplified herein.

FIG. 26A-D shows the chemical structure (FIG. 26A) and the heat processed pellet (FIG. 26B-D) of an opioid antagonist-NSAID heterodimer (naltrexone-DMC-indomethacin, Compound 55) exemplified herein.

FIG. 27A-D shows the chemical structure (FIG. 27A) and the heat processed pellet (FIG. 27B-D) of a steroid-statin heterodimer (hydrocortisone-succ-simvastatin, Compound 58) exemplified herein.

FIG. 28A-D shows the chemical structure (FIG. 28A) and the heat processed pellet (FIG. 28B-D) of a steroid-kinase inhibitor heterodimer (hydrocortisone-succ-axitinib, Compound 56) exemplified herein.

FIG. 29A-D shows the chemical structure (FIG. 29A) and the heat processed pellet (FIG. 29B-D) of a steroid-NSAID heterodimer (dexamethasone-hex-naproxen, Compound 59) exemplified herein.

FIG. 30A-D shows the chemical structure (FIG. 30A) and the heat processed pellet (FIG. 30B-D) of a steroid-kinase inhibitor heterodimer (dexamethasone-TEG-axitinib, Compound 54) exemplified herein.

### DETAILED DESCRIPTION OF THE INVENTION

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other some embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

“Amino” refers to the —NH₂ radical.

“Cyano” refers to the —CN radical.

“Nitro” refers to the —NO₂ radical.

“Oxo” refers to the =O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In some embodiments, an alkyl comprises one to thirteen carbon atoms (e.g., C₁-C₁₃ alkyl). In some embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R², —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In some embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. In some instances, an aryl provided herein is a multicyclic ring system, such as a multicyclic ring system of a compound provided in Table 1 or Table 2). The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R²)₂, —R^{b}—O—R^{c}—C(O)N(R²)₂, —R^{b}—N(R^{a})C(O)OR^{a}, —R^{b}—N(R²)C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, ortrifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In some instances, a cycloalkyl provided herein is a fused or bridged ring system, such as a fused or bridged ring system of a compound provided in Table 1 or Table 2). Unless stated otherwise specifically in the specification, “carbocyclyl” and “cycloalkyl” are used interchangeably herein. In some embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R², —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkenyl” refers to a radical of the formula —R^{c}-carbocyclyl where R^{c} is an alkenylene chain as defined above. The alkenylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, “heterocyclyl” and “heterocycloalkyl” are used interchangeably herein. Unless stated otherwise specifically in the specification, the heterocyclyl radical is a monocyclic, bicyclic, tricyclic or tetracyclic ring system, which optionally includes fused or bridged ring systems. In some instances, a heterocyclyl provided herein is a fused or bridged ring system (e.g., bicyclic, tricyclic or tetracyclic ring system), such as a fused or bridged ring system of a compound provided in Table 1 or Table 2). The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R², —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R¹ is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some instances, an heteroaryl provided herein is a fused or bridged ring system, such as a fused or bridged ring system of a compound provided in Table 1 or Table 2). The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobe nzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in some embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

The compounds disclosed herein, reference to any atom includes reference to isotopes thereof. For example, reference to H includes reference to any isotope thereof, such as a ¹H, ²H, ³H, or mixtures thereof.

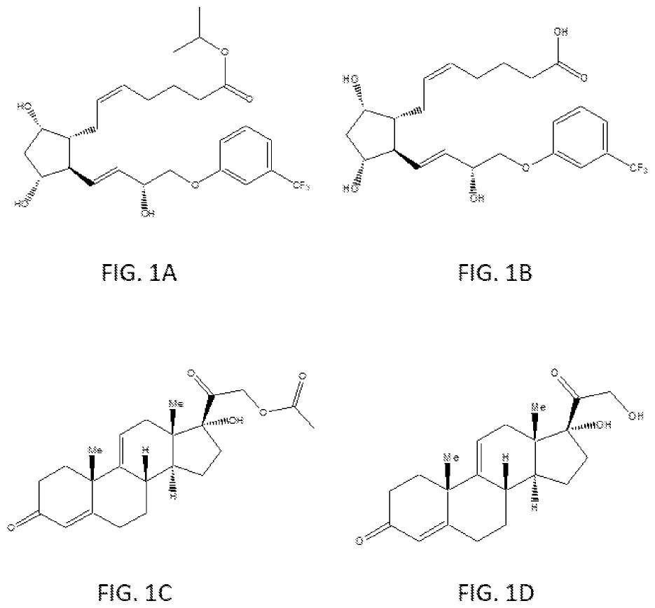

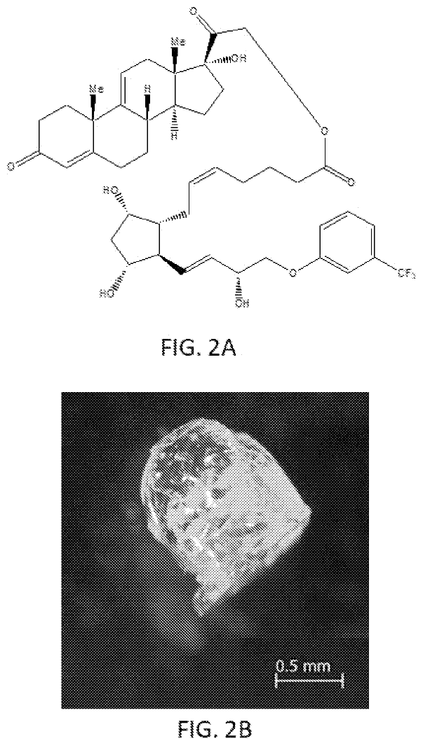

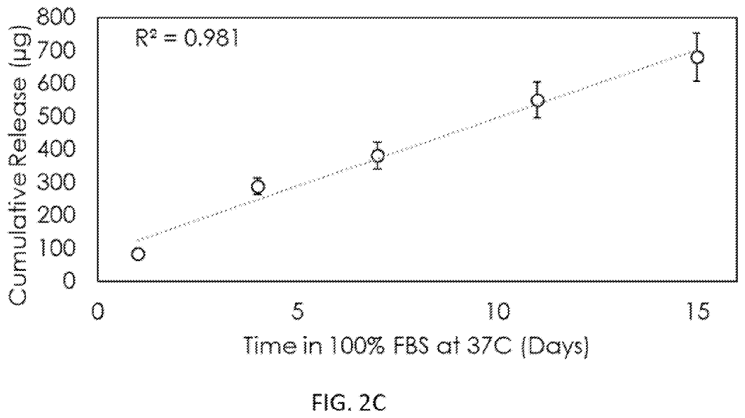

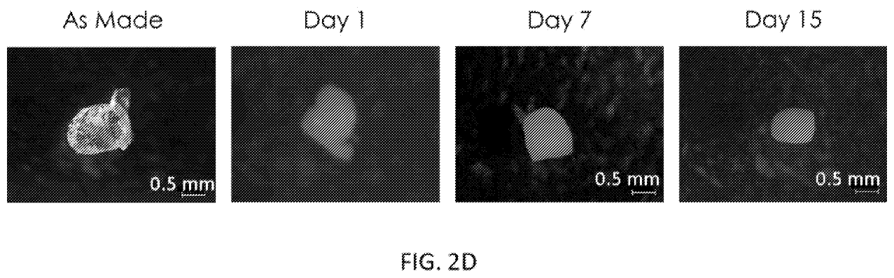

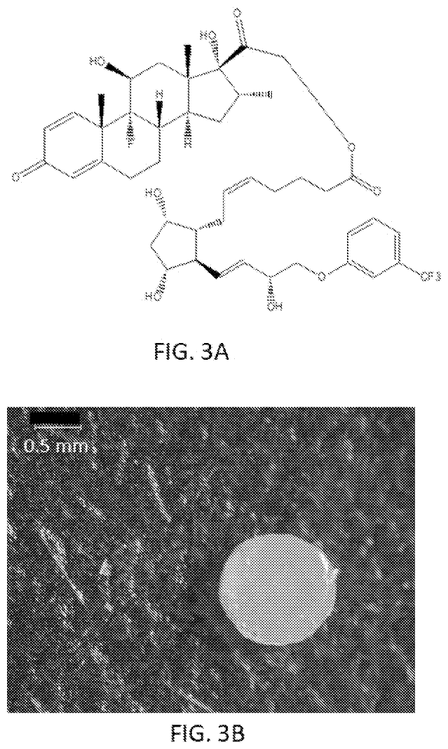

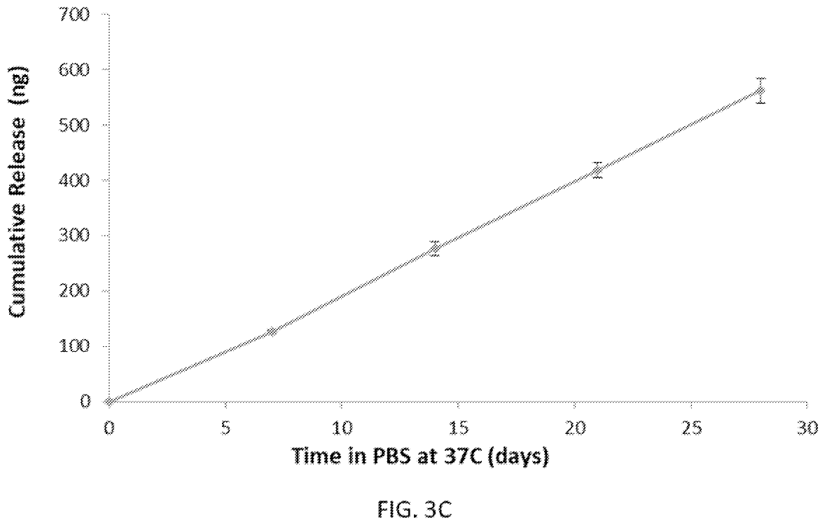

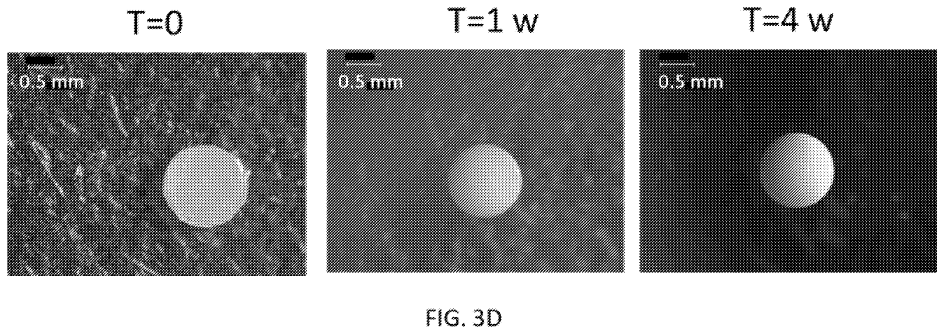

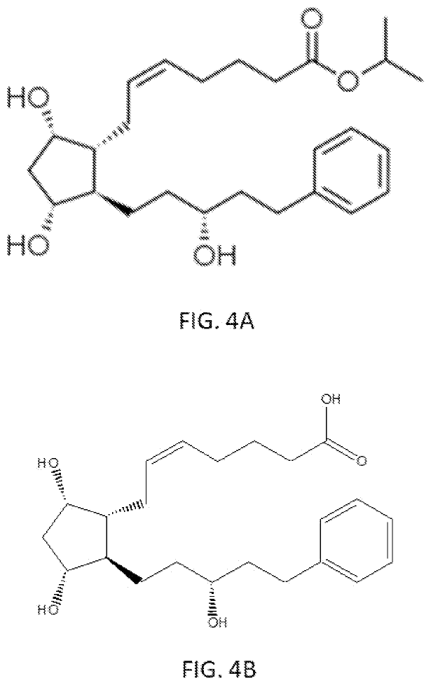

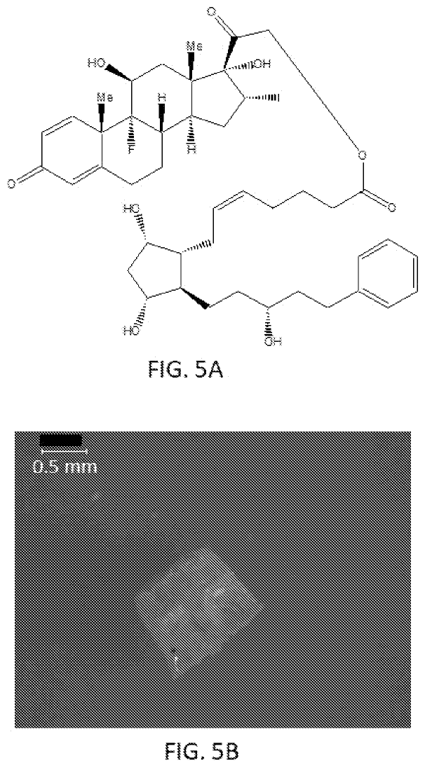

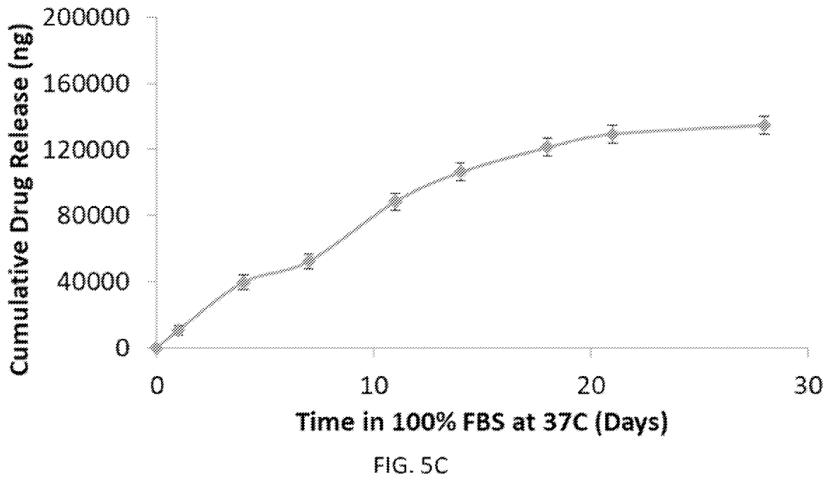

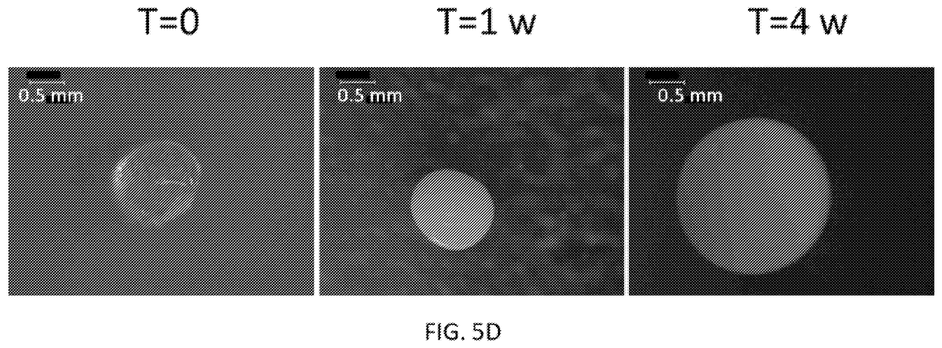

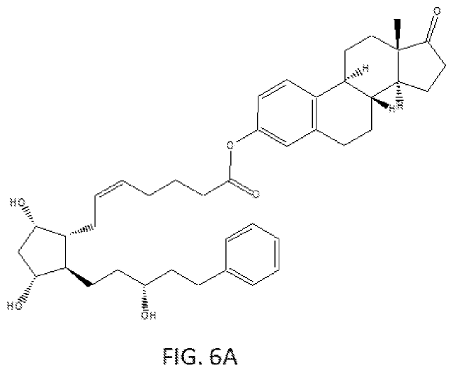

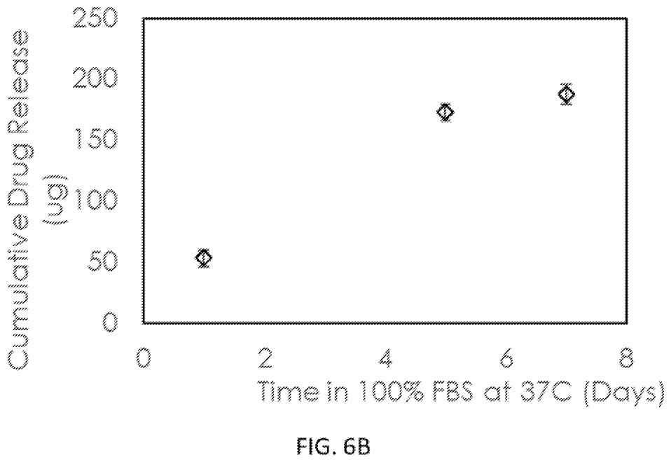

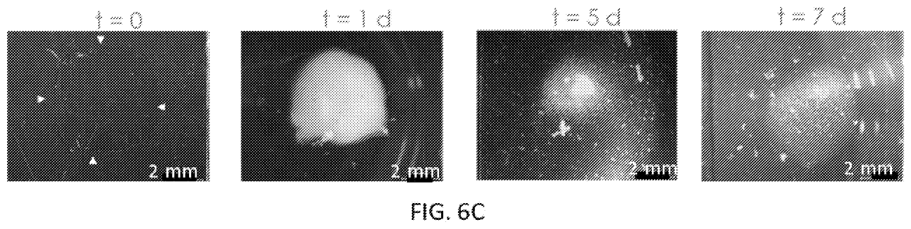

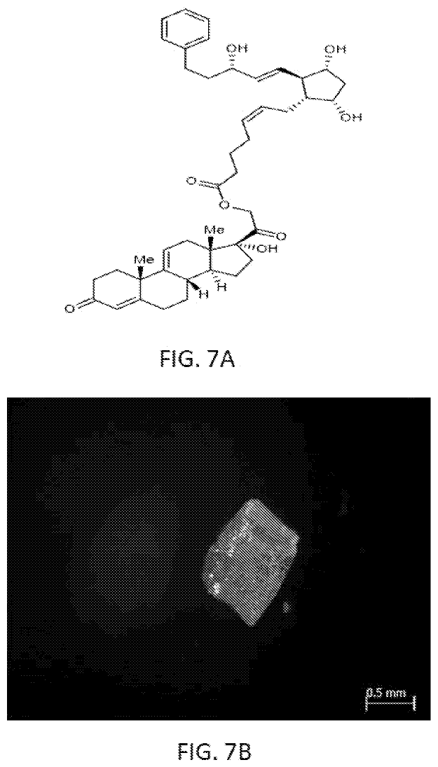

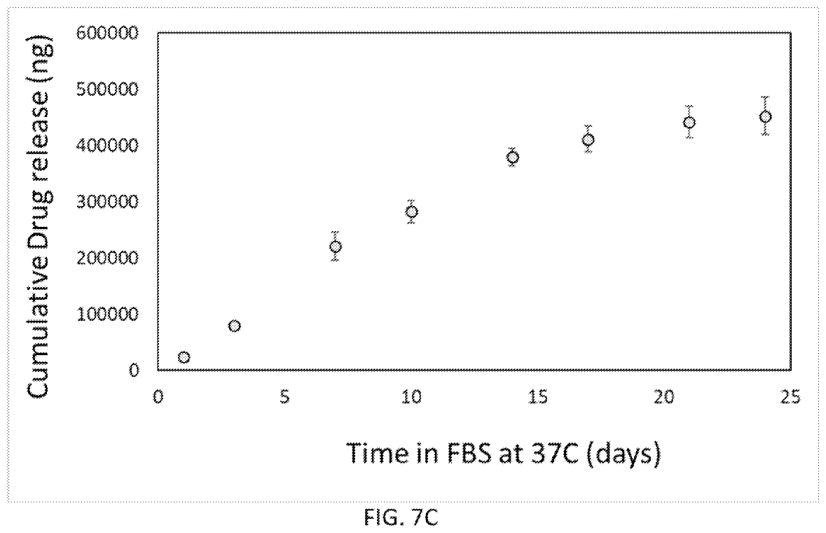

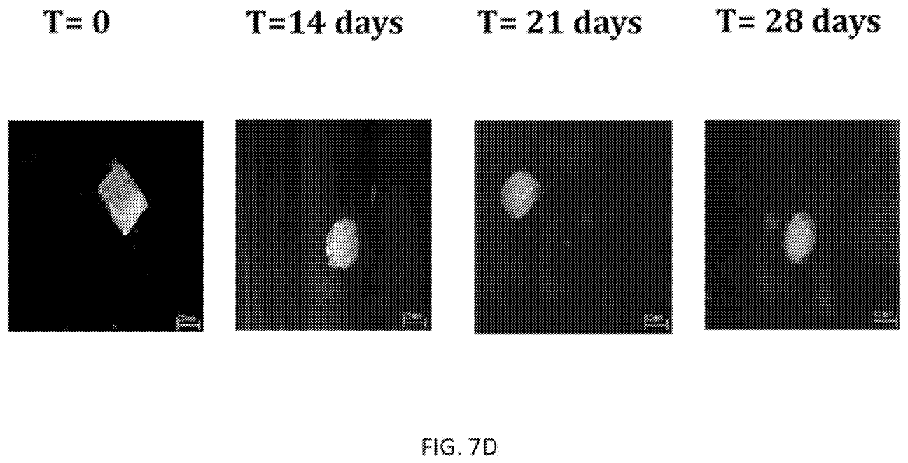

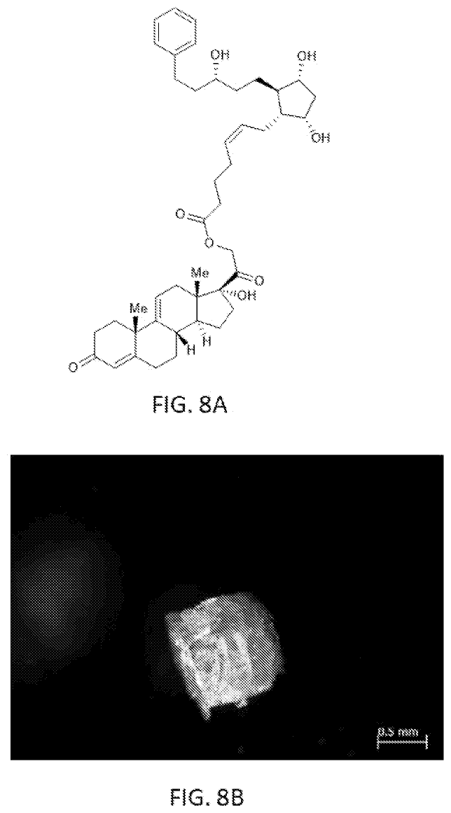

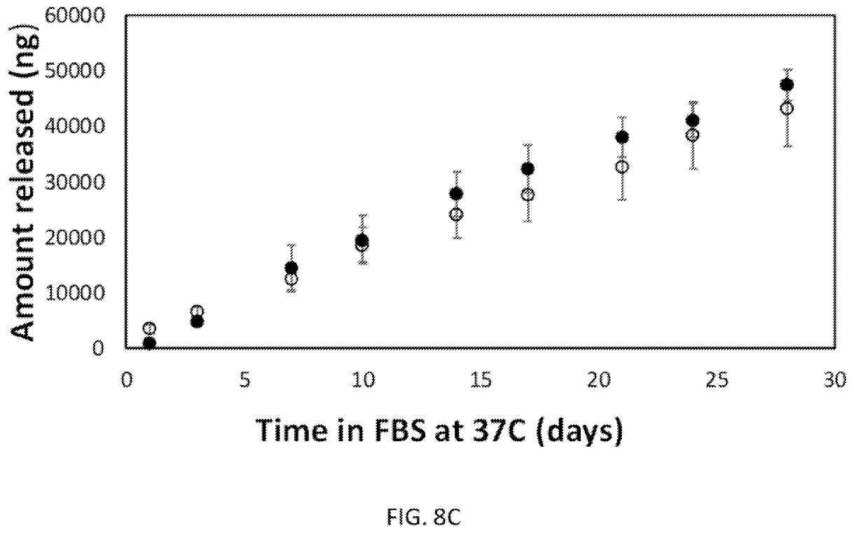

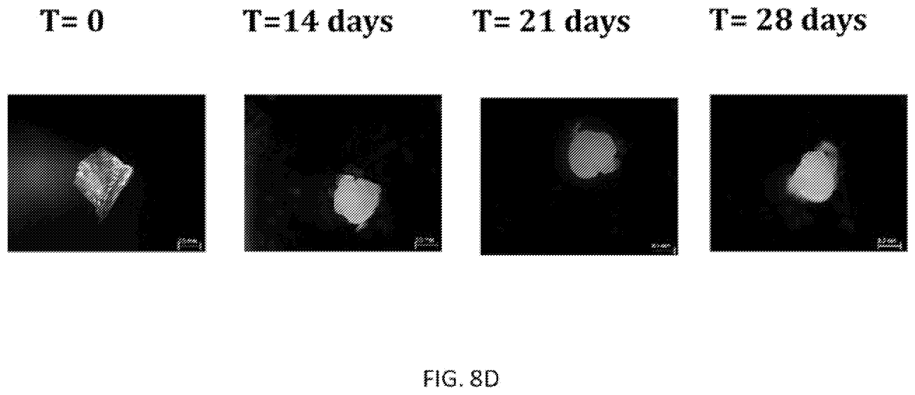

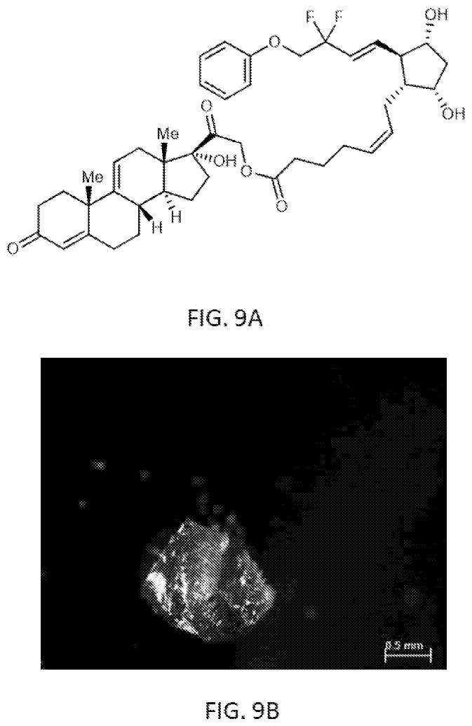

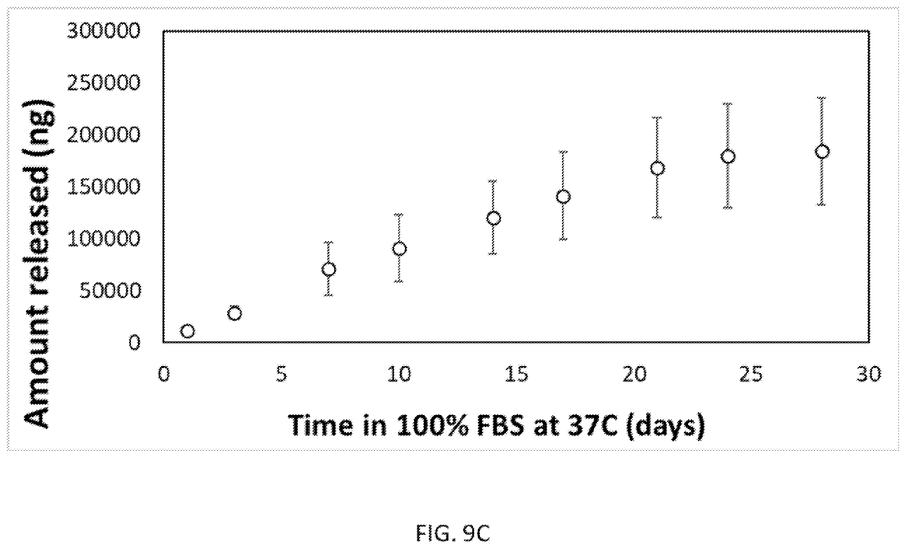

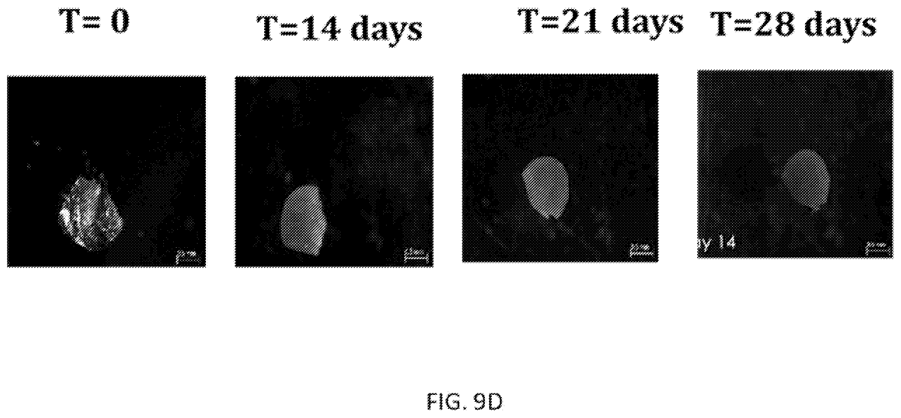

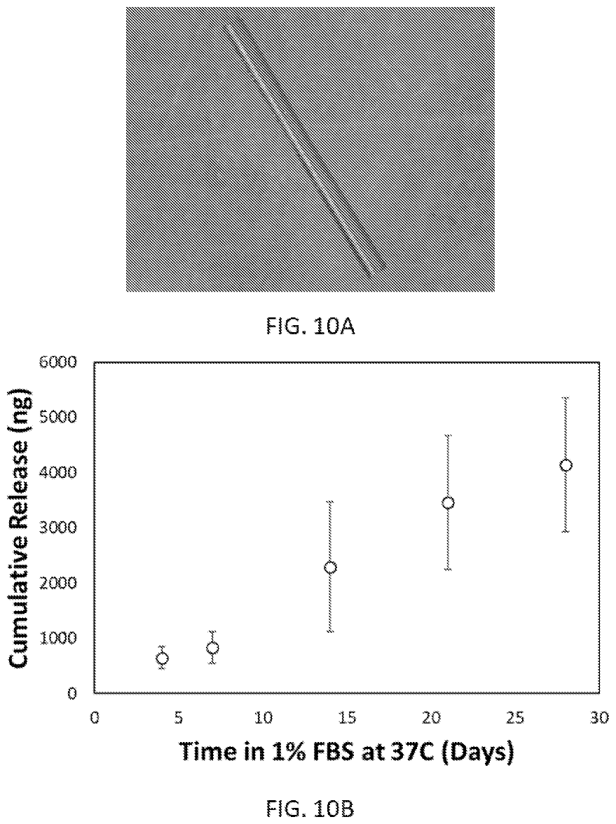

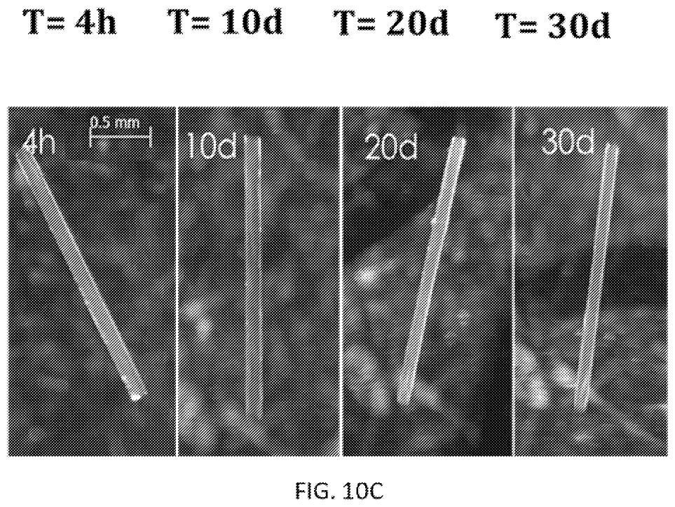

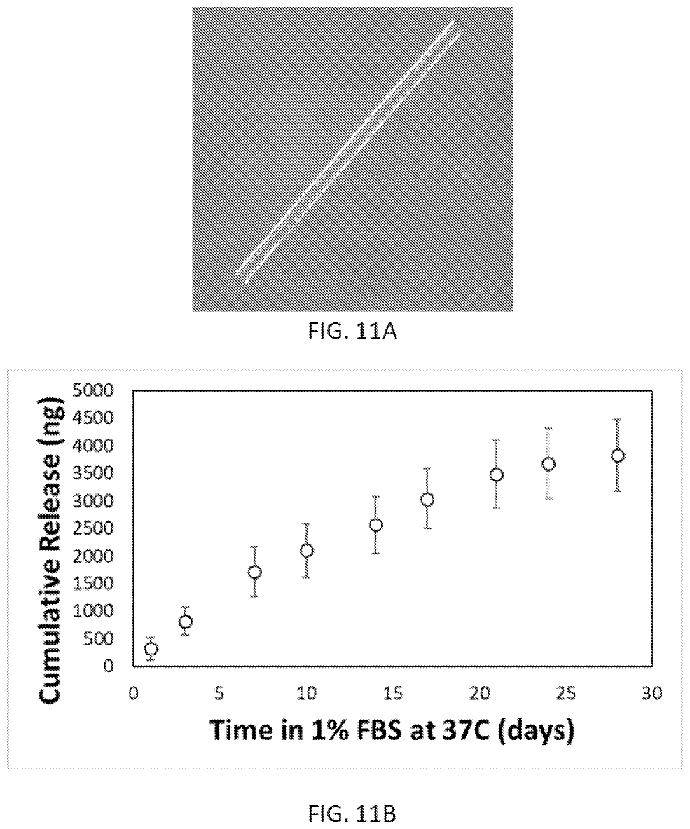

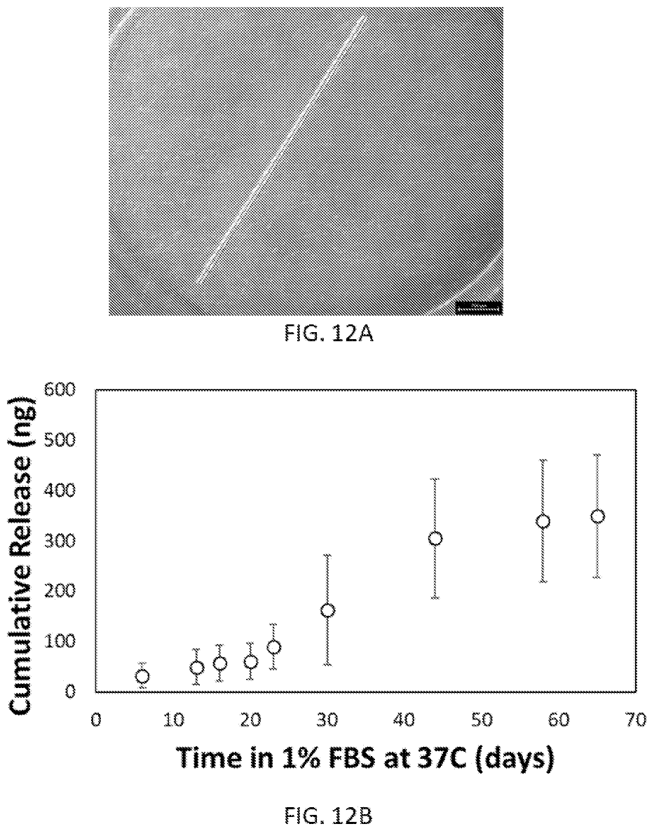

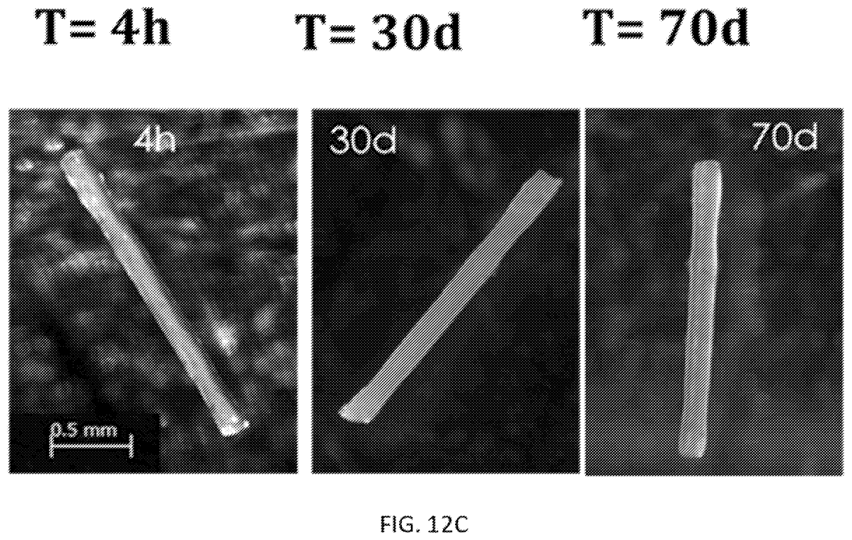

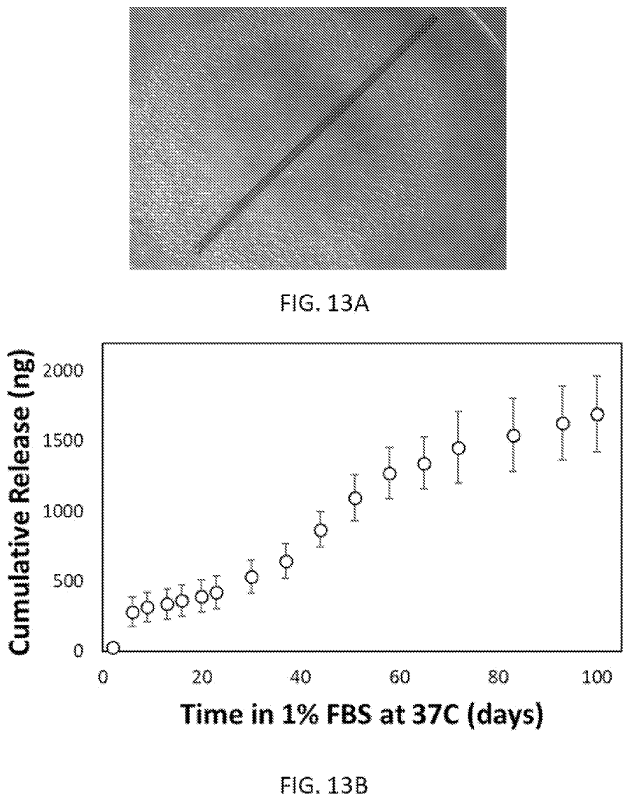

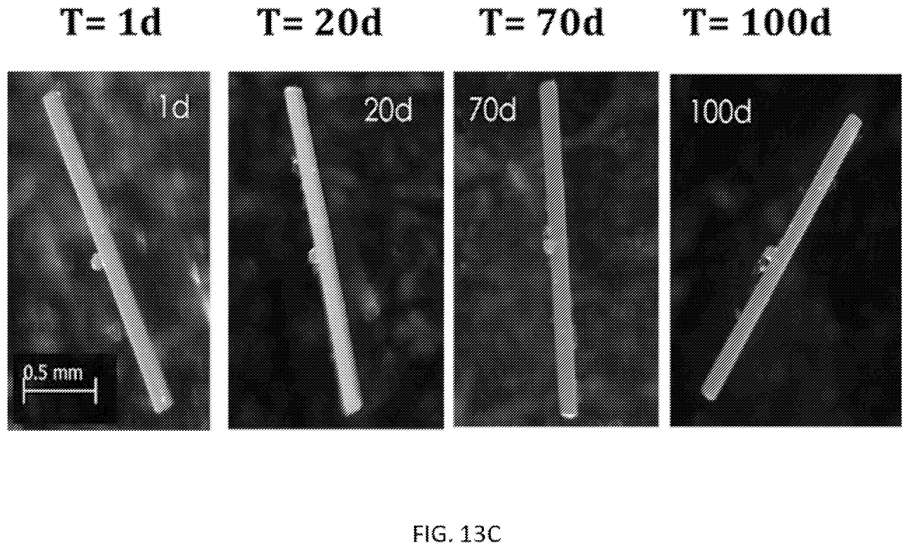

Generally, recitations of “anecortave” herein refer to “anecortave” in the desacetate form of FIG. 1B; however, where applicable, recitations of “anecortave” include disclosure of each of the “desacetate” and “acetate” forms.

The term “opioid” or “opiate,” as used interchangeably herein, generally refers to a ligand that binds to an opioid receptor, such as, for example, the delta (δ)-opioid receptor (DOR), the kappa (κ)-opioid receptor (KOR), mu (μ)-opioid receptor (MOR), nociceptin opioid receptor (NOR), zeta (ζ)-opioid receptor (ZOR), or any combination thereof. In some embodiments, the opioid is an opioid agonist, an opioid antagonist, or a mixed opioid agonist/antagonist of an opioid receptor. In some embodiments, the opioid agonist is a partial opioid agonist or an inverse opioid agonist. In some embodiments, the opioid is an opioid radical. In some embodiments, the opioid radical is joined to a radical of a therapeutically active agent by a linker, as described herein, forming an opioid dimer. In some embodiments, the opioid dimer is a heterodimer as described above. In some embodiments, an opioid radical is joined to a second radical, which is not an opioid radical, such as, for example, a radical of a therapeutically active agent (e.g., a steroid), by a linker described herein, forming an opioid heterodimer. In some embodiments, a first opioid radical, such as, for example, a partial opioid agonist, is joined to a second opioid radical, which is a different opioid radical than the first opioid radical, such as, for example, an opioid antagonist, by a linker described herein, forming a heterodimer.

The term “pellet,” as used herein, refers to the shape of the pharmaceutical compositions of the disclosure that is rounded, spherical, cylindrical, or a combination thereof. In some embodiments, the pellet has a mean diameter from about 0.2 to 5 mm, e.g., from about 0.2 to 1 mm, from about 0.2 to 2 mm, from about 0.3 to 3 mm, from about 1.5 to 5 mm, from about 2 to 5 mm, from about 2.5 to 5 mm, from about 3 to 5 mm, from about 3.5 to 5 mm, from about 4 to 5 mm, or from about 4.5 to 5 mm.

“Pharmaceutically acceptable salt” includes both acid and base addition salts. A pharmaceutically acceptable salt of any one of the pharmacological agents described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuricacid, nitricacid, phosphoric acid, hydroiodicacid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

In some instances, producing a final medicinal product from an active pharmaceutical ingredient (API) that is a solid or a liquid at room temperature is an extensive and costly process. Pharmaceutical formulation of an API that is a liquid at room temperature often requires, for example, excipients (e.g., stabilizers, additives, adjuvants, etc.) or conjugation to another molecule (e.g., a polymer) to stabilize and/or produce a processable and/or storable product. Instead, APIs that are solids at room temperature are often used for pharmaceutical formulations, avoiding the additional processing and formulation for producing medicinal products from liquid APIs; albeit, still incurring significant cost to formulation to a final medicinal product. Therefore, the cost of formulating solid or liquid APIs as well as the limited processability of liquid or solid APIs limit the efficacy and/or adoption of potentially beneficial therapeutics.

Furthermore, patient compliance is an often unresolved issue in the clinic. In some instances, modified-release pharmaceuticals can improve patient compliance. For example, extended-release (ER) dosage forms, such as sustained-release (SR) or controlled-release (CR) dosage forms, may facilitate compliance with a therapeutic regimen in some instances. SR and CR dosage forms are generally designed to liberate an API at a certain rate, such as to maintain a particular drug concentration over a period of time. For example, SR maintains drug release over a sustained period but not at a constant rate, while CR maintains drug release over a sustained period at a more consistent (e.g., nearly constant) rate (e.g., zero-order). Despite their ability to extend the dosing of an active, such dosage forms can be difficult to develop. Moreover, such dosage forms often include controlled release excipients (e.g., polymers) and/or controlled release matrices to facilitate controlled release. In the case of liquid or otherwise low melting point active agents, controlled release formulations can be even more difficult to develop. Moreover, even in the best circumstances, many controlled release forms have limited durations of active release (e.g., 24-hour release windows), so patient compliance remains an issue.

Provided in certain embodiments herein are processable compounds that address the burden of medicinal product formulation as well as patient compliance. In certain embodiments, compounds described herein are solids at body temperature (e.g., about 37° C., or lower). In certain embodiments, compounds provided herein comprise a first group or radical (e.g., a structure provided in any one of Formula (I), Formula (I′), Formula (IA), Formula (IB), Formula (IC), Formula (ID), Formula (II′), Formula (IIA), Formula (IIB), Formula (III′), Formula (IV′), Formula (V′), Formula (VII), Formula (VII-A), or Formula (VII-B)) (e.g., covalently) joined (e.g., conjugated) to a second group. In some embodiments, the first group is a radical of Formula (I′), Formula (IA), Formula (IB), Formula (IC), Formula (ID), Formula (II′), Formula (IIA), Formula (IIB), Formula (III′), Formula (IV′), Formula (V′), Formula (VII), Formula (VII-A), or Formula (VII-B). In some embodiments, the second group is a group that is itself not processable itself in free form (e.g., has a melting temperature that is higher than its degradation temperature, is generally insoluble or too soluble, such as in aqueous media, or is otherwise not suited for processing). In certain embodiments, the second group is a group that is not processable in dimer form (e.g., when conjugated directly to itself or via a linker, such as described herein). In certain embodiments, the second group is a group that is processable in dimer form (e.g., when conjugated directly to itself or via a linker, such as described herein). In some embodiments, the second group is a group that has a melting point and/or glass transition temperature of less than 50° C., less than 40° C., less than 37° C., or the like. Generally, such compounds, even if solid at room temperature, may not be suitable for use as implants due to the possibility of melting or deformation in a physiological environment. In some embodiments, the compound is formed into an implantable article (e.g., a pellet), such as using methods described herein (e.g., as described in the examples). In some embodiments, the implantable article has a (e.g., zero-order) controlled release rate over an extended period (e.g., 1 week, 2 weeks, 3 weeks, 4 weeks, 6 weeks, 12 weeks, 52 weeks, or more) in an aqueous medium (e.g., a buffer solution, serum, biological environment (e.g., in the eye), in vivo, or the like). In some embodiments, a compound provided herein (or implant comprising such a compound) is administered to an individual suffering an acute or a chronic disease or condition (e.g., as a therapy for the acute or chronic disease or condition) in any suitable manner (e.g., route of administration, such as by implanting, and/or frequency of dosing), such as a single dose or a series of doses (e.g., once or twice every 1 week, 2 weeks, 3 weeks, 4 weeks, 6 weeks, 12 weeks, 52 weeks, or more).

In certain instances, compounds (e.g., conjugates) provided herein are used to improve treatment options and/or patient compliance for acute diseases and/or disorders. In some instances, processable compounds described herein are used to improve treatment options and/or patient compliance for chronic diseases and/or disorders. In some embodiments, the processable compounds described herein are used to improve treatment options and/or patient compliance for ophthalmology, neurology, cardiology, post-surgical medicine, urology, orthopedics, and pain management.

Provided herein are compounds (e.g., conjugates) that are processable (e.g., into an article or implant). A processable compound is a compound that can be processed with heat or solvent to form a solid, such as with little (e.g., less than 20 wt. %, less than 10 wt. %, or less than 5 wt. %) or no addition of further excipient. In certain instances, the solid prepared following processing is an amorphous solid or a solid having a highly amorphous morphology (e.g., as discussed in more detail herein). In some instances, the solid prepared following processing starts as an amorphous solid, and, upon further processing (e.g., addition to an aqueous environment), the amorphous solid forms a (e.g., partially or substantially) crystalline solid. In some instances, the (e.g., partially or substantially) crystalline solid has suitable mechanical properties, such as to release (e.g., through surface erosion) a compound and/or radical (in its free form) provided herein. In certain instances, a processable compound provided herein is a solid at room (e.g., 20° C.) and/or physiological temperature (e.g., 37° C.). In some instances, the compound is thermally processable, such as having a melt or glass transition temperature of at least 37° C., at least 40° C., at least 50° C., at least 60° C., at least 70° C., at least 100° C., or the like. In some embodiments, it is beneficial for the compound to be solid at room temperature, but processable at a temperature that is not prohibitively high. In some embodiments, a compound provided herein has a melt and/or glass transition temperature of less than 200° C., less than 150° C., less than 140° C., less than 130° C., less than 125° C., less than 120° C., or the like.

In some embodiments, the compounds have any suitable morphology, such as to facilitate processing and/or pharmacodynamic effects (e.g., release profile). In certain embodiments, the compound (or implant or pharmaceutical composition comprising the compound) is amorphous (or comprises a highly amorphous content). In some embodiments, a compound (e.g., morphology) provided herein is a solid, such as at a physiological temperature (e.g., having a melting point (Tₘ) and/or glass transition temperature (T_{g}) of at least 37° C.). In some embodiments, the compound is a crystalline solid, film, glass, or amorphous solid (e.g., at a temperature of at least 37° C.). In some instances, such as when the compound is further processed (e.g., introduced to an aqueous environment), the compound (or composition, article, implant, or coating comprising the compound) provided herein has a crystallinity of 15% or more (e.g., determined by PXRD, DSC, or polarized light microscopy). In some embodiments, the compound (or composition, article, implant, or coating comprising the compound) has a crystallinity of at most 15% (e.g., determined by PXRD, DSC, or polarized light microscopy). In some embodiments, the compound (or composition, article, implant, or coating comprising the compound) has a crystallinity of at most about 15% or less (e.g., determined by PXRD, DSC, or polarized light microscopy) after processing the compound into an article or implant provided herein (e.g., via extrusion processing described herein) but has a crystallinity of 15% or more after further processing (e.g., addition to an aqueous environment). In some embodiments, the compound (or composition, article, implant, or coating comprising the compound) is substantially non-crystalline (e.g., determined by PXRD, DSC, or polarized light microscopy). In some embodiments, the compound (or composition, article, implant, or coating comprising the compound) is amorphous (e.g., determined by PXRD, DSC, or polarized light microscopy). In some embodiments, the compound (e.g., morphology) has a thermal melting point (Tₘ) that is greater than or equal to the glass transition temperature (T_{g}). In some embodiments, the compound has a melting point of at least 37° C. In some embodiments, the compound (e.g., morphology) has a melting point of at least 100° C. In some embodiments, either one or both of the first and/or second radicals (or (e.g., active) fragments or metabolites thereof) of the compounds (e.g., drug conjugates) and (e.g., active) agents are released (e.g., in their free form), the release being controlled release and/or extended release. In some embodiments, either one or both of the first and/or second radicals of the compounds and agents are released (e.g., in their free form) for at least 15 days (e.g., in solution, buffer solution, serum, biological environment, in vivo, or the like).

Described in certain embodiments herein are processable agents (e.g., compounds) formed from a processable group (e.g., a radical that makes a non-processable radical processable when linked or joined thereto) and a non-processable moiety (e.g., a radical that, if in its free form, would not be processable, such as by thermal techniques, e.g., because of a melting point that is below a physiological temperature). In some embodiments, the processable agents described herein are processable into a solid (e.g., at a temperature of at least 20° C., 25° C., 30° C., 37° C., or more). In some embodiments, provided herein are compounds useful in therapies for treating acute, chronic, or both disease or condition. In some instances, the conjugates provided herein represent a significant advance in the art, e.g., as processable compounds suitable for being formed into or formulated into controlled and/or extended release articles, implants, coatings, or other pharmaceutical compositions that are beneficial for treating acute and/or chronic diseases or disorders, such as with infrequent (e.g., a single, or weekly, monthly, or less frequent) administration.

Provided in some embodiments is a compound comprising a first radical and a second radical. In some embodiments, the first radical has a structure represented by Formula (I′):

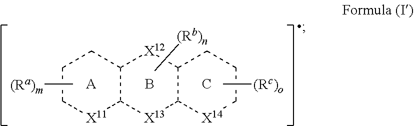

In some embodiments, [symbol] is a single bond or a double bond. In some embodiments, each R^{a}, R^{b}, and R^{c} are independently selected the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted. In some embodiments, any one of R^{a}, R^{b}, or R^{c} are taken together with another of R^{a}, R^{b}, or R^{c} to form a substituted or an unsubstituted cycloalkyl, a substituted or an unsubstituted heterocycloalkyl, a substituted or an unsubstituted aryl, or a substituted or an unsubstituted heteroaryl. In some embodiments, any one of R^{a}, R^{b}, or R^{c} are taken together with another of R^{a}, R^{b}, or R^{c} to form a substituted or an unsubstituted cycloalkyl or heterocycloalkyl. In some embodiments, X¹¹, X¹², X¹³, and X¹⁴ are each independently selected from the group consisting of a bond and Q_{y}, wherein each Q is independently selected from the group consisting of —O—, —NR—, —S(R)ₓ—, and —C(R)_{z}—. In some embodiments, each x is independently 0-5. In some embodiments, each y is independently 1-3. In some embodiments, each z is independently 1 or 2. In some embodiments, each R is independently selected from the group consisting of H, halogen, alkyl, heteroalkyl, cycloalkyl, heterocycloalkyl, alkoxy, amino, hydroxy, and thiol, or is taken together with another R to form an oxo. In some embodiments, each of m, n, and o are independently 0-6. In some embodiments, the second radical being a therapeutically active agent (or drug) (e.g., in its free form) and the first radical (e.g., a radical of a carrier) being different than the second radical. In some embodiments, the first radical and the second radical being attached to a linker (e.g., that links the first radical and the second radical). In some embodiments, either the first radical, the second radical, or both the first radical and the second radical is not a steroid. In some embodiments, the compound is a pharmaceutically-acceptable salt or solvate.

In some embodiments, any one of R^{a}, R^{b}, or R^{c} are taken together with another of R^{a}, R^{b}, or R^{c} to form a substituted or an unsubstituted cycloalkyl, a substituted or an unsubstituted heterocycloalkyl, a substituted or an unsubstituted aryl, or a substituted or an unsubstituted heteroaryl.

In some embodiments, R^{a}, R^{b}, and R^{c} are described elsewhere herein.

In some embodiments, X¹¹, X¹², X¹³, and X¹⁴ are described elsewhere herein.

In some embodiments, Q is described elsewhere herein.

In some embodiments, x is described elsewhere herein.

In some embodiments, y is described elsewhere herein.

In some embodiments, z is described elsewhere herein.

In some embodiments, R is described elsewhere herein.

In some embodiments, the first radical is a radical of a natural compound. In some embodiments, the first radical is naturally occurring in nature. In some embodiments, the first radical is naturally occurring in the body.

In some embodiments, the first radical is a radical described herein, such as D1.

In some embodiments, the first radical is described elsewhere herein.

In some embodiments, the second radical is a radical described herein, such as D2.

In some embodiments, the second radical is described elsewhere herein.

In some embodiments, the linker is described elsewhere herein (e.g., L, L¹, or L²).

In some embodiments, the linker (e.g., L) is not a bond.

Provided in some embodiments herein is a compound having a structure represented by Formula (I): D1-L-D2. In some embodiments, D1 is a radical of a carrier. In some embodiments, D2 is a radical of an active agent (or drug). In some embodiments, L is -(Q¹-M-Q²)-. In some embodiments, Q¹ and Q² are each independently absent or (C═X¹)X². In some embodiments, X¹ is O or S. In some embodiments, X² is O, S, or NR¹. In some embodiments, R¹ is hydrogen or C₁-C₆ alkyl. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl). In some embodiments, either the first radical, the second radical, or both the first radical and the second radical is not a steroid.

In some embodiments, D1 is a radical of a carrier.

In some embodiments, D1 is a radical of an inactive agent.

In some embodiments, D1 is a radical of a non-medicinal agent.

In some embodiments, D1 (e.g., the radical of an inactive agent or a non-medicinal agent) is an agent that (in its free form) does not provide a significant, measurable, and/or direct therapeutic effect and/or benefit (e.g., to an individual (e.g., in need thereof)). In some embodiments, D1 (e.g., the radical of an inactive agent or a non-medicinal agent) is an agent that (in its free form) does not provide a therapeutic effect and/or benefit (e.g., to an individual (e.g., in need thereof)).

In some embodiments, D1 (the carrier agent) (e.g., in its free form) is not a therapeutically active agent (or drug).

In some embodiments, D1 (the carrier agent) (e.g., in its free form) is a therapeutically active agent (or drug).

In some embodiments, D1 is a radical of a natural compound. In some embodiments, D1 is naturally occurring in nature. In some embodiments, D1 is naturally occurring in the body.

In some embodiments, D1 comprises a three-ring core structure. In some embodiments, D1 comprises a three-ring core structure having a structure represented by Formula (I′), wherein the three-ring core structure is a processable (carrier) group (e.g., a processable group described herein).

In some embodiments, D1 comprises a three-ring core structure. In some embodiments, D1 comprises a four-ring core structure having a structure represented by Formula (I′)), wherein the four-ring core structure is a processable (carrier) group (e.g., a processable group described herein).

In some embodiments, D1 comprises a five-ring core structure. In some embodiments, D1 comprises a five-ring core structure having a structure represented by Formula (I′), wherein the five-ring core structure is a processable (carrier) group (e.g., a processable group described herein).

In some embodiments, D1 comprises a six-ring core structure. In some embodiments, D1 comprises a six-ring core structure having a structure represented by Formula (I′), wherein the six-ring core structure is a processable (carrier) group (e.g., a processable group described herein).

In some embodiments, D1 has a structure represented by Formula (I′).

In some embodiments, D1 comprises a core structure with three or more fused rings.

In some embodiments, D1 has a 3-ring structure, a 4-ring structure, a 5-ring structure, or a 6-ring structure.

In some embodiments, D1 has a 3-ring structure (e.g., riboflavin (vitamin B₂), santonic acid, tetrahydrocannabiorcol, or the like).

In some embodiments, D1 has a 4-ring structure (e.g., cucurbalsaminol A, lanosterol, lepidolide, panaxatriol, annonamine, boldine, (−)-Stepholidine, or the like).

In some embodiments, D1 has a 5-ring structure (e.g., ursolic acid, amyrin, isoarborinol, boswellic acid, cycloartenol, oleanolic acid, plicadin, pukatiene, or the like).

In some embodiments, D1 has a 6-ring structure (e.g., dichapetalin, chamaecydin, or the like).

In some embodiments, D1 is a radical of a steroid. In some embodiments, D1 is a radical of an anti-inflammatory steroid (e.g., dexamethasone, hydrocortisone, or triamcinolone). In some embodiments, D1 is a radical of a benign steroid (e.g., cholesterol, cholic acid, or deoxycholic acid)). In some instances, a benign steroid is a (steroid) carrier. In some instances, a benign steroid is a steroid that (in its free form) does not provide a significant therapeutic effect and/or benefit (e.g., to an individual (e.g., in need thereof)). In some embodiments, D1 is a radical of a steroid. In some embodiments, D1 is a radical of an angiostatic steroid (e.g., anecortave).

In some embodiments, D1 is a radical of: an angiotensin-converting-enzyme (ACE) inhibitor (e.g., enalapril, captopril, cilazapril, fosinopril, lisinopril, moexipril, perindopril, quinapril, ramipril, trandolapril, zofenopril, or the like), an immunosuppressant (e.g., everolimus, tacrolimus, or the like), an angiotensin II receptor blocker (e.g., candesartan, eprosartan, irbesartan, losartan, olmesartan, telmisartan, valsartan, azilsartan, or the like), an atypical antipsychotic (e.g., paliperidone or the like), a human immunodeficiency virus (HIV) integrase inhibitor (e.g., dolutegravir or the like), a tyrosine kinase inhibitor (e.g., axitinib or the like), a beta-3-adrenergic agonist (e.g., mirabegron or the like), a 1,3-benzoxazole (e.g., tafamidis or the like), a statin (rosuvastatin, simvastatin, or the like), a vasopressin receptor antagonist (e.g., tolvaptan or the like), an antineoplastic (e.g., belzutifan, dichapetalin, or the like), a dopamine agonist (e.g., rotigotine or the like), an acetyl cholinesterase inhibitor (e.g., donepezil, galantamine, or the like), an anti-epileptic (e.g., valproic acid or the like), an opioid agonist (e.g., oxycodone, hydrocodone, or the like), an opioid antagonist (e.g., naltrexone, naloxone, nalmefene, or the like), an antimuscarinic (e.g., fesoterodine, tolterodine, or the like), an anticholinergic (e.g., darifenacin or the like), a vasodilator (e.g., treprostinil or the like), an anti-arrhythmic (e.g., propafenone or the like), an nonsteroidal anti-inflammatory drug (NSAID) (e.g., naproxen, bromfenac, celecoxib, indomethacin, or the like), an antidepressant (e.g., paroxetine, bupropion, nortriptyline, or the like), a vitamin (e.g., a vitamin D₃ (e.g., calcifediol or the like)), an antihistamine (e.g., cetirizine, desloratadine, or the like), a triterpenoid topoisomerase inhibitor (e.g., betulinic acid or the like), a triterpenoid (e.g., acetoxolone or the like), a steroid (e.g., a mineralocorticoid (e.g., fludrocortisone or the like)), nicotinic acetylcholine receptor agonist (e.g., varenicline or the like), a fatty acid synthase (FAS) inhibitor (e.g., bicycol or the like), a carbonic anhydrase inhibitor (e.g., dorzolamide or the like), an alpha-adrenergic agonist (e.g., brimonidine), a sympathomimetic (e.g., epinephrine), or any combination thereof.

In some embodiments, D1 is any radical (e.g., carrier radical) provided herein (e.g., any carrier provided in Table 1 or Table 2).

In some embodiments, D1 is described elsewhere herein, such as in Table 1 or Table 2.

In some embodiments, D2 is a radical of an active agent. In some embodiments, D2 is a radical of a therapeutically active agent or a drug. In some embodiments, D2 is a radical of a therapeutically active agent. In some embodiments, D2 is a radical of a drug.

In some embodiments, D2 is a radical of: an angiotensin-converting-enzyme (ACE) inhibitor (e.g., enalapril, captopril, cilazapril, fosinopril, lisinopril, moexipril, perindopril, quinapril, ramipril, trandolapril, zofenopril, or the like), an immunosuppressant (e.g., everolimus, tacrolimus, or the like), an angiotensin II receptor blocker (e.g., candesartan, eprosartan, irbesartan, losartan, olmesartan, telmisartan, valsartan, azilsartan, or the like), an atypical antipsychotic (e.g., paliperidone or the like), a human immunodeficiency virus (HIV) integrase inhibitor (e.g., dolutegravir or the like), a tyrosine kinase inhibitor (e.g., axitinib or the like), a beta-3-adrenergic agonist (e.g., mirabegron or the like), a 1,3-benzoxazole (e.g., tafamidis or the like), a statin (rosuvastatin, simvastatin, or the like), a vasopressin receptor antagonist (e.g., tolvaptan or the like), an antineoplastic (e.g., belzutifan, dichapetalin, or the like), a dopamine agonist (e.g., rotigotine or the like), an acetyl cholinesterase inhibitor (e.g., donepezil, galantamine, or the like), an anti-epileptic (e.g., valproic acid or the like), an opioid agonist (e.g., oxycodone, hydrocodone, or the like), an opioid antagonist (e.g., naltrexone, naloxone, nalmefene, or the like), an antimuscarinic (e.g., fesoterodine, tolterodine, or the like), an anticholinergic (e.g., darifenacin or the like), a vasodilator (e.g., treprostinil or the like), an anti-arrhythmic (e.g., propafenone or the like), an nonsteroidal anti-inflammatory drug (NSAID) (e.g., naproxen, bromfenac, celecoxib, indomethacin, or the like), an antidepressant (e.g., paroxetine, bupropion, nortriptyline, or the like), a vitamin (e.g., a vitamin D₃ (e.g., calcifediol or the like)), an antihistamine (e.g., cetirizine, desloratadine, or the like), a triterpenoid topoisomerase inhibitor (e.g., betulinic acid or the like), a triterpenoid (e.g., acetoxolone or the like), a mineralocorticoid (e.g., fludrocortisone or the like), nicotinic acetylcholine receptoragonist (e.g., varenicline or the like), a fatty acid synthase (FAS) inhibitor (e.g., bicycol or the like), a carbonic anhydrase inhibitor (e.g., dorzolamide or the like), an alpha-adrenergic agonist (e.g., brimonidine), a sympathomimetic (e.g., epinephrine), a Rho-associated kinase (ROCK) inhibitor (e.g., fasudil, hydroxyfasudil, ripasudil, netarsudil, belumosudil, verosudil, or thiazovivin), a prostaglandin (e.g., latanoprost, latanoprost acid, travoprost, travoprost acid, tafluprost, tafluprost acid, bimatoprost, or bimatoprost acid), or any combination thereof.

In some embodiments, the second radical or D2 is a radical of: a nonsteroidal anti-inflammatory drug (NSAID) (e.g., naproxen, bromfenac, celecoxib, indomethacin, or the like), a tyrosine kinase inhibitor (e.g., axitinib or the like), a statin (e.g., rosuvastatin, simvastatin, or the like), an antineoplastic (e.g., belzutifan, dichapetalin, fluorouracil, or the like), or any combination thereof.

In some embodiments, the second radical or D2 is a radical of a prostaglandin (e.g., latanoprost, latanoprost acid, travoprost, travoprost acid, tafluprost, tafluprost acid, bimatoprost, or bimatoprost acid).

In some embodiments, D1 and D2 are each independently a radical of: an angiotensin-converting-enzyme (ACE) inhibitor (e.g., enalapril, captopril, cilazapril, fosinopril, lisinopril, moexipril, perindopril, quinapril, ramipril, trandolapril, zofenopril, or the like), an immunosuppressant (e.g., everolimus, tacrolimus, or the like), an angiotensin II receptor blocker (e.g., candesartan, eprosartan, irbesartan, losartan, olmesartan, telmisartan, valsartan, azilsartan, or the like), an atypical antipsychotic (e.g., paliperidone or the like), a human immunodeficiency virus (HIV) integrase inhibitor (e.g., dolutegravir or the like), a tyrosine kinase inhibitor (e.g., axitinib or the like), a beta-3-adrenergic agonist (e.g., mirabegron or the like), a 1,3-benzoxazole (e.g., tafamidis or the like), a statin (rosuvastatin, simvastatin, or the like), a vasopressin receptor antagonist (e.g., tolvaptan or the like), an antineoplastic (e.g., belzutifan, dichapetalin, or the like), a dopamine agonist (e.g., rotigotine or the like), an acetyl cholinesterase inhibitor (e.g., donepezil, galantamine, or the like), an anti-epileptic (e.g., valproic acid or the like), an opioid agonist (e.g., oxycodone, hydrocodone, or the like), an opioid antagonist (e.g., naltrexone, naloxone, nalmefene, or the like), an antimuscarinic (e.g., fesoterodine, tolterodine, or the like), an anticholinergic (e.g., darifenacin or the like), a vasodilator (e.g., treprostinil or the like), an anti-arrhythmic (e.g., propafenone or the like), an nonsteroidal anti-inflammatory drug (NSAID) (e.g., naproxen, bromfenac, celecoxib, indomethacin, or the like), an antidepressant (e.g., paroxetine, bupropion, nortriptyline, or the like), a vitamin (e.g., a vitamin D₃ (e.g., calcifediol or the like)), an antihistamine (e.g., cetirizine, desloratadine, or the like), a triterpenoid topoisomerase inhibitor (e.g., betulinic acid or the like), a triterpenoid (e.g., acetoxolone or the like), a mineralocorticoid (e.g., fludrocortisone or the like), nicotinic acetylcholine receptor agonist (e.g., varenicline or the like), a fatty acid synthase (FAS) inhibitor (e.g., bicycol or the like), a carbonic anhydrase inhibitor (e.g., dorzolamide or the like), an alpha-adrenergic agonist (e.g., brimonidine), a sympathomimetic (e.g., epinephrine), a Rho-associated kinase (ROCK) inhibitor (e.g., fasudil, hydroxyfasudil, ripasudil, netarsudil, belumosudil, verosudil, or thiazovivin), a prostaglandin (e.g., latanoprost, latanoprost acid, travoprost, travoprost acid, tafluprost, tafluprost acid, bimatoprost, or bimatoprost acid), or any combination thereof.

In some embodiments, D2 comprises a three-ring core structure. In some embodiments, D2 comprises a three-ring core structure having a structure represented by Formula (I′)).

In some embodiments, D2 comprises a three-ring core structure. In some embodiments, D2 comprises a four-ring core structure having a structure represented by Formula (I′)).

In some embodiments, D2 comprises a five-ring core structure. In some embodiments, D2 comprises a five-ring core structure having a structure represented by Formula (I′)).

In some embodiments, D2 comprises a six-ring core structure. In some embodiments, D2 comprises a six-ring core structure having a structure represented by Formula (I′)).

In some embodiments, D2 has a structure represented by Formula (I′).

In some embodiments, D2 comprises a core structure with three or more fused rings.

In some embodiments, D2 has a 3-ring structure, a 4-ring structure, a 5-ring structure, or a 6-ring structure.

In some embodiments, D2 is a radical of a steroid. In some embodiments, D2 is a radical of an anti-inflammatory steroid (e.g., dexamethasone, hydrocortisone, or triamcinolone).

In some embodiments, D2 has a structure represented by Formula (I′).

In some embodiments, D1 and D2 comprises a three-ring core structure (e.g., having a structure represented by Formula (I′))).

In some embodiments, D1 and D2 has a structure represented by Formula (I′).

In some embodiments, D2 is a radical of any therapeutic agent (or drug) provided herein.

In some embodiments, D2 is any therapeutic agent (or drug) provided in Table 2.

In some embodiments, D2 is described elsewhere herein, such as in Table 2.

In some instances, the linker (e.g., L) is a diradical. In some instances, the diradical is a molecular species (e.g., an organic compound) with two electrons occupying degenerate molecular orbitals.

In some embodiments, the linker (e.g., L) comprises one or more linker groups, each linker group being independently selected from any linker or linker group provided herein (e.g., any linker or linker group provided in Table 3).

In some embodiments, the linker (e.g., L) is any linker or linker group provided herein (e.g., any linker or linker group provided in Table 3).

In some embodiments, L (the linker) is described elsewhere herein, such as in Table 3.

In some embodiments, Q¹ and Q² are each independently absent, C═O, C=S, (C═O)O, (C═O)S, (C═O)N, or (C═S)S.

In some embodiments, Q¹ and Q² are described elsewhere herein.

In some embodiments, X¹ and X² are described elsewhere herein.

In some embodiments, R¹ is described elsewhere herein.

In some embodiments, M is substituted or unsubstituted alkyl (e.g., C₁-C₆ alkyl), substituted or unsubstituted heteroalkyl (e.g., C₁-C₆ heteroalkyl), or substituted or unsubstituted aryl. In some embodiments, M is substituted or unsubstituted alkyl (e.g., C₁-C₆ alkyl (e.g., alkyl-carbocyclyl-alkyl)) or substituted or unsubstituted heteroalkyl (e.g., C₁-C₆ heteroalkyl).

In some embodiments, M are described elsewhere herein.

In some embodiments, L is —(CH₂CH₂O)—, —(CH₂CH₂O)₂—(CH₂CH₂O)₃—, -methyl-cyclohexyl-methyl-, —CH₂—, —CH₂CH₂—, —CH₂CH₂CH₂—, —CH₂CH₂CH₂CH₂—, —CH₂CH₂CH₂CH₂CH₂—, or —CH₂CH₂CH₂CH₂CH₂CH₂—.

In some embodiments, D1 is a (e.g., hydroxyl) radical of a compound provided in Table 1.

TABLE 1

| Name | Structure |
|---|---|
| Ursolic acid | 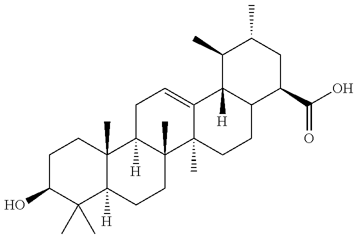 |
| Amyrin | 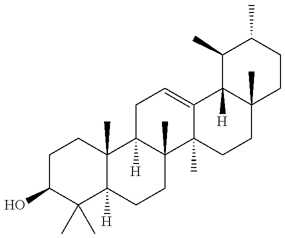 |
| Isoarborinol | 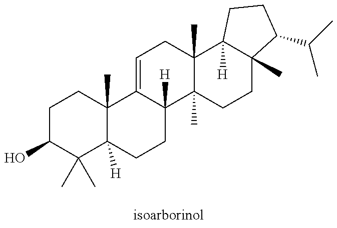 |
| Boswellic acid | 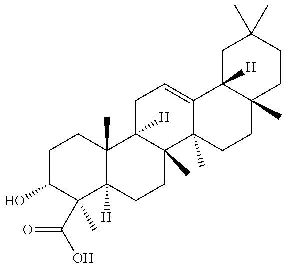 |
| Chamaecydin | 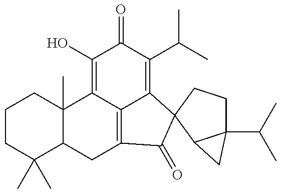 [SMILES: CC(C)C1=C2C3=C(CC4C(C)(C)CCCC4(C)C3=C(O)C1=O)C(=O)C21CCC2(C(C)C)CC12] |
| Cucurbalsaminol A | 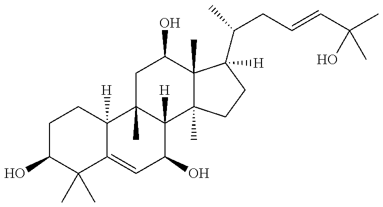 |
| Cycloartenol | 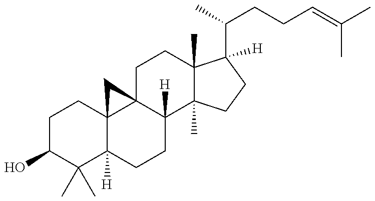 |
| Lanosterol | 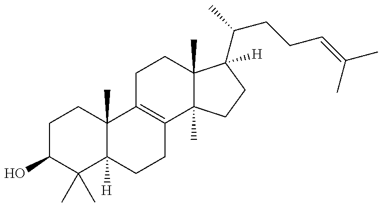 [SMILES: CC(C)=CCC[C@@H](C)[C@H]1CC[C@@]2(C)C3=C(CC[C@]12C)[C@@]1(C)CC[C@H](O)C(C)(C)[C@@H]1CC3] |
| Dichapetalin | 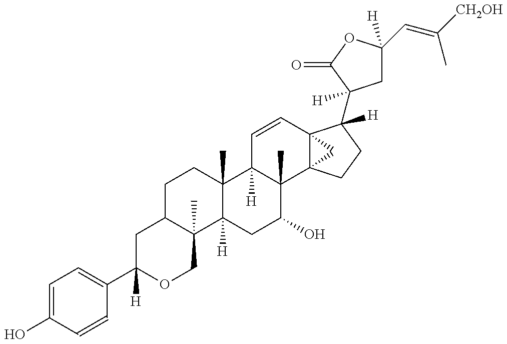 |
| Oleanolic acid | 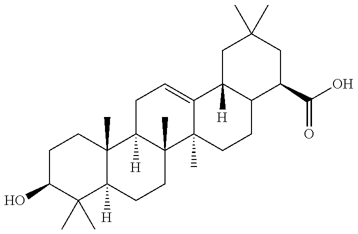 |
| Lepidolide | 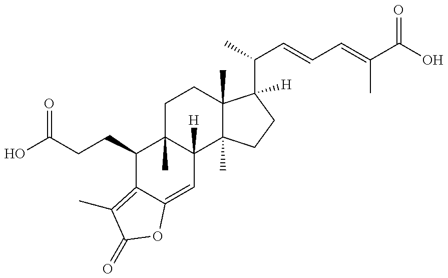 |
| Panaxatriol | 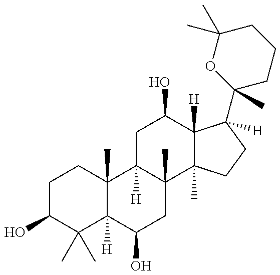 |
| Riboflavin (vitamin B2) | 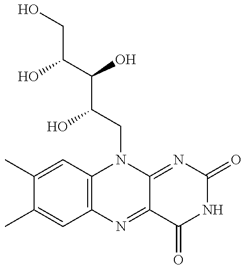 [SMILES: Cc1cc2nc3c(=O)[nH]c(=O)nc-3n(C[C@H](O)[C@H](O)[C@H](O)CO)c2cc1C] |
| Santonic acid | 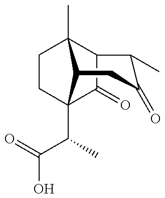 |
| Tetrahydrocannabiorcol | 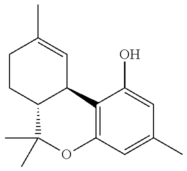 |
| Plicadin | 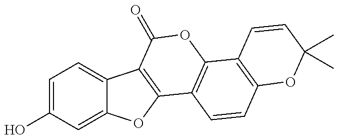 [SMILES: CC1(C)C=Cc2c(ccc3c2oc(=O)c2c4ccc(O)cc4oc32)O1] |
| Annonamine | 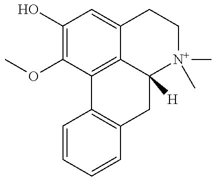 |
| Boldine | 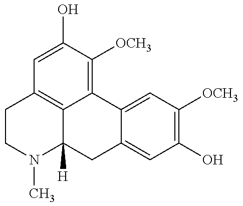 [SMILES: COc1cc2c(cc1O)C[C@H]1c3c(cc(O)c(OC)c3-2)CCN1C] |
| Pukatiene | 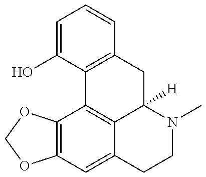 [SMILES: CN1CCc2cc3c(c4c2[C@H]1Cc1cccc(O)c1-4)OCO3] |
| (−)-Stepholidine | 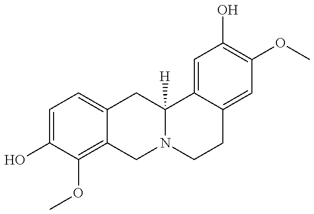 [SMILES: COc1cc2c(cc1O)[C@@H]1Cc3ccc(O)c(OC)c3CN1CC2] |
| Hydrocortisone | 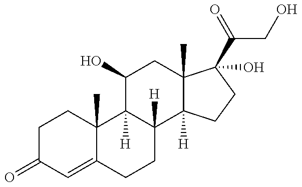 |
| Dexamethasone | 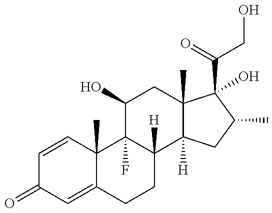 |
| Anecortave desacetate | 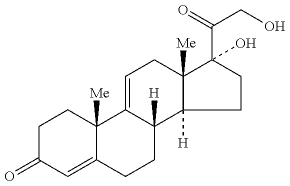 |

In some embodiments, DE is a (e.g., hydroxyl, carboxyl, thiol, or amino) radical of a compound provided in Table 2.

In some embodiments, 02 is a (e.g., hydroxyl, carboxyl, thiol, or amino) radical of a compound provided in Table 2.

In some embodiments, 02 is a steroid radical (e.g., a hydroxyl radical, a carboxylate radical, or a phosphate radical of a steroid) and D2 is a (e.g., hydroxyl, carboxyl, thiol, or amino) radical of a compound provided in Table 2.

In some embodiments, D1 and 02 are each independently a (e.g., hydroxyl, carboxyl, thiol, or amino) radical of a compound provided in Table 2.

TABLE 2

| Drug class | Therapeutic agent |
|---|---|
| ACE inhibitors | Enalapril, Captopril, Cilazapril, |
| | Fosinopril, Lisinopril, Moexipril, |
| | Perindopril, Quinapril, Ramipril, |
| | Trandolapril, Zofenopril |
| Immunosuppresant | Everolimus, tacrolimus |
| Angiotensin II Receptor | Candesartan, Eprosartan, Irbesartan, |
| Blockers | Losartan, Olmesartan, Telmisartan, |
| | Valsartan, azilsartan |
| Atypical antipsychotic | Paliperidone |
| HIV integrase inhibitor | Dolutegravir |
| Tyrosine kinase inhibitor | Axitinib |
| Beta-3-adrenergic agonist | Mirabegron |
| 1,3-benzoxazoles | Tafamidis |
| Statins | Rosuvistatin, Simvastatin |
| Vasopressin receptor antagonists | Tolvaptan |
| Antineoplastic | Belzutifan |
| Dopamine agonist | Rotigotine |
| Acetyl cholinesterase inhibitor | Donepezil |
| Anti-epilepsy | Valproic acid |
| Acetyl cholinesterase inhibitor | Galantamine |
| Opioid analgesic | Oxycodone, hydrocodone |
| Opioid analgesic | Tapentadol, Tramadol |
| Opioid antagonist | Naltrexone, Naloxone, Nalmefene |
| Antimuscarinic | Fesoterodine |
| Anticholinergic | Darifenacin |
| Antimuscarinic | Tolterodine |
| Vasodilator | Treprostinil |
| Anti-arrhythmic | Propafenone |
| NSAID | Naproxen, Bromfenac, celecoxib, |
| | Indomethacin |
| Antidepressant | Paroxetine, Bupropion |
| Vitamin D deficiency | Calcifediol |
| Antihistamine | Cetirizine |
| Triterpenoid topoisomerase | Betulinic acid |
| inhibitor | |
| Triterpenoid | Acetoxolone |
| Anticancer | Dichapetalin, Fluorouracil |
| Mineralcorticoid | Fludrocortisone |
| tetracyclic antidepressant | Amoxapine |
| Tobacco use disorder | Varenicline |
| Antihistamine | Desloratidine, Cetirizine |
| Antidepressant/ADHD | Nortriptyline |
| FAS/FASL inhibitor | Bicycol |
| Carbonic anhydrase inhibitor | Dorzolamide |
| Alpha-adrenergic agonist | Brimonidine |
| Sympatomimetic agent | Epinephrine |
| Rho-associated kinase (ROCKwe) | fasudil, hydroxyfasudil, ripasudil, |
| inhibitor | netarsudil, belumosudil, verosudil, or |
| | thiazovivin |

In some embodiments, D1 or D2 is a radical of a compound represented by a structure:

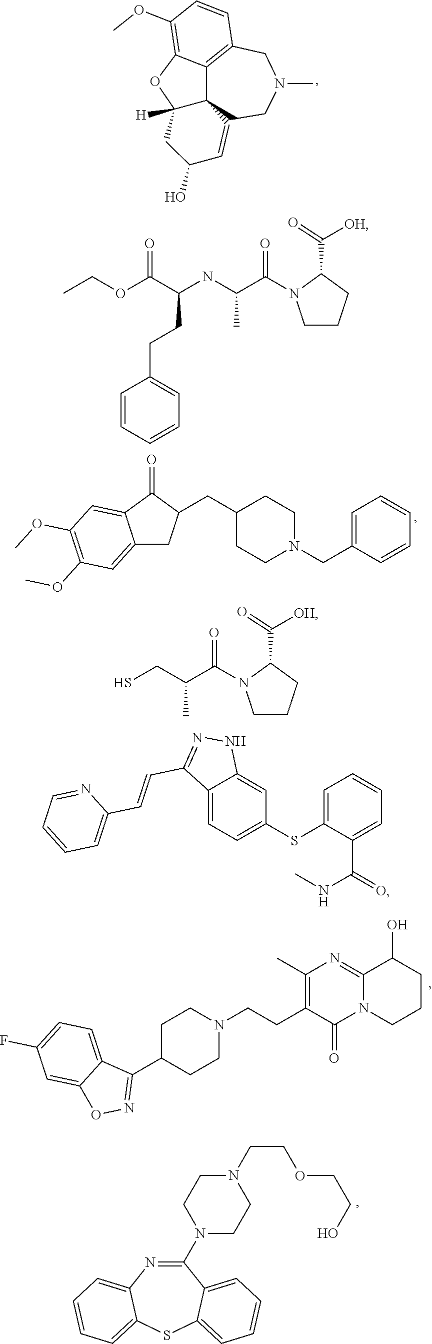

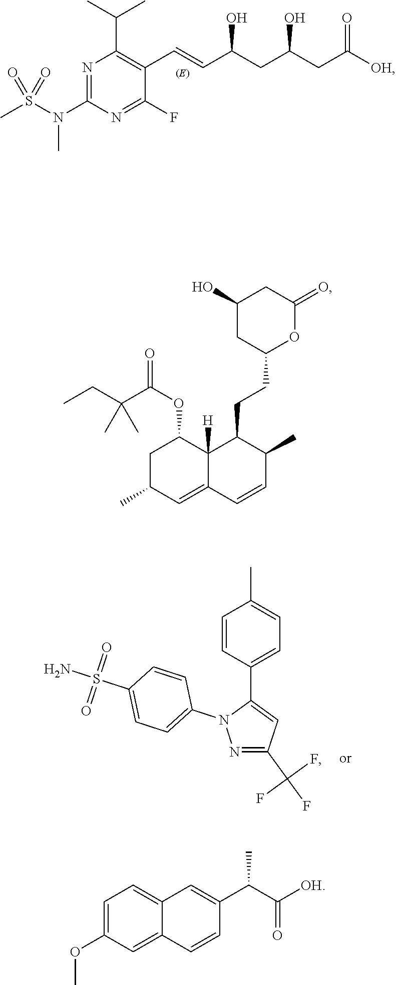

In some embodiments, the linker (e.g., L) comprises or is represented by a structure provided in Table 3. In some embodiments, the linker (e.g., L) comprises or is a diradical derived from a compound (e.g., compound name) provided in Table 3. The linkers in Table 3 are intended to provide examples of linkers described in the disclosure. Other linkers, such as described elsewhere here (a bond, C═O, etc.), are also contemplated.

TABLE 3

| Structure | Derived from [compound name] |
|---|---|
| 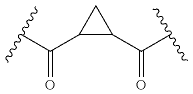 [SMILES: *C(=O)C1CC1C(*)=O] | cyclopropane-1,2-dicarboxylic acid |
| 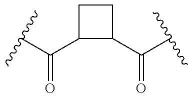 [SMILES: *C(=O)C1CCC1C(*)=O] | cyclobutane-1,2-dicarboxylic acid |
| 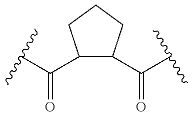 [SMILES: *C(=O)C1CCCC1C(*)=O] | cyclopentane-1,2-dicarboxylic acid |
| 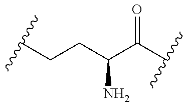 [SMILES: *CC[C@H](N)C(*)=O] | homoserine |
| 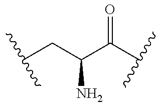 [SMILES: *C[C@H](N)C(*)=O] | serine |
| 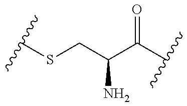 [SMILES: *SC[C@H](N)C(*)=O] | cysteine |
| 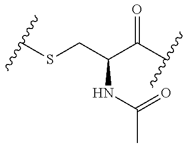 [SMILES: *SC[C@H](NC(C)=O)C(*)=O] | N-acetyl cysteine |
| 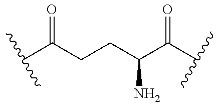 [SMILES: *C(=O)CC[C@H](N)C(*)=O] | glutamic acid |
| 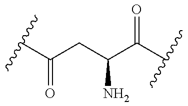 [SMILES: *C(=O)C[C@H](N)C(*)=O] | aspartic acid |
| 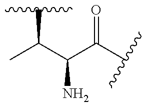 [SMILES: *C(=O)[C@@H](N)[C@H](*)C] | threonine |
| 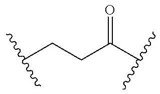 [SMILES: *CCC(*)=O] | 3-hydroxypropanoic acid |
| 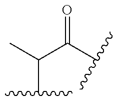 [SMILES: *C(=O)C(*)C] | lactic acid |
| 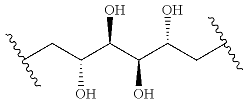 [SMILES: *C[C@@H](O)[C@@H](O)[C@H](O)[C@H](O)C*] | mannitol |
| 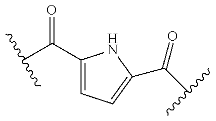 [SMILES: *C(=O)c1ccc(C(*)=O)[nH]1] | 2,5-pyrroledicarboxylic acid |
| 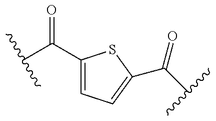 [SMILES: *C(=O)c1ccc(C(*)=O)s1] | 2,5-Thiophenedicarboxylic acid |
| 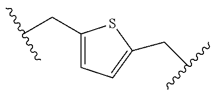 [SMILES: *Cc1ccc(C*)s1] | thiophene-2,5-diyldimethanol |
| 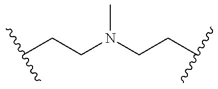 [SMILES: *CCN(C)CC*] | N-Methyldiethanolamine |
| 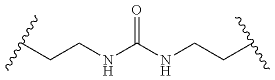 [SMILES: *CCNC(=O)NCC*] | 1,3-Bis(2-hydroxyethyl)urea |
| 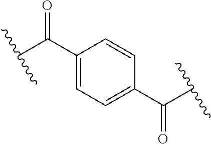 [SMILES: *C(=O)c1ccc(C(*)=O)cc1] | terephthalic acid |
| 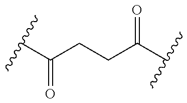 [SMILES: *C(=O)CCC(*)=O] | succinic acid |
| 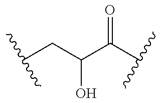 [SMILES: *CC(O)C(*)=O] | Glyceric acid |
| 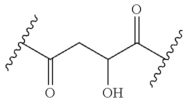 [SMILES: *C(=O)CC(O)C(*)=O] | Malic acid |
| 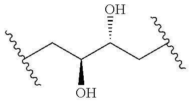 [SMILES: *C[C@@H](O)[C@@H](O)C*] | Erythritol |
| 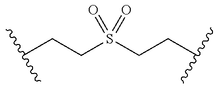 [SMILES: *CCS(=O)(=O)CC*] | BIS(2-HYDROXYETHYL)SULFONE |
| 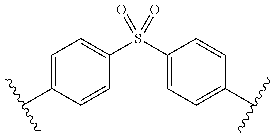 [SMILES: *c1ccc(S(=O)(=O)c2ccc(*)cc2)cc1] | 4,4′-Sulfonyldiphenol |
| 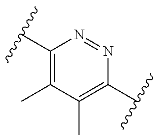 [SMILES: *c1nnc(*)c(C)c1C] | 4,5-Dimethylpyridazine-3,6-diol |
| 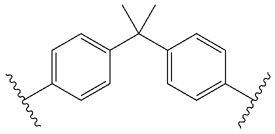 [SMILES: *c1ccc(C(C)(C)c2ccc(*)cc2)cc1] | Bisphenol A |
| 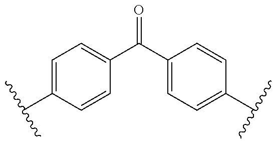 [SMILES: *c1ccc(C(=O)c2ccc(*)cc2)cc1] | 4,4′-Dihydroxybenzophenone |
| 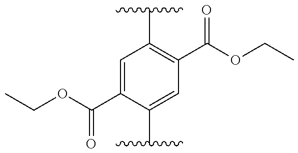 [SMILES: *c1cc(C(=O)OCC)c(*)cc1C(=O)OCC] | Diethyl 2,5-dihydroxyterephthalate |
| 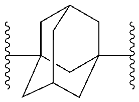 [SMILES: *C12CC3CC(C1)CC(*)(C3)C2] | 1,3-Adamantanediol |
| 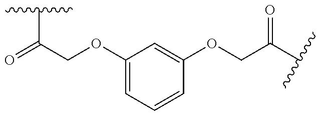 [SMILES: *C(=O)COc1cccc(OCC(*)=O)c1] | Resorcinol-o,o′-diacetic acid |
| 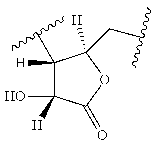 [SMILES: *C[C@H]1OC(=O)[C@H](O)[C@@H]1*] | D-(+)-Ribonic gamma-lactone |
| 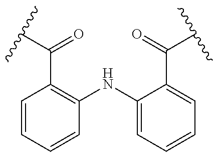 [SMILES: *C(=O)c1ccccc1Nc1ccccc1C(*)=O] | 2,2′-Iminodibenzoic acid |
| 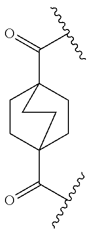 [SMILES: *C(=O)C12CCC(C(*)=O)(CC1)CC2] | Bicyclo[2.2.2]octane-1,4-dicarboxylic acid |
| 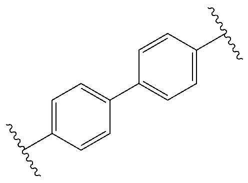 [SMILES: *c1ccc(-c2ccc(*)cc2)cc1] | 4,4′-Dihydroxybiphenyl |
| 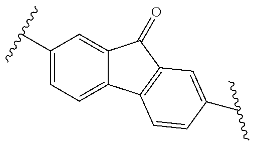 [SMILES: *c1ccc2c(c1)C(=O)c1cc(*)ccc1-2] | 2,7-Dihydroxy-9-fluorenone |
| 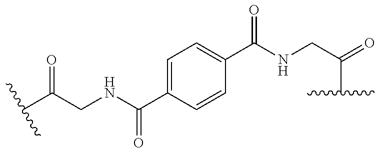 [SMILES: *C(=O)CNC(=O)c1ccc(C(=O)NCC(*)=O)cc1] | 2-[[4- (Carboxymethylcarbamoyl)benzoyl]amino] acetic acid |
| 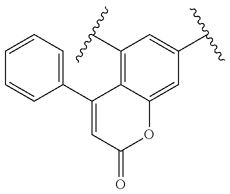 [SMILES: *c1cc(*)c2c(-c3ccccc3)cc(=O)oc2c1] | 5,7-Dihydroxy-4-phenylcoumarin |
| 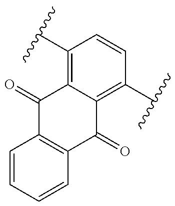 [SMILES: *c1ccc(*)c2c1C(=O)c1ccccc1C2=O] | Quinizarin |
| 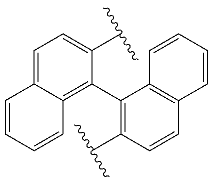 [SMILES: *c1ccc2ccccc2c1-c1c(*)ccc2ccccc12] | 1,1′-Bi-2-naphthol |
| 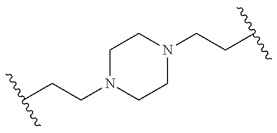 [SMILES: *CCN1CCN(CC*)CC1] | 1,4-Bis(2-hydroxyethyl)piperazine |
| 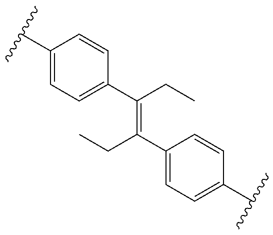 [SMILES: *c1ccc(/C(CC)=C(\CC)c2ccc(*)cc2)cc1] | Diethylstilbestrol, mixture of cis and trans |
| 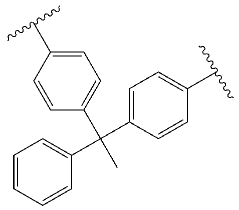 [SMILES: *c1ccc(C(C)(c2ccccc2)c2ccc(*)cc2)cc1] | 4,4′-(1-Phenylethylidene)bisphenol |
| 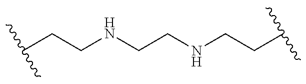 [SMILES: *CCNCCNCC*] | N,N′-Bis(2-hydroxyethyl)ethylenediamine |
| 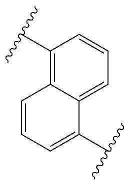 [SMILES: *c1cccc2c(*)cccc12] | 1,5-Dihydroxynaphthalene |
| 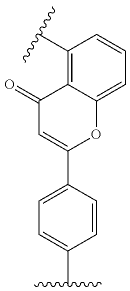 [SMILES: *c1ccc(-c2cc(=O)c3c(*)cccc3o2)cc1] | 4′,5-Dihydroxyflavone |
| 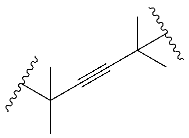 [SMILES: *C(C)(C)C#CC(*)(C)C] | 2,5-Dimethyl-3-hexyne-2,5-diol |
| 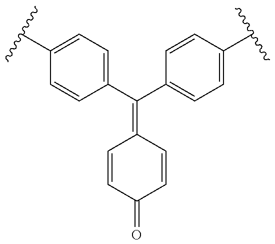 [SMILES: *c1ccc(C(=C2C=CC(=O)C=C2)c2ccc(*)cc2)cc1] | p-Rosolic acid |
| 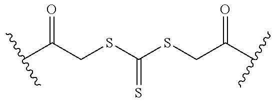 [SMILES: *C(=O)CSC(=S)SCC(*)=O] | Bis(carboxymethyl)trithiocarbonate |
| 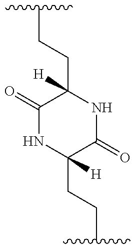 [SMILES: *CC[C@@H]1NC(=O)[C@H](CC*)NC1=O] | cis-3,6-Bis(2-hydroxyethyl)piperazine-2,5- dione |
| 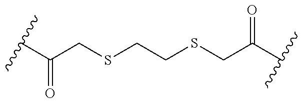 [SMILES: *C(=O)CSCCSCC(*)=O] | 2,2′-(Ethylenedithio)diacetic acid |
| 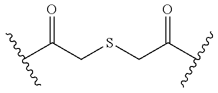 [SMILES: *C(=O)CSCC(*)=O] | 2,2′-Thiodiacetic acid |
| 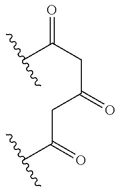 [SMILES: *C(=O)CC(=O)CC(*)=O] | 1,3-Acetonedicarboxylic acid |
| 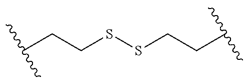 [SMILES: *CCSSCC*] | 2-Hydroxyethyl disulfide |

In some embodiments, D1 is a radical of any compound provided in Table 1 or Table 2, D2 is a radical of any compound provided in Table 1), and the linker (e.g., L) is or comprises one or more linker groups, each linker group being independently selected from any linker or linker group provided in Table 3).

In some embodiments, D1 is an angiostatic steroid (e.g., anecortave).

In some embodiments, D1 is a benign steroid (e.g., cholesterol).

In some embodiments, D1 is a corticosteroid (e.g., glucocorticoid or mineralcorticoid), a sex steroid, a neurosteroid, an aminosteroid, or a secosteroid. In some embodiments, D1 is a corticosteroid.

In some embodiments, D1 is dexamethasone.

In some embodiments, D1 is hydrocortisone.

In some embodiments, D1 is anecortave (e.g., anecortave desacetate).

In some embodiments, D2 is a prostaglandin. In some embodiments, D2 is an lOP lowering prostaglandin. In some embodiments, D2 is latanoprost, latanoprost acid, travoprost, travoprost acid, tafluprost, tafluprost acid, bimatoprost, or bimatoprost acid

In some embodiments, L is a hydrolyzable linker.

In some embodiments, L comprises one or more linker group, each linker group being independently selected from the group consisting of a bond, substituted or unsubstituted alkyl, substituted or unsubstituted heteroalkyl, substituted or unsubstituted alkoxy, substituted or unsubstituted aryl, or L is substituted or unsubstituted heteroaryl. In some embodiments, L comprises one or more linker group, each linker group being independently selected from the group consisting of substituted or unsubstituted alkyl, substituted or unsubstituted heteroalkyl, substituted or unsubstituted alkoxy, substituted or unsubstituted aryl, or L is substituted or unsubstituted heteroaryl. In some embodiments, L is substituted or unsubstituted alkyl, substituted or unsubstituted heteroalkyl, substituted or unsubstituted alkoxy, or substituted or unsubstituted aryl. In some embodiments, L is a bond. In some embodiments, L is substituted or unsubstituted alkyl. In some embodiments, Lis substituted or unsubstituted heteroalkyl. In some embodiments, L is substituted or unsubstituted alkoxy. In some embodiments, L is substituted or unsubstituted aryl. In some embodiments, L is substituted or unsubstituted heteroaryl.

In some embodiments, the linker (e.g., L, L¹, or L²) is a bond.

In some embodiments, the linker (e.g., L, L¹, or L²) is not a bond.

In some embodiments, the linker (e.g., L, L¹, or L²) is attached to any hydroxyl group of any agent (e.g., D1 or D2) provided herein, such as a hydroxyl, a carboxylate, a phosphate, or an enolizable ketone of any agent (e.g., D1 or D2) provided herein.

In some embodiments, the linker (e.g., L, L¹, or L²) is attached to any thiol or hydroxyl group of any agent (e.g., D1 or D2) provided herein, such as a thiol, a hydroxyl, or a carboxylate of any agent (e.g., D1 or D2) provided herein.

In some embodiments, the linker (e.g., L, L¹, or L²) is attached to hydroxyl group of any agent (e.g., D1 or D2) provided herein, such as a hydroxyl of any agent (e.g., D1 or D2) provided herein.

In some instances, the carboxylate (radical) of an agent (e.g., a radical of an agent) provided herein is a carboxylate (radical) of D1 or D2, such that the point of attachment of D1 or D2 to the linker (e.g., L, L¹, or L²) is through a carboxylate (radical).

In some instances, the hydroxyl (radical) of an agent (e.g., a radical of an agent) provided herein is a hydroxyl (radical) of D1 or D2, such that the point of attachment of D1 or D2 to the linker (e.g., L, L¹, or L²) is through a hydroxyl (radical).

In some embodiments, the linker (e.g., L, L¹, or L²) is attached to any thiol group of any agent (e.g., D1 or D2) provided herein.

In some instances, the thiol (radical) of an agent (e.g., a radical of an agent) provided herein is a thiol (radical) of D1 or D2, such that the point of attachment of D1 or D2 to the linker (e.g., L, L¹, or L²) is through a thiol (radical).

In some embodiments, either or both of D1 or D2 are attached to L or L¹ through a hydroxyl radical of the D1 or D2.

In some embodiments, D1 and D2 are attached to L or L¹ through a hydroxyl radical of the D1 or D2.

In some embodiments, D1 and D2 are attached to L through a hydroxyl radical of the D1 or D2.

In some embodiments, D1 and D2 are attached to L¹ through a hydroxyl radical of the D1 or D2.

Unless stated specifically otherwise herein, each instance of radical indicates that a hydrogen (i.e., a hydrogen radical (H●)) is removed from a free form of a compound provided herein, such as any agent described herein (steroid, prostaglandin, etc.). In some instances, the radical is a hydroxyl radical. In some instances, the removal of the hydrogen radical from the compound provided herein, such as any agent (steroid, prostaglandin, etc.) described herein, provides a radical of an agent (steroid, prostaglandin, etc.) that is taken together with any point of a linker provided herein (e.g., L, L¹, or L²) to form a bond (e.g., between the linker and the radical of the agent).

In some instances, the removal of the hydrogen radical from the compound provided herein, such as any steroid described herein, provides a radical of a steroid that is taken together with any point of a linker provided herein (e.g., L, L¹, or L²) to form a bond (e.g., between the linker and the steroid radical).

In some instances, the removal of the hydrogen radical from the compound provided herein, such as any prostaglandin described herein, provides a radical of a prostaglandin that is taken together with any point of a linker provided herein (e.g., L, L¹, or L²) to form a bond (e.g., between the linker and the prostaglandin radical).

Provided in some embodiments herein is a compound having a structure represented by Formula (ID): D1-L-D2. In some embodiments, D1 is a steroid radical. In some embodiments, D2 is a prostaglandin radical. In some embodiments, L is -(Q¹-M-Q²)-. In some embodiments, Q¹ and Q² are each independently absent or (C═X¹)X². In some embodiments, X¹ is O or S. In some embodiments, X² is O, S, or NR¹. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl). In some embodiments, the compound is a pharmaceutically-acceptable salt or solvate.

Provided in some embodiments herein is a compound having a structure represented by Formula (IV): D1-L²-D2. In some embodiments, D1 is a steroid radical. In some embodiments, D2 is a substituted prostaglandin radical. In some embodiments, L² is any linker provided herein. In some embodiments, L² is a bond.

In some embodiments, D1 is an angiostatic steroid (e.g., anecortave).

In some embodiments, D1 is a benign steroid (e.g., cholesterol).

In some embodiments, D1 is a corticosteroid (e.g., glucocorticoid or mineralcorticoid), a sex steroid, a neurosteroid, an aminosteroid, or a secosteroid. In some embodiments, D1 is a corticosteroid.

In some embodiments, D1 is anecortave (e.g., anecortave desacetate).

In some embodiments, D2 is an IOP lowering prostaglandin.

In some embodiments, L is a hydrolyzable linker.

In some embodiments, L comprises one or more linker group, each linker group being independently selected from the group consisting of a bond, substituted or unsubstituted alkyl, substituted or unsubstituted heteroalkyl, substituted or unsubstituted alkoxy, substituted or unsubstituted aryl, or L is substituted or unsubstituted heteroaryl. In some embodiments, L comprises one or more linker group, each linker group being independently selected from the group consisting of substituted or unsubstituted alkyl, substituted or unsubstituted heteroalkyl, substituted or unsubstituted alkoxy, substituted or unsubstituted aryl, or L is substituted or unsubstituted heteroaryl. In some embodiments, L is substituted or unsubstituted alkyl, substituted or unsubstituted heteroalkyl, substituted or unsubstituted alkoxy, or substituted or unsubstituted aryl. In some embodiments, L is a bond. In some embodiments, L is substituted or unsubstituted alkyl. In some embodiments, Lis substituted or unsubstituted heteroalkyl. In some embodiments, L is substituted or unsubstituted alkoxy. In some embodiments, L is substituted or unsubstituted aryl. In some embodiments, L is substituted or unsubstituted heteroaryl.

In some embodiments, the linker (e.g., L, L¹, or L²) is a bond.

In some embodiments, the linker (e.g., L, L¹, or L²) is not a bond.

In some embodiments, the linker (e.g., L, L¹, or L²) is attached to any hydroxyl group of any steroid provided herein, such as a hydroxyl (e.g., at the C11-, C17-, C21-position), a carboxylate, a phosphate, or an enolizable ketone (e.g., at the C1-position) of any steroid provided herein.

In some embodiments, the linker (e.g., L, L¹, or L²) is attached to any thiol or hydroxyl group of any prostaglandin provided herein, such as a thiol, a hydroxyl (e.g., at the C1-, C9-, C11-, C15-position), or a carboxylate of any prostaglandin provided herein.

In some embodiments, the linker (e.g., L, L¹, or L²) is attached to hydroxyl group of any prostaglandin provided herein, such as a hydroxyl (e.g., at the C1-, C9-, C11-, C15-position) of any prostaglandin provided herein.

In some instances, the C1-position of a prostaglandin (radical) provided herein is a carboxylic acid (radical). In some instances, the carboxylate group of a prostaglandin (radical) provided herein is a carboxylate (radical) of D2, such that the point of attachment of D2 to the linker (e.g., L, L¹, or L²) is through a carboxylate (radical).

In some instances, the C1-position of a prostaglandin (radical) provided herein is methyl substituted with oxo and hydroxyl (radical). In some instances, the hydroxyl group of a prostaglandin (radical) provided herein is a hydroxyl (radical) of D2, such that the point of attachment of D2 to the linker (e.g., L, L¹, or L²) is through a hydroxyl (radical).

In some embodiments, the linker (e.g., L, L¹, or L²) is attached to any thiol group of any prostaglandin provided herein.

In some instances, the thiol group of a prostaglandin (radical) provided herein is a thiol (radical) of D2, such that the point of attachment of D2 to the linker (e.g., L, L¹, or L²) is through a thiol (radical).

In some embodiments, either or both of D1 or D2 are attached to L or L¹ through a hydroxyl radical of the D1 or D2.

In some embodiments, D1 and D2 are attached to L or L¹ through a hydroxyl radical of the D1 or D2.

In some embodiments, D1 and D2 are attached to L through a hydroxyl radical of the D1 or D2.

In some embodiments, D1 and D2 are attached to L¹ through a hydroxyl radical of the D1 or D2.

Provided in certain embodiments herein is a compound comprising a first radical and a second radical, the first radical comprising the structure represented by Formula (I′):

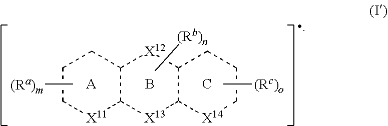

In certain embodiments, [symbol] is a single bond or a double bond. In some embodiments, each R^{a}, R^{b}, and R^{c} is independently selected from the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted. In certain embodiments, any one of R^{a}, R^{b}, or R^{c} are taken together with another of R^{a}, R^{b}, or R^{c} to form a substituted or an unsubstituted cycloalkyl or heterocycloalkyl. In some embodiments, X¹¹, X¹², X¹³, and X¹⁴ are each independently selected from the group consisting of a bond and Q_{y}, wherein each Q is independently selected from the group consisting of —O—, —NR—, —S(R)ₓ—, and —C(R)₂—. In some embodiments, y is 1-3. In certain embodiments, each x is independently 0-5. In some embodiments, each z is independently 1 or 2 (e.g., depending on degree of saturation). In certain embodiments, each of m, n, and o are independently 0-6. In certain embodiments, each R is independently selected from the group consisting of hydrogen, halogen, alkyl, heteroalkyl, cycloalkyl, heterocycloalkyl, alkoxy, amino (e.g., dihydroamino, alkylamino, or arylamino), hydroxy, and thiol (e.g., wherein the alkyl, heteroalkyl, cycloalkyl, or heterocycloalkyl is optionally substituted), or each R is taken together with another R to form an oxo. In some embodiments, the second radical is a therapeutically active agent (or drug). In certain embodiments, the first radical (e.g., steroid) is different than the second radical (e.g., prostaglandin). In some embodiments, either the first radical, the second radical, or both the first radical and the second radical is not a steroid. Also provided in certain embodiments herein are pharmaceutically-acceptable salts or solvates of a compound of Formula (ID).

In some embodiments, X¹¹ is Q¹. In some embodiments, X¹² is a bond. In some embodiments, X¹³ is Q₂. In some embodiments, X¹⁴ is Q¹. In some embodiments, X¹¹ and X¹⁴ are each Q¹. In some embodiments, X¹² is a bond and X¹³ is Q². In some embodiments, Q is —C(R)₁— or —C(R)₂—. In some embodiments, X¹¹ and X¹⁴ are each independently —C(R)₁— or —C(R)₂—. In some embodiments, X¹² is a bond and X¹³ is —C(R)₂C(R)₂—, —C(R)C(R)₂—, or —C(R)C(R)—. In some embodiments, X¹¹ and X¹⁴ are each —C(R)₂—, and X¹² is a bond and X¹³ is —C(R)₂C(R)₂— or —C(R)C(R)₂—.

In some embodiments, each R is independently hydrogen, halogen, alkyl, heteroalkyl, hydroxy, amino (e.g., dihydroamino, alkylamino, or arylamino), or taken together with another R to form an oxo. In some embodiments, each R is independently hydrogen, halogen, alkyl, hydroxy, or taken together with another R to form an oxo. In some embodiments, each R is independently hydrogen or halogen. In some embodiments, each R is independently hydrogen or alkyl. In some embodiments, each R is independently hydrogen or hydroxy. In some embodiments, each R is independently hydrogen or taken together with another R to form an oxo.

In some embodiments, the alkyl, heteroalkyl, cycloalkyl, heterocycloalkyl, alkoxy, amino (e.g., dihydroamino, alkylamino, or arylamino), hydroxy, or thiol of R is optionally substituted. In some embodiments, R combines with one of R^{a}, R^{b}, or R^{c} to form an optionally substituted cycloalkyl or an optionally substituted heterocycloalkyl. In some embodiments, R is amino and combines with one of R^{a}, R^{b}, or R^{c} to form an optionally substituted heterocycloalkyl. In some embodiments, R is amino and combines with one of R^{a} or R^{c} to form a heterocycloalkyl substituted with optionally substituted alkyl.

In some embodiments, provided herein is a compound comprising a first radical and a second radical, the first radical comprising a structure of Formula (IA):

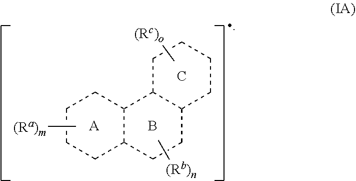

In some embodiments, [symbol] is a single bond or a double bond). In some embodiments, each R^{a}, R^{b}, and R^{c} are independently selected from the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted. In some embodiments, each of m, n, and o are independently 0-6. In some embodiments, any one of R^{a}, R^{b}, or R^{c} are taken together with another of R^{a}, R^{b}, or R^{c} to form a substituted or an unsubstituted cycloalkyl or heterocycloalkyl. In some embodiments, the second radical is a therapeutically active agent (or drug) and the first radical (e.g., steroid) being different than the second radical (e.g., prostaglandin). In some embodiments, either the first radical, the second radical, or both the first radical and the second radical is not a steroid. Also provided in certain embodiments herein are pharmaceutical salts or solvates of a compound of Formula (IA).

In some embodiments, provided herein is a compound comprising a first radical and a second radical, the first radical comprising a structure of Formula (IA):

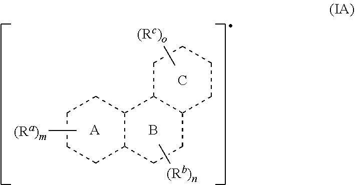

wherein:
   [symbol] is a single bond or a double bond;
   each R^{a}, R^{b}, and R^{c} are independently selected the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted;
      or any one of R², R^{b}, or R^{c} are taken together with another of R^{a}, R^{b}, or R^{c} to form a substituted or an unsubstituted cycloalkyl or heterocycloalkyl;
   each of m, n, and o are independently 0-6;
the second radical is a therapeutically active agent (or drug) and the first radical (e.g., steroid) being different than the second radical (e.g., prostaglandin);
the first radical and the second radical being attached to a linker (e.g., that links the first radical and the second radical);
the first radical and the second radical being attached to a linker (e.g., that links the first radical and the second radical);
wherein, either the first radical, the second radical, or both the first radical and the second radical is not a steroid,
or a pharmaceutically-acceptable salt or solvate thereof.

In some embodiments, both the first radical and the second radical have a structure of Formula (I′) or Formula (IA). In some embodiments, the first radical has a structure of Formula (I′) or Formula (IA) and the second radical does not have a structure of Formula (I′) or Formula (IA). In some embodiments, the structure of Formula (I′) or Formula (IA) has a melt and/or glass transition temperature at a temperature of at least 20° C. (e.g., at least 25° C., at least 30° C., at least 37° C., at least 40° C., at least 50° C., at least 100° C., or more) in its free form.

In some embodiments, n is 4. In some embodiments, n is 3. In some embodiments, n is 2. In some embodiments, n is 1. In some embodiments, each R^{b} is independently hydrogen, halogen, alkyl, heteroalkyl, hydroxy, amino (e.g., dihydroamino, alkylamino, or arylamino), or taken together with another R^{b} to form an oxo. In some embodiments, each R^{b} is independently hydrogen, halogen, alkyl, hydroxy, or taken together with another R^{b} to form an oxo. In some embodiments, each R^{b} is independently hydrogen or halogen. In some embodiments, each R^{b} is independently hydrogen or alkyl. In some embodiments, each R^{b} is independently hydrogen or hydroxy. In some embodiments, each R^{b} is independently hydrogen or taken together with another R^{b} to form an oxo.

In some embodiments, the alkyl, heteroalkyl, cycloalkyl, heterocycloalkyl, alkoxy, amino (e.g., dihydroamino, alkylamino, or arylamino), hydroxy, or thiol of R^{b} is optionally substituted. In some embodiments, R^{b} combines with one of R^{a}, R^{b}, or R^{c} to form an optionally substituted cycloalkyl or an optionally substituted heterocycloalkyl. In some embodiments, R^{b} is amino and combines with one of R^{a} or R^{c} to form an optionally substituted heterocycloalkyl. In some embodiments, R^{b} is amino and combines with one of R^{a} or R^{c} to form a heterocycloalkyl substituted with optionally substituted alkyl.

In some embodiments, one of R^{c} is taken together with another R^{c} to form an optionally substituted cycloalkyl or an optionally substituted heterocycloalkyl. In some embodiments, one of R^{c} is taken together with another R^{c} to form an optionally substituted cycloalkyl. In some embodiments, one of R^{c} is taken together with another R^{c} to form a cycloalkyl substituted with one or more substituent, each substituent selected from the group consisting of oxo, halogen, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, alkoxy, amino, thiol, or heterocycloalkyl is optionally substituted.

In some embodiments, R^{a}, R^{b}, and R^{c} (e.g., of Formula (I′) or Formula (IA)) are each optionally and independently substituted with one or more groups, each group independently selected from —OH, oxo, alkyl (e.g., alkenyl), heteroalkyl, cycloalkyl, or alkoxy, wherein the alkyl, heteroalkyl, cycloalkyl, or alkoxy, is further optionally substituted. In certain embodiments, the alkyl, heteroalkyl, cycloalkyl, or heterocycloalkyl of each R^{a}, R^{b}, or R^{c} (e.g., of Formula (I′) or Formula (IA)) is, independently, substituted or not substituted. In some embodiments, each group is independently not substituted or substituted with any one or more substituent described herein. In specific embodiments, each group is independently not substituted or substituted with one or more substituent, wherein each substituent is selected from the group consisting of —OH, oxo, alkyl, heteroalkyl, cycloalkyl, or alkoxy, wherein the alkyl, heteroalkyl, cycloalkyl, or alkoxy, is further optionally substituted.

In some embodiments, the substituted or unsubstituted cycloalkyl or heterocycloalkyl (e.g., of Formula (I′) or Formula (IA)) are each optionally and independently substituted with one or more groups, each group independently selected from —OH, oxo, alkyl (e.g., alkenyl, alkynyl), —S-alkyl, —NH-alkyl, halogen, heteroalkyl, cycloalkyl, or alkoxy, wherein the alkyl (e.g., —S-alkyl, —NH-alkyl), heteroalkyl, cycloalkyl, or alkoxy, is further optionally substituted. In certain embodiments, substituted or unsubstituted cycloalkyl or heterocycloalkyl are, independently, substituted or not substituted. In some embodiments, each group is independently not substituted or substituted with any one or more substituent described herein. In specific embodiments, each group is independently not substituted or substituted with one or more substituent, wherein each substituent is selected from the group consisting of —OH, oxo, alkyl (e.g., alkenyl, alkynyl), —S-alkyl, —NH-alkyl, halogen, heteroalkyl, cycloalkyl, oralkoxy, wherein the alkyl (e.g., —S-alkyl, —NH-alkyl), heteroalkyl, cycloalkyl, or alkoxy, is further optionally substituted. In some embodiments, the cycloalkyl (e.g., of Formula (I′) or Formula (IA)) is substituted with oxo, —OH, optionally substituted alkyl, or optionally substituted alkoxy. In some embodiments, the alkyl is substituted with one or more halogen, oxo, —OH, alkyl (e.g., alkenyl), —S-alkyl, —NH— alkyl, alkoxy, wherein the alkyl (e.g., —S-alkyl, —NH-alkyl) or alkoxy is further optionally substituted. In some embodiments, the alkyl is methyl.

Provided in some embodiments here is a compound comprising a first radical and a second radical, the first radical comprising a structure of Formula (IF):

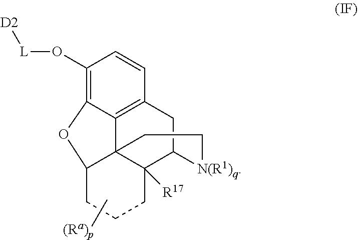

In some embodiments, [symbol] is a single bond or a double bond. In some embodiments, each R^{a} is independently selected the group consisting of hydrogen, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted. In some embodiments, or a first R^{a} is taken together with another R^{a} to form oxo, a substituted or an unsubstituted cycloalkyl or heterocycloalkyl. In some embodiments, each R¹ is independently H, alkyl, heteroalkyl, cycloalkyl, or heterocycloalkyl, wherein the alkyl, heteroalkyl, cycloalkyl, or heterocycloalkyl are optionally substituted. In some embodiments, R¹⁷ is H or —OH. In some embodiments, p is 0-8. In some embodiments, q is 1 or 2. In some embodiments, D2 is a radical of an active agent (e.g., a therapeutically active agent) (or drug). In some embodiments, L is -(Q¹-M-Q²)-. In some embodiments, Q¹ and Q² are each independently absent or (C═X¹)X². In some embodiments, X¹ is O or S. In some embodiments, X² is O, S, or NR¹. In some embodiments, R¹ is hydrogen or C₁-C₆ alkyl. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl). In some embodiments, wherein D2 is not an opioid. In some embodiments, the compound is a pharmaceutically acceptable salt or solvate.

In some embodiments, R¹ is substituted alkyl.

In some embodiments, R¹⁷ is hydroxyl.

In some embodiments, R^{a} is hydrogen.

In some embodiments, provided herein is a compound comprising a first radical and a second radical, the first radical comprising a structure of Formula (IB):

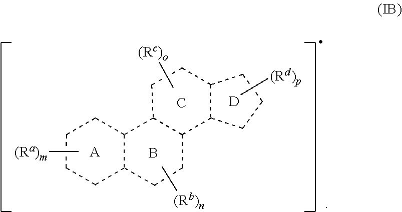

In some embodiments, [symbol] is a single bond or a double bond). In some embodiments, each R^{a}, R^{b}, R^{c}, and R^{d} are independently selected from the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted. In some embodiments, each of m, n, o, and p are independently 0-6. In some embodiments, any one of R^{a}, R^{b}, R^{c}, and R^{d} are taken together with another of R^{a}, R^{b}, R^{c}, and R^{d} to form a substituted or an unsubstituted cycloalkyl or heterocycloalkyl. In some embodiments, the second radical is a therapeutically active agent (or drug) and the first radical (e.g., steroid) being different than the second radical (e.g., prostaglandin). In some embodiments, either the first radical, the second radical, or both the first radical and the second radical is not a steroid. Also provided in certain embodiments herein are pharmaceutical salts or solvates of a compound of Formula (IB).

In some embodiments, Ring B of any one of Formula (I′), Formula (IA), or Formula (IB) is an optionally substituted cycloalkyl. In some embodiments, Ring B of any one of Formula (I′), Formula (IA), or Formula (IB) does not comprise a heteroatom within the ring (e.g., Ring B is optionally substituted cycloalkyl). In some embodiments, Ring B of any one of Formula (I′), Formula (IA), or Formula (IB) comprises only single bonds. In some embodiments, Ring B of any one of Formula (I′), Formula (IA), or Formula (IB) comprises at least one double bond. In some embodiments, Ring B of any one of Formula (I′), Formula (IA), or Formula (IB) is attached to at least one ring (e.g., Ring A and/or Ring C) that comprises at least one double bond. In some embodiments, Ring A comprises at least one double bond. In some embodiments, Ring C comprises at least one double bond. In some embodiments, Ring A and Ring C each independently comprise at least one double bond. In some embodiments, Ring B of any one of Formula (I′), Formula (IA), or Formula (IB) is attached to at least one ring (e.g., Ring A and or Ring C) that is aromatic. In some embodiments, Ring B of any one of Formula (I′) or Formula (IA) is aromatic. In some embodiments, Ring A, Ring B, and Ring C of any one of Formula (I′) or Formula (IA) are each aromatic.

In some embodiments, m is 4. In some embodiments, m is 3. In some embodiments, m is 2. In some embodiments, m is 1. In some embodiments, n is 3. In some embodiments, n is 2. In some embodiments, n is 1. In some embodiments, n is 0. In some embodiments, o is 5. In some embodiments, o is 4. In some embodiments, o is 3. In some embodiments, o is 2. In some embodiments, o is 1. In some embodiments, p is 3. In some embodiments, p is 2. In some embodiments, p is 1.

In some embodiments, each R^{a}, R^{b}, R^{c}, and R^{d} are independently selected from the group consisting of oxo, halogen, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, alkoxy, amino (e.g., dihydroamino, alkylamino, or arylamino), hydroxy, thiol, or heterocycloalkyl is optionally substituted. In some embodiments, each R^{a} is independently selected from —OH, oxo, halogen, alkyl, or alkoxy, wherein the alkyl or alkoxy is optionally substituted. In some embodiments, each R^{b} is independently selected from —OH, oxo, halogen, or optionally substituted alkyl. In some embodiments, each R^{c} is independently selected from —OH, oxo, or optionally substituted alkyl. In some embodiments, each R^{d} is independently selected from —OH, oxo, alkyl (e.g., alkenyl or alkynyl), heteroalkyl, or each R^{d} is taken together to form an oxo, wherein the alkyl or heteroalkyl is optionally substituted. In some embodiments, the substituted alkyl of R^{d} is —COOH, —(C═O)alkyl, —(C═O)Oalkyl, —O(C═O)Oalkyl, —(C═O)Salkyl, wherein the alkyl is optionally substituted with —OH or halogen. In some embodiments, one R^{d} is taken together with another R^{d} to form a substituted or unsubstituted cycloalkyl or heterocycloalkyl.

In some embodiments, the alkyl of any one of R^{a}, R^{b}, R^{c}, or R^{d} is C₁-C₃ alkyl. In some embodiments, the alkyl of any one of R^{a}, R^{b}, R^{c}, or R^{d} is substituted with oxo and further optionally substituted with alkyl, hydroxy, halogen, heteroalkyl, alkoxy, thioether, wherein the alkyl, alkoxy, thioether, or heteroalkyl is further optionally substituted. In some embodiments, the alkoxy of any one of R^{a}, R^{b}, R^{c}, or R^{d} is C₁-C₃ alkoxy.

In some embodiments, Ring A is aromatic. In some embodiments, Ring A comprises at least one double bond. In some embodiments, Ring A comprises one double bond. In some embodiments, Ring A comprises two double bonds. In some embodiments, Ring B comprises at least one double bond. In some embodiments, Ring B comprises one double bond. In some embodiments, Ring C comprises one double bond. In some embodiments, Ring D comprises one double bond. In some embodiments, Ring A comprises at least one double bond and each of Ring B, Ring C, and Ring D consist of single bonds. In some embodiments, Ring A is aromatic and each of Ring B, Ring C, and Ring D consist of single bonds. In some embodiments, Ring A comprises at least one double bond and at least one of Ring B, Ring C, or Ring D comprises a double bond. In some embodiments, Ring A is aromatic and at least one of Ring B, Ring C, or Ring D comprises a double bond. In some embodiments, Ring A comprises at least one double bond and Ring B comprises a double bond. In some embodiments, Ring A comprises at least one double bond and Ring C comprises a double bond. In some embodiments, Ring A comprises at least one double bond and Ring D comprises a double bond.

In some embodiments, provided herein is a compound comprising a first radical and a second radical, the first radical comprising a structure of Formula (IC):

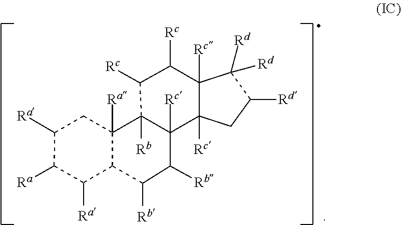

In some embodiments, [symbol] is a single bond or a double bond. In some embodiments, R^{a} is hydrogen, —OH, or oxo. In some embodiments, each R^{a′} is independently selected from hydrogen, —OH, halogen, C₁-C₃ alkyl, and alkoxy. In some embodiments, R^{a″} is absent, hydrogen, or C₁-C₃ alkyl. In some embodiments, R^{b} is absent, hydrogen, halogen, or C₁-C₃ alkyl. In some embodiments, R^{b′} is hydrogen, halogen, —OH, oxo, or C₁-C₃ alkyl. In some embodiments, R^{b″} is hydrogen or —OH. In some embodiments, each R^{c} is independently hydrogen, —OH, oxo, or C₁-C₃ alkyl. In some embodiments, each R^{c′} is independently hydrogen or C₁-C₃ alkyl. In some embodiments, R^{c″ is hydrogen, —OH, C}₁-C₃ alkyl, or —C(═O)H. In some embodiments, each R^{d} is independently hydrogen, —OH, —COOH, alkyl (e.g., alkylene, alkenyl, or alkynyl), heteroalkyl, or each R^{d} is taken together to form an oxo, wherein the alkyl or heteroalkyl is optionally substituted. In some embodiments, R^{d′} is hydrogen, —OH, C₁-C₃ alkyl (e.g., alkylene or alkenyl), or heteroalkyl. In some embodiments, one R^{d} is taken together with R^{d′} to form a substituted or unsubstituted cycloalkyl or heterocycloalkyl. In some embodiments, either the first radical, the second radical, or both the first radical and the second radical is not a steroid. In some embodiments, the second radical is a therapeutically active agent (or drug) and the first radical (e.g., steroid) being different than the second radical (e.g., prostaglandin). Also provided in certain embodiments herein are pharmaceutical salts or solvates of a compound of Formula (IC).

In some embodiments, the structure of any one of Formula (I′), Formula (IA), Formula (IB), or Formula (IC) consists of single bonds. In some embodiments, the structure of any one of Formula (I′), Formula (IA), Formula (IB), or Formula (IC) comprises at least one double bond. In some embodiments, the structure of any one of Formula (I′), Formula (IA), Formula (IB), or Formula (IC) comprises one double bond. In some embodiments, the structure of any one of Formula (I′), Formula (IA), Formula (IB), or Formula (IC) comprises two double bonds. In some embodiments, the structure of any one of Formula (I′), Formula (IA), Formula (IB), or Formula (IC) comprises three double bonds. In some embodiments, the structure of any one of Formula (I′), Formula (IA), Formula (IB), or Formula (IC) comprises at least one aromatic ring. In some embodiments, the structure of any one of Formula (I′), Formula (IA), Formula (IB), or Formula (IC) comprises one aromatic ring.

In some embodiments, R^{a} is —OH. In some embodiments, R^{a} is —OH and attached to a fully saturated cycloalkyl. In some embodiments, R^{a} is —OH and attached to an aryl. In some embodiments, R^{a} is oxo. In some embodiments, R^{a} is oxo and is adjacent to at least one double bond. In some embodiments, R^{a} is oxo and is adjacent to one double bond. In some embodiments, R^{a} is oxo and is adjacent to two double bonds.

In some embodiments, each R^{a′} is independently hydrogen or halogen (e.g., fluoro or chloro). In some embodiments, each R^{a′} is independently hydrogen or C₁-C₃ alkyl. In some embodiments, each R^{a′} is independently hydrogen or C₁-C₃ alkoxy. In some embodiments, each R^{a′} is attached to a single bond. In some embodiments, each R^{a′} is hydrogen. In some embodiments, at least one R^{a′} is attached to a double bond. In some embodiments, one R^{a′} is attached to a double bond. In some embodiments, each R^{a′} is attached to a double bond. In some embodiments, each R^{a′} is independently hydrogen or C₁-C₃ alkyl and attached to a single bond.

In some embodiments, each R^{a′} is independently hydrogen or halogen, and one R^{a′} is attached to a double bond. In some embodiments, each R^{a′} is hydrogen and attached to a single bond. In some embodiments, each R^{a′} is hydrogen and attached to a double bond. In some embodiments, each R^{a′} is attached to an aryl and independently hydrogen or C₁-C₃ alkoxy. In some embodiments, each R^{a′} is hydrogen and attached to an aryl.

In some embodiments, R^{a″} is absent. In some embodiments, R^{a″} is hydrogen. In some embodiments, R^{a″} is C₁-C₃ alkyl. In some embodiments, R^{a} is —OH or oxo, each R^{a′} is independently hydrogen or C₁-C₃ alkyl, and R^{a″} is C₁-C₃ alkyl. In some embodiments, R^{a} is —OH or oxo, each R^{a′} is independently hydrogen or C₁-C₃ alkoxy, and R^{a″} is C₁-C₃ alkyl. In some embodiments, R^{a} is oxo, each R^{a′} is independently hydrogen or halogen (e.g., fluoro or chloro), and R^{a″} is CrC₃ alkyl (e.g., methyl). In some embodiments, R^{a} is oxo, each R^{a′} is independently hydrogen or C₁-C₃ alkyl (e.g., methyl), and R^{a″} is C₁-C₃ alkyl (e.g., methyl). In some embodiments, R^{a} is oxo, each R^{a″} is hydrogen, and R^{a″} is C₁-C₃ alkyl (e.g., methyl). In some embodiments, R^{a} is —OH, each R^{a″} is independently hydrogen or C₁-C₃ alkoxy (e.g., methoxy), and R^{a″} is absent. In some embodiments, R^{a} is —OH, each R^{a″} is hydrogen, and R^{a″} is C₁-C₃ alkyl (e.g., methyl). In some embodiments, R^{a} is —OH, each R^{a″} is hydrogen, and R^{a″} is absent.

In some embodiments, R^{b} is absent. In some embodiments, R^{b} is hydrogen. In some embodiments, R^{b} is halogen (e.g., fluoro or chloro). In some embodiments, R^{b′} is hydrogen. In some embodiments, R^{b′} is halogen (e.g., fluoro or chloro). In some embodiments, R^{b′} is —OH. In some embodiments, R^{b′} is -oxo. In some embodiments, R^{b′} is C₁-C₃ alkyl (e.g., methyl). In some embodiments, R^{b′} is hydrogen, halogen (e.g., fluoro or chloro), or C₁-C₃ alkyl (e.g., methyl) and attached to a single bond. In some embodiments, R^{b′} is hydrogen or C₁-C₃ alkyl (e.g., methyl) and attached to a double bond. In some embodiments, R^{b″} is hydrogen. In some embodiments, R^{b″} is —OH.

In some embodiments, R^{b} is hydrogen or halogen (e.g., fluoro or chloro), R^{b′} is hydrogen, halogen (e.g., fluoro or chloro), or C₁-C₃ alkyl (e.g., methyl), and R^{b″} is hydrogen. In some embodiments, R^{b} is hydrogen or halogen (e.g., fluoro or chloro), R^{b′} is hydrogen or halogen (e.g., fluoro or chloro), and R^{b}) is hydrogen. In some embodiments, R^{b} is halogen (e.g., fluoro or chloro), R^{b′} is halogen (e.g., fluoro or chloro), and R^{b″} is hydrogen. In some embodiments, R^{b} is halogen (e.g., fluoro or chloro), R^{b′} is hydrogen, and R^{b″} is hydrogen. In some embodiments, R^{b} is hydrogen, R^{b′} is halogen (e.g., fluoro or chloro), and R^{b″} is hydrogen. In some embodiments, R^{b} is hydrogen, R^{b′} is C₁-C₃ alkyl (e.g., methyl), and R^{b″} is hydrogen. In some embodiments, R^{b} is hydrogen, R^{b′} is hydrogen, and R^{b″} is —OH. In some embodiments, R^{b} is hydrogen, R^{b′} is oxo, and R^{b″} is hydrogen. In some embodiments, R^{b}, R^{b′}, and R^{b″} are each hydrogen.

In some embodiments, each R^{c} is independently hydrogen or —OH. In some embodiments, each R^{c} is independently hydrogen or oxo. In some embodiments, each R^{c} is hydrogen. In some embodiments, each R^{c′} is hydrogen. In some embodiments, each R^{c′} is C₁-C₃ alkyl. In some embodiments, R^{c″} is hydrogen. In some embodiments, R^{c″} is C₁-C₃ alkyl. In some embodiments, R^{c″} is —C(═O)H.

In some embodiments, each R^{c} is hydrogen, each R^{c′} is hydrogen, and R^{c″} is C₁-C₃ alkyl. In some embodiments, each R^{c} is independently hydrogen or —OH, each R^{c′} is hydrogen, and R^{c″} is C₁-C₃ alkyl. In some embodiments, each R^{c} is independently hydrogen or oxo, each R^{c′} is hydrogen, and R^{c″} is C₁-C₃ alkyl. In some embodiments, each R^{c} is independently hydrogen or —OH, each R^{c′} is hydrogen, and R^{c″} is —C(═O)H. In some embodiments, each R^{c} is independently hydrogen or —OH, each R^{c′ is C}₁-C₃ alkyl, and R^{c″} is hydrogen.

In some embodiments, one R^{d} is absent and R^{d′} and the other R^{d} are attached to a double bond. In some embodiments, the R^{d′} and the other R^{d} attached to a double bond are each hydrogen.

In some embodiments, one R^{d} is hydrogen and the other R^{d} is —OH, —COOH, alkyl (e.g., alkylene, alkenyl, or alkynyl), heteroalkyl, wherein the alkyl or heteroalkyl is optionally substituted. In some embodiments, one R^{d} is alkyl and the other R^{d} is —OH, —COOH, alkyl (e.g., alkylene, alkenyl, or alkynyl), heteroalkyl, wherein the alkyl or heteroalkyl is optionally substituted. In some embodiments, one R^{d} is optionally substituted alkoxy and the other R^{d} is —OH, —COOH, alkyl (e.g., alkylene, alkenyl, or alkynyl), heteroalkyl, wherein the alkyl or heteroalkyl is optionally substituted. In some embodiments, one R^{d} is —OH and the other R^{d} is —COOH, alkyl (e.g., alkylene, alkenyl, or alkynyl), heteroalkyl, wherein the alkyl or heteroalkyl is optionally substituted. In some embodiments, each R^{d} is independently hydrogen or —OH. In some embodiments, each R^{d} is independently optionally substituted alkyl or —OH. In some embodiments, each R^{d} is independently —COOH or —OH. In some embodiments, each R^{d} is independently —COOH or optionally substituted alkoxy. In some embodiments, each R^{d} is taken together to form an oxo. In some embodiments, each R^{d} is taken together to form an optionally substituted alkenyl. In some embodiments, the alkenyl is substituted with —COOH and alkyl. In some embodiments, the alkyl comprises saturated and unsaturated carbon bonds. In some embodiments, each R^{d} is independently optionally substituted alkyl or hydrogen. In some embodiments, the alkyl consists of saturated carbon bonds. In some embodiments, the alkyl is substituted with C₁-C₃ alkyl and alkyl further substituted with —COOH. In some embodiments, the alkyl is substituted with C₁-C₃ alkyl and alkyl further substituted with —OH.

In some embodiments, R^{d′} is hydrogen. In some embodiments, R^{d′} is —OH. In some embodiments, R^{d′} is C₁-C₃ alkyl (e.g., alkylene or alkenyl). In some embodiments, the C₁-C₃ alkyl is methyl. In some embodiments, the C₁-C₃ alkyl is CHCH. In some embodiments, R^{d′} is heteroalkyl. In some embodiments, the heteroalkyl is —O(C═O)C₁-C₃ alkyl.

In some embodiments, one R^{d} is taken together with R^{d′} to form an optionally substituted cycloalkyl or optionally substituted heterocycloalkyl. In some embodiments, one R^{d} is taken together with R^{d′} to form a heterocycloalkyl substituted with one or more alkyl groups. In some embodiments, one R^{d} is optionally substituted alkyl and the other R^{d} is taken together with R^{d′} to form a heterocycloalkyl substituted with one or more alkyl groups. In some embodiments, the alkyl is substituted with oxo and —OH. In some embodiments, the alkyl is substituted with oxo and alkyl further substituted with halogen (e.g., fluoro or chloro). In some embodiments, the heterocycloalkyl is an optionally substituted dioxolane. In some embodiments, the optionally substituted dioxolane is 2,2-dimethyl-1,3-dioxolane. In some embodiments, the optionally substituted dioxolane is 1,4-dioxaspiro[4.4]nonane.

In some embodiments, each R^{d} is independently hydrogen or optionally substituted alkyl and R^{d′} is hydrogen. In some embodiments, each R^{d} is independently hydrogen or optionally substituted alkyl and R^{d′} is C₁-C₃ alkyl. In some embodiments, each R^{d} is independently optionally substituted alkyl and R^{d′} is hydrogen. In some embodiments, each R^{d} is independently —OH or optionally substituted alkyl and R^{d′} is hydrogen. In some embodiments, each R^{d} is independently —COOH or optionally substituted alkoxy and R^{d′} is hydrogen. In some embodiments, each R^{d} is independently —OH or optionally substituted alkyl and R^{d′} is C₁-C₃ alkyl. In some embodiments, each R^{d} is independently —OH or optionally substituted alkyl and R^{d′} is —OH. In some embodiments, each R^{d} is independently —OH or optionally substituted alkyl and R^{d′} is alkyl (e.g., alkenyl). In some embodiments, each R^{d} is independently hydrogen or —OH and R^{d′} is hydrogen.

In some embodiments, each R^{d} is independently —OH or —COOH and R^{d′} is hydrogen. In some embodiments, each R^{d} and R^{d′} are hydrogen. In some embodiments, each R^{d} is optionally substituted alkenyl and R^{d′} is optionally substituted alkoxy. In some embodiments, each R^{d} is taken together to form an oxo and R^{d′} is hydrogen. In some embodiments, one R^{d} is optionally substituted alkyl and the other R^{d} is taken together with R^{d′} is to form an optionally substituted heterocycloalkyl.

In some embodiments, the alkyl or heteroalkyl of R^{d} or R^{d′} is substituted with one or more of the group consisting of—SH, —OH, —COOH, oxo, halogen, amino (e.g., dihydroamino, alkylamino, or arylamino), alkyl (e.g., alkenyl, alkynyl), heteroalkyl, ester, amide, sulfonic acid, and sulfone. In some embodiments, one R^{d} is taken together with R^{d′} to form substituted heterocycloalkyl.

In some embodiments, the alkyl of R^{d} is substituted with oxo and alkyl further substituted with hydroxyl. In some embodiments, the alkyl of R^{d} is substituted with oxo and alkyl further substituted with halogen (e.g., fluorine or chlorine). In some embodiments, the alkyl of R^{d} is substituted with oxo and C₁-C₃ alkyl. In some embodiments, the alkyl of R^{d} is substituted with oxo and alkyl further substituted with alkoxy further substituted with oxo and C₁-C₃ alkyl. In some embodiments, the alkyl of R^{d} is substituted with alkyl and alkyl further substituted with oxo and amino further substituted with alkyl further substituted with sulfonic acid. In some embodiments, the alkyl of R^{d} is substituted with oxo and thiol (e.g., thioether) further substituted with C₁-C₃ alkyl further substituted with halogen (e.g., fluorine or chlorine). In some embodiments, the alkyl of R^{d} is substituted with —OH. In some embodiments, the alkyl of R^{d} is substituted with oxo and hydroxyl (e.g., ether) further substituted with C₁-C₃ alkyl further substituted with halogen (e.g., fluorine or chlorine). In some embodiments, the alkoxy of R^{d} is substituted with oxo and alkoxy further substituted with alkyl.

In some embodiments, the C₁-C₃ alkyl is methyl, ethyl, propyl, isopropyl, butyl, or tert-butyl. In some embodiments, the C₁-C₃ alkyl is methyl. In some embodiments, the C₁-C₃ alkoxy is methoxy, ethyoxy, propyoxy, or isopropoxy. In some embodiments, the C₁-C₃ alkyl is methoxy.

In some embodiments, the first radical and the second radical are joined by a linker (e.g., a bond). In some embodiments, the first radical is joined to the second radical through any one of R^{a}, R^{b}, R^{c}, or R^{d} of the first radical. In some embodiments, the first radical is joined to the second radical through any one of R^{a}, R^{b}, R^{c}, or R^{d}, and the R^{a}, R^{b}, R^{c}, or R^{d} through which the first radical is joined to the second radical comprises a hydroxyl radical (e.g., when together with the linker or second radical (where the linker is a bond), forms an ether), a thiol radical (e.g., when together with the linker or second radical (where the linker is a bond), forms a thioether), or a carboxylate radical (e.g., when taken together with the linker or second radical (where the linker is a bond), forms an ester or carbonate). In some embodiments, the connection between the thiol radical forms a thioester, a disulfide, or a thiocarbonate. In some embodiments, the connection between the carboxylate radical forms an anhydride. In some embodiments, the first radical is joined to the second radical through any one of R^{a}, R^{b}, R^{c}, or R^{d}, and the R^{a}, R^{b}, R^{c}, or R^{d} through which the first radical is joined to the second radical comprises an amino radical (e.g., when together with the linker or second radical (where the linker is a bond), forms an amide, carbamate, or thiocarbamate).

In some embodiments, the R^{a}, R^{b}, R^{c}, or R^{d} through which the first radical is joined to the second radical comprises a hydroxyl radical which together with the linker or with the second radical forms an ether. In some embodiments, the R^{a}, R^{b}, R^{c}, or R^{d} through which the first radical is joined to the second radical comprises a thiol radical which together with the linker or the second radical forms a thioether. In some embodiments, the R^{a}, R^{b}, R^{c}, or R^{d} through which the first radical is joined to the second radical comprises a carboxylate radical which together with the linker or the second radical forms an ester or a carbonate.

In some embodiments, the first radical has a structure of Formula (I′), Formula (IA), Formula (IB), or Formula (IC) and the second radical does not have a structure of Formula (I′), Formula (IA), Formula (IB), or Formula (IC). In some embodiments, the structure of Formula (I′), Formula (IA), Formula (IB), or Formula (IC) has a melt and/or glass transition temperature at a temperature of at least 20° C. (e.g., at least 25° C., at least 30° C., at least 37° C., at least 40° C., at least 50° C., at least 100° C., or more) in its free form.

In some embodiments, both the first radical and the second radical consist of the three-membered ring system of Formula (I′), Formula (IA), Formula (IB), or Formula (IC). In some embodiments, the first radical is a central nervous system (CNS) agent. In some embodiments, the radical of Formula (I′), Formula (IA), Formula (IB), or Formula (IC) is a steroid, an opioid agonist, an opioid antagonist, an adrenergic receptor antagonist (e.g., β-blocker, α-1 blocker), or a serotonergic antagonist (e.g., serotonin 5-HT3 receptor antagonist). In some embodiments, the first radical is an anti-inflammatory agent, an anti-psychotic agent (e.g., typical anti-psychotic, atypicalantipsychotic, schizophrenia, or the like), or the like. In some embodiments, the IOP lowering agent is a beta-blocker. In some embodiments, the beta-blocker is timolol.

In some embodiments, the second radical is an intraocular pressure (IOP) lowering agent. In some embodiments, the first radical is an anti-inflammatory agent and the second radical is an intraocular pressure (IOP) lowering agent. In some embodiments, the first radical is an (e.g., angiostatic) steroid (e.g., anecortave) or benign steroid (e.g., cholesterol) and the second radical is an IOP lowering agent. In some embodiments, the IOP lowering agent is a prostaglandin.

In some embodiments, the first radical is a solid (e.g., having a melting point of at least 30° C.) in its free form. In some embodiments, the second radical is a liquid (e.g., having a melting point of less than 30° C.) in its free form. In some embodiments, the first radical is a steroid (e.g., dexamethasone, anecortave, etc.). In some embodiments, the steroid is a corticosteroid (e.g., glucocorticoid or mineralcorticoid), a sex steroid, a neurosteroid, an aminosteroid, or a secosteroid. In some embodiments, the second radical is not a steroid (or does not have a structure of any one of Formula (I′), Formula (IA), Formula (IB), or Formula (IC)). In some embodiments, the first radical is a steroid and the second radical is a prostaglandin.

In some embodiments, the second radical has a structure of Formula (II′):

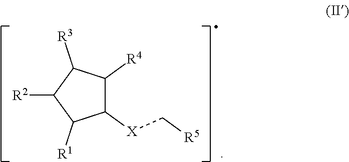

In some embodiments, [symbol] is a single bond or a double bond. In some embodiments, R¹, R², R³, R⁴, and R⁵ are each independently selected from one or more of the group consisting of hydrogen, oxo, halo, alkyl, heteroalkyl, cycloalkyl, heterocycloalkyl, alkoxy, amino (e.g., dihydroamino, alkylamino, or arylamino), hydroxyl, and thiol, wherein the alkyl, heteroalkyl, cycloalkyl, and heterocycloalkyl is optionally substituted. In some embodiments, any one of R¹, R², R³, R⁴, or R⁵ are taken together to form an optionally substituted cycloalkyl or heterocycloalkyl. In some embodiments, X is selected from the group consisting of —O—, —NR—, —S(R)ₐ—, and —C(R)_{b}—. In some embodiments, a is independently 0-2. In some embodiments, b is independently 1 or 2. Also provided in certain embodiments herein are pharmaceutical salts or solvates of a compound of Formula (II′).

In some embodiments, X is S, —C(R)₁—, or —C(R)₂—. In some embodiments, X is S attached to a single bond. In some embodiments, X is —CH— or —CH₂—.

In some embodiments, R⁴ is alkyl substituted with one or more groups, each group being independently selected from —C(═O)OC₁-C₃alkyl, —COOH, —CONH₂, —CONHC₁-C₃alkyl, and/or alkyl (e.g., alkylene or alkenyl). In some embodiments, R⁴ is alkyl substituted with —COOH. In some embodiments, R⁴ is alkyl substituted with —C(═O)OC₁-C₃alkyl. In some embodiments, R⁴ is alkyl substituted with —CONH₂. In some embodiments, R⁴ is alkyl substituted with —CONHC₁-C₃alkyl. In some embodiments, the alkyl of R⁴ comprises at least one double bond. In some embodiments, the alkyl of R⁴ comprises one double bond. In some embodiments, the alkyl of R⁴ comprises two double bonds. In some embodiments, the two double bonds form an allene.

In some embodiments, R⁵ is alkyl substituted with one or more groups, each group being independently selected from halogen, —OH, oxo, alkyl (e.g., alkynyl), alkoxy, aryl, and aryloxy, wherein alkyl (e.g., alkynyl), aryl, or aryloxy is optionally substituted. In some embodiments, the alkyl is substituted with one or more groups, each group being independently selected from halogen, —OH, oxo, alkyl (e.g., alkynyl), aryl, or aryloxy, wherein the alkyl (e.g., alkynyl), aryl, or aryloxy is optionally substituted. In some embodiments, the aryl or aryloxy is substituted with one or more halogen groups. In some embodiments, the aryl or aryloxy is unsubstituted.

In some embodiments, the second radical has a structure of Formula (IIA):

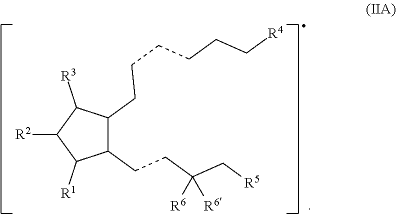

In some embodiments, each [symbol] is independently a single bond or a double bond. In some embodiments, R¹, R², R³, R⁴, and R⁵ are each independently selected from one or more of the group consisting of hydrogen, oxo, halo, alkyl, heteroalkyl, cycloalkyl, heterocycloalkyl, alkoxy, amino (e.g., dihydroamino, alkylamino, or arylamino), hydroxyl, and thiol, wherein the alkyl, heteroalkyl, cycloalkyl, and heterocycloalkyl is optionally substituted.

In some embodiments, Formula (IIA) comprises three double bonds. In some embodiments, Formula (IIA) comprises two double bonds. In some embodiments, Formula (IIA) comprises one double bond.

In some embodiments, R¹ and R³ are each independently —OH or oxo. In some embodiments, R¹ is oxo, R² is hydrogen, and R³ is —OH. In some embodiments, R³ is oxo, R² is hydrogen, and R¹ is —OH. In some embodiments, R¹ and R³ are each —OH and R² is hydrogen.

In some embodiments, R⁴ is —C(═O)OC₁-C₃alkyl, —COOH, —CONH₂, or —CONHC₁-C₃alkyl. In some embodiments, R⁴ is —COOH. In some embodiments, R⁴ is —CONH₂.

In some embodiments, R⁵ is alkyl or aryloxy, wherein the alkyl and aryloxy are optionally substituted. In some embodiments, R⁵ is alkyl. In some embodiments, the alkyl is butyl or hexyl. In some embodiments, R⁵ is alkyl substituted with optionally substituted aryl or optionally substituted alkyl. In some embodiments, the alkyl is substituted with unsubstituted aryl. In some embodiments, the alkyl is substituted with alkyl (e.g., but-2-yne). In some embodiments, R⁵ is unsubstituted aryloxy. In some embodiments, R⁵ is aryloxy substituted with one or more alkyl (e.g., —CF₃) or halo (fluoro or chloro) groups. In some embodiments, the aryloxy is substituted with —CF₃.

In some embodiments, R⁶ and R^{6′} are each fluoro. In some embodiments, R⁶ is H or methyl and R^{6′} is —OH. In some embodiments, R⁶ is H and R^{6′} is —OH. In some embodiments, R⁶ is methyl and R^{6′} is —OH. In some embodiments, R⁶ and R^{6′} are taken together to form an oxo.

In some embodiments, R³ and R⁴ of Formula (II′) are taken together to form an optionally substituted cycloalkyl or heterocycloalkyl. In some embodiments, R³ and R⁴ of Formula (II′) are taken together to form heterocycloalkyl substituted with optionally substituted alkyl (e.g., alkenyl). In some embodiments, R³ and R⁴ of Formula (II′) are taken together to form a heterocycloalkyl substituted with alkyl (e.g., alkenyl) substituted with —COOH or —C(═O)OC₁-C₃alky. In some embodiments, the heterocycloalkyl is substituted with alkyl further substituted with —COOH or—C(═O)OC₁-C₃alkyl. In some embodiments, the heterocycloalkyl is substituted with alkenyl further substituted with alkyl, which is further substituted with —COOH or —C(═O)OC₁-C₃alkyl. In some embodiments the alkyl or alkenyl is substituted with —COOH.

In some embodiments, the second radical has a structure of Formula (IIB):

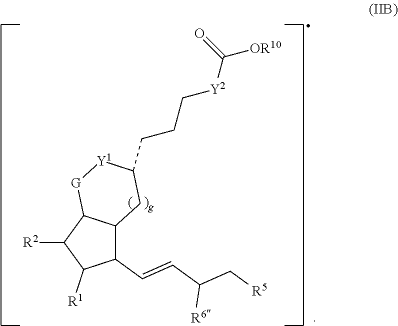

In some embodiments, [symbol] is a single bond or a double bond. In some embodiments, R¹, R², and R⁵ are each independently selected from one or more of the group consisting of hydrogen, oxo, halo, alkyl, heteroalkyl, cycloalkyl, heterocycloalkyl, alkoxy, amino (e.g., dihydroamino, alkylamino, or arylamino), hydroxyl, and thiol, wherein the alkyl, heteroalkyl, cycloalkyl, and heterocycloalkyl is optionally substituted. In some embodiments, R^{6″} is oxo or —OH. In some embodiments, Y¹ and Y² are each independently a bond or alkylene. In some embodiments, G is O or CH₂. In some embodiments, g is 1 or 2. In some embodiments, R¹⁰ is alkyl or H. Also provided in certain embodiments herein are pharmaceutical salts or solvates of a compound of Formula (IIB).

In some embodiments, G is O and Y¹ is a bond. In some embodiments, Y² is methylene. In some embodiments, g is 1. In some embodiments, G is O and Y¹ is a alkylene. In some embodiments, Y² is a bond. In some embodiments, g is 2.

In some embodiments, R¹ is oxo or —OH. In some embodiments, R¹ is —OH. In some embodiments, R¹ is —OH and R² is hydrogen. In some embodiments, R^{6″} is —OH. In some embodiments, R⁵ is alkyl. In some embodiments, R⁵ is substituted aryl.

In some embodiments, G is O, Y¹ is a bond, Y² is methylene, g is 1, R¹ is —OH, R² is hydrogen, R⁵ is alkyl, R^{6″} is —OH, and R¹⁰ is H. In some embodiments, G is O, Y¹ is a methylene, Y² is a bond, g is 2, R¹ is —OH, R² is hydrogen, R⁵ is substituted aryl, R^{6″} is —OH, and R¹⁰ is H or C₁-C₃alkyl.

In some embodiments, R⁵ is selected from one or more of the group consisting of —O—, —OH, halogen, alkyl (e.g., alkynyl), aryl, wherein the alkyl (e.g., alkynyl) and aryl are optionally substituted with one or more of alkyl (e.g., fluoroalkyl), halogen, and —OH. In some embodiments, R⁵ is optionally substituted aryl or optionally substituted —O-aryl. In some embodiments, R⁵ is alkyl or aryloxy, wherein the alkyl and optionally substituted aryloxy. In some embodiments, R⁵ is alkyl. In some embodiments, the alkyl is butyl or hexyl. In some embodiments, R⁵ is unsubstituted aryloxy. In some embodiments, the aryloxy is substituted with one or two —F. In some embodiments, R⁵ is an aryl or O-aryl, each of which is unsubstituted. In some embodiments, R⁵ is an aryl or O-aryl, each of which is substituted with one or more of halogen or haloalkyl (e.g., trifluoroalkyl, e.g., trifluoromethyl).

In certain embodiments, provided herein is a compound comprising a first radical or a second radical, wherein the first radical has a structure of any one of Formula (I′), Formula (IA), Formula (IB), or Formula (IC) and the second radical has a structure of any one of Formula (II′), Formula (IIA), or Formula (IIB). In some embodiments, the first radical (e.g., having a structure of any one of Formula (I′), Formula (IA), Formula (IB), or Formula (IC)) and the second radical (e.g., Formula (II′), Formula (IIA), or Formula (IIB)) are joined by a linker (e.g., hydrolyzable linker). In some embodiments, the linker is a bond.

In certain embodiments, provided herein is a compound comprising a steroid. In some embodiments, provided herein is a compound comprising a prostaglandin. In some embodiments, provided herein is a compound comprising a linker (e.g., hydrolyzable linker). In some embodiments, the linker adjoins (e.g., covalently) the steroid and the prostaglandin. Also provided in certain embodiments herein are pharmaceutical salts or solvates of a compound.

In some embodiments, a compound provided herein comprises a steroid attached to a second agent (e.g., a prostaglandin) through an optional linker (as such, forming a heteroalkyl bond (e.g., an ester, a carbonate, etc.), such as, whereby upon cleavage (e.g., hydrolysis) of the heteroalkyl bond, the steroid and/or second agent are released in their free form. In some embodiments, a steroid radical (e.g., a first radical) provided herein (e.g., a hydroxyl radical (e.g., anecortave desacetate radical)) is attached to an optional linker or a second radical (e.g., a prostaglandin radical) (e.g., a hydroxyl radical, a carboxylic radical, etc.) provided herein to form a compound provided herein.

In certain aspects, provided herein is a compound having the structure of Formula (III′):

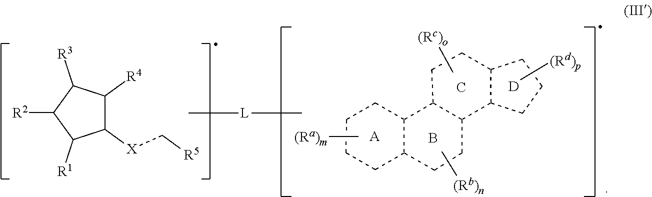

In some embodiments, [symbol] is a single bond or a double bond. In some embodiments, each R^{a}, R^{b}, R^{c}, and R^{d} are independently selected from the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, alkynyl, heteroalkyl, cycloalkyl, or heterocycloalkyl is optionally substituted. In some embodiments, any one of R^{a}, R^{b}, R^{c}, and R^{d} are taken together with another of R^{a}, R^{b}, R^{c}, and R^{d} to form an substituted or unsubstituted cycloalkyl or heterocycloalkyl. In some embodiments, each of m, n, o, and p are independently 0-6. In some embodiments, R¹, R², R³, R⁴, and R⁵ are each independently selected from one or more of the group consisting of hydrogen, oxo, halo, alkyl, heteroalkyl, cycloalkyl, heterocycloalkyl, alkoxy, amino (e.g., dihydroamino, alkylamino, or arylamino), hydroxyl, and thiol, wherein the alkyl, heteroalkyl, cycloalkyl, and heterocycloalkyl is optionally substituted. In some embodiments, L is a linker. Also provided in certain embodiments herein are pharmaceutical salts or solvates of a compound of Formula (III′).

In some embodiments, the optional substitution of any one of the groups of Formula (III′) are as provided elsewhere herein (e.g., as in for Formula (IB) or Formula (II′)).

In certain aspects, provided herein is a compound having the structure of Formula (IV′):

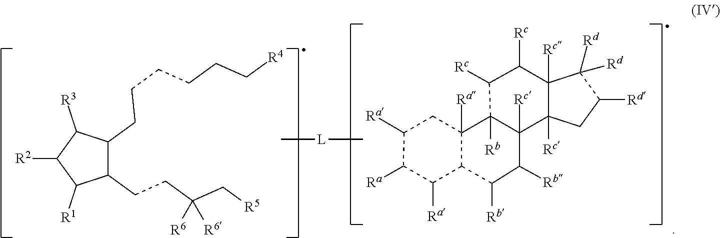

In some embodiments, [symbol] is a single bond or a double bond. In some embodiments, R^{a} is hydrogen, —OH, or oxo. In some embodiments, each R^{a″} is independently selected from hydrogen, —OH, halogen, C₁-C₃ alkyl, and alkoxy. In some embodiments, R^{a″} is absent, hydrogen, or C₁-C₃ alkyl. In some embodiments, R^{b} is absent, hydrogen, halogen, or C₁-C₃ alkyl. In some embodiments, R^{b′} is hydrogen, halogen, —OH, oxo, or C₁-C₃ alkyl. In some embodiments, R^{b″} is hydrogen or —OH. In some embodiments, each R^{c} is independently hydrogen, —OH, oxo, or C₁-C₃ alkyl. In some embodiments, each R^{c″} is independently hydrogen or C₁-C₃ alkyl. In some embodiments, R^{c″} is hydrogen, —OH, C₁-C₃ alkyl, or —C(═O)H. In some embodiments, each R^{d} is independently hydrogen, —OH, —COOH, alkyl (e.g., alkylene, alkenyl, or alkynyl), heteroalkyl, or each R^{d} is taken together to form an oxo, wherein the alkyl or heteroalkyl is optionally substituted. In some embodiments, R^{d′} is hydrogen, —OH, C₁-C₃ alkyl (e.g., alkylene or alkenyl), or heteroalkyl. In some embodiments, one R^{d} is taken together with R^{d′} to form a substituted or unsubstituted cycloalkyl or heterocycloalkyl. In some embodiments, R¹, R², R³, R⁴, and R⁵ are each independently selected from one or more of the group consisting of hydrogen, oxo, halo, alkyl, heteroalkyl, cycloalkyl, heterocycloalkyl, alkoxy, amino (e.g., dihydroamino, alkylamino, or arylamino), hydroxyl, and thiol, wherein the alkyl, heteroalkyl, cycloalkyl, and heterocycloalkyl is optionally substituted. In some embodiments, R⁶ and R^{6′} are each independently hydrogen, halogen, alkyl, or R⁶ and R^{6′} are taken together to form an oxo. Also provided in certain embodiments herein are pharmaceutical salts or solvates of a compound of Formula (IV′).

In certain aspects, provided herein is a compound having the structure of Formula (V′):

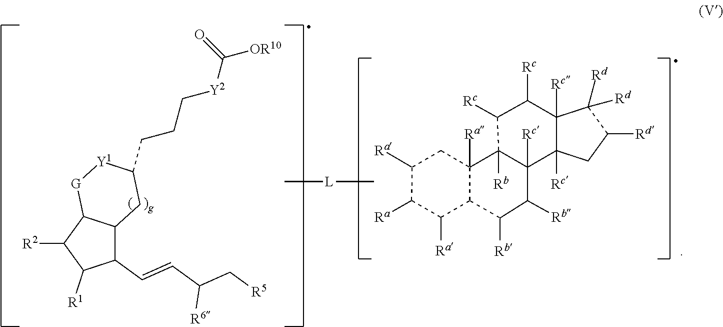

In some embodiments, [symbol] is a single bond or a double bond. In some embodiments, R^{a} is hydrogen, —OH, or oxo. In some embodiments, each R^{a′} is independently selected from hydrogen, —OH, halogen, C₁-C₃ alkyl, and alkoxy. In some embodiments, R^{a″} is absent, hydrogen, or C₁-C₃ alkyl. In some embodiments, R^{b} is absent, hydrogen, halogen, or C₁-C₃ alkyl. In some embodiments, R^{b′} is hydrogen, halogen, —OH, oxo, or C₁-C₃ alkyl. In some embodiments, R^{b″} is hydrogen or —OH. In some embodiments, each R^{c} is independently hydrogen, —OH, oxo, or C₁-C₃ alkyl. In some embodiments, each R^{c′ is independently hydrogen or C}₁-C₃ alkyl. In some embodiments, R^{c″ is hydrogen, —OH, C}₁-C₃ alkyl, or —C(═O)H. In some embodiments, each R^{d} is independently hydrogen, —OH, —COOH, alkyl (e.g., alkylene, alkenyl, or alkynyl), heteroalkyl, or each R^{d} is taken together to form an oxo, wherein the alkyl or heteroalkyl is optionally substituted. In some embodiments, R^{d′} is hydrogen, —OH, C₁-C₃ alkyl (e.g., alkylene or alkenyl), or heteroalkyl. In some embodiments, one R^{d} is taken together with R^{d′} to form a substituted or unsubstituted cycloalkyl or heterocycloalkyl. In some embodiments, R¹, R², and R⁵ are each independently selected from one or more of the group consisting of hydrogen, oxo, halo, alkyl, heteroalkyl, cycloalkyl, heterocycloalkyl, alkoxy, amino (e.g., dihydroamino, alkylamino, or arylamino), hydroxyl, and thiol, wherein the alkyl, heteroalkyl, cycloalkyl, and heterocycloalkyl is optionally substituted. In some embodiments, R^{6″} is oxo or —OH. In some embodiments, Y¹ and Y² are each independently a bond or alkylene. In some embodiments, G is O or CH₂. In some embodiments, g is 1 or 2. In some embodiments, R¹⁰ is alkyl or H. In some embodiments, L is a linker. Also provided in certain embodiments herein are pharmaceutical salts or solvates of a compound of Formula (V′).

In some embodiments, the first radical is a hydroxyl radial. In some embodiments, the second radical is a hydroxyl radical. In some embodiments, the first radical is a carboxyl radical. In some embodiment, the second radical is a carboxyl radical.

Provided in some embodiments herein is a compound comprising a structure of Formula (IE):

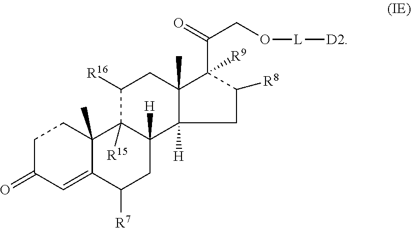

In some embodiments, [symbol] is a single bond or a double bond. In some embodiments, R⁷ is hydrogen or halogen. In some embodiments, R⁸ is hydrogen or C₁-C₄ alkyl. In some embodiments, R⁹ is absent, hydrogen, or hydroxyl. In some embodiments, R¹⁵ is absent, hydrogen, or halogen. In some embodiments, R¹⁶ is hydrogen or hydroxyl. In some embodiments, D2 is a radical of an active agent (e.g., a therapeutically active agent) (or drug). In some embodiments, D2 is a radical of an active agent (e.g., a therapeutically active agent) (or drug). In some embodiments, Q¹ and Q² are each independently absent or (C═X¹)X². In some embodiments, X¹ is O or S. In some embodiments, X² is O, S, or NR¹. In some embodiments, R¹ is hydrogen or C₁-C₆ alkyl. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl). In some embodiments, wherein D2 is not a steroid. In some embodiments, the compound is a a pharmaceutically acceptable salt or solvate.

In some embodiments, R⁷ is hydrogen.

In some embodiments, R⁸ is methyl.

In some embodiments, R⁸ is hydrogen.

In some embodiments, R⁹ is hydroxyl.

In some embodiments, R¹⁵ is fluoro.

In some embodiments, R¹⁵ is absent.

In some embodiments, R¹⁵ is hydrogen.

In some embodiments, R¹⁶ is hydroxyl.

In some embodiments, R¹⁶ is hydrogen.

In some embodiments, the compound has a structure of Formula (IE-i):

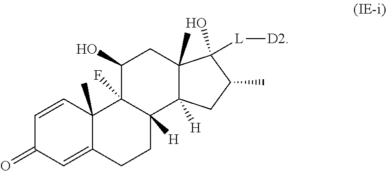

In some embodiments, the compound has a structure of Formula (IE-ii):

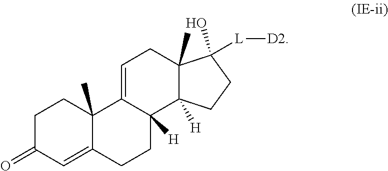

In some embodiments, the compound has a structure of Formula (IE-iii):

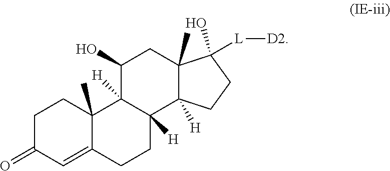

In certain embodiments, provided herein is a compound having the structure of Formula (VI):

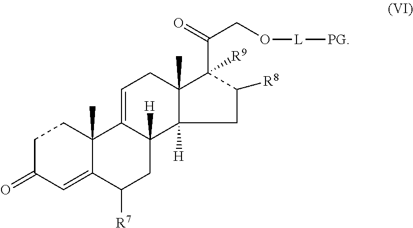

In some embodiments, [symbol] is a single bond or a double bond. In some embodiments, R⁷ is hydrogen or halogen. In some embodiments, R⁷ is hydrogen. In some embodiments, R⁷ is fluoro. In some embodiments, R⁸ is a hydrogen or a C₁-C₄ alkyl. In some embodiments, R⁸ is hydrogen. In some embodiments, R⁸ is methyl, ethyl, propyl or butyl. In some embodiments, R⁸ is methyl, ethyl, or butyl. In some embodiments, R⁸ is methyl. In some embodiments, R⁷ is hydrogen and R⁸ is methyl. In some embodiments, R⁹ is absent, hydrogen or hydroxyl. In some embodiments, R⁹ is hydrogen. In some embodiments, R⁹ is hydroxyl. In some embodiments, R⁹ is absent. In some embodiments, L is a linker. In some embodiments, L is a linker described herein. In some embodiments, Lis a bond. In some embodiments, L comprises one or more linker group, each linker group being independently selected from the group consisting of an alkylene, cycloalkylene and —O—. In some embodiments, PG is a prostaglandin radical. Also provided in certain embodiments herein are pharmaceutically-acceptable salts or solvates of a compound of Formula (VI).

In some embodiments, a compound provided herein has the structure of formula (VI-A):

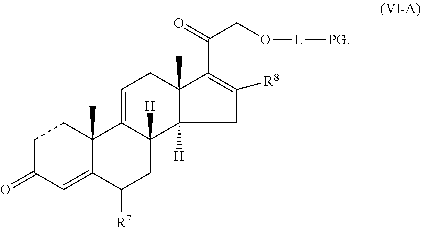

In some embodiments, [symbol] is a single bond or a double bond. In some embodiments, R⁷ is hydrogen or halogen. In some embodiments, R⁷ is hydrogen. In some embodiments, R⁷ is fluoro. In some embodiments, R⁸ is a hydrogen or a C₁-C₄ alkyl. In some embodiments, R⁸ is hydrogen. In some embodiments, R⁸ is methyl, ethyl, propyl or butyl. In some embodiments, R¹ is methyl, ethyl, or butyl. In some embodiments, R⁸ is methyl. In some embodiments, R⁷ is hydrogen and R⁸ is methyl. In some embodiments, L is a linker. In some embodiments, L is a bond. In some embodiments, L comprises one or more linker group, each linker group being independently selected from the group consisting of an alkylene, cycloalkylene or —O—. In some embodiments, PG is a prostaglandin radical. Also provided in certain embodiments herein are pharmaceutically-acceptable salts or solvates of a compound of Formula (VI-A).

In some embodiments, a compound provided herein has the structure of formula (VI-B):

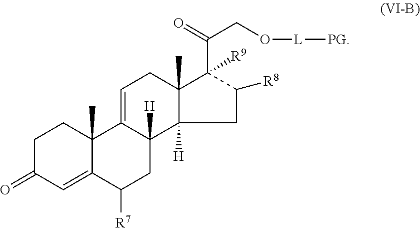

In some embodiments, [symbol] is a single bond or a double bond. In some embodiments, R⁷ is hydrogen or halogen. In some embodiments, R⁷ is hydrogen. In some embodiments, R⁷ is fluoro. In some embodiments, R⁸ is a hydrogen or a C₁-C₄ alkyl. In some embodiments, R⁸ is hydrogen. In some embodiments, R⁸ is methyl, ethyl, propyl or butyl. In some embodiments, R⁸ is methyl, ethyl, or butyl. In some embodiments, R⁸ is methyl. In some embodiments, R⁷ is hydrogen and R⁸ is methyl. In some embodiments, R⁹ is absent, hydrogen or hydroxyl. In some embodiments, R⁹ is hydrogen. In some embodiments, R⁹ is hydroxyl. In some embodiments, R⁹ is absent. In some embodiments, L is a linker. In some embodiments, L is a bond. In some embodiments, L comprises one or more linker group, each linker group being independently selected from the group consisting of an alkylene, cycloalkylene or —O—. In some embodiments, PG is a prostaglandin radical. Also provided in certain embodiments herein are pharmaceutically-acceptable salts or solvates of a compound of Formula (VI-B).

In some embodiments, a compound provided herein has the structure of formula (VI-C):

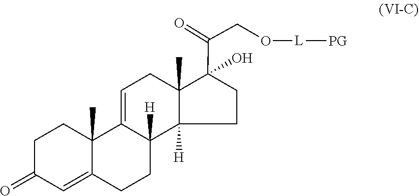

In some embodiments, L is a linker. In some embodiments, L is a bond. In some embodiments, L comprises one or more linker group, each linker group being independently selected from the group consisting of an alkylene, cycloalkylene or —O—. In some embodiments, PG is a prostaglandin radical. Also provided in certain embodiments herein are pharmaceutically-acceptable salts or solvates of a compound of Formula (VI-B).

In certain embodiments, provided herein is a compound having a prostaglandin (PG) radical of the formula (VII).

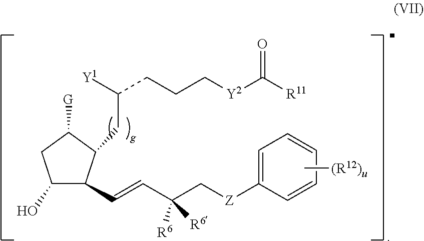

In some embodiments, [symbol] is a single bond or a double bond. In some embodiments, G is OH and Y¹ is hydrogen. In some embodiments, G together with Y¹ form —O—CH₂—. In some embodiments, Y² is a bond or —CH₂—. In some embodiments, g is 1 or 2. In some embodiments, Z is —O— or —CH₂—. In some embodiments, R⁶ and R^{6′} are each independently hydrogen, halogen or OH. In some embodiments, R¹¹ is —OR¹³, or —NR^{13′}R^{13″}. In some embodiments, R¹³, R^{13′} and R^{13″} are each independently hydrogen or a C₁-C₃ alkyl. In some embodiments, each R¹² is independently halogen or haloalkyl. In some embodiments, u is 0-5. In some embodiments, R⁶ and R^{6′} are each fluoro. In some embodiments, R⁶ is OH and R^{6′} is hydrogen. In some embodiments, Z is —O—. In some embodiments, Z is —CH₂—. In some embodiments, each R¹² is F and u is 2. In some embodiments, CF₃ and u is 1. In some embodiments, u is 0. In some embodiments, u is 1. In some embodiments, u is 2. In some embodiments, u is 3. In some embodiments, u is 4. In some embodiments, u is 5. In some embodiments, R¹¹ is OH. In some embodiments, R¹¹ is —NHCH₂CH₃. In some embodiments, R¹¹ is —OCH(CH₃)₂.

In some embodiments, the prostaglandin (PG) radical provided herein has the formula (VII-A):

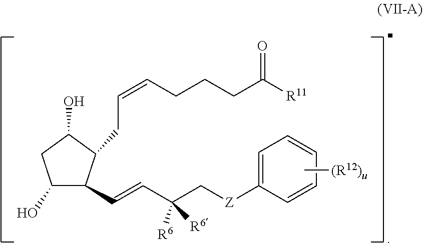

In some embodiments, Z is —O— or —CH₂—. In some embodiments, R⁶ and R^{6′} are each independently hydrogen, halogen or OH. In some embodiments, R¹¹ is —OR¹³, or —NR^{13′}R^{13″}. In some embodiments, R¹³, R^{13′} and R^{13″} are each independently hydrogen or a C₁-C₃ alkyl. In some embodiments, each R¹² is independently halogen or haloalkyl. In some embodiments, u is 0-5. In some embodiments, R⁶ and R^{6′} are each independently fluoro. In some embodiments, R⁶ is OH and R^{6′} is hydrogen. In some embodiments, Z is —O—. In some embodiments, Z is —CH₂—. In some embodiments, R¹² is F and u is 2. In some embodiments, CF₃ and u is 1. In some embodiments, u is 0. In some embodiments, u is 1. In some embodiments, u is 2. In some embodiments, u is 3. In some embodiments, u is 4. In some embodiments, u is 5. In some embodiments, R¹¹ is OH. In some embodiments, R¹¹ is —NHCH₂CH₃. In some embodiments, R¹¹ is —OCH(CH₃)₂.

In some embodiments, the prostaglandin (PG) radical has the formula (VII-B):

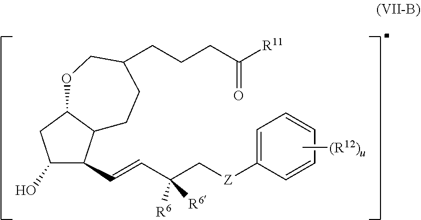

In some embodiments, Z is —O— or —CH₂—. In some embodiments, R⁶ and R^{6′} are each independently hydrogen, halogen or OH. In some embodiments, R¹¹ is —OR¹³, or —NR^{13′}R^{13″}. In some embodiments, R¹³, R^{13′} and R^{13″} are each independently hydrogen or a C₁-C₃ alkyl. In some embodiments, each R¹² is independently halogen or haloalkyl. In some embodiments, u is 0-5. In some embodiments, R⁶ and R^{6′} are each independently fluoro. In some embodiments, R⁶ is OH and R^{6′} is hydrogen. In some embodiments, Z is —O—. In some embodiments, Z is —CH₂—. In some embodiments, R¹² is F and u is 2. In some embodiments, CF₃ and u is 1. In some embodiments, u is 0. In some embodiments, u is 1. In some embodiments, u is 2. In some embodiments, u is 3. In some embodiments, u is 4. In some embodiments, u is 5. In some embodiments, R¹¹ is OH. In some embodiments, R¹¹ is —NHCH₂CH₃. In some embodiments, R¹¹ is —OCH(CH₃)₂.

In some embodiments, the radical (e.g., a PG radical) is attached to a linker (e.g., L) provided herein. In some embodiments, the PG radical is attached to a linker (e.g., L) and the linker is further attached to a steroid radical (e.g., provided herein). In some embodiments, R¹¹ is a radical (e.g., a hydroxyl radical or an amino radical) attached to a linker (e.g., a radical of a linker) provided herein (e.g., and the linker (e.g., another radical of the linker) is further attached to a steroid radical provided herein). In some embodiments, R⁶ or R^{6′} is a radical (e.g., a hydroxyl radical) attached to a linker (e.g., a radical of a linker) provided herein (e.g., and the linker (e.g., another radical of the linker) is further attached to a steroid radical provided herein).

In certain embodiments, provided herein is a compound having the structure of Formula (VIII):

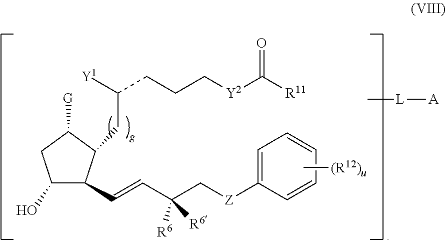

In some embodiments, [symbol] is a single bond or a double bond. In some embodiments, L is a linker. In some embodiments, A is a steroid radical. In some embodiments, G is OH and Y¹ is hydrogen. In some embodiments, G together with Y¹ form —O—CH₂—. In some embodiments, Y² is a bond or —CH₂—. In some embodiments, g is 1 or 2. In some embodiments, Z is —O— or —CH₂—. In some embodiments, R⁶ and R^{6′} are each independently hydrogen, halogen or OH. In some embodiments, R¹ is —OR¹³ or —NR^{13′}R^{13″}. In some embodiments, each R¹² is independently halogen or haloalkyl. In some embodiments, R¹³, R^{13′} and R^{13″} are each independently hydrogen or a C₁-C₃ alkyl. In some embodiments, u is 0-5. In some embodiments, R⁶ and R^{6′} are each independently fluoro. In some embodiments, R⁶ is OH and R^{6′} is hydrogen. In some embodiments, Z is —O—. In some embodiments, Z is —CH₂—. In some embodiments, R¹² is F and u is 2. In some embodiments, R¹² is CF₃ and u is 1. In some embodiments, u is 0. In some embodiments, u is 1. In some embodiments, u is 2. In some embodiments, u is 3. In some embodiments, u is 4. In some embodiments, u is 5. In some embodiments, R¹¹ is OH. In some embodiments, R¹¹ is —NHCH₂CH₃. In some embodiments, R¹¹ is —OCH(CH₃)₂. Provided in certain embodiments herein are pharmaceutically-acceptable salts or solvates of a compound of Formula (VIII).

In certain embodiments, provided herein is a compound having the structure of Formula (VIII-A):

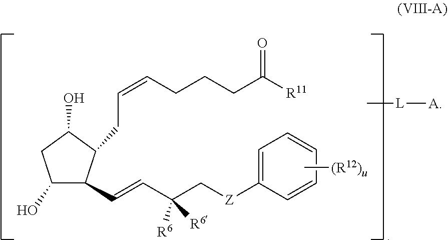

In some embodiments, L is a linker. In some embodiments, A is a steroid radical. In some embodiments, Z is —O— or —CH₂—. In some embodiments, R⁶ and R^{6′} are each independently hydrogen, halogen or OH; R¹¹ is —OR¹³, or —NR^{13′}R^{13″}. In some embodiments, each R¹² is independently halogen or haloalkyl. In some embodiments, R¹³, R^{13′}, and R^{13″} are each independently hydrogen or a C₁-C₃ alkyl. In some embodiments, u is 0-5. In some embodiments, R⁶ and R^{6′} are each independently fluoro. In some embodiments, R⁶ is OH and R^{6′} is hydrogen. In some embodiments, Z is —O—. In some embodiments, Z is —CH₂—. In some embodiments, R¹² is F and u is 2. In some embodiments, R¹² is CF₃ and u is 1. In some embodiments, u is 0. In some embodiments, u is 1. In some embodiments, u is 2. In some embodiments, u is 3. In some embodiments, u is 4. In some embodiments, u is 5. In some embodiments, R¹¹ is OH. In some embodiments, R¹¹ is —NHCH₂CH₃. In some embodiments, R¹¹ is —OCH(CH₃)₂. Provided in certain embodiments herein are pharmaceutically-acceptable salts or solvates of a compound of Formula (VIII-A).

In certain embodiments, provided herein is a compound having the structure of Formula (VIII-B):

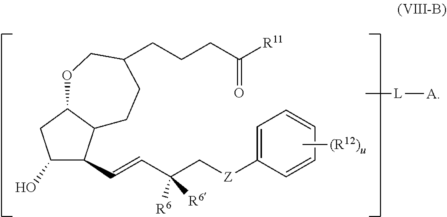

In some embodiments, L is a linker. In some embodiments, A is a steroid radical. In some embodiments, Z is —O— or —CH₂—. In some embodiments, R⁶ and R^{6′} are each independently hydrogen, halogen or OH. In some embodiments, R¹¹ is —OR¹³, or —NR^{13′}R^{13″}. In some embodiments, each R¹² is independently halogen or haloalkyl. In some embodiments, R¹³, R^{13′} and R^{13″} are each independently hydrogen or a C₁-C₃ alkyl. In some embodiments, u is 0-5. In some embodiments, R⁶ and R^{6′} are each independently fluoro. In some embodiments, R⁶ is OH and R^{6′} is hydrogen. In some embodiments, Z is —O—. In some embodiments, Z is —CH₂—. In some embodiments, R¹² is F and u is 2. In some embodiments, R¹² is CF₃ and u is 1. In some embodiments, u is 0. In some embodiments, u is 1. In some embodiments, u is 2. In some embodiments, u is 3. In some embodiments, u is 4. In some embodiments, u is 5. In some embodiments, R¹¹ is OH. In some embodiments, R¹¹ is —NHCH₂CH₃. In some embodiments, R¹¹ is —OCH(CH₃)₂. Provided in certain embodiments herein are pharmaceutically-acceptable salts or solvates of a compound of Formula (VIII-B).

In some embodiments, R¹¹ is a radical (e.g., a hydroxyl radical or an amino radical) attached to L. In some embodiments, R⁶ or R^{6′} is a radical (e.g., a hydroxyl radical) attached to L. In some embodiments, L is a bond. In some embodiments, L comprises one or more linker group, each linker group being independently selected from the group consisting of an alkylene, cycloalkylene or —O—.

In certain embodiments, A has the structure:

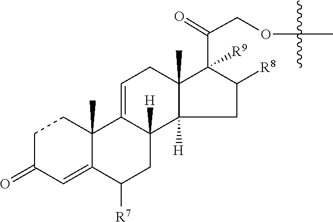

In some embodiments, [symbol] is a single bond or a double bond. In some embodiments, R⁷ is hydrogen or halogen. In some embodiments, R⁸ is a hydrogen or a C₁-C₄ alkyl. In some embodiments, R⁹ is absent, hydrogen or hydroxyl. In some embodiments, R⁷ is hydrogen. In some embodiments, R⁷ is fluoro. In some embodiments, R⁸ is hydrogen. In some embodiments, R⁸ is methyl, ethyl, propyl or butyl. In some embodiments, R⁸ is methyl, ethyl or butyl. In some embodiments, R⁸ is methyl. In some embodiments, R⁹ is hydroxyl.

In certain embodiments, A has the structure:

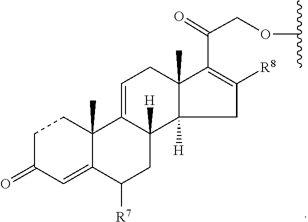

In some embodiments, [symbol] is a single bond or a double bond. In some embodiments, R⁷ is hydrogen or halogen. In some embodiments, R⁸ is a hydrogen or a C₁-C₄ alkyl. In some embodiments, R⁹ is absent, hydrogen or hydroxyl. In some embodiments, R⁷ is hydrogen. In some embodiments, R⁷ is fluoro. In some embodiments, R⁸ is hydrogen. In some embodiments, R⁸ is methyl, ethyl, propyl or butyl. In some embodiments, R⁸ is methyl, ethyl or butyl. In some embodiments, R⁸ is methyl.

In certain embodiments, A has the structure:

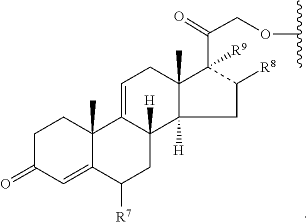

In some embodiments, [symbol] is a single or a double bond. In some embodiments, R⁷ is hydrogen or halogen. In some embodiments, R⁸ is a hydrogen or a C₁-C₄ alkyl. In some embodiments, R⁹ is absent, hydrogen or hydroxyl. In some embodiments, R⁷ is hydrogen. In some embodiments, R⁷ is fluoro. In some embodiments, R⁸ is hydrogen. In some embodiments, R⁸ is methyl, ethyl, propyl or butyl. In some embodiments, R⁸ is methyl, ethyl or butyl. In some embodiments, R⁸ is methyl. In some embodiments, R⁹ is hydroxyl.

In certain embodiments, A has the structure:

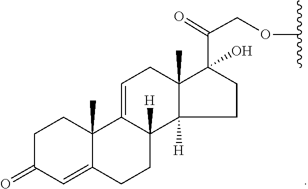

In certain embodiments, provided herein is a compound having the structure of Formula (IX):

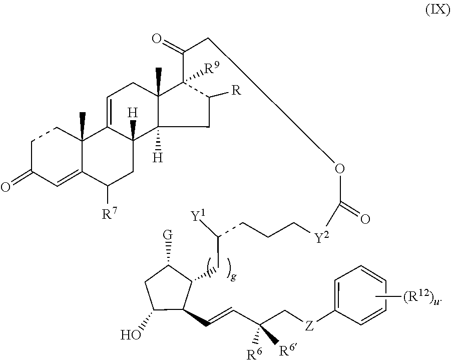

In certain embodiments, each [symbol] is independently a single bond or a double bond. In some embodiments, G is OH and Y¹ is hydrogen. In some embodiments, G together with Y¹ form —O—CH₂—. In some embodiments, Y² is a bond or —CH₂—. In some embodiments, g is 1 or 2. In some embodiments, Z is —O— or —CH₂—. In some embodiments, R⁶ and R^{6′} are each independently hydrogen, halogen or OH. In some embodiments, each R¹² is independently halogen or haloalkyl. In some embodiments, u is 0-5. In some embodiments, Z is —O— or —CH₂—. In some embodiments, R⁶ and R^{6′} are each independently hydrogen, halogen or OH. In some embodiments, each R¹² is independently halogen or haloalkyl. In some embodiments, R⁶ and R^{6′} are each fluoro. In some embodiments, R⁶ is OH and R^{6′} is hydrogen. In some embodiments, Z is —O—. In some embodiments, Z is —CH₂—. In some embodiments, R¹² is F and u is 2. In some embodiments, R¹² is CF₃ and u is 1. In some embodiments, u is 0. In some embodiments, u is 1. In some embodiments, u is 2. In some embodiments, u is 3. In some embodiments, u is 4. In some embodiments, u is 5. Also provided in certain embodiments herein are pharmaceutically-acceptable salts or solvates of a compound of Formula (IX).

In certain embodiments, provided herein is a compound having the structure of Formula (X):

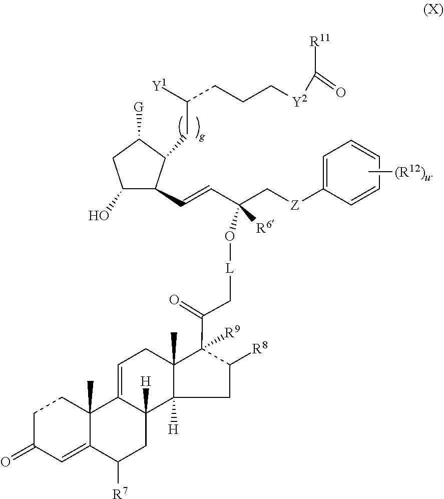

In certain embodiments, each [symbol] is independently a single bond or a double bond. In some embodiments, L is a linker. In some embodiments, L is a bond. In some embodiments, L is —(C═O)—, or —O—(C═O)—. In some embodiments, G is OH and Y¹ is hydrogen. In some embodiments, G together with Y¹ form —O—CH₂—. In some embodiments, Y² is a bond or —CH₂—. In some embodiments, g is 1 or 2. In some embodiments, Z is —O— or —CH₂—. In some embodiments, R⁶ and R^{6′} are each independently hydrogen, halogen or OH. In some embodiments, each R¹² is independently halogen or haloalkyl. In some embodiments, u is 0-5. In some embodiments, Z is —O— or —CH₂—. In some embodiments, R⁶ and R^{6′} are each independently hydrogen, halogen or OH. In some embodiments, each R¹² is independently halogen or haloalkyl. In some embodiments, R⁶ and R^{6′} are each independently fluoro. In some embodiments, R⁶ is OH and R^{6′} is hydrogen. In some embodiments, Z is —O—. In some embodiments, Z is —CH₂—. In some embodiments, R¹² is F and u is 2. In some embodiments, R¹² is CF₃ and u is 1. In some embodiments, u is 0. In some embodiments, u is 1. In some embodiments, u is 2. In some embodiments, u is 3. In some embodiments, u is 4. In some embodiments, u is 5. Also provided in certain embodiments herein are pharmaceutically-acceptable salts or solvates of a compound of Formula (X).

Provided in some embodiments herein is a compound having a structure represented by Formula (II):

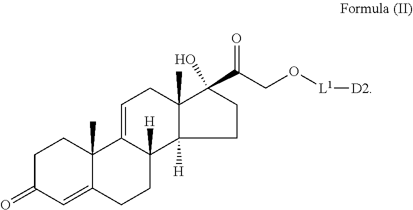

In some embodiments, D2 is a prostaglandin radical. In some embodiments, L¹ is -(Q¹-M-Q²)-. In some embodiments, Q¹ and Q² are each independently absent or (C═X¹)X². In some embodiments, X¹ is O or S. In some embodiments, X² is O, S, or NR¹. In some embodiments, R¹ is hydrogen or C₁-C₆ alkyl. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl). In some embodiments, the compound is a pharmaceutically acceptable salt or solvate.

In some embodiments, either or both of D1 or D2 are attached to L or L¹ through a hydroxyl radical of the D1 or D2. In some embodiments, D1 and D2 are attached to L or L¹ through a hydroxyl radical of the D1 or D2. In some embodiments, D1 and D2 are attached to L through a hydroxyl radical of the D1 or D2. In some embodiments, D1 and D2 are attached to L¹ through a hydroxyl radical of the D1 or D2.

In some embodiments, D2 is selected from the group consisting of latanoprost, latanoprost acid, travoprost, travoprost acid, tafluprost, tafluprost acid, bimatoprost, bimatoprost acid, sepetaprost, sepetaprost acid, 7-[(1R,2R,3R,5S)-3,5-dihydroxy-2-[(3R)-3-hydroxy-5-phenylpentyl]cyclopentyl]-4-(3-thioxo-3H-1,2-dithiol-5-yl)phenyl ester, 5Z-heptenoic acid, latanoprostene bunod, and (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl 6-(nitrooxy)hexanoate, or a fragment or radical of any of the foregoing.

In some embodiments, D2 is selected from the group consisting of substituted latanoprost, substituted latanoprost acid, substituted travoprost, substituted travoprost acid, substituted tafluprost, substituted tafluprost acid, substituted bimatoprost, substituted bimatoprost acid, substituted sepetaprost, substituted sepetaprost acid, substituted 7-[(1R,2R,3R,5S)-3,5-dihydroxy-2-[(3R)-3-hydroxy-5-phenylpentyl]cyclopentyl]-4-(3-thioxo-3H-1,2-dithiol-5-yl)phenyl ester, 5Z-heptenoic acid, substituted latanoprostene bunod, and substituted (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl 6-(nitrooxy)hexanoate, or a fragment or radical of any of the foregoing. In some embodiments, D2 is substituted latanoprost, substituted bimatoprost (e.g., bimatoprost substituted with substituted alkyl, substituted travoprost, or an acid or radical thereof.

In some embodiments, D2 is a radical represented by a structure of Formula (III):

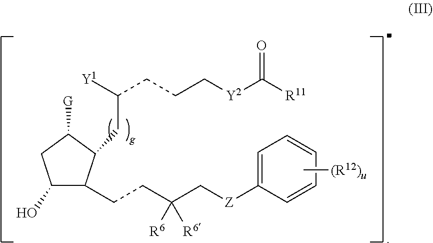

In some embodiments, each [symbol] is independently a single bond or a double bond. In some embodiments, G is OH. In some embodiments, Y¹ is hydrogen. In some embodiments, or G is taken together with Y¹ to form —O—CH₂—. In some embodiments, Y² is a bond or alkylene (e.g., —CH₂—). In some embodiments, g is 1 or 2. In some embodiments, Z is —O— or alkylene (e.g., —CH₂—). In some embodiments, R⁶ and R^{6′} are each independently hydrogen, halogen, alkyl, —OR¹⁴, or R⁶ and R^{6′} are taken together to form an oxo (e.g., R⁶ and R^{6′} are each independently hydrogen, halogen, or OH). In some embodiments, R¹⁴ is hydrogen, unsubstituted or substituted alkyl (e.g., substituted or unsubstituted C₁-C₆ alkyl (e.g., alkyl substituted with NO₂ or alkyl substituted with oxo and ONO₂)), or a point of attachment to any linker provided herein (e.g., L, L¹, L²). In some embodiments, R¹¹ is —OR¹³ or —NR^{13a}R¹³. In some embodiments, R¹³, R^{13a}, and R^{13b} are each independently selected from the group consisting of hydrogen, unsubstituted or substituted alkyl (e.g., substituted or unsubstituted C₁-C₆ alkyl (e.g., alkyl substituted with NO₂ or alkyl substituted with oxo and ONO₂)), substituted or unsubstituted aryl (e.g., aryl substituted with (cyclic) heteroalkyl (e.g., aryl substituted with 3H-1,2-dithiole-3-thione)), or a point of attachment to any linker provided herein (e.g., L, L¹, L²). In some embodiments, each R¹² is independently halogen (e.g., fluoro or chloro) or alkyl (e.g., haloalkyl (e.g., CF₃)). In some embodiments, u is 0-5.

In some embodiments, G is OH.

In some embodiments, Y¹ is hydrogen.

In some embodiments, G is taken together with Y¹ to form —O—CH₂—.

In some embodiments, Y² is a bond. In some embodiments, Y² is (e.g., C₁-C₃) alkylene (e.g., —CH₂—). In some embodiments, Y² is —CH₂—.

In some embodiments, g is 1. In some embodiments, g is 2.

In some embodiments, G is OH, Y¹ is hydrogen, Y² is —CH₂—, and g is 1.

In some embodiments, R¹³ is hydrogen, unsubstituted or substituted alkyl (e.g., substituted or unsubstituted C₁-C₆ alkyl (e.g., alkyl substituted with NO₂ or alkyl substituted with oxo and ONO₂)), substituted or unsubstituted aryl (e.g., aryl substituted with (cyclic) heteroalkyl (e.g., aryl substituted with 3H-1,2-dithiole-3-thione)), or a point of attachment to any linker provided herein (e.g., L, L¹, L²).

In some embodiments, R¹³, R^{13a}, and R^{13b} are each independently selected from the group consisting of hydrogen, substituted or unsubstituted C₁-C₆ alkyl, or a point of attachment to any linker provided herein (e.g., L, L¹, L²).

In some embodiments, R¹⁴ is unsubstituted or substituted alkyl. In some embodiments, R¹⁴ is substituted alkyl. In some embodiments, R¹⁴ is alkyl substituted with NO₂ or alkyl substituted with oxo and ONO₂.

In some embodiments, R¹⁴ is hydrogen or a point of attachment to any linker provided herein (e.g., L, L¹, L²).

In some embodiments, D2 is a radical represented by a structure of Formula (III-A):

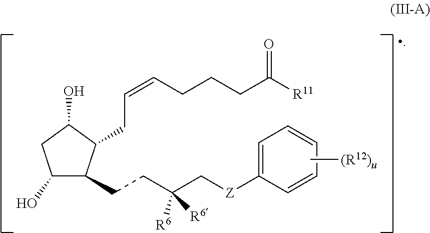

In some embodiments, [symbol] a singlebond or a double bond. In some embodiments, Z is —O— or —CH₂—. In some embodiments, R⁶ and R^{6′} are each independently hydrogen, halogen, or —OR¹⁴. In some embodiments, R¹⁴ is hydrogen or a point of attachment to any linker provided herein (e.g., L, L¹, L²). In some embodiments, R¹¹ is —OR¹³ or —NR^{13a}R^{13b}. In some embodiments, R¹³, R^{13a}, and R^{13b} are each independently hydrogen, C₁-C₃ alkyl, or a point of attachment to any linker provided herein (e.g., L, L¹, L²). In some embodiments, each R¹² is independently halogen (e.g., fluoro or chloro) or alkyl (e.g., haloalkyl (e.g., CF₃)). In some embodiments, u is 0-5.

In some embodiments, G together with Y¹ forms —O—CH₂—, and g is 2, and D2 is a radical represented by a structure of formula (III-B):

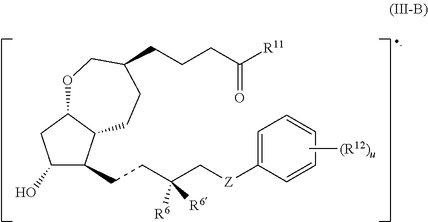

In some embodiments, [symbol] a single bond.

In some embodiments, [symbol] a double bond.

In some embodiments, Z is —O—.

In some embodiments, Z is —CH₂—.

In some embodiments, u is 0-2. In some embodiments, u is 0. In some embodiments, u is 1. In some embodiments, u is 2.

In some embodiments, R¹² is CF₃ and u is 1.

In some embodiments, R¹² is F and u is 2.

In some embodiments, R⁶ and R^{6′} are each independently hydrogen or halogen. In some embodiments, R⁶ and R^{6′} are each independently halogen.

In some embodiments, R⁶ and R^{6′} are each independently fluoro.

In some embodiments, R^{6′} is hydrogen.

In some embodiments, R⁶ is —OR¹⁴.

In some embodiments, R¹⁴ is hydrogen.

In some embodiments, R¹⁴ is a point of attachment to any linker provided herein (e.g., L, L¹, L²).

In some embodiments, R⁶ is —OR¹⁴ and R¹⁴ is a point of attachment to any linker provided herein (e.g., L, L¹, L²).

In some embodiments, R⁶ and R^{6′} are each independently hydrogen or —OR¹⁴.

In some embodiments, R⁶ is OH and R^{6′} is hydrogen.

In some embodiments, R⁶ is —OR¹⁴, R¹⁴ is a point of attachment to any linker provided herein (e.g., L, L¹, L²), and R^{6′} is hydrogen.

In some embodiments, R¹ is OH or NH(C₁-C₃ alkyl). In some embodiments, R¹ is OH. In some embodiments, R¹¹ is —NHCH₂CH₃.

In some embodiments, R¹ is —OR¹³.

In some embodiments, R¹³, R^{13a}, and R^{13b} are each independently selected from the group consisting of hydrogen, substituted or unsubstituted C₁-C₆ alkyl, or a point of attachment to any linker provided herein (e.g., L, L¹, L²).

In some embodiments, R¹ is —OR¹³ and R³ is a point of attachment to any linker provided herein (e.g., L, L¹, L²).

In some embodiments, R¹³ is hydrogen.

In some embodiments, R¹³ is unsubstituted or substituted alkyl. In some embodiments, R¹³ is substituted alkyl. In some embodiments, R¹³ is alkyl substituted with NO₂ or alkyl substituted with oxo and ONO₂.

In some embodiments, R¹³ is substituted or unsubstituted aryl. In some embodiments, R¹³ is substituted aryl. In some embodiments, R¹³ is aryl substituted with (cyclic) heteroalkyl. In some embodiments, R¹³ is aryl substituted with 3H-1,2-dithiole-3-thione.

In some embodiments, R^{13a} and R^{13b} are each independently hydrogen or unsubstituted C₁-C₆ alkyl. In some embodiments, R^{13a} and R^{13b} are hydrogen. In some embodiments, R^{13a} and R^{13b} are each independently unsubstituted C₁-C₃ alkyl.

In some embodiments, R¹¹ is —OR¹³ and R¹³ is a point of attachment to any linker provided herein (e.g., L, L¹, L²), R⁶ is OH, and R^{6′} is hydrogen.

In some embodiments, R¹ is OH, R⁶ is —OR¹⁴, R¹⁴ is a point of attachment to any linker provided herein (e.g., L, L¹, L²), and R^{6′} is hydrogen.

In some embodiments, D2 is latanoprost, bimatoprost, travoprost, or an acid or radical thereof. In some embodiments, D2 is a latanoprost radical. In some embodiments, D2 is a bimatoprost radical. In some embodiments, D2 is a travoprost radical.

In some embodiments, D2 is latanoprost substituted with substituted alkyl, or an acid or radical thereof. In some embodiments, D2 is latanoprost substituted with one or more alkyl substituent, each alkyl substituent being independently selected from the group consisting of oxo and aryl.

In some embodiments, D2 is bimatoprost substituted with substituted alkyl, or an acid or radical thereof. In some embodiments, D2 is latanoprost substituted with one or more alkyl substituent, each alkyl substituent being independently selected from the group consisting of oxo and aryl.

In some embodiments, D2 is travoprost substituted with substituted alkyl, or an acid or radical thereof. In some embodiments, D2 is latanoprost substituted with one or more alkyl substituent, each alkyl substituent being independently selected from the group consisting of oxo and aryl)).

In some embodiments, D2 is a C1-latanoprost radical, a C15-latanoprost radical, a C1-bimatoprost radical, a C15-bimatoprost radical, a C1-travoprost radical, or a C15-travoprost radical.

In some embodiments, C#, where # is a number, indicates the carbon atom (e.g., C1 or C15) of a compound provided herein (e.g., of a steroid, a prostaglandin, or a radical thereof) to which a hydroxy (radical (e.g., R⁶, R^{6′}, or R¹¹ (e.g., R⁶ being —OR¹⁴ and R¹⁴ being the point of attachment to any linker provided herein (e.g., L, L¹, L²); and R¹ being —OR¹³ and R¹³ being the point of attachment to any linker provided herein (e.g., L, L¹, L²)))) resides.

In some embodiments, D2 is a substituted C1-latanoprost radical, a substituted C15-latanoprost radical, a substituted C1-bimatoprost radical, a substituted C15-bimatoprost radical, a substituted C1-travoprost radical, or a substituted C15-travoprost radical.

In some embodiments, D2 is C1-bimatoprost (radical) substituted at C15 with substituted alkyl (e.g., alkyl substituted with one or more alkyl substituent, each alkyl substituent being independently selected from the group consisting of oxo and aryl).

In some embodiments, D2 is C1-bimatoprost (radical) substituted at C15 with alkyl substituted with oxo.

In some embodiments, D2 is C1-bimatoprost (radical) substituted at C15 with alkyl substituted with oxo and aryl.

In some embodiments, D2 is C1-bimatoprost (radical) substituted at C9 and C11 with substituted alkyl (e.g., alkyl substituted with oxo), the C9 and C11 being taken together with the substituted alkyl (e.g., to form a substituted heterocyclyl).

In some embodiments, D2 is C1-bimatoprost (radical) substituted at C9 and C11 with alkyl substituted with oxo, the C9 and C11 being taken together with the alkyl substituted with oxo (e.g., to form a substituted heterocyclyl).

In some embodiments, D2 is C1-latanoprost (radical) substituted at C15 with substituted alkyl (e.g., alkyl substituted with one or more alkyl substituent, each alkyl substituent being independently selected from the group consisting of oxo and aryl).

In some embodiments, D2 is C1-latanoprost (radical) substituted at C15 with alkyl substituted with oxo.

In some embodiments, D2 is C1-latanoprost (radical) substituted at C15 with alkyl substituted with oxo and aryl.

In some embodiments, D2 is C1-latanoprost (radical) substituted at C9 and C11 with substituted alkyl (e.g., alkyl substituted with oxo), the C9 and C11 being taken together with the substituted alkyl (e.g., to form a substituted heterocyclyl).

In some embodiments, D2 is C1-latanoprost (radical) substituted at C9 and C11 with alkyl substituted with oxo, the C9 and C11 being taken together with the alkyl substituted with oxo (e.g., to form a substituted heterocyclyl).

In some embodiments, D1 is a steroid (e.g., dexamethasone, anecortave (e.g., anecortave desacetate), etc.). In some embodiments, D1 is or is derived from anecortave (e.g., anecortave acetate or anecortave desacetate). In some embodiments, D1 is a steroid (e.g., dexamethasone, anecortave (e.g., anecortave desacetate), etc.). In some embodiments, the steroid is a corticosteroid (e.g., glucocorticoid or mineralcorticoid), a sex steroid, a neurosteroid, an aminosteroid, or a secosteroid. In some embodiments, D2 is not a steroid.

In some embodiments, the steroid is a glucocorticoid. In some embodiments, the glucocorticoid is selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, loprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol, or the like.

In some embodiments, the steroid is a mineralocorticoid. In some embodiments, the mineralocorticoid is selected from the group consisting of aldosterone, fludrocortisone, deoxycorticosterone, and corticosterone, or the like. In some embodiments, the mineralocorticoid is canrenone (e.g., potassium canrenoate), drospirenone, eplerenone, spirolactone, or a metabolite thereof (e.g., 7α-thiomethylspironolactone, canrenone, 6β-hydroxy-7α-thiomethylspironolactone, and 7α-thiospironolactone).

In some embodiments, the steroid is an anabolic steroid. In some embodiments, the anabolic steroid is selected from the group consisting of androisoxazole, androstenediol, bolandiol, bolasterone, clostebol, ethylestrenol, formyldienolone, 4-hydroxy-19-nortestosterone, methandriol, methenolone, methyltrienolone, nandrolone, norbolethone, oxymesterone, stenbolone, and trenbolone, or the like.

In some embodiments, the steroid is an androgenic steroid. In some embodiments, the androgenic steroid is selected from the group consisting of boldenone, fluoxymesterone, mestanolone, mesterolone, methandrostenolone, 17-methyltestosterone, 17-α-methyltestosterone 3-cyclopentyl enol ether, norethandrolone, normethandrone, oxandrolone, oxymesterone, oxymetholone, prasterone, stanlolone, stanozolol, testosterone, testosterone 17-chloral hemiacetal, testosterone proprionate, testosterone enanthate tiomesterone dehydroepiandrosterone (DHEA), androstenedione, androstenediol, androsterone, dihydrotestosterone (DHT), and androstanolone, or the like.

In some embodiments, the steroid is a progestin steroid. In some embodiments, the progestin steroid is selected from the group consisting of progesterone, norethisterone, norethisterone acetate, gestodene, levonorgestrel, allylestrenol, anagestone, desogestrel, dimethisterone, dydrogesterone, ethisterone, ethynodiol, ethynodiol diacetate, etonogestrel, gestodene, ethinylestradiol, haloprogesterone, 17-hydroxy-16-methylene-progesterone, 17-alpha-hydroxyprogesterone, lynestrenol, medroxyprogesterone, melengestrol, norethindrone, norethynodrel, norgesterone, gestonorone, norgestimate, norgestrel, levonorgestrel, norgestrienone, norvinisterone, pentagestrone, MENT (7-methyl-19-testosterone); norelgestromin, and trimigestone drospirenone, tibolone, and megestrol, or the like.

In some embodiments, the steroid is an estrogen steroid. In some embodiments, the estrogen steroid is selected from the group consisting of estradiol, estrone, eguilenin, equilin, estradiol benzoate, estriol, ethinyl estradiol, mestranol, moxestrol, mytatrienediol, quinestradiol, and quinestrol, or the like.

In some embodiments, the steroid is selected from the group consisting of abiraterone, cyproterone acetate, dutasteride, enzalutamide, finasteride, galeterone, fusidic acid, cholesterol, 11-deoxycortisol, 11-deoxycorticosterone, pregnenolone, cholic acid, chenodeoxycholic acid, ursodeoxycholic acid, obeticholic acid, tetrahydrocortisone, tetrahydrodeoxycortisol, tetrahydrocorticosterone, 5α-dihydrocorticosterone, 5α-dihydropregesterone, flugestone, prebediolone, chlormadinone acetate, medrogestone, and segesterone acetate, or the like.

In some embodiments, the steroid is an anti-angiogenic or an intraocular pressure (IOP) lowering steroid. In some embodiments, the angiostatic) lowering steroid is selected from the group consisting of anecortave acetate, anecortave (e.g., anecortave desacetate), 11-epicortisol, 17α-hydroxyprogesterone, tetrahydrocortexolone, and tetrahydrocortisol, or the like. In some embodiments, the angiostatic steroid is anecortave desacetate.

In some embodiments, the steroid is a cholic acid-related bile acid steroid. In some embodiments, the cholic acid-related bile acid steroid is selected from the group consisting of deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, and tauroursodeoxycholic acid, or the like.

In some embodiments, the steroid is a neurosteroid. In some embodiments, the neurosteroid is selected from the group consisting of alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, and 24(S)-hydroxycholesterol, or the like.

In some embodiments, the steroid is a steroid pheromone. In some embodiments, the steroid pheromone is selected from the group consisting of androstadienol, androstadienone, androstenol, androstenone, estratetraenol, 5-dehydroprogesterone, 6-dehydro-retroprogesterone, allopregnanolone, and hydroxyprogesterone caproate, or the like.

In some embodiments, the steroid is a steroid metabolite. In some embodiments, the steroid metabolite is selected from the group consisting of tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, and progesterone, or the like.

In some embodiments, the steroid is a progestin. In some embodiments, the progestin is selected from the group consisting of allopregnone-3α,20α-diol, allopregnone-3β,20β-diol, allopregnane-3β,21-diol-11,20-dione, allopregnane-3β,17α-diol-20-one, 3,20-allopregnanedione,3β,11β,17α,20β,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,20β-triol, allopregnane-3β,17α,21-triol-11,20-dione, allopregnane-3β,11β,21-triol-20-one, allopregnane-3β,17α,21-triol-20-one, allopregnane-3α-ol-20-one, allopregnane-3β-ol-20-one, pregnanediol, 3,20-pregnanedione, 4-pregnene-20,21-diol-3,11-dione, 4-pregnene-11β,17α,20β,21-tetrol-3-one, 4-pregnene-17α,20β,21-triol-3,11-dione, 4-pregnene-17α,20β,21-triol-3-one, and pregnenolone, or the like.

In some embodiments, D1 is a (e.g., hydroxyl or carboxyl) radical of a compound selected from the group consisting of:

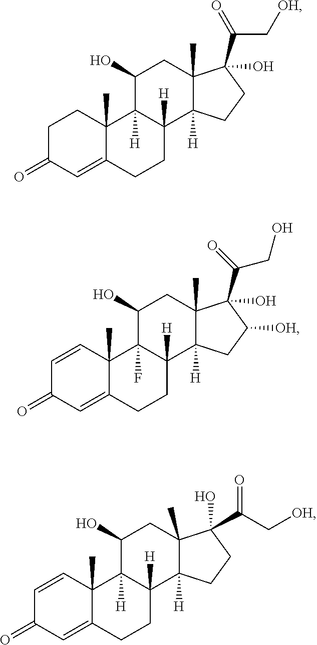

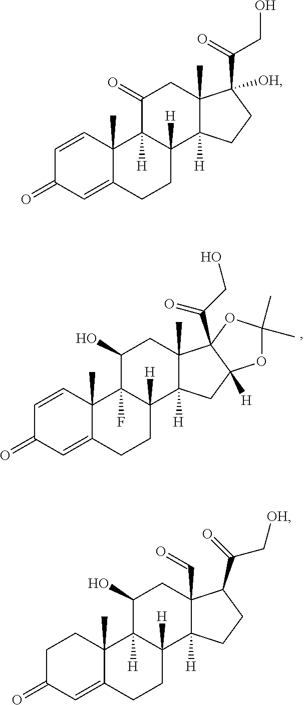

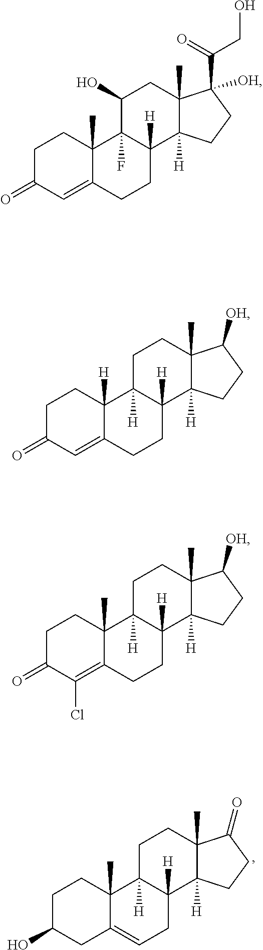

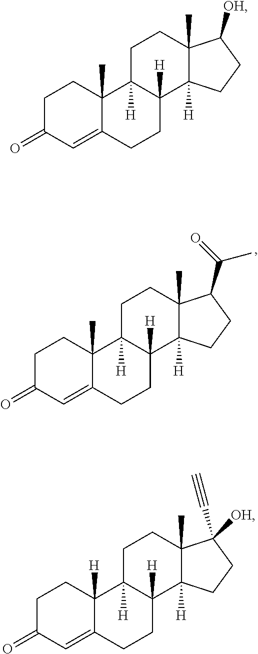

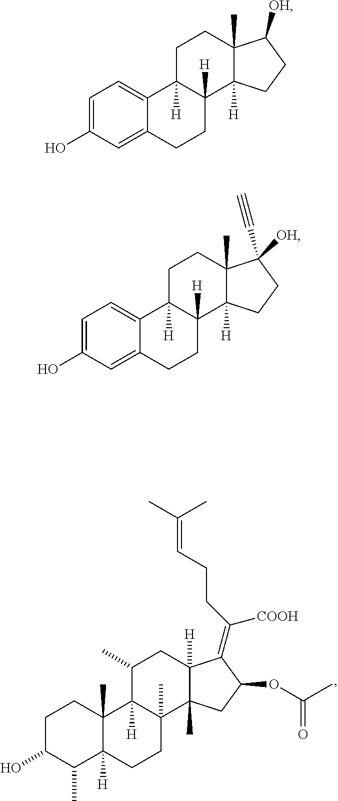

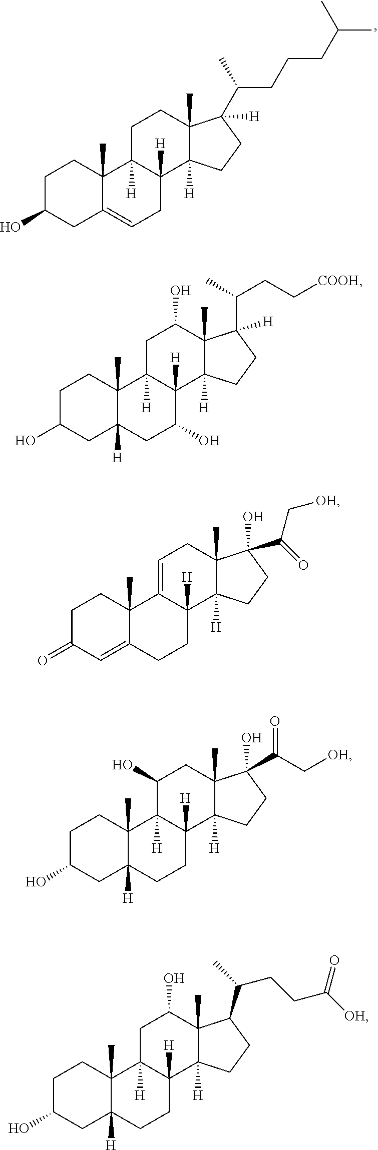

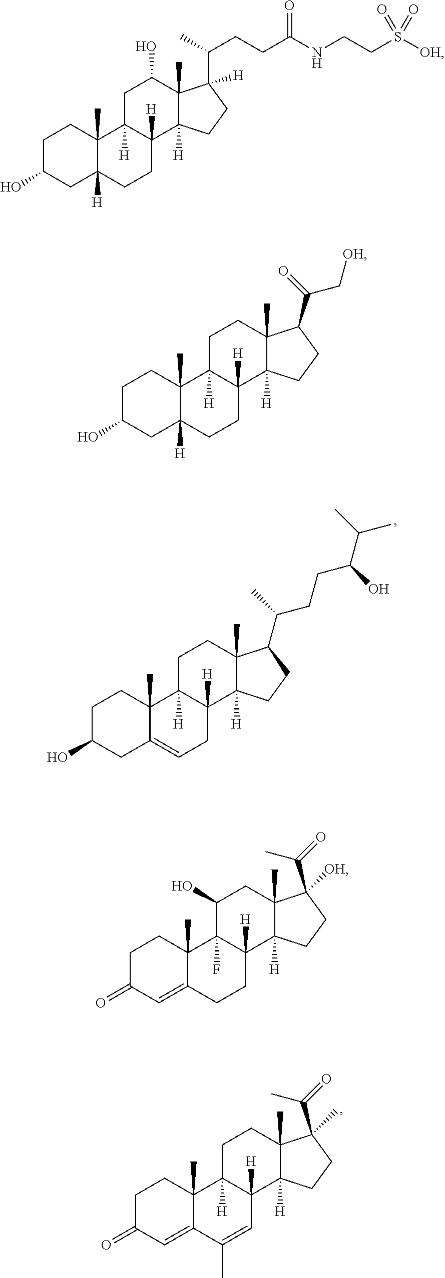

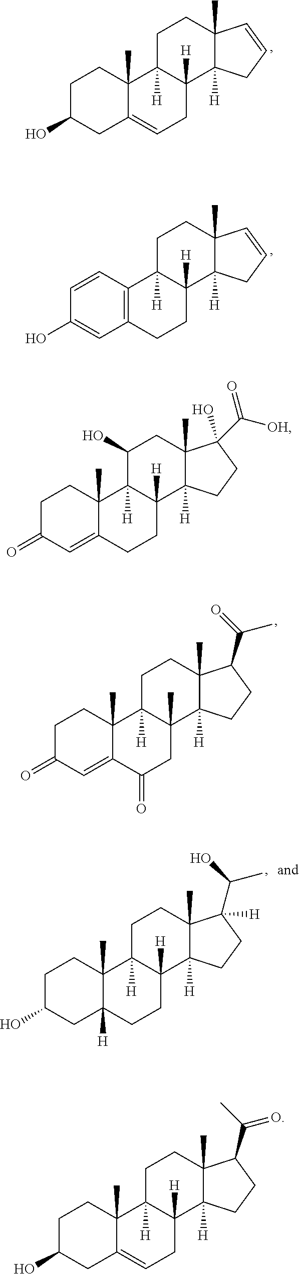

In some embodiments, the D2 is a (e.g., hydroxyl or carboxyl) radical of a compound selected from the group consisting of:

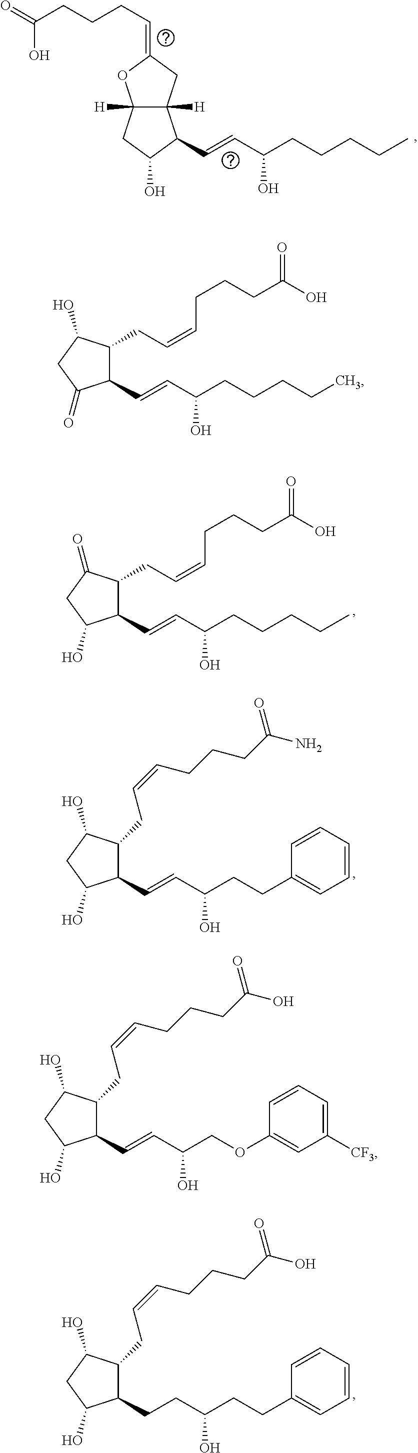

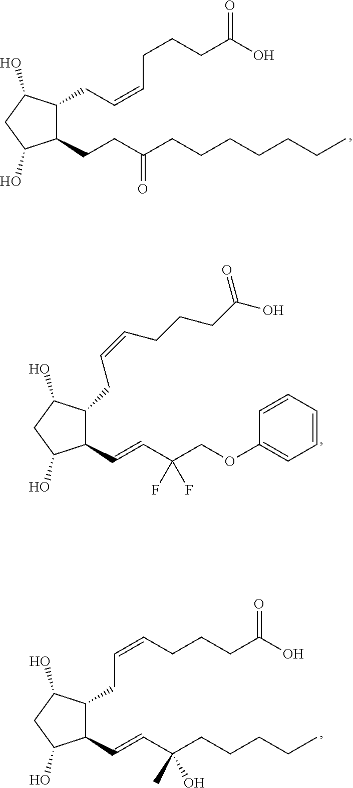

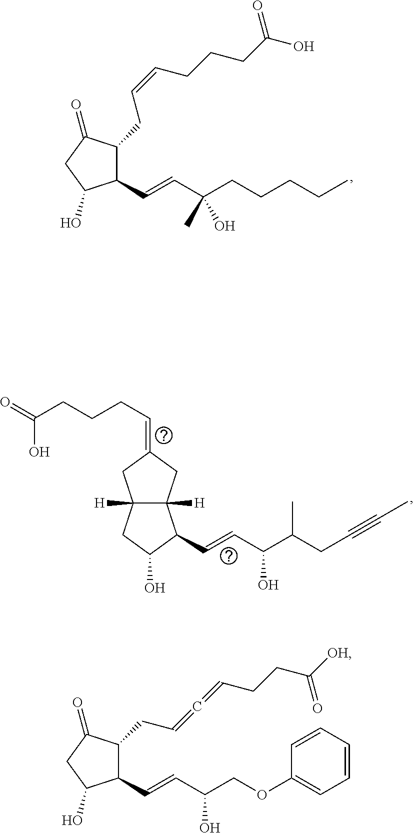

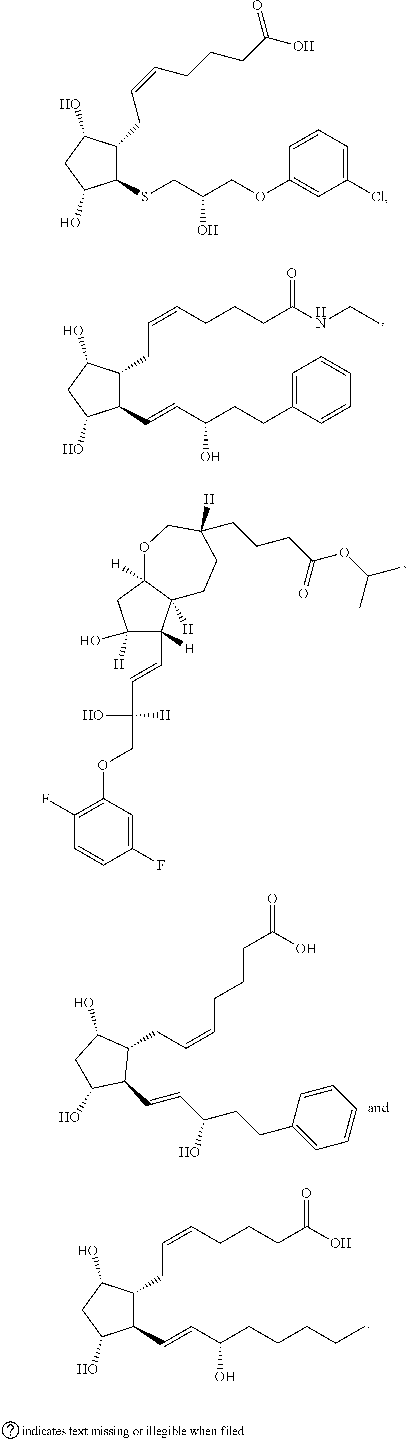

In some embodiments D2 is a (e.g. hydroxyl or carboxyl) radical of a compound selected from the group consisting of:

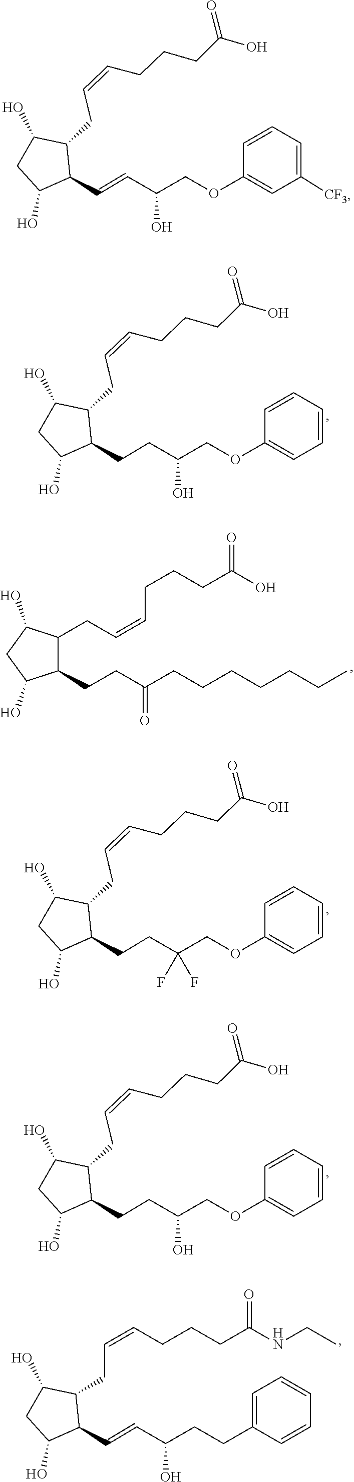

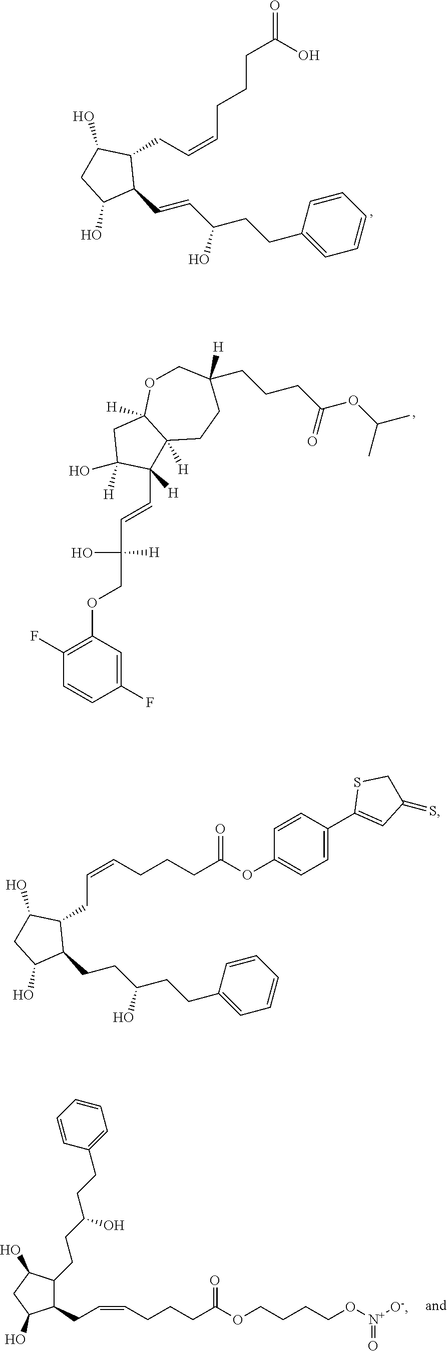

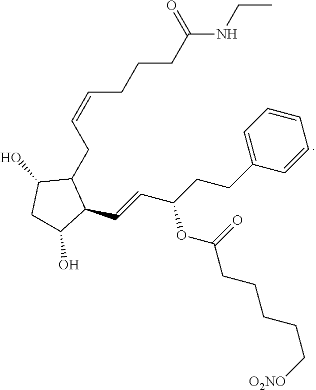

In some embodiments, D2 is a prostaglandin. In some embodiments, the prostaglandin is selected from the group consisting of latanoprost, latanoprost acid, travoprost, travoprost acid, tafluprost, tafluprost acid, bimatoprost, bimatoprost acid, sepetaprost, sepetaprost acid, 7-[(1R,2R,3R,5S)-3,5-dihydroxy-2-[(3R)-3-hydroxy-5-phenylpentyl]cyclopentyl]-4-(3-thioxo-3H-1,2-dithiol-5-yl)phenyl ester, 5Z-heptenoic acid, latanoprostene bunod, and (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl 6-(nitrooxy)hexanoate, or a fragment or radical of any of the foregoing.

In some embodiments, Q¹ and Q² are each independently absent, C═O, C=S, (C═O)O, (C═O)S, (C═O)N, or (C═S)S. In some embodiments, Q¹ and Q² are each independently absent, C═O, or (C═O)O.

In some embodiments, Q¹ is absent, C═O, C=S, (C═O)O, (C═O)S, (C═O)N, or (C═S)S. In some embodiments, Q¹ is C═O or (C═O)O.

In some embodiments, Q¹ is absent.

In some embodiments, Q¹ is C═O.

In some embodiments, Q¹ is C=S.

In some embodiments, Q¹ is (C═O)O.

In some embodiments, Q¹ is (C═O)S.

In some embodiments, Q¹ is (C═O)N.

In some embodiments, Q¹ is (C═S)S.

In some embodiments, Q² is absent, C═O, C=S, (C═O)O, (C═O)S, (C═O)N, or (C═S)S. In some embodiments, Q² is C═O or (C═O)O.

In some embodiments, Q² is absent.

In some embodiments, Q² is C═O.

In some embodiments, Q² is C=S.

In some embodiments, Q² is (C═O)O.

In some embodiments, Q² is (C═O)S.

In some embodiments, Q² is (C═O)N.

In some embodiments, Q² is (C═S)S.

In some embodiments, Q¹ and Q² are absent.

In some embodiments, Q¹ and Q² are C═O.

In some embodiments, Q¹ and Q² are C=S.

In some embodiments, Q¹ and Q² are (C═O)O.

In some embodiments, Q¹ and Q² are (C═O)S.

In some embodiments, Q¹ and Q² are (C═O)N.

In some embodiments, Q¹ and Q² are (C═S)S.

In some embodiments, Q¹ and Q² are each independently C═O or (C═O)O.

In some embodiments, Q¹ is C═O and Q² is absent.

In some embodiments, Q¹ is absent and Q² is C═O.

In some embodiments, Q¹ is (C═O)O and Q² is absent.

In some embodiments, Q¹ is absent and Q² is (C═O)O.

In some embodiments, Q¹ is C═O and Q² is (C═O)S.

In some embodiments, Q¹ is (C═O)S and Q² is C═O.

In some embodiments, Q¹ is C=O and Q² is (C═S)S.

In some embodiments, Q¹ is (C═S)S and Q² is C═O.

In some embodiments, Q¹ is C=O and Q² is (C═O)N.

In some embodiments, Q¹ is (C═O)N and Q² is C═O.

Unless stated specifically otherwise herein, each instance of (C═X¹)X² includes a specific and explicit recitation of (C═X¹)X² and X²(C═X¹). Likewise, unless stated specifically otherwise herein, each instance of (C═O)O includes a specific and explicit recitation of (C═O)O and O(C═O); each instance of (C═O)N includes a specific and explicit recitation of (C═O)N and N(C═O); each instance of (C═O)S includes a specific and explicit recitation of (C═O)S and S(C═O); and each instance of (C═S)S includes a specific and explicit recitation of (C═S)S and S(C═S). In some instances, X² (e.g., O, N, or S) taken together with any steroid, prostaglandin, or linker provided herein to form a C—X² bond (e.g., C—O, C—S, or C—N).

In some embodiments, M is substituted or unsubstituted alkyl (e.g., C₁-C₆ alkyl), substituted or unsubstituted heteroalkyl (e.g., C₁-C₆ heteroalkyl), or substituted or unsubstituted aryl.

In some embodiments, M is substituted or unsubstituted alkyl (e.g., C₁-C₆ alkyl). In some embodiments, M is substituted or unsubstituted C₁-C₆ alkyl. In some embodiments, M is substituted (e.g., C₁-C₆ alkyl) alkyl. In some embodiments, M is substituted alkyl (e.g., C₁-C₆ alkyl), the alkyl being substituted with one or more substituent, each substituent being independently selected from the group consisting of oxo, halo, alkyl, and heteroalkyl (e.g., —NHCOCH₃). In some embodiments, M is alkyl (e.g., C₁-C₆ alkyl) substituted with oxo. In some embodiments, M is alkyl (e.g., C₁-C₆ alkyl) substituted with one or more —NHCOCH₃. In some embodiments, M is —CH(NHCOCH₃)CH₂—. In some embodiments, M is unsubstituted alkyl (e.g., C—Cs alkyl). In some embodiments, M is —(CH₂)ₘ—, m being 1-10. In some embodiments, M is —CH₂—, —CH₂CH₂—, or —CH₂CH₂CH₂—.

In some embodiments, M is substituted or unsubstituted heteroalkyl (e.g., C₁-C₆ heteroalkyl). In some embodiments, M is substituted or unsubstituted C₁-C₆ heteroalkyl. In some embodiments, M is unsubstituted heteroalkyl (e.g., C₁-C₆ heteroalkyl). In some embodiments, M is —SCH₂CH₂—.

In some embodiments, M is substituted or unsubstituted aryl. In some embodiments, M is unsubstituted aryl. In some embodiments, M is unsubstituted phenyl.

In some embodiments, M is alkyl (e.g., methyl) substituted with oxo and Q¹ and Q² are absent.

In some embodiments, Q¹ and Q² are (C═O)O and M is —CH₂CH₂CH₂—.

In some embodiments, Q¹ and Q² are C=O and M is —CH₂CH₂— or phenyl.

In some embodiments, Q¹ is C═O, Q² is absent, and M is —CH₂—, —CH₂CH₂—, —CHCH₃—, or —CH(NHCOCH₃)CH₂—.

In some embodiments, Q¹ is C=O, Q² is absent, and M is —SCH₂CH₂—.

In some embodiments, Q¹ is C═O, Q² is S(C═O), and M is —CH₂CH₂—.

In some embodiments, Q¹ is C═O, Q² is S(C═S), and M is —CH₂CH₂—.

In some embodiments, Q¹ is C═O, Q² is N(C═O), and M is —CH₂CH₂—.

In some embodiments, the linker (e.g., L, L¹, or L²) is a bond, C═O, —C═OCH₂CH₂C═O—, —C=OphenylC═O—, —C═OCH₂CH₂—, —(C═O)OCH₂CH₂CH₂O(C═O)—, —C═OCH(NHCOCH₃)CH₂—, —C═OCH₂O(C═O)—, —(C═O)OCH₂C═O—, —C═OCH(CH₃)O(C═O)—, —(C═O)OCH(CH₃)C═O—, —C═OCH₂CH₂S—, —C═OCH₂CH₂SC═O—, —C═OCH₂CH₂SC═S—, or —C═OCH₂CH₂NHC═O—.

In some embodiments, the linker (e.g., L, L¹, or L²) is C═O, —C═OCH₂CH₂C═O—, —C=OphenylC═O—, —C═OCH₂CH₂—, —(C═O)OCH₂CH₂CH₂O(C═O)—, —C═OCH(NHCOCH₃)CH₂—, —C═OCH₂O(C═O)—, —(C═O)OCH₂C═O—, —C═OCH(CH₃)O(C═O)—, —(C═O)OCH(CH₃)C═O—, —C═OCH₂CH₂S—, —C═OCH₂CH₂SC═O—, —C═OCH₂CH₂SC═S—, or —C═OCH₂CH₂NHC═O—.

In some embodiments, the linker (e.g., L, L¹, or L²) is —C═OCH₂CH₂C═O—, —C═OphenylC═O—, —C═OCH₂CH₂—, —(C═O)OCH₂CH₂CH₂O(C═O)—, —C═OCH(NHCOCH₃)CH₂—, —C═OCH₂O(C═O)—, —(C═O)OCH₂C═O—, —C═OCH(CH₃)O(C═O)—, —(C═O)OCH(CH₃)C═O—, —C═OCH₂CH₂S—, —C═OCH₂CH₂SC═O—, —C═OCH₂CH₂SC═S—, or —C═OCH₂CH₂NHC═O—.

In some embodiments, the linker (e.g., L, L¹, or L²) is C═O.

In some embodiments, the linker (e.g., L, L¹, or L²) is a bond.

In some embodiments, a hydroxyl radical or a carboxylate radical of any one of Formula (I′), Formula (IA), Formula (IB), Formula (IC), Formula (II′), Formula (IIA), Formula (IIB), Formula (III′), Formula (IV′), Formula (V′), Formula (VII), Formula (VII-A), or Formula (VII-B) is attached to a hydroxyl radical or a carboxylate radical of another of any one of Formula (I′), Formula (IA), Formula (IB), Formula (IC), Formula (II′), Formula (IIA), Formula (IIB), Formula (III′), Formula (IV′), Formula (V′), Formula (VII), Formula (VII-A), or Formula (VII-B) through a linker. In some embodiments, a hydroxyl radical of any one of Formula (I′), Formula (IA), Formula (IB), Formula (IC), Formula (II′), Formula (IIA), Formula (IIB), Formula (III′), Formula (IV′), Formula (V′), Formula (VII), Formula (VII-A), or Formula (VII-B) is attached to a hydroxyl radical of any one of Formula (I′), Formula (IA), Formula (IB), Formula (IC), Formula (II′), Formula (IIA), Formula (IIB), Formula (III′), Formula (IV′), Formula (V′), Formula (VII), Formula (VII-A), or Formula (VII-B) through a linker. In some embodiments, a carboxylate radical of any one of Formula (I′), Formula (IA), Formula (IB), Formula (IC), Formula (II′), Formula (IIA), Formula (IIB), Formula (III′), Formula (IV′), Formula (V′), Formula (VII), Formula (VII-A), or Formula (VII-B) is attached to a hydroxyl radical of any one of Formula (I′), Formula (IA), Formula (IB), Formula (IC), Formula (II′), Formula (IIA), Formula (IIB), Formula (III′), Formula (IV′), Formula (V′), Formula (VII), Formula (VII-A), or Formula (VII-B) through a linker.

In some embodiments, a carboxylate radical of any steroid described herein is attached to a carboxylate radical of any prostaglandin described herein through a linker.

In some embodiments, a hydroxyl radical of any steroid described herein is attached to a hydroxyl radical of any prostaglandin described herein through a linker.

In some embodiments, a carboxyl radical of any steroid described herein is attached to a hydroxyl radical of any prostaglandin described herein through a linker.

In some embodiments, a hydroxyl radical of any steroid described herein is attached to a carboxyl radical of any prostaglandin described herein through a linker.

In some embodiments, any one of R^{a}, R^{a′}, R^{b}, R^{b′}, R^{b″}, R^{c}, R^{c″}, R^{d}, or R^{d′} is an ester radical, a hydroxyl radical, or a carboxylate radical, and any one of R¹, R², R³, R⁴, R⁶, R⁶, R^{6′}, R^{6″}, or R¹⁰ is an amide radical, a thiol radical, a hydroxyl radical, or a carboxylate radical. In some embodiments, any one of R^{a}, R^{a′}, R^{b}, R^{b′}, R^{b″}, R^{c}, R^{c″}, R^{d}, or R^{d′} is an ester radical, a hydroxyl radical, or a carboxylate radical, and any one of R¹, R², R³, R⁴, R⁵, R⁶, R^{6′}, R^{6″}, or R¹⁰ is a hydroxyl radical or a carboxylate radical. In some embodiments, any radical of R^{a}, R^{a″}, R^{b}, R^{b′}, R^{b″}, R^{c}, R^{c″}, R^{d}, or R^{d′} is adjoined to any radical of R¹, R², R³, R⁴, R⁶, R⁶, R^{6′}, R^{6″}, or R¹⁰ by a linker. In some embodiments, any radical of R^{a}, R^{a″}, R^{b}, R^{b′}, R^{b″}, R^{c}, R^{c″}, R^{d}, or R^{d′} is adjoined to any radical of R⁴, R⁶, R⁶, R^{6′}, R^{6″}, or R¹⁰ by a linker. In some embodiments, any radical of R^{a}, R^{b}, R^{c}, or R^{d} is adjoined to any radical of R⁴, R⁶, R⁶, R^{6′}, R^{6″}, or R¹⁰ by a linker. In some embodiments, any radical of R^{d} or R^{d′} is adjoined to any radical of R⁴, R⁵, R⁶, R^{6′}, R^{6″}, or R¹⁰ by a linker. In some embodiments, a radical of R^{d} is adjoined to any radical of R⁴, R⁶, R⁶, R^{6′}, R^{6″}, or R¹⁰ by a linker. In some embodiments, a radical of R^{d} is adjoined to a radical of R⁴ or R¹⁰ by a linker. In some embodiments, a radical of R^{d} is adjoined to any radical of R⁶, R^{6′}, or R^{6″} by a linker.

In some embodiments, the linker is a bond. In some embodiments, the linker is not a bond. In some embodiments, the linker is C═O.

In some embodiments, the linker is a bond, alkyl, heteroalkyl, or alkoxy, wherein the alkyl, heteroalkyl, or alkoxy is optionally substituted. In some embodiments, the alkyl, heteroalkyl, or alkoxy are each independently substituted with one or more substituent, each substituent being independently selected from the group consisting of oxo, —O— (e.g., hydroxyl or alkoxy), —S— (e.g., thiol or thioalkoxy), silicone, amino, alkyl, heteroalkyl, cycloalkyl, heterocycloalkyl, aryl or heteroaryl, wherein the alkyl, heteroalkyl, cycloalkyl, heterocycloalkyl, aryl or heteroaryl are optionally further substituted (e.g., with halogen or hydroxyl). In some embodiments, the linker is alkyl (alkylene) and the alkyl (alkylene) is substituted with one or more groups selected from —OH, halo, oxo, alkyl, heteroalkyl, cycloalkyl, and heterocycloalkyl.

In some embodiments, the linker comprises at least one oxo. In some embodiments, the linker comprises two oxo groups. In some embodiments, the linker comprises one or more ester, carbonate, anhydride, carbamate, ester, or any combination thereof. In some embodiments, the linker comprises at least one carbamate. In some embodiments, the linker comprises at least one carbonate. In some embodiments, the linker comprises at least one ester. In some embodiments, the linker comprises two or more esters.

In some embodiments, the linker comprises one or more linker groups, each linker group being independently selected from the group consisting of —O—, —S—, optionally substituted alkylene (e.g., alkenyl, alkynyl, branched (e.g., polypropylene), haloalkyl), optionally substituted heteroalkylene (e.g., polyTHF), and optionally substituted cycloalkylene. In some embodiments, the linker comprises one or more linker groups, each linker group being independently selected from the group consisting of alkyl, alkoxy, and cycloalkyl, wherein the alkyl, alkoxy, or cycloalkyl are optionally substituted.

In some embodiments, the linker comprises one or more linker groups selected from —O—S—, unsubstituted alkylene, (CH₂CH₂)ₙ, (CHCH)ₙ, O(CH₂CH₂O)ₙ, (CH₂CH₂O)ₙ, and (CH(CH₃)C(═O)O)ₙ, wherein n is 1-20. In some embodiments, the linker is unsubstituted alkylene, (CH₂CH₂)ₙ, (CHCH)ₙ, O(CH₂CH₂O)ₙ, (CH₂CH₂O)ₙ, (CH(CH₃)C(═O)O)ₙ, or (CH₂CH₂)ₙC=O(CH(CH₃)C(═O)O)ₙ, wherein n is 1-20.

In some embodiments, the linker is alkyl (alkylene) substituted with one or more groups selected from —OH, halo, oxo, alkyl, heteroalkyl, cycloalkyl, and heterocycloalkyl. In some embodiments, the linker is unsubstituted alkyl (alkylene). In some embodiments, the linker is heteroalkyl (heteroalkylene) substituted with one or more groups selected from halo or alkyl. In some embodiments, the linker is unsubstituted heteroalkyl (heteroalkylene). In some embodiments, the linker is selected from the group consisting of: —(CR₂)_{y}—, —O(CR₂)_{y}O—, —O(CR₂)_{y}—, —(CR₂)_{y}O—, and —O(CR₂CR₂O)_{y}—, wherein y is 1-10 and each R is independently selected from the group consisting of H, halogen, alkyl, or is taken together with another R to form an optionally substituted cycloalkyl. In some embodiments, each R is independently selected from H, alkyl, or is taken together with another R to form an optionally substituted cycloalkyl. In some embodiments, the one or more R is taken together with one or more other R to form a bridged cycloalkyl (e.g., a bridged cycloalkylene).

In some embodiments, a hydroxyl radical or a carboxylate radical of any one of Formula (IA), Formula (IB), Formula (IC), or Formula (ID) is attached to a hydroxyl radical or a carboxylate radical of another of any one of Formula (IA), Formula (IB), Formula (IC), or Formula (ID) through a linker. In some embodiments, a hydroxyl radical or a carboxylate radical of any one of Formula (IA), Formula (IB), Formula (IC), or Formula (ID) is attached to a hydroxyl radical or a carboxylate radical of any one of Formula (II), Formula (II′), Formula (IIA), or Formula (IIB) through a linker. In some embodiments, a hydroxyl radical of any one of Formula (IA), Formula (IB), Formula (IC), or Formula (ID) is attached to a hydroxyl radical of any one of Formula (II), Formula (II′), Formula (IIA), or Formula (IIB) through a linker. In some embodiments, a hydroxyl radical of any one of Formula (IA), Formula (IB), Formula (IC), or Formula (ID) is attached to a carboxylate radical of any one of Formula (II′), Formula (IIA), or Formula (IIB) through a linker. In some embodiments, a carboxylate radical of any one of Formula (IA), Formula (IB), Formula (IC), or Formula (ID) is attached to a hydroxyl radical of any one of Formula (II), Formula (II′), Formula (IIA), or Formula (IIB) through a linker. In some embodiments, a carboxylate radical of any one of Formula (IA), Formula (IB), Formula (IC), or Formula (ID) is attached to a carboxylate radical of any one of Formula (II), Formula (II′), Formula (IIA), or Formula (IIB) through a linker. In some embodiments, the linker is a bond. In some embodiments, the linker is oxo.

In some embodiments, any one of R^{a}, R^{a′}, R^{b}, R^{b′}, R^{b″}, R^{c}, R^{c″}, R^{d}, or R^{d′} is an ester radical, a hydroxyl radical, or a carboxylate radical, and any one of R¹, R², R³, R⁴, R¹, R⁶, R^{6′}, R^{6″}, or R¹⁰ is an amide radical, a thiol radical, a hydroxyl radical, or a carboxylate radical. In some embodiments, any one of R^{a}, R^{a′}, R^{b}, R^{b′}, R^{b″}, R^{c}, R^{c″}, R^{d}, or R^{d′} is an ester radical, a hydroxyl radical, or a carboxylate radical, and any one of R¹, R², R³, R⁴, R⁵, R⁶, R^{6′}, R^{6″}, or R¹⁰ is a hydroxyl radical or a carboxylate radical. In some embodiments, any radical of R^{a}, R^{a′}, R^{b}, R^{b′}, R^{b″}, R^{c}, R^{c″}, R^{d}, or R^{d′} is adjoined to any radical of R¹, R², R³, R⁴, R⁵, R⁶, R^{6′}, R^{6″}, or R¹⁰ by a linker. In some embodiments, any radical of R^{a}, R^{a′}, R^{b}, R^{b′}, R^{b″}, R^{c}, R^{c″}, R^{d}, or R^{d′} is adjoined to any radical of R⁴, R⁵, R⁶, R^{6′}, R^{6″}, or R¹⁰ by a linker. In some embodiments, any radical of R^{a}, R^{b}, R^{c}, or R^{d} is adjoined to any radical of R⁴, R¹, R⁶, R^{6′}, R^{6″}, or R¹⁰ by a linker. In some embodiments, any radical of R^{d} or R^{d′} is adjoined to any radical of R⁴, R⁵, R⁶, R^{6′}, R^{6″}, or R¹⁰ by a linker. In some embodiments, a radical of R^{d} is adjoined to any radical of R⁴, R⁶, R^{6′}, R^{6″}, or R¹⁰ by a linker. In some embodiments, a radical of R^{d} is adjoined to a radical of R⁴ or R¹⁰ by a linker. In some embodiments, a radical of of R^{d} is adjoined to any radical of R⁶, R^{6′}, or R^{6″} by a linker. In some embodiments, the linker is oxo. In some embodiments, the linker is a bond.

In some embodiments, the linker is a bond, alkyl, heteroalkyl, or alkoxy, wherein the alkyl, heteroalkyl, or alkoxy is optionally substituted. In some embodiments, the alkyl, heteroalkyl, or alkoxy are each independently substituted with one or more groups, each group being independently selected from the group consisting of —O—, —S—, silicone, amino, optionally substituted alkyl (e.g., alkenyl, alkynyl, branched (e.g., polypropylene), haloalkyl), optionally substituted heteroalkyl (e.g, polyTHF), and optionally substituted cycloalkyl. In some embodiments, the linker is alkyl (alkylene) and the alkyl (alkylene) is substituted with one or more groups selected from —OH, halo, oxo, alkyl, heteroalkyl, cycloalkyl, and heterocycloalkyl. In some embodiments, the linker is an unsubstituted alkyl (alkylene). In some embodiments, the linker is heteroalkyl (heteroalkylene) and the heteroalkyl (heteroalkylene) is substituted with one or more groups selected from halo or alkyl. In some embodiments, the linker is unsubstituted heteroalkyl (heteroalkylene). In some embodiments, the linker is a bond.

In some embodiments, the linker comprises one or more linker group, each linker group being independently selected from a bond, alkyl, cycloalkyl, heteroalkyl, or alkoxy, wherein the alkyl, cycloalkyl, heteroalkyl, or alkoxy is optionally substituted. In some embodiments, the linker is a bond, alkyl, cycloalkyl, heteroalkyl, or alkoxy, wherein the alkyl, cycloalkyl, heteroalkyl, or alkoxy is optionally substituted. In some embodiments, the alkyl, cycloalkyl, heteroalkyl, or alkoxy are each independently substituted with one or more substitutent, each substituent being independently selected from the group consisting of —O— (e.g., —OH), —S— (e.g., —SH), silicone, amino, optionally substituted alkyl (e.g., alkenyl, alkynyl, branched (e.g., polypropylene), haloalkyl), optionally substituted heteroalkyl (e.g, polyTHF), and optionally substituted cycloalkyl. In some embodiments, the linker comprises one or more linker group, each linker group being independently selected from alkyl (alkylene) and cycloalkyl (cycloalkylene). In some embodiments, the linker is alkyl (alkylene) or cycloalkyl (cycloalkylene). In some embodiments, the alkyl (alkylene) or cycloalkyl (cycloalkylene) is unsubstituted or substituted with one or more substituent, each substituent being independently selected from the group consisting of —OH, halo, oxo, alkyl, heteroalkyl, cycloalkyl, and heterocycloalkyl. In some embodiments, the linker comprises an unsubstituted or substituted alkylene-cycloalkylene-alkylene.

In some embodiments, the linker comprises at least one oxo. In some embodiments, the linker is oxo. In some embodiments, the linker comprises at least one carbamate. In some embodiments, the linker is a carbamate. In some embodiments, the linker comprises at least one ester. In some embodiments, the linker is an ester.

In some embodiments, the linker comprises one or more linker groups selected from oxo, —O—, —S—, unsubstituted alkylene, C═O(CH₂CH₂)ₙC=O, C═O(CHCH)ₙC=O, C═O(OCH₂CH₂O)ₙC=O, O(CH₂CH₂O)ₙ, and C═O(CH₂CH₂O)ₙ, (CH(CH₃)C(═O)O)ₙ, wherein n is 1-20. In some embodiments, the linker is a bond, unsubstituted alkylene, C═O(CH₂CH₂)ₙC=O, C═O(CHCH)ₙC=O, C═O(OCH₂CH₂O)ₙC=O, O(CH₂CH₂O)ₙ, C═O(CH₂CH₂O)ₙ, (CH(CH₃)C(═O)O)ₙ, and C═O(CH₂CH₂)ₙC=O(CH(CH₃)C(═O)O)ₙ, wherein n is 1-20. In some embodiments, n is 1-10. In some embodiments, n is 6. In some embodiments, n is 5. In some embodiments, n is 4. In some embodiments, n is 3. In some embodiments, n is 2. In some embodiments, n is 1.

In some embodiments, the linker comprises one or more linker group, each linker group being independently selected from the group consisting of a bond, —O—, —O(C═O)—, —O(C═O)—O—, —S—, unsubstituted alkylene, unsubstituted cycloalkylene, C═O(CH₂CH₂)ₙC=O, C═O(CHCH)ₙC=O, C═O(OCH₂CH₂O)ₙC=O, O(CH₂CH₂O)ₙ, and C═O(CH₂CH₂O)ₙ, and (CH(CH₃)C(═O)O)ₙ, wherein n is 1-20. In some embodiments, the linker is a bond, unsubstituted alkylene, unsubstituted alkylene-cycloalkylene-alkylene, C═O(CH₂CH₂)ₙC=O, C═O(CHCH)ₙC=O, C═O(OCH₂CH₂O)ₙC=O, O(CH₂CH₂O)ₙ, and C═O(CH₂CH₂O)ₙ, (CH(CH₃)C(═O)O)ₙ, C═O(CH₂CH₂)ₙC=O(CH(CH₃)C(═O)O)ₙ, wherein n is 1-20. In some embodiments, n is 1-10. In some embodiments, n is 6. In some embodiments, n is 5. In some embodiments, n is 4. In some embodiments, n is 3. In some embodiments, n is 2. In some embodiments, n is 1.

In some embodiments, the linker is hydrolyzed in a buffered solution. In some embodiments, the linker is hydrolytically labile. In some embodiments, the linker is hydrolyzed by water. In some embodiments, the linker is hydrolyzed by an enzyme. In some embodiments, the enzyme is a hydrolase (e.g., a protease or an esterase). In some embodiments, the enzyme is an esterase.

In some instances, longer linkers (e.g., at least 3-4 (e.g., carbon) atoms between an —O—) are preferred because, e.g., in some instances, shorter linkers result in increased melting point, increased T_{g}, increased crystallinity, decreased processability, or any combination thereof. In some instances, compounds comprising linkers having 7 or more carbon atoms (e.g., between an —O—) are not processable (e.g., because the compounds lack enough rigidity to form a sufficient crystal lattice).

In some embodiments, —(CH₂)X-cycloalkyl-(CH₂)_{y}—, wherein x and y are each independently 0-3. In some embodiments, x and y are each independently 0-2. In some embodiments, x and y are each independently 0 or 1. In some embodiments, x and y are each 0. In some embodiments, x and y are each 1.

In some embodiments, the linker is hydrolyzed in a buffered solution. In some embodiments, the linker is hydrolyzed by an enzyme. In some embodiments, the enzyme is a hydrolase (e.g., a protease or an esterase).

In some embodiments, the linker is a non-hydrolyzable linker (e.g., consisting of non-hydrolyzable bonds, such as, for example, alkylene, alkoxy, or the like (e.g., (CH₂CH₂)ₙ, (CHCH)ₙ, O(CH₂CH₂O)ₙ, (CH₂CH₂O)ₙ, or the like, wherein n is 1-20)). In some instances, a composition (e.g., an article, an implant, or a coating) comprising a compound with a non-hydrolyzable linker reduces off-target effects in the individual.

In some embodiments, the linker comprises one or more amino acid. In some embodiments, the linker is a peptide (e.g., an oligopeptide or a polypeptide).

In some embodiments, a linker provided herein provides a therapeutic effect in an individual (e.g., an individual (e.g., in need thereof) administered a composition provided herein (e.g., comprising a compound provided herein)). In some embodiments, the linker is released from a compound provided herein and provides a therapeutic effect in an individual (e.g., an individual (e.g., in need thereof) administered a composition provided herein (e.g., comprising a compound provided herein)). In some embodiments, D1, D2, and a linker provided herein (e.g., in their free form) are released from a compound provided herein. In some embodiments, D1, D2, and a linker provided herein (e.g., in theirfree form) are released from a compound provided herein and provides a therapeutic effect in an individual (e.g., an individual (e.g., in need thereof) administered a composition provided herein (e.g., comprising a compound provided herein)).

In certain instances, a composition provided herein comprises a linker comprising at least one group (e.g., not an ether, an amine, or the like) that is configured to release a radical in its free form or a conjugate provided herein. In some instances, an ether group confers stability to a conjugate provided herein, thereby providing properties that are not suitable for drug delivery applications provided herein. In some instances, an ether group confers stability to a conjugate provided herein, thereby providing properties that are suitable for drug delivery applications provided herein. In some embodiments, provided herein is a compound that is conjugated through a labile linking group such as, for example, an ester and/or a carbonate.

In some embodiments, the compound does not have the structure:

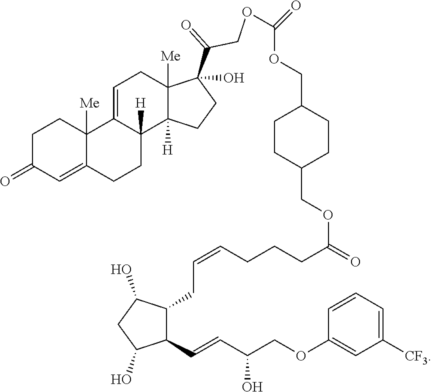

In some embodiments, the compound does not have the structure:

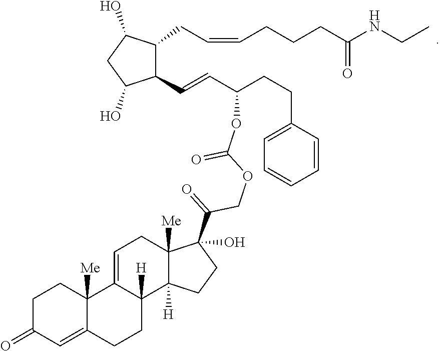

In some embodiments, the compound does not have the structure:

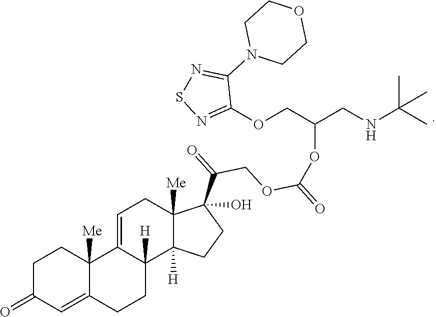

In some embodiments, the compound does not have the structure:

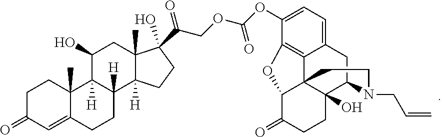

In some embodiments, the compound does not have the structure:

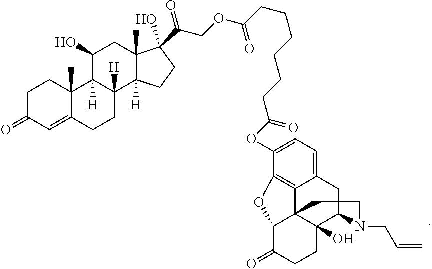

In some embodiments, the compound does not have the structure:

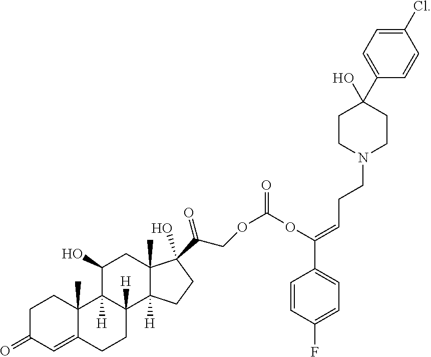

In some embodiments, the compound does not have the structure:

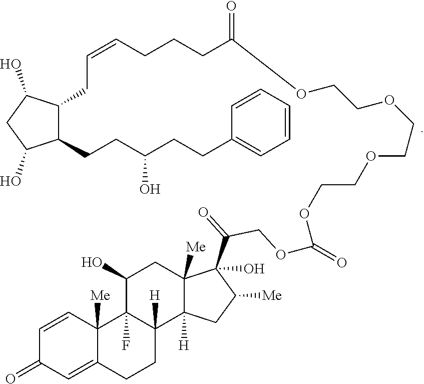

In certain embodiments, the disclosure provides a compound, or pharmaceutically acceptable salt thereof, having a structure provided in Table 4. In some embodiments, the compound, or pharmaceutically acceptable salt thereof, provided in Table 4 is a solid at a temperature of at least 20° C. (e.g., at least 30 C, at least 37 C, at least 40 C, at least 50 C, at least 70 C, at least 100 C, or the like). In some embodiments, the compound, or pharmaceutically acceptable salt thereof, provided in Table 4 is processable at a temperature of at least 20° C. (e.g., as described in the examples). In some embodiments, the compound, or pharmaceutically acceptable salt thereof, provided in Table 4 is processable into an article or implant (e.g., machined, molded, emulsion-processed, electrospun, electrosprayed, blow molded, or extruded to form a fiber, fiber mesh, woven fabric, non-woven fabric, film, surface coating, pellet, cylinder, rod, microparticle, nanoparticle, or another shaped article) at a temperature of at least 20° C. In some embodiments, the compound, or pharmaceutically acceptable salt thereof, provided in Table 4 comprises both a first radical and a second radical. In some embodiments, the compound in Table 4 is processable when the first radical, and the second radical are joined by a linker. In some embodiments, the linker is a bond.

TABLE 4

| Compound | | |
|---|---|---|
| Number | Structure | Name |
| 1 | 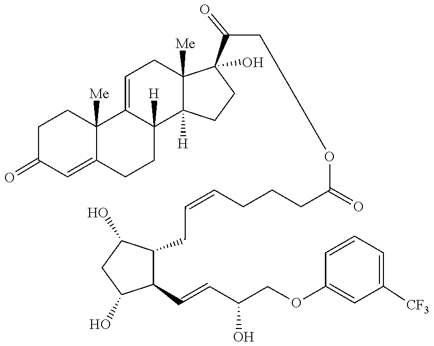 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 2 | 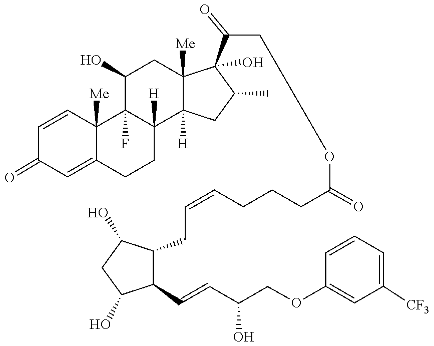 | 2- ((8S,9R,10S,11S,13S,14S, 16R,17R)-9-fluoro-11,17- dihydroxy-10,13,16- trimethyl-3-oxo- 6,7,8,9,10,11,12,13,14,15, 16,17-dodecahydro- 3H- cyclopenta[a]phenanthren- 17-yl)-2-oxoethyl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 3 | 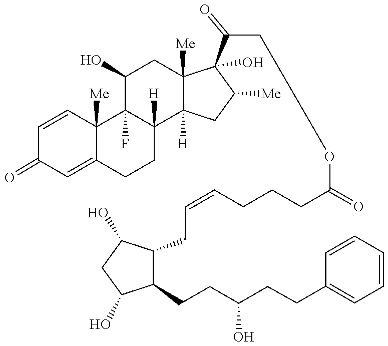 | 2- ((8S,9R,10S,11S,13S,14S, 16R,17R)-9-fluoro-11,17- dihydroxy-10,13,16- trimethyl-3-oxo- 6,7,8,9,10,11,12,13,14,15, 16,17-dodecahydro- 3H- cyclopenta[a]phenanthren- 17-yl)-2-oxoethyl (Z)-7- ((1R,2R,3R,5S)-3,5- dihydroxy-2-((R)-3- hydroxy-5- phenylpentyl)cyclopentyl) hept-5-enoate |
| 4 | 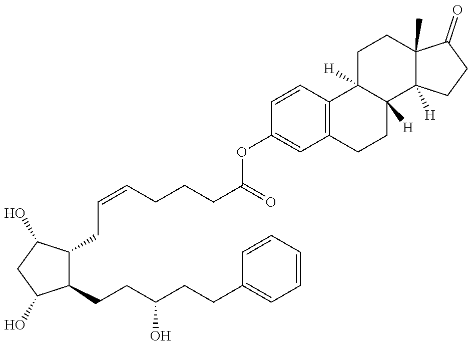 [SMILES: C[C@]12CC[C@@H]3c4ccc(OC(=O)CCC/C=C\C[C@@H]5[C@@H](CC[C@@H](O)CCc6ccccc6)[C@H](O)C[C@@H]5O)cc4CC[C@H]3[C@@H]1CCC2=O] | (8R,9S,13S,14S)-13- methyl-17-oxo- 7,8,9,11,12,13,14,15,16, 17-decahydro-6H- cyclopenta[a]phenanthren- 3-yl (Z)-7- ((1R,2R,3R,5S)-3,5- dihydroxy-2-((R)-3- hydroxy-5- phenylpentyl)cyclopentyl) hept-5-enoate |
| 5 | 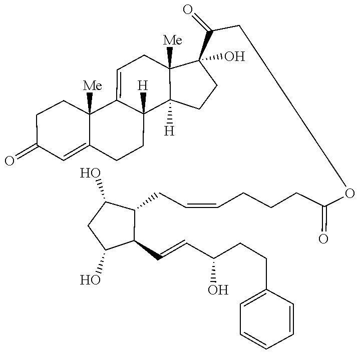 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (Z)-7- ((1R,2R,3R,5S)-3,5- dihydroxy-2-((S,E)-3- hydroxy-5-phenylpent-1- en-1-yl)cyclopentyl)hept- 5-enoate |
| 6 | 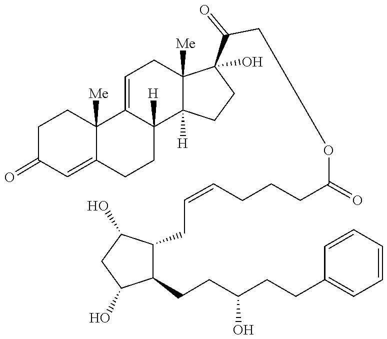 | 2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethyl (Z)-7- ((1R,2R,3R,5S)-3,5- dihydroxy-2-((R)-3- hydroxy-5- phenylpentyl)cyclopentyl) hept-5-enoate |
| 7 | 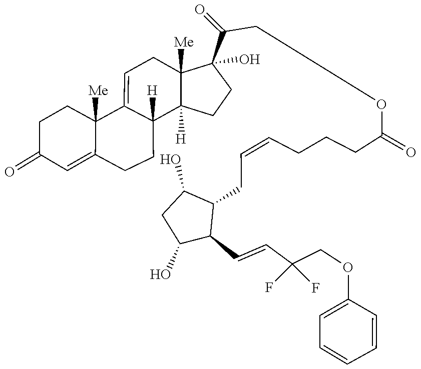 | 2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethyl (Z)-7- ((1R,2R,3R,5S)-2-((E)-3,3- difluoro-4-phenoxybut-1- en-1-yl)-3,5- dihydroxycyclopentyl) hept-5-enoate |
| 8 | 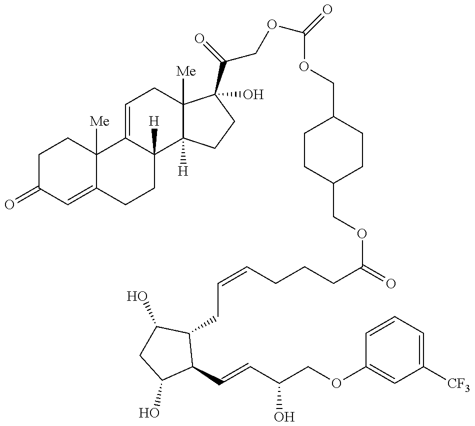 | (4-((((2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2- oxoethoxy)carbonyl)oxy) methyl)cyclohexyl)methyl (Z)-7-((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 9 | 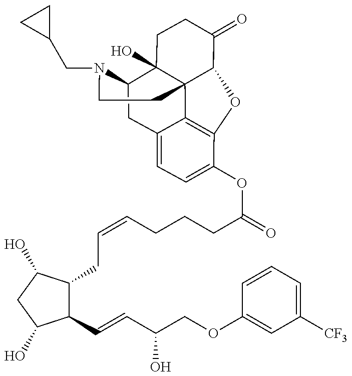 [SMILES: O=C(CCC/C=C\C[C@@H]1[C@@H](/C=C/[C@@H](O)COc2cccc(C(F)(F)F)c2)[C@H](O)C[C@@H]1O)Oc1ccc2c3c1O[C@H]1C(=O)CC[C@@]4(O)[C@@H](C2)N(CC2CC2)CC[C@]314] | (4R,4aS,7aR,12bS)-3- (cyclopropylmethyl)-4a- hydroxy-7-oxo- 2,3,4,4a,5,6,7,7a- octahydro-1H-4,12- methanobenzofuro[3,2- e]isoquinolin-9-yl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 10 | 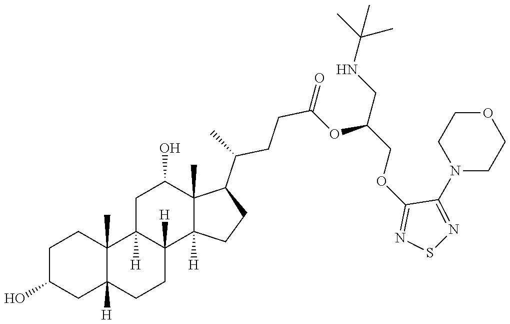 | (S)-1-(tert-butylamino)- 3-((4-morpholino-1,2,5- thiadiazol-3- yl)oxy)propan-2-yl (R)-4- ((3R,5R,8R,9S,10S,12S, 13R,14S,17R)-3,12- dihydroxy-10,13- dimethylhexadecahydro- 1H- cyclopenta[a]phenanthren- 17-yl)pentanoate |
| 11 | 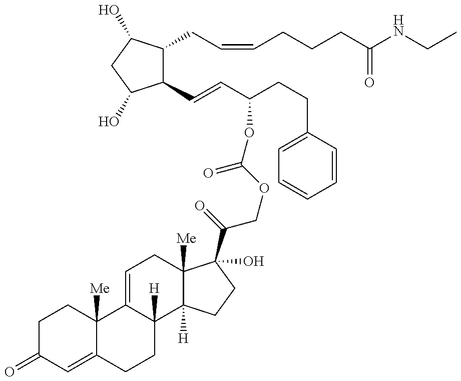 | (S,E)-1-((1R,2R,3S,5R)-2- ((Z)-7-(ethylamino)-7- oxohept-2-en-1-yl)-3,5- dihydroxycyclopentyl)-5- phenylpent-1-en-3-yl (2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl) carbonate |
| 12 | 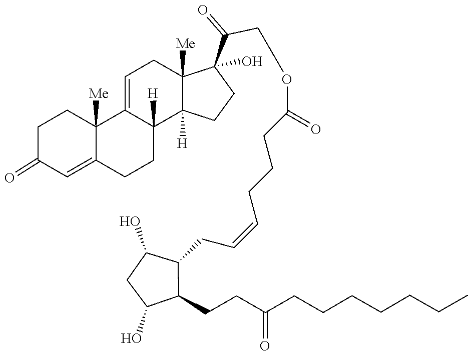 | 2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethyl (Z)-7- ((1R,2R,3R,5S)-3,5- dihydroxy-2-(3- oxodecyl)cyclopentyl) hept-5-enoate |
| 13 | 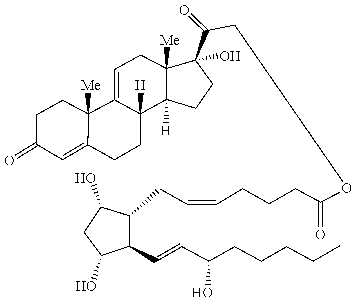 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (Z)-7- ((1R,2R,3R,5S)-3,5- dihydroxy-2-((S,E)-3- hydroxyoct-1-en-1- yl)cyclopentyl)hept-5- enoate |
| 14 | 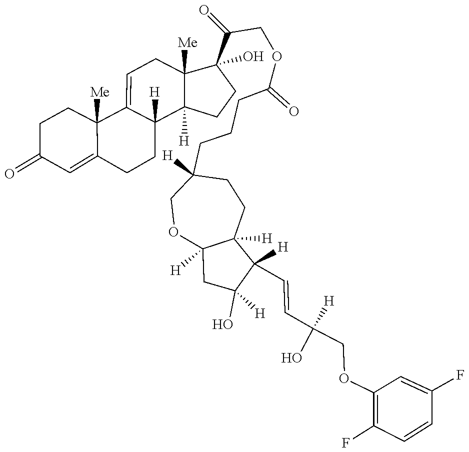 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl 4- ((3R,5aS,6S,7S,8aR)-6- ((S,E)-4-(2,5- difluorophenoxy)-3- hydroxybut-1-en-1-yl)-7- hydroxyoctahydro-2H- cyclopenta[b]oxepin-3- yl)butanoate |
| 15 | 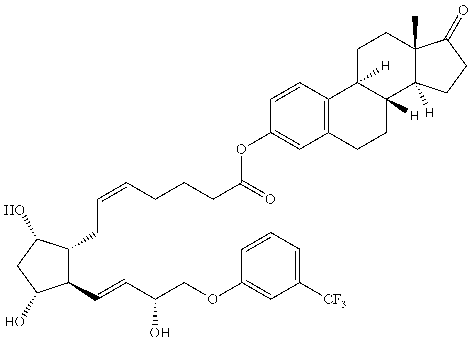 | (8R,9S,13S,14S)-13- methyl-17-oxo- 7,8,9,11,12,13,14,15,16, 17-decahydro-6H- cyclopenta[a]phenanthren- 3-yl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 16 | 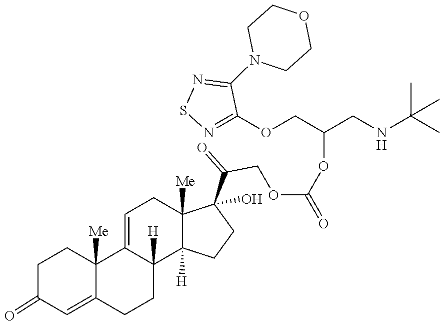 | 1-(tert-butylamino)-3- ((4-morpholino-1,2,5- thiadiazol-3- yl)oxy)propan-2-yl (2- ((85,10S,13S,14S, 17R)- 17-hydroxy-10,13- dimethyl-3-oxo- 2,3,6,7,8,10,12,13,14,15, 16,17-dodecahydro-1H- cyclopenta[a]phenanthre n-17-yl)-2-oxoethyl) carbonate |
| 17 | 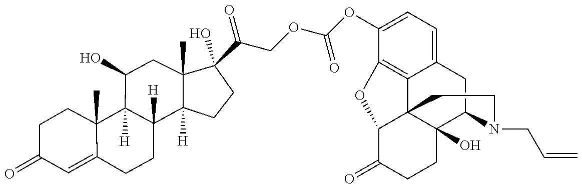 | (4R,4aS,7aR,12bS)-3- allyl-4a-hydroxy-7-oxo- 2,3,4,4a,5,6,7,7a- octahydro-1H-4,12- methanobenzofuro[3,2- e]isoquinolin-9-yl (2- ((8S,9S,10R,11S,13S,14S, 17R)-11,17-dihydroxy- 10,13-dimethyl-3-oxo- 2,3,6,7,8,9,10,11,12,13, 14,15,16,17- tetradecahydro-1H- cyclopenta[a]phenanthren- 17-yl)-2-oxoethyl) carbonate |
| 18 | 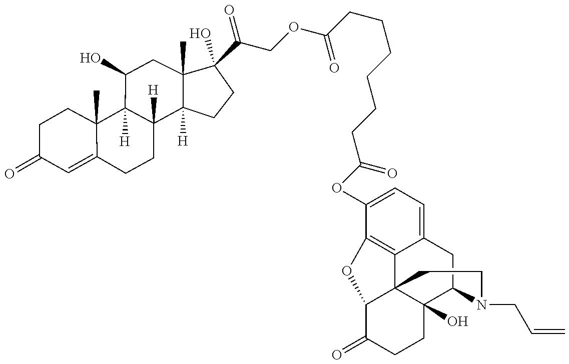 | 1-((4R,4aS,7aR,12bS)-3- allyl-4a-hydroxy-7-oxo- 2,3,4,4a,5,6,7,7a- octahydro-1H-4,12- methanobenzofuro[3,2- e]isoquinolin-9-yl) 8-(2- ((8S,9S,10R,11S,13S,14S, 17R)-11,17-dihydroxy- 10,13-dimethyl-3-oxo- 2,3,6,7,8,9,10,11,12,13, 14,15,16,17- tetradecahydro-1H- cyclopenta[a]phenanthren- 17-yl)-2-oxoethyl) octanedioate |
| 19 | 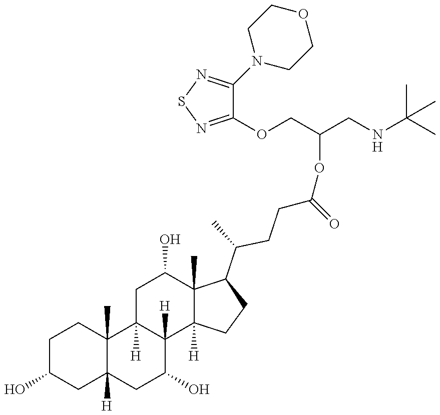 | 1-(tert-butylamino)-3- ((4-morpholino-1,2,5- thiadiazol-3- yl)oxy)propan-2-yl (4R)- 4- ((3R,5S,7R,8R,9S,10S,12S, 13R,14S,17R)-3,7,12- trihydroxy-10,13- dimethylhexadecahydro- 1H- cyclopenta[a]phenanthren- 17-yl)pentanoate |
| 20 | 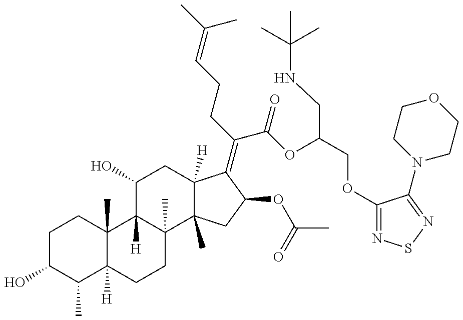 | 1-(tert-butylamino)-3- ((4-morpholino-1,2,5- thiadiazol-3- yl)oxy)propan-2-yl (Z)-2- ((3R,4S,5S,8S,9S,10S,11R, 13R,14S,16S)-16- acetoxy-3,11-dihydroxy- 4,8,10,14- tetramethylhexadecahydro- 17H- cyclopenta[a]phenanthren- 17-ylidene)-6- methylhept-5-enoate |
| 21 | 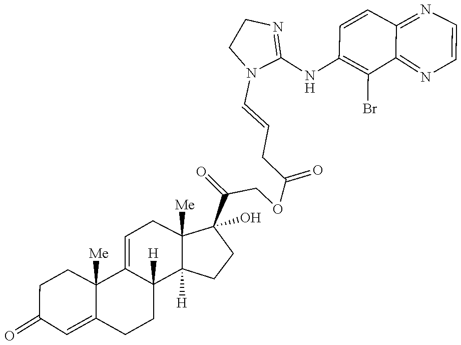 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (E)-4- (2-((5-bromoquinoxalin- 6-yl)amino)-4,5-dihydro- 1H-imidazol-1-yl)but-3- enoate |
| 22 | 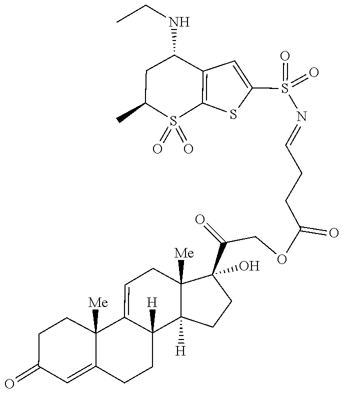 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (E)-4- ((((4S,6S)-4-(ethylamino)- 6-methyl-7,7-dioxido- 5,6-dihydro-4H- thieno[2,3-b]thiopyran- 2- yl)sulfonyl)imino) butanoate |
| 23 | 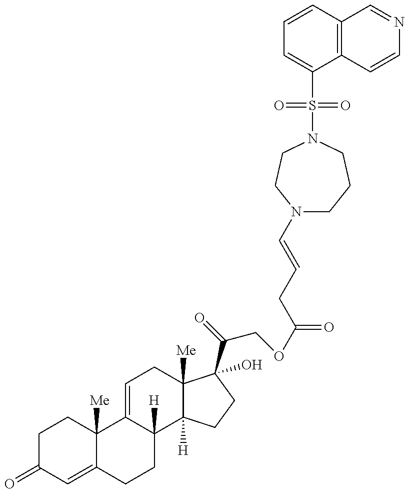 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (E)-4- (4-(isoquinolin-5- ylsulfonyl)-1,4-diazepan- 1-yl)but-3-enoate |
| 24 | 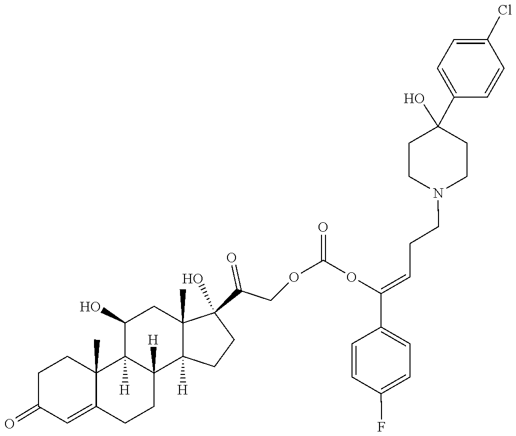 | (Z)-4-(4-(4- chlorophenyl)-4- hydroxypiperidin-1-yl)-1- (4-fluorophenyl)but-1- en-1-yl (2- ((8S,9S,10R,11S,13S,14S, 17R)-11,17-dihydroxy- 10,13-dimethyl-3-oxo- 2,3,6,7,8,9,10,11,12,13, 14,15,16,17- tetradecahydro-1H- cyclopenta[a]phenanthren- 17-yl)-2-oxoethyl) carbonate |

In some embodiments, the disclosure provides a compound (e.g., conjugate, such as a heterodimeric conjugate), or pharmaceutically acceptable salt thereof, having a structure provided in Table 5. In other embodiments, parent conjugate compounds that do not form a processable solid are shown in Table 5. In some embodiments, the compound, or pharmaceutically acceptable salt thereof, provided in Table 5 is not a solid at a temperature of at least 20° C. In some embodiments, the compound, or pharmaceutically acceptable salt thereof, provided in Table 5 is not processable at a temperature of at least 20° C. In some embodiments, the compound, or pharmaceutically acceptable salt thereof, provided in Table 5 is not processable into an article, as described herein, at a temperature of at least 20° C.

In some embodiments, the compound, or pharmaceutically acceptable salt thereof, provided in Table 5 comprises both a first radical and a second radical. In some embodiments, the heterodimeric conjugate in Table 5 is not processable when the first radical and the second radical are joined by a linker. In some embodiments, the linker is not a bond. In some embodiments, the linker is alkyl, heteroalkyl, or alkoxy, wherein the alkyl, heteroalkyl, or alkoxy is optionally substituted with one or more groups, each group being independently selected from the group consisting of a bond, —O—, —S—, silicone, amino, optionally substituted alkyl (e.g., alkenyl, alkynyl, branched (e.g., polypropylene), haloalkyl), optionally substituted heteroalkyl (e.g, polyTHF), and optionally substituted cycloalkyl.

TABLE 5

| Compound | | |
|---|---|---|
| Number | Structure | Name |
| 25 | 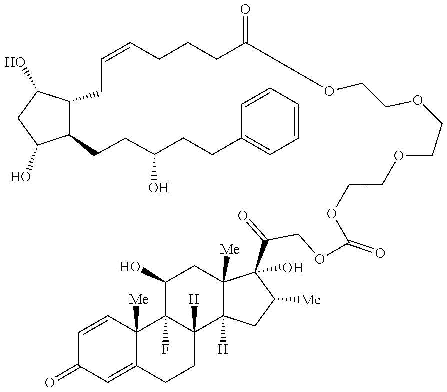 | 1- ((8S,9R,10S,11S,13S,14S, 16R,17R)-9-fluoro-11,17- dihydroxy-10,13,16- trimethyl-3-oxo- 6,7,8,9,10,11,12,13,14,15, 16,17-dodecahydro- 3H- cyclopenta[a]phenanthren- 17-yl)-1,4-dioxo- 3,5,8,11- tetraoxatridecan-13-yl (Z)-7-((1R,2R,3R,5S)-3,5- dihydroxy-2-((R)-3- hydroxy-5- phenylpentyl)cyclopentyl) hept-5-enoate |
| 26 | 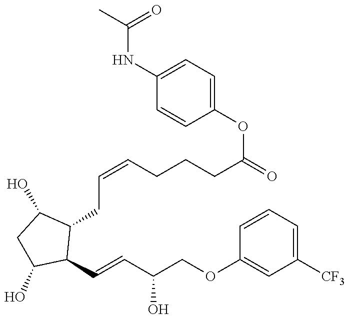 [SMILES: CC(=O)Nc1ccc(OC(=O)CCC/C=C\C[C@@H]2[C@@H](/C=C/[C@@H](O)COc3cccc(C(F)(F)F)c3)[C@H](O)C[C@@H]2O)cc1] | 4-acetamidophenyl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 27 | 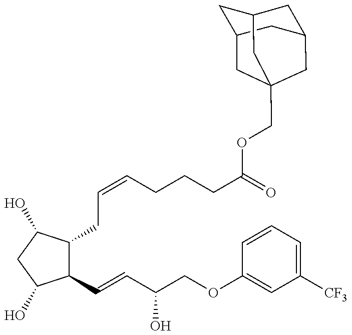 [SMILES: O=C(CCC/C=C\C[C@@H]1[C@@H](/C=C/[C@@H](O)COc2cccc(C(F)(F)F)c2)[C@H](O)C[C@@H]1O)OCC12CC3CC(CC(C3)C1)C2] | ((3R,5R,7R)-adamantan- 1-yl)methyl (Z)-7- ((1R,2R,3R,5S)-3,5- dihydroxy-2-((R,E)-3- hydroxy-4-(3- (trifluoromethyl)phenoxy) but-1-en-1- yl)cyclopentyl) hept-5- enoate |
| 28 | 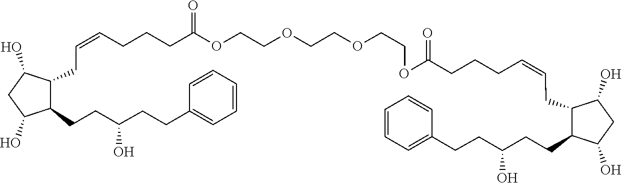 [SMILES: O=C(CCC/C=C\C[C@@H]1[C@@H](CC[C@@H](O)CCc2ccccc2)[C@H](O)C[C@@H]1O)OCCOCCOCCOC(=O)CCC/C=C\C[C@H]1[C@H](CC[C@H](O)CCc2ccccc2)[C@@H](O)C[C@H]1O] | 2-(2-(2-(((Z)-7- ((1R,2R,3R,5S)-3,5- dihydroxy-2-((R)-3- hydroxy-5- phenylpentyl)cyclopentyl) hept-5- enoyl)oxy)ethoxy)ethoxy) ethyl (Z)-7- ((1S,2S,3S,5R)-3,5- dihydroxy-2-((S)-3- hydroxy-5- phenylpentyl)cyclopentyl) hept-5-enoate |
| 29 | 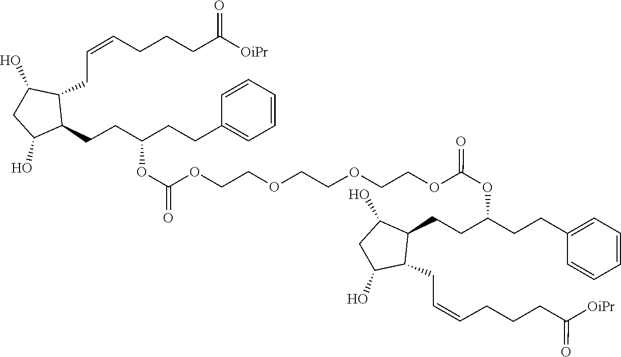 [SMILES: CC(C)OC(=O)CCC/C=C\C[C@@H]1[C@@H](CC[C@H](CCc2ccccc2)OC(=O)OCCOCCOCCOC(=O)O[C@H](CCc2ccccc2)CC[C@H]2[C@H](C/C=C\CCCC(=O)OC(C)C)[C@H](O)C[C@@H]2O)[C@H](O)C[C@@H]1O] | propyl (Z)-7- ((1R,2R,3R,5S)-2- ((3R,18S)-20- ((1S,2S,3R,5S)-3,5- dihydroxy-2-((Z)-7-oxo-7- propoxyhept-2-en-1- yl)cyclopentyl)-5,16- dioxo-3,18-diphenethyl- 4,6,9,12,15,17- hexaoxaicosyl)-3,5- dihydroxycyclopentyl) hept-5-enoate |
| 30 | 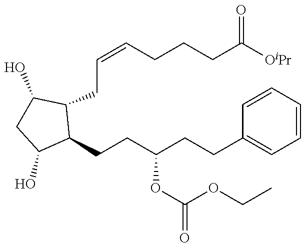 [SMILES: CCOC(=O)O[C@@H](CCc1ccccc1)CC[C@@H]1[C@@H](C/C=C\CCCC(=O)OC(C)C)[C@@H](O)C[C@H]1O] | propyl (Z)-7- ((1R,2R,3R,5S)-2-((R)-3- ((ethoxycarbonyl)oxy)-5- phenylpentyl)-3,5- dihydroxycyclopentyl) hept-5-enoate |

In some embodiments, a composition provided herein (e.g., an article, an implant, or a coating) comprises a compound, or pharmaceutically acceptable salt thereof, provided in Table 6. In some embodiments, the compound comprises both a first radical and a second radical, which when combined are processable (e.g., wherein the radicals may or may not be processable themselves, when in their free form). In some embodiments, the composition provided herein (e.g., an article, an implant, or a coating) comprises a compound, or pharmaceutically acceptable salt thereof, having a structure provided in Table 6. In some embodiments, the compound, or pharmaceutically acceptable salt thereof, provided in Table 6 is a solid at a temperature of at least 20° C. (e.g., at least 30 C, at least 37 C, at least 40 C, at least 50 C, at least 70 C, at least 100 C, or the like). In some embodiments, the compound, or pharmaceutically acceptable salt thereof, provided in Table 6 is processable at a temperature of at least 20° C. (e.g., as described in the examples). In some embodiments, the compound, or pharmaceutically acceptable salt thereof, provided in Table 6 is processable into an article or implant (e.g., machined, molded, emulsion-processed, electrospun, electrosprayed, blow molded, or extruded to form a fiber, fiber mesh, woven fabric, non-woven fabric, film, surface coating, pellet, cylinder, rod, microparticle, nanoparticle, or another shaped article) at a temperature of at least 20° C.

In some embodiments, the compound, or pharmaceutically acceptable salt thereof, provided in Table 6 comprises a first radical and a second radical. In some embodiments, the first radical is a steroid radical and the second radical is a prostaglandin radical. In some embodiments, the compound in Table 6 is processable when the first radical (e.g., the steroid radical) and the second radical (e.g., the prostaglandin radical) are joined by a linker (e.g., wherein the linker is not a bond). In some embodiments, the linker is (C═O), (—C═OCH₂CH₂C═O—), (—C═OphenylC═O—), (—C═OCH₂CH₂—), (—(C═O)OCH₂CH₂CH₂O(C═O)—), (—C═OCH(NHCOCH₃)CH₂—), (—C═OCH₂O(C═O)—), (—(C═O)OCH₂C═O—), (—C═OCH(CH₃)O(C═O)—), (—(C═O)OCH(CH₃)C═O—), (—C═OCH₂CH₂S—), (—C═OCH₂CH₂SC═O—), (—C═OCH₂CH₂SC═S—), or (—C═OCH₂CH₂NHC═O—).

TABLE 6

| Compound | | |
|---|---|---|
| Number | Structure | Name |
| 31 | 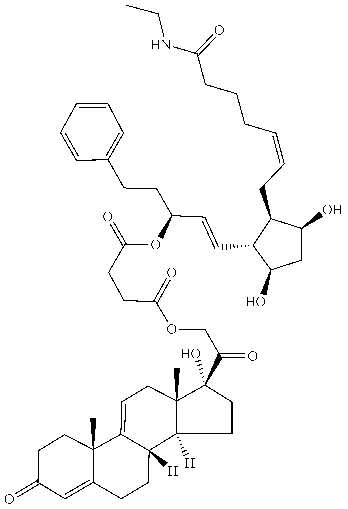 [SMILES: CCNC(=O)CCC/C=C\C[C@@H]1[C@@H](/C=C/[C@H](CCc2ccccc2)OC(=O)CCC(=O)OCC(=O)[C@@]2(O)CC[C@H]3[C@@H]4CCC5=CC(=O)CC[C@]5(C)C4=CC[C@@]32C)[C@H](O)C[C@@H]1O] | (S,E)-1-((1R,2R,3S,5R)-2- ((Z)-7-(ethylamino)-7- oxohept-2-en-1-yl)-3,5- dihydroxycyclopentyl)-5- phenylpent-1-en-3-yl (2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl) succinate |
| 32 | 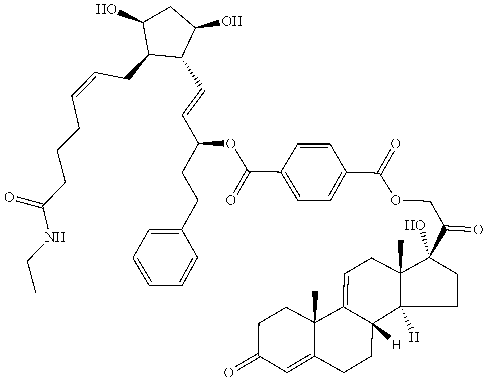 [SMILES: CCNC(=O)CCC/C=C\C[C@@H]1[C@@H](/C=C/[C@H](CCc2ccccc2)OC(=O)c2ccc(C(=O)OCC(=O)[C@@]3(O)CC[C@H]4[C@@H]5CCC6=CC(=O)CC[C@]6(C)C5=CC[C@@]43C)cc2)[C@H](O)C[C@@H]1O] | (S,E)-1-((1R,2R,3S,5R)-2- ((Z)-7-(ethylamino)-7- oxohept-2-en-1-yl)-3,5- dihydroxycyclopentyl)-5- phenylpent-1-en-3-yl (2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl) terephthalate |
| 33 | 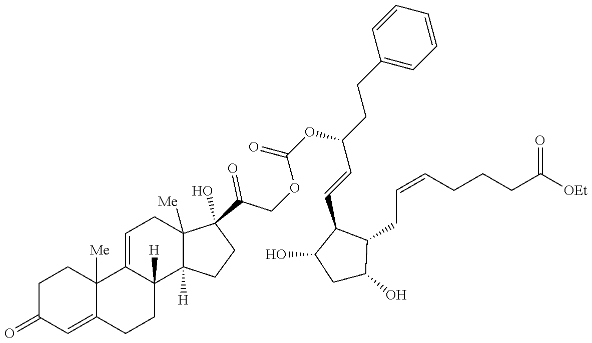 | Ethyl (Z)-7- ((1S,2S,3S,5R)-3,5- dihydroxy-2-((3R,E)-3- (((2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2- oxoethoxy)carbonyl)oxy)- 5-phenylpent-1-en-1- yl)cyclopentyl)hept-5- enoate |
| 34 | 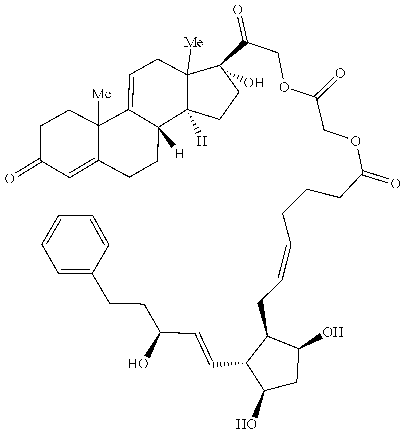 [SMILES: CC12CCC(=O)C=C1CC[C@@H]1C2=CCC2(C)[C@H]1CC[C@@H](O)[C@@H]2C(=O)COC(=O)COC(=O)CCC/C=C\C[C@@H]1[C@@H](/C=C/[C@@H](O)CCc2ccccc2)[C@H](O)C[C@@H]1O] | 2-(2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethoxy)-2- oxoethyl (Z)-7- ((1R,2R,3R,5S)-3,5- dihydroxy-2-((S,E)-3- hydroxy-5-phenylpent-1- en-1-yl)cyclopentyl)hept- 5-enoate |
| 35 | 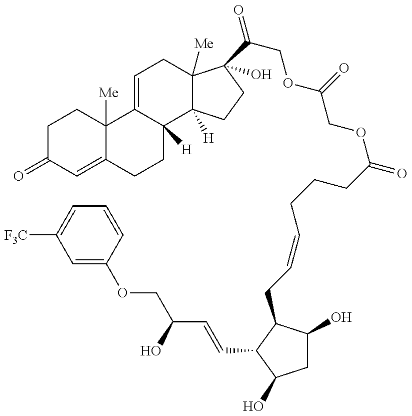 [SMILES: CC12CCC(=O)C=C1CC[C@@H]1C2=CCC2(C)[C@H]1CC[C@]2(O)C(=O)COC(=O)COC(=O)CCC/C=C\C[C@@H]1[C@@H](/C=C/[C@@H](O)COc2cccc(C(F)(F)F)c2)[C@H](O)C[C@@H]1O] | 2-(2-((8S,14S,17R)-17- hydroxy-10,13-dimethyl- 3-oxo- 2,3,6,7,8,10,12,13,14,15, 16,17-dodecahydro-1H- cyclopenta[alphenanthren- 17-yl)-2-oxoethoxy)-2- oxoethyl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 36 | 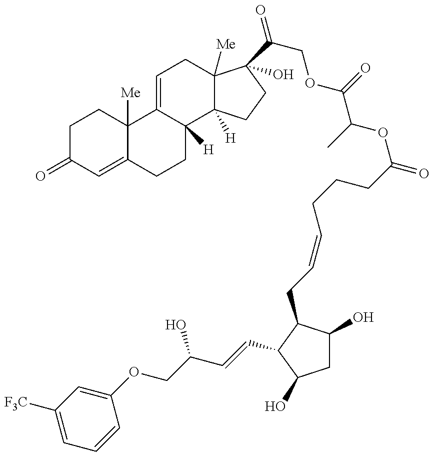 [SMILES: CC(OC(=O)CCC/C=C\C[C@@H]1[C@@H](/C=C/[C@@H](O)COc2cccc(C(F)(F)F)c2)[C@H](O)C[C@@H]1O)C(=O)OCC(=O)[C@@]1(O)CC[C@H]2[C@@H]3CCC4=CC(=O)CCC4(C)C3=CCC21C] | 1-(2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethoxy)-1- oxopropan-2-yl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 37 | 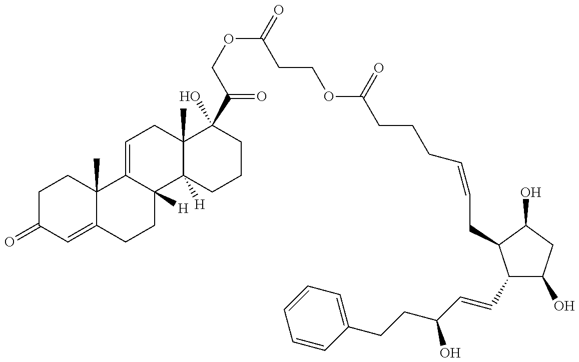 [SMILES: C[C@]12CCC(=O)C=C1CC[C@@H]1C2=CC[C@@]2(C)[C@H]1CCC[C@]2(O)C(=O)COC(=O)CCOC(=O)CCC/C=C\C[C@@H]1[C@@H](/C=C/[C@@H](O)CCc2ccccc2)[C@H](O)C[C@@H]1O] | 3-(2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethoxy)-3- oxopropyl (Z)-7- ((1R,2R,3R,5S)-3,5- dihydroxy-2-((S,E)-3- hydroxy-5-phenylpent-1- en-1-yl)cyclopentyl)hept- 5-enoate |
| 38 | 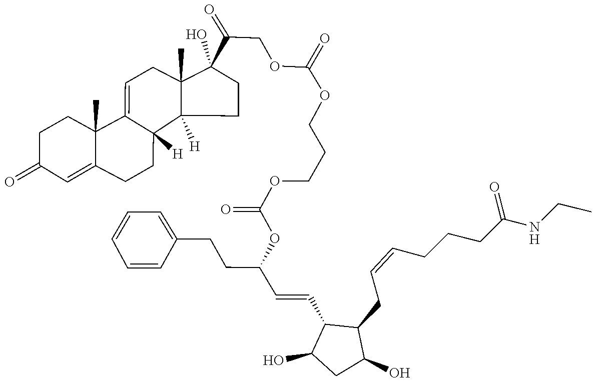 [SMILES: CCNC(=O)CCC/C=C\C[C@@H]1[C@@H](/C=C/[C@H](CCc2ccccc2)OC(=O)OCCCOC(=O)OCC(=O)[C@@]2(O)CC[C@H]3[C@@H]4CCC5=CC(=O)CC[C@]5(C)C4=CC[C@@]32C)[C@H](O)C[C@@H]1O] | (S,E)-1-((1R,2R,3S,5R)-2- ((Z)-7-(ethylamino)-7- oxohept-2-en-1-yl)-3,5- dihydroxycyclopentyl)-5- phenylpent-1-en-3-yl (2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl) propane-1,3-diyl bis(carbonate) |
| 39 | 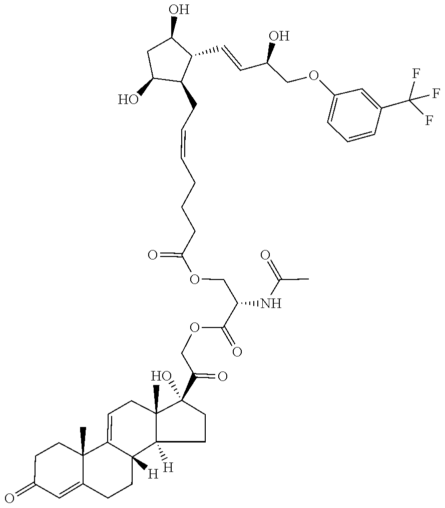 [SMILES: CC(=O)N[C@@H](COC(=O)CCC/C=C\C[C@@H]1[C@@H](/C=C/[C@@H](O)COc2cccc(C(F)(F)F)c2)[C@H](O)C[C@@H]1O)C(=O)OCC(=O)[C@@]1(O)CC[C@H]2[C@@H]3CCC4=CC(=O)CC[C@]4(C)C3=CC[C@@]21C] | (S)-2-acetamido-3-(2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethoxy)-3- oxopropyl (Z)-7- ((1R,2R,3R,5S)-3,5- dihydroxy-2-((R,E)-3- hydroxy-4-(3- (trifluoromethyl)phenoxy) but-1-en-1- yl)cyclopentyl)hept-5- enoate |
| 40 | 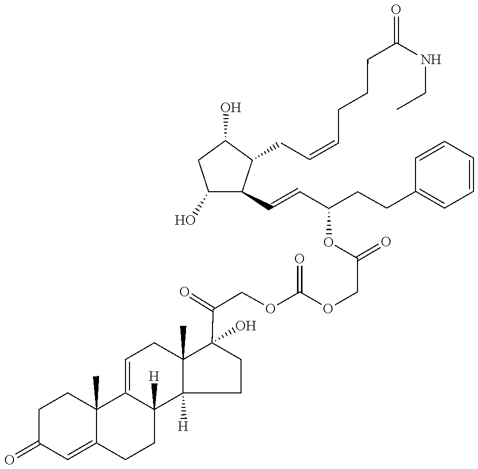 | (S,E)-1-((1R,2R,3S,5R)-2- ((Z)-7-(ethylamino)-7- oxohept-2-en-1-yl)-3,5- dihydroxycyclopentyl)-5- phenylpent-1-en-3-yl 2- (((2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2- oxoethoxy)carbonyl)oxy) acetate |
| 41 | 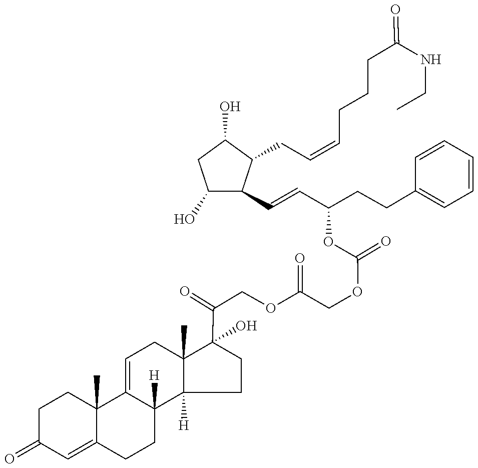 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl 2- (((((S,E)-1-((1R,2R,3S,5R)- 2-((Z)-7-(ethylamino)-7- oxohept-2-en-1-yl)-3,5- dihydroxycyclopentyl)-5- phenylpent-1-en-3- yl)oxy)carbonyl)oxy) acetate |
| 42A | 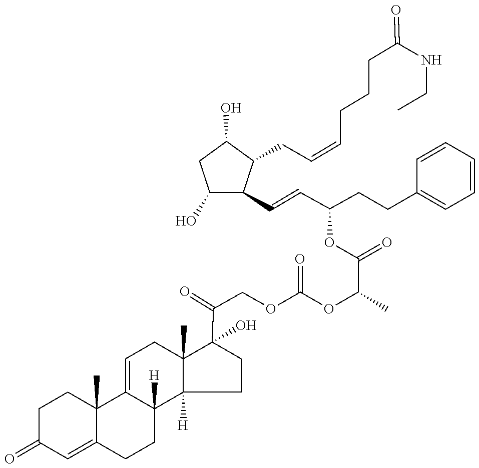 | (S,E)-1-((1R,2R,35,5R)-2- ((Z)-7-(ethylamino)-7- oxohept-2-en-1-yl)-3,5- dihydroxycyclopentyl)-5- phenylpent-1-en-3-yl (S)- 2-(((2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2- oxoethoxy)carbonyl)oxy) propanoate |
| 42B | 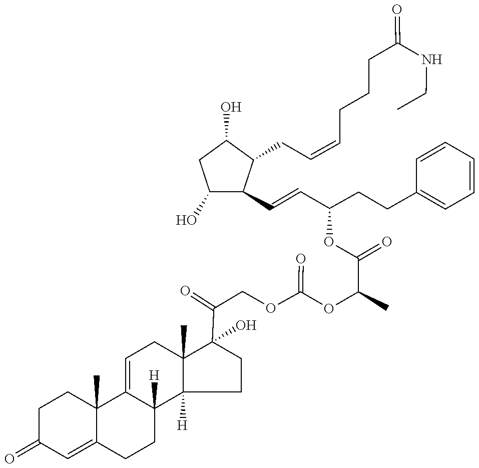 | (S,E)-1-((1R,2R,3S,5R)-2- ((Z)-7-(ethylamino)-7- oxohept-2-en-1-yl)-3,5- dihydroxycyclopentyl)-5- phenylpent-1-en-3-yl (R)- 2-(((2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2- oxoethoxy)carbonyl)oxy) propanoate |
| 43A | 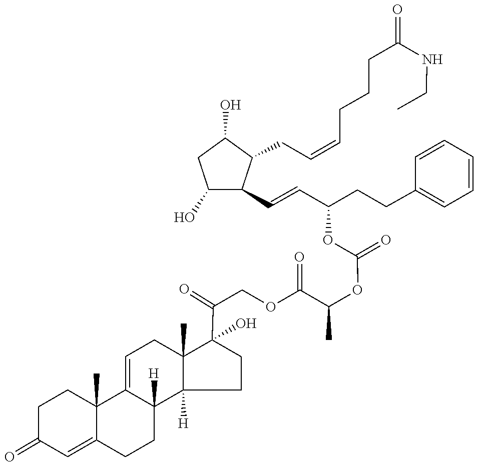 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (S)-2- (((((S,E)-1-((1R,2R,3S,5R)- 2-((Z)-7-(ethylamino)-7- oxohept-2-en-1-yl)-3,5- dihydroxycyclopentyl)-5- phenylpent-1-en-3- yl)oxy)carbonyl)oxy) propanoate |
| 43B | 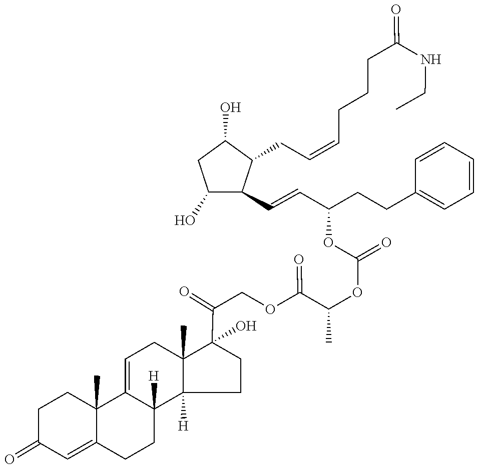 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (R)-2- (((((S,E)-1-((1R,2R,3S,5R)- 2-((Z)-7-(ethylamino)-7- oxohept-2-en-1-yl)-3,5- dihydroxycyclopentyl)-5- phenylpent-1-en-3- yl)oxy)carbonyl)oxy) propanoate |
| 44 | 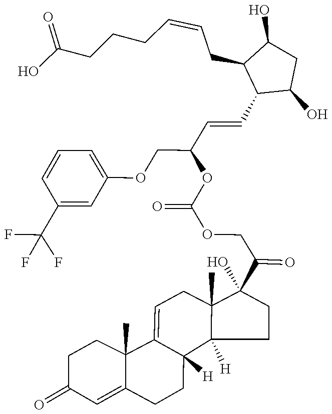 [SMILES: C[C@]12CCC(=O)C=C1CC[C@@H]1C2=CC[C@@]2(C)[C@H]1CC[C@]2(O)C(=O)COC(=O)O[C@H](/C=C/[C@@H]1[C@@H](C/C=C\CCCC(=O)O)[C@@H](O)C[C@H]1O)COc1cccc(C(F)(F)F)c1] | (Z)-7-((1R,2R,3R,5S)-3,5- dihydroxy-2-((R,E)-3-(((2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2- oxoethoxy)carbonyl)oxy)- 4-(3- (trifluoromethyl)phenoxy) but-1-en-1- yl)cyclopentyl)hept-5- enoic acid |
| 45 | 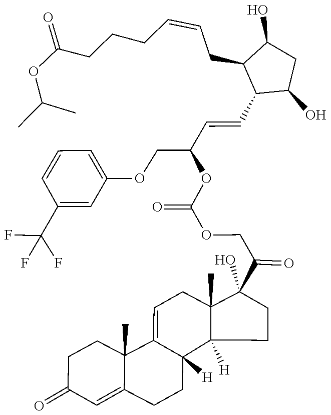 [SMILES: CC(C)OC(=O)CCC/C=C\C[C@@H]1[C@@H](/C=C/[C@H](COc2cccc(C(F)(F)F)c2)OC(=O)OCC(=O)[C@@]2(O)CC[C@H]3[C@@H]4CCC5=CC(=O)CC[C@]5(C)C4=CC[C@@]32C)[C@H](O)C[C@@H]1O] | isopropyl (Z)-7- ((1R,2R,3R,5S)-3,5- dihydroxy-2-((R,E)-3-(((2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2- oxoethoxy)carbonyl)oxy)- 4-(3- (trifluoromethyl)phenoxy) but-1-en-1- yl)cyclopentyl)hept-5- enoate |
| 46 | 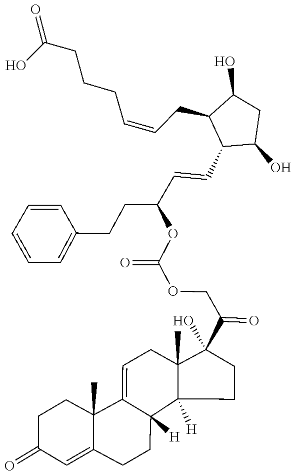 [SMILES: C[C@]12CCC(=O)C=C1CC[C@@H]1C2=CC[C@@]2(C)[C@H]1CC[C@]2(O)C(=O)COC(=O)O[C@H](/C=C/[C@@H]1[C@@H](C/C=C\CCCC(=O)O)[C@@H](O)C[C@H]1O)CCc1ccccc1] | (Z)-7-((1R,2R,3R,5S)-3,5- dihydroxy-2-((S,E)-3-(((2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2- oxoethoxy)carbonyl)oxy)- 5-phenylpent-1-en-1- yl)cyclopentyl)hept-5- enoic acid |
| 47 | 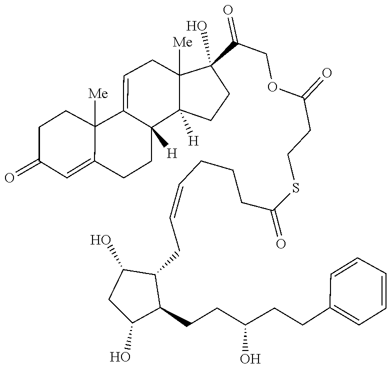 [SMILES: CC12CCC(=O)C=C1CC[C@@H]1C2=CCC2(C)[C@H]1CC[C@]2(O)C(=O)COC(=O)CCSC(=O)CCC/C=C\C[C@@H]1[C@@H](CC[C@@H](O)CCc2ccccc2)[C@H](O)C[C@@H]1O] | 2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethyl 3- (((Z)-7-((1R,2R,3R,5S)- 3,5-dihydroxy-2-((R)-3- hydroxy-5- phenylpentyl)cyclopentyl) hept-5- enoyl)thio)propanoate |
| 48 | 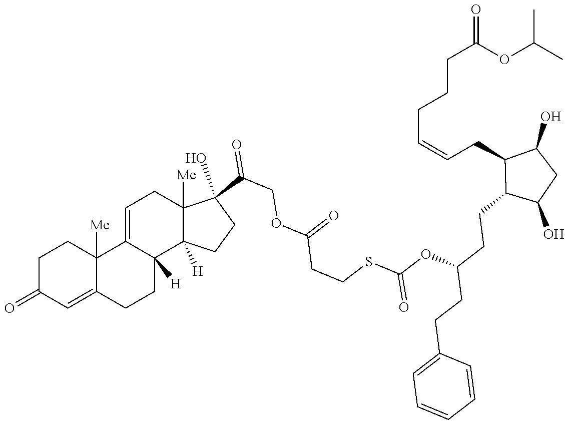 [SMILES: CC(C)OC(=O)CCC/C=C\C[C@@H]1[C@@H](CC[C@H](CCc2ccccc2)OC(=O)SCCC(=O)OCC(=O)[C@@]2(O)CC[C@H]3[C@@H]4CCC5=CC(=O)CCC5(C)C4=CCC32C)[C@H](O)C[C@@H]1O] | isopropyl (Z)-7- ((1R,2R,3R,5S)-3,5- dihydroxy-2-((3R)-3-((((3- (2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethoxy)-3- oxopropyl)thio)carbonyl) oxy)-5- phenylpentyl)cyclopentyl) hept-5-enoate |
| 49 | 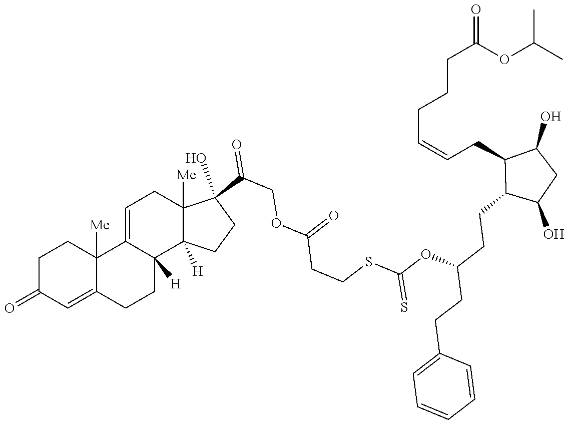 [SMILES: CC(C)OC(=O)CCC/C=C\C[C@@H]1[C@@H](CC[C@H](CCc2ccccc2)OC(=S)SCCC(=O)OCC(=O)[C@@]2(O)CC[C@H]3[C@@H]4CCC5=CC(=O)CCC5(C)C4=CCC32C)[C@H](O)C[C@@H]1O] | isopropyl (Z)-7- ((1R,2R,3R,5S)-3,5- dihydroxy-2-((3R)-3-((((3- (2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethoxy)-3- oxopropyl)thio) carbonothioyl)oxy)-5- phenylpentyl)cyclopentyl) hept-5-enoate |
| 50 | 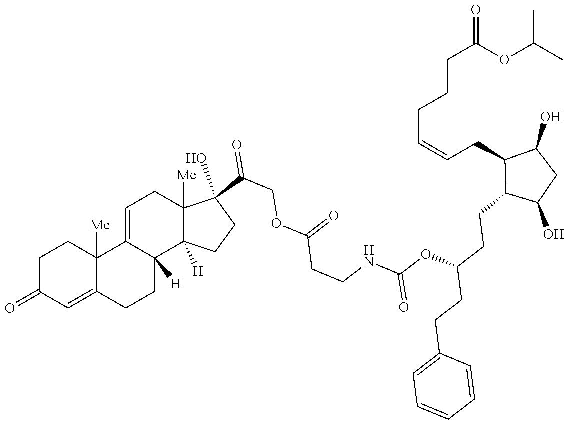 [SMILES: CC(C)OC(=O)CCC/C=C\C[C@@H]1[C@@H](CC[C@H](CCc2ccccc2)OC(=O)NCCC(=O)OCC(=O)[C@@]2(O)CC[C@H]3[C@@H]4CCC5=CC(=O)CCC5(C)C4=CCC32C)[C@H](O)C[C@@H]1O] | isopropyl (Z)-7- ((1R,2R,3R,5S)-3,5- dihydroxy-2-((3R)-3-(((3- (2-((8S,14S,17R)-17- hydroxy-10,13-dimethyl- 3-oxo- 2,3,6,7,8,10,12,13,14,15, 16,17-dodecahydro-1H- cyclopenta[a]phenanthren- 17-yl)-2-oxoethoxy)-3- oxopropyl)carbamoyl) oxy)-5- phenylpentyl)cyclopentyl) hept-5-enoate |

In some embodiments, a composition provided herein (e.g., an article, an implant, or a coating) comprises a compound, or pharmaceutically acceptable salt thereof, provided in Table 7. In some embodiments, the compound comprises both a first radical and a second radical, which when combined are processable (e.g., wherein the radicals may or may not be processable themselves, when in their free form). In some embodiments, the composition provided herein (e.g., an article, an implant, or a coating) comprises a compound, or pharmaceutically acceptable salt thereof, having a structure provided in Table 7. In some embodiments, the compound, or pharmaceutically acceptable salt thereof, provided in Table 7 is a solid at a temperature of at least 20° C. (e.g., at least 30 C, at least 37 C, at least 40 C, at least 50 C, at least 70 C, at least 100 C, or the like). In some embodiments, the compound, or pharmaceutically acceptable salt thereof, provided in Table 7 is processable at a temperature of at least 20° C. (e.g., as described in the examples). In some embodiments, the compound, or pharmaceutically acceptable salt thereof, provided in Table 7 is processable into an article or implant (e.g., machined, molded, emulsion-processed, electrospun, electrosprayed, blow molded, or extruded to form a fiber, fiber mesh, woven fabric, non-woven fabric, film, surface coating, pellet, cylinder, rod, microparticle, nanoparticle, or another shaped article) at a temperature of at least 20° C.

In some embodiments, the compound, or pharmaceutically acceptable salt thereof, provided in Table 7 comprises a first radical and a second radical. In some embodiments, the first radical is a steroid radical and the second radical is a prostaglandin radical. In some embodiments, the compound in Table 7 is processable when the first radical (e.g., the steroid radical) and the second radical (e.g., the prostaglandin radical) are joined by a linker. In some embodiments, the linker is a bond.

TABLE 7

| Compound | | |
|---|---|---|
| Number | Structure | Name |
| 51 | 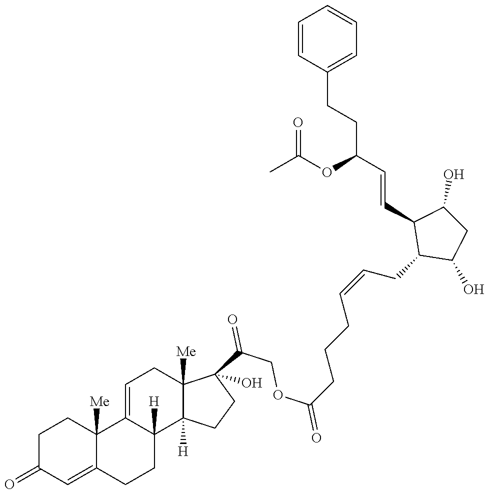 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (Z)-7- ((1R,2R,3R,5S)-2-((S,E)-3- acetoxy-5-phenylpent-1- en-1-yl)-3,5- dihydroxycyclopentyl) hept-5-enoate |
| 52 | 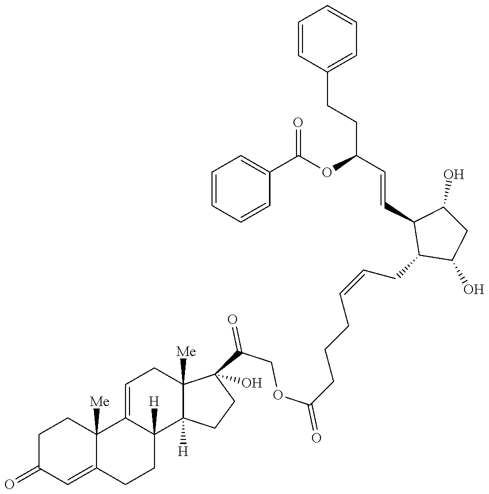 | (S,E)-1-((1R,2R,3S,5R)- 3,5-dihydroxy-2-((Z)-7-(2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethoxy)-7- oxohept-2-en-1- yl)cyclopentyl)-5- phenylpent-1-en-3-yl benzoate |
| 53 | 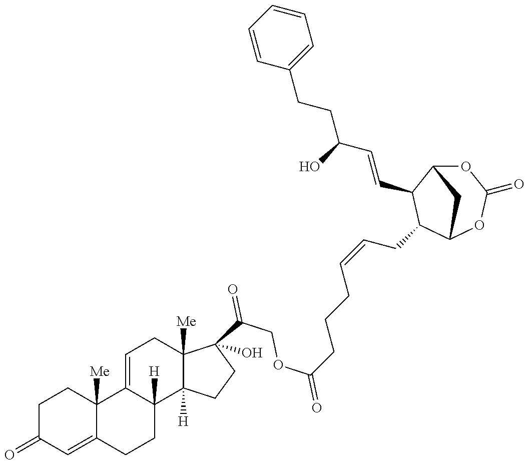 | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (Z)-7- ((1R,5S,6R,7R)-7-((S,E)-3- hydroxy-5-phenylpent-1- en-1-yl)-3-oxo-2,4- dioxabicyclo[3.2.1]octan- 6-yl)hept-5-enoate |

In some embodiments, a composition provided herein (e.g., an article, an implant, or a coating) comprises a compound, or pharmaceutically acceptable salt thereof, provided in Table 8. In some embodiments, the compound comprises both a first radical and a second radical, which when combined are processable (e.g., wherein the radicals may or may not be processable themselves, when in their free form). In some embodiments, the compound, or pharmaceutically acceptable salt thereof, provided in 8 is a solid at a temperature of at least 20° C. (e.g., at least 30 C, at least 37 C, at least 40 C, at least 50 C, at least 70 C, at least 100 C, or the like). In some embodiments, the compound, or pharmaceutically acceptable salt thereof, provided in Table 8 is processable at a temperature of at least 20° C. (e.g., as described in the examples). In some embodiments, the compound, or pharmaceutically acceptable salt thereof, provided in Table 8 is processable into an article or implant (e.g., machined, molded, emulsion-processed, electrospun, electrosprayed, blow molded, or extruded to form a fiber, fiber mesh, woven fabric, non-woven fabric, film, surface coating, pellet, cylinder, rod, microparticle, nanoparticle, or another shaped article) at a temperature of at least 20° C. In some embodiments, the compound, or pharmaceutically acceptable salt thereof, provided in Table 8 comprises both a first radical and a second radical. In some embodiments, the compound in Table 8 is processable when the first radical, and the second radical are joined by a linker. In some embodiments, the linker is a bond.

In some embodiments, the compound, or pharmaceutically acceptable salt thereof, provided in Table 8 comprises a first radical and a second radical. In some embodiments, the first radical is a steroid radical and the second radical is a prostaglandin radical. In some embodiments, the compound in Table 8 is processable when the first radical (e.g., the steroid radical) and the second radical (e.g., the prostaglandin radical) are joined by a linker (e.g., wherein the linker is not a bond). In some embodiments, the linker is (C═O), (—(C═O)O(CH₂O)₃(C═O)—), (—(C═O)OCH₂(C₆H₄)CH₂—), (—(C═O)O(CH₂)₆—), or (—C═OCH₂CH₂C═O—).

TABLE 8

| Compound | | |
|---|---|---|
| Number | Structure | Name |
| 54 | 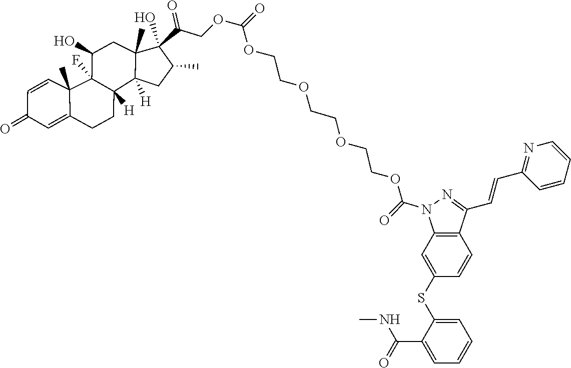 [SMILES: CNC(=O)c1ccccc1Sc1ccc2c(/C=C/c3ccccn3)nn(C(=O)OCCOCCOCCOC(=O)OCC(=O)[C@@]3(O)[C@H](C)C[C@H]4[C@@H]5CCC6=CC(=O)C=C[C@]6(C)[C@@]5(F)[C@@H](O)C[C@@]43C)c2c1] | 1- ((8S,9R,10S,11S,13S,14S, 16R,17R)-9-fluoro-11,17- dihydroxy-10,13,16- trimethyl-3-oxo- 6,7,8,9,10,11,12,13,14,1 5,16,17-dodecahydro- 3H- cyclopenta[a]phenanthren- 17-yl)-1,4-dioxo- 3,5,8,11- tetraoxatridecan-13-yl 6- ((2- (methylcarbamoyl)phenyl) thio)-3-((E)-2-(pyridin-2- yl)vinyl)-1H-indazole-1- carboxylate |
| 55 | 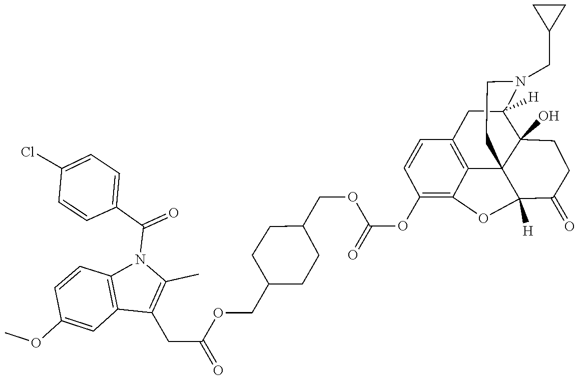 [SMILES: COc1ccc2c(c1)c(CC(=O)OCC1CCC(COC(=O)Oc3ccc4c5c3O[C@H]3C(=O)CC[C@@]6(O)[C@@H](C4)CN(CC4CC4)CC[C@]536)CC1)c(C)n2C(=O)c1ccc(Cl)cc1] | (4-((((((4R,4aS,7aR,12bS)- 3-(cyclopropylmethyl)- 4a-hydroxy-7-oxo- 2,3,4,4a,5,6,7,7a- octahydro-1H-4,12- methanobenzofuro[3,2- e]isoquinolin-9- yl)oxy)carbonyl)oxy) methyl)cyclohexyl)methyl 2- (1-(4-chlorobenzoyl)-5- methoxy-2-methyl-1H- indol-3-yl)acetate |
| 56 | 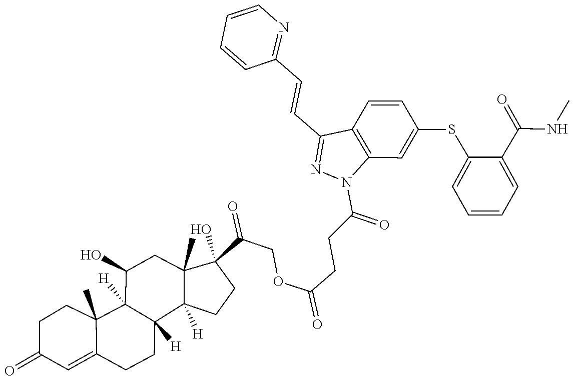 [SMILES: CNC(=O)c1ccccc1Sc1ccc2c(/C=C/c3ccccn3)nn(C(=O)CCC(=O)OCC(=O)[C@@]3(O)CC[C@H]4[C@@H]5CCC6=CC(=O)CC[C@]6(C)[C@H]5[C@@H](O)C[C@@]43C)c2c1] | 2- ((8S,9S,10R,11S,13S,14S, 17R)-11,17-dihydroxy- 10,13-dimethyl-3-oxo- 2,3,6,7,8,9,10,11,12,13, 14,15,16,17- tetradecahydro-1H- cyclopenta[a]phenanthren- 17-yl)-2-oxoethyl 4-(6- ((2- (methylcarbamoyl)phenyl) thio)-3-((E)-2-(pyridin-2- yl)vinyl)-1H-indazol-1-yl)- 4-oxobutanoate |
| 57 | 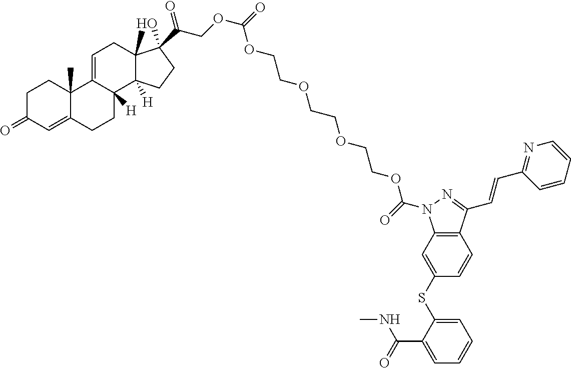 [SMILES: CNC(=O)c1ccccc1Sc1ccc2c(/C=C/c3ccccn3)nn(C(=O)OCCOCCOCCOC(=O)OCC(=O)[C@@]3(O)CC[C@H]4[C@@H]5CCC6=CC(=O)CC[C@]6(C)C5=CC[C@@]43C)c2c1] | 1-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-1,4-dioxo- 3,5,8,11- tetraoxatridecan-13-yl 6- ((2- (methylcarbamoyl)phenyl) thio)-3-((E)-2-(pyridin-2- yl)vinyl)-1H-indazole-1- carboxylate |
| 58 | 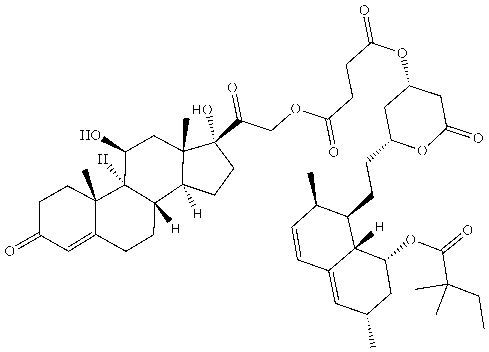 [SMILES: CCC(C)(C)C(=O)O[C@@H]1C[C@H](C)C=C2C=C[C@@H](C)[C@@H](CC[C@@H]3C[C@H](OC(=O)CCC(=O)OCC(=O)[C@@]4(O)CC[C@H]5[C@@H]6CCC7=CC(=O)CC[C@]7(C)[C@H]6[C@@H](O)C[C@@]54C)CC(=O)O3)[C@@H]21] | 2- ((8S,9S,10R,11S,13S,14S, 17R)-11,17-dihydroxy- 10,13-dimethyl-3-oxo- 2,3,6,7,8,9,10,11,12,13, 14,15,16,17- tetradecahydro-1H- cyclopenta[a]phenanthren- 17-yl)-2-oxoethyl ((2R,4S)-2-(2- ((1R,2R,6S,8R,8aS)-8- ((2,2- dimethylbutanoyl)oxy)- 2,6-dimethyl- 1,2,6,7,8,8a- hexahydronaphthalen-1- yl)ethyl)-6- oxotetrahydro-2H-pyran- 4-yl) succinate |
| 59 | 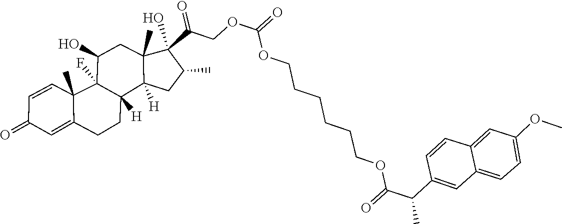 [SMILES: COc1ccc2cc([C@H](C)C(=O)OCCCCCCOC(=O)OCC(=O)[C@@]3(O)[C@H](C)C[C@H]4[C@@H]5CCC6=CC(=O)C=C[C@]6(C)[C@@]5(F)[C@@H](O)C[C@@]43C)ccc2c1] | 6-(((2- ((8S,9R,10S,11S,13S,14S, 16R,17R)-9-fluoro-11,17- dihydroxy-10,13,16- trimethyl-3-oxo- 6,7,8,9,10,11,12,13,14, 15,16,17-dodecahydro- 3H- cyclopenta[a]phenanthren- 17-yl)-2- oxoethoxy)carbonyl)oxy) hexyl (S)-2-(6- methoxynaphthalen-2- yl)propanoate |

Provided in some embodiments herein are articles or implants that demonstrate unique properties for drug release applications. In some embodiments, the release of a (e.g., steroid or prostaglandin) radical in its free form or a compound provided herein from an article or implant provided herein is beneficial (e.g., for drug release applications). In some embodiments, a composition provided herein has near zero-order release kinetics for an extended period of time (e.g., 1 day or more, 2 days or more, 3 days or more, 4 days or more, 5 days or more, 6 days or more, 7 days or more, 14 days or more, or 30 days or more) (e.g., in an individual in need thereof). In some embodiments, a composition provided herein releases at least one radical in its free form or a compound provided herein by surface erosion from the article (e.g., pellet and coating) or implant provided herein.

In certain instances, provided herein is an article, implant, or coating comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8.

In some embodiments, a compound, or pharmaceutically acceptable salt thereof, provided herein, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, is a symmetrical compound (e.g., the first radical and the second radical have the same biological function (e.g., alone or in combination treat the same indication (e.g., described herein)) in their free form). In some embodiments, the symmetrical compound comprises a first radical and a second radical that each have the same biological function (e.g., alone or in combination treat the same indication (e.g., described herein (e.g., glaucoma))) in their free form. In some embodiments, the symmetrical compound comprises a first radical and a second radical that are each a IOP lowering agent in their free form. In some embodiments, the symmetrical compound comprises a first radical and a second radical that (in combination and in their free from) treat an indication described herein (e.g., glaucoma).

In some embodiments, a compound, or pharmaceutically acceptable salt thereof, provided herein, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, is an asymmetrical compound (e.g., the first radical and the second radical have a different structure, a different biological function, or a combination thereof). In some embodiments, the asymmetrical compound comprises a first radical and a second radical that each have the same biological function (e.g., alone or in combination treat the same indication (e.g., described herein (e.g., glaucoma))))) in their free form. In some embodiments, the asymmetrical compound comprises a first radical and a second radical that each have a different biological function in their free form. In some embodiments, the asymmetrical compound comprises a first radical and a second radical that are each an anti-inflammatory agent in their free form. In some embodiments, the asymmetrical compound comprises a first radical and a second radical that are each an IOP lowering agent in their free form. In some embodiments, the asymmetrical compound comprises a first radical and a second radical that (in combination and in their free from) treat an indication described herein. In some embodiments, the asymmetrical compound comprises a first radical that is a steroid in its free form and a second radical that is an IOP lowering agent in its free form. In some embodiments, the first radical and the second radical have a different structure (e.g., as a radical or in their free form). In some embodiments, the asymmetrical compound comprises a first radical and a second radical that (in combination and in their free from) treat an indication described herein (e.g., glaucoma).

In some embodiments, a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, provided herein is a solid, such as at a physiological temperature (e.g., having a melting point (Tₘ) and/or glass transition temperature (T_{g}) of at least 37° C.). In some embodiments, the solid is a crystalline solid, a film, a glass, or an amorphous solid (e.g., at a temperature of at least 37° C.). In some embodiments, the compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, provided herein is not an oil.

In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, has a crystallinity of at most 15% (e.g., determined by PXRD, DSC, or polarized light microscopy). In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, is substantially crystalline (e.g., determined by PXRD, DSC, or polarized light microscopy). In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, is crystalline (e.g., determined by PXRD, DSC, or polarized light microscopy). In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, is amorphous (e.g., determined by PXRD, DSC, or polarized light microscopy).

In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, has a thermal melting point (Tₘ) that is greater than or equal to the glass transition temperature (T_{g}). In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, has a melting point of less than or equal to either one or both of the first and/or second radicals (e.g., in their free form) of the compound.

In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, has a melting point of at least 37° C. In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, has a melting point of at least 100° C. In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, has a melting point of at least 160° C. In some embodiments, an article, implant, or coating provided herein, comprising a compound, or pharmaceutically acceptable salt thereof, having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, has a melting point of at most 220° C.

In some embodiments, the composition (e.g., article) provided herein replaces a dosing regimen series for any (e.g., ocular) disease, disorder, or condition (e.g., glaucoma).

In some embodiments, the composition (e.g., article) provided herein comprises less than 5 wt. %, less than 2 wt. %, or less than 1 wt. % of a controlled release excipient. In some embodiments, the composition (e.g., article) provided herein is free of a controlled release excipient.

In some embodiments, the implant, article, or coating comprises at least 50 wt. % (at least 60 wt. %, at least 70 wt. %, at least 80 wt. %, at least 90 wt. %, at least 95 wt. %, at least 98 wt. %, or the like) of a compound provided herein. In certain instances, the implant or article releases a (e.g., active) group therefrom, such as when implanted into or otherwise administered to an individual (or when placed into an aqueous medium (e.g., aqueous buffer solution), serum, or other physiological medium, such as at a physiological temperature, such as 37° C.). In some instances, a (e.g., active) group released is the free form of the first radical, the second radical, and/or the therapeutic agent. In certain instances, a (e.g., active) group released from article, implant, or coating is an active fragment or metabolite of the first and/or second radical. In some embodiments, the implant, article, or coating undergoes surface erosion to release a compound, the first radical, and/or the second radical (or an (e.g., active) fragment or radical thereof). In some embodiments, the first radical and the second radical are released from the pharmaceutical implant, article, or coating at near zero-order in solution (e.g., buffer solution, serum, biological environment, in vivo, or the like). In some embodiments, the first radical and the second radical (or an (e.g., active) fragment or metabolite thereof) are released from the pharmaceutical implant, article, or coating at 37° C. in 100% bovine serum or at 37° C. in phosphate buffered saline (PBS) at a rate such that t₁₀ is greater than or equal to 1/10 of t₅₀.

In some instances, t₅₀ is the time at which 50% of the releasable drug has been released from a composition (e.g., an article, an implant, or a coating) provided herein. In some instances, time t₁₀ is, correspondingly, the time at which 10% of the releasable drug has been released from a composition (e.g., an article, an implant, or a coating) provided herein. In some instances, such as when the release curve is perfectly linear, t₁₀=⅕ of t₅₀. In some instances, such as when there is an initial burst of released drug, to is much less than ⅕ of t₅₀. In some instances, t₁₀ can be equal to or greater than 1/10 of t₅₀. Drug release from a composition (e.g., an article, an implant, or a coating) provided herein can be measured at 37° C. in 100% bovine serum, or at 37° C. in PBS (phosphate buffered saline), as described in the Examples.

In some embodiments, the pharmaceutical implant, article, coating, or composition is biodegradable.

In some embodiments, the pharmaceutical implant, article, coating, or composition is at least partially biodegradable.

In some embodiments, the pharmaceutical implant, article, coating, or composition is non-biodegradable.

In some embodiments, a compound (a compound having the structure of any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, or a pharmaceutically acceptable salt or solvate thereof) further comprises an amount of a free form of any radical provided herein, or a combination thereof, such as a free form of a radical having the (e.g., steroid and/or prostaglandin) structure of any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, wherein the free form (e.g., —COO— or —O— thereof) has a negative charge (e.g., as —O⁻) or an H (e.g., as —OH), rather than being connected to a linker and/or other (first or second) radical). In some embodiments, a compound provided herein comprises a (e.g., weight or molar) ratio of a compound provided herein to a free form of any radical provided herein, or a combination thereof, such as a free form of a radical having the structure of any one of Formula (I), Formula (I-A), or Formula (I-B), or a pharmaceutically acceptable salt thereof (e.g., wherein the free form (e.g., —COO— or —O— thereof) has a negative charge (e.g., as —O⁻) or an H (e.g., as —OH)), rather than being connected to a linker and/or other (first or second) radical, of about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such 0% to about 20%, 0% to about 10%, about 0.1% to about 10%, about 0.1% to about 5%, less than 5%, less than 2.5%, less than 2%, or the like (percentages being weight/weight or mole/mole percentages). Further, in some instances, compounds provided herein release free form of any radical provided herein, or a combination thereof, such as a free form of a structure of a compound having the structure ofany one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8 (e.g., wherein the free form (e.g., —COO— or —O— thereof) has a negative charge (e.g., as —O—) or H (e.g., as —OH), rather than being connected to a linker and/or other (first or second) radical)), such as when administered to an individual (e.g., ocular, subcutaneous, or topical administration).

In certain instances, a composition provided herein further comprises an amount of a free form of a radical, such as having the structure represented by any one of Formula (I), Formula (I′), Formula (IA), Formula (IB), Formula (IC), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, or the like (such as wherein the free form is the radical, wherein R is a negative charge or an H). In some embodiments, a composition provided herein comprises a (e.g., weight or molar) ratio of a compound provided herein to a free form of a radical having the structure represented by any one of Formula (I), Formula (I′), Formula (IA), Formula (IB), Formula (IC), Formula, (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, or a pharmaceutically acceptable salt thereof (e.g., wherein R is a negative charge or an H) is about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such as about 0% to about 20%, 0% to about 10%, about 0.1% to about 10%, about 0.1% to about 5%, less than 5%, less than 2.5%, less than 2%, or the like (percentages being weight/weight or mole/mole percentages). Further, in some instances, compositions provided herein release free form of a radical of a compound having the structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, (e.g., wherein R is a negative charge or H), such as when administered to an individual (e.g., ocular (e.g., intraocular), subcutaneous, intramuscular administration, or intraspinal administration).

In some embodiments, less than or equal to 20% (w/w) (e.g., less than 20% w/w, less than 15% w/w, less than 10% w/w, or less than 5% w/w) of D1 and/or D2 (e.g., as a percentage of the total article or implant) are released from the article or implant in their free form at 37° C. in PBS over a period 5 days or more (e.g., more than 5 days, more than 7 days, or more than 10 days). In some embodiments, less than or equal to 20% (w/w) (e.g., less than 20% w/w, less than 15% w/w, less than 10% w/w, or less than 5% w/w) of the at least one therapeutic agent, D1, and/or D2 (e.g., as a percentage of the total article or implant) are released from the article or implant in their free form at 37° C. in PBS over a period 5 days or more (e.g., more than 5 days, more than 7 days, or more than 10 days).

In some embodiments, the article or implant has a melting point of at least 37° C. (e.g., at least 100° C., at least 160° C., or at least 200° C.). In some instances, the article or implant has a melting point of at most 220° C. In some instances, an article or implant with a melting point greater than 220° C. decomposes (e.g., and is not processable into an article (e.g., a pellet) or implant provided herein) subsequent to heat processing or solvent processing methods provided herein. In some instances, the article or implant provided herein has a melting point that is less than or equal to either one or both of the first and/or second radicals (e.g., in their free form) of the article or implant.

In some embodiments, the release profile of the article or implant provided herein is modified by the linker (e.g., L). For example, in certain instances, a linker that is less susceptible to hydrolysis (e.g., steric effects, electronic effects, etc.) provides an article or implant that releases (e.g., from the article or implant) an compound provide herein (e.g., a radical thereof in its free form) at a slower (e.g., extended-release) rate compared to an article or implant that is more susceptible to hydrolysis (e.g., providing an article or implant that releases (e.g., from the article or implant) compound provided herein (e.g., a radical thereof in its free form) at a faster (e.g., immediate-release) rate).

In some embodiments, either one or both of the first and/or second radicals of a composition provided herein (e.g., an article, implant, or coating), having a structure represented by any one of Formula (I), Formula (ID), Formula (IE), Formula (IE-i), Formula (IE-ii), Formula (IE-iii), Formula (IF), Formula (II), Formula (II′), Formula (IA), Formula (IB), Formula (IC), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, is released (e.g., in their free form) from the composition, the release being controlled release (e.g., near zero-order) and/or extended release. In some embodiments, either one or both of the first and/or second radicals of a composition provided herein (e.g., an article, implant, or coating), having a structure represented by any one of Formula (I), Formula (ID), Formula (IE), Formula (IE-i), Formula (IE-ii), Formula (IE-iii), Formula (IF), Formula (II), Formula (II′), Formula (IA), Formula (IB), Formula (IC), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, is released (e.g., in theirfree form) from the composition for at least 15 days (e.g., a buffer solution, serum, biological environment (e.g., in the eye), in vivo, or the like).

In some instances, a compound provided herein is administered as a pure (e.g., greater than 98 wt. %, greater than 99 wt. %, about 100 wt. %) chemical. In other embodiments, a composition described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

In some embodiments, provided herein is a pharmaceutical composition comprising any compound provided herein, such as a compound that has a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8, or a pharmaceutically acceptable salt thereof, and at least one pharmaceutically acceptable excipient.

In some embodiments, the compound is formed into a surface coating, a drug depot, an article, an implant, or other material or form described herein.

In some embodiments, the compound is formed into an implantable article or an implant. In some instances, the implantable article or the implant consists essentially of the compound.

In some embodiments, the compound is in the form of a coating. In some embodiments, the compound is a coating on the article body. In some embodiments, the compound coats at least one surface of the article body. In some embodiments, the compound is a coating on at least partially covers the article body. In some embodiments, the compound is in the form of a coating, the coating being on (e.g., at least partially covering) at least one surface of the article body.

In some embodiments, the article body is an implant. In some embodiments, the article body is an implantable device. In some embodiments, the article body is an implantable medical device.

In some embodiments, the article body is a contact lens, a microshunt device, a microinvasive glaucoma surgery (MIGS) device, an intraocular lenses, or the like.

The article body can be a medical device and the surface coating resides on the surface of the medical device. For example, the article body can be blood dwelling medical device (e.g., a heart valve, vascular stent, endovascular coil, or catheter), urine dwelling medical device (e.g., a drainage catheter or ureteral stent), and/or subcutaneously dwelling medical device (e.g., an implantable sensor). In some embodiments, the implantable medical device can be blood dwelling medical device (e.g., a heart valve, vascular stent, endovascular coil, or catheter), urine dwelling medical device (e.g., a drainage catheter or ureteral stent), and/or subcutaneously dwelling medical device (e.g., an implantable sensor). The article body or implantable medical device can be an implantable device selected from the group consisting of prostheses pacemakers, electrical leads, defibrillators, artificial hearts, ventricular assist devices, anatomical reconstruction prostheses, artificial heart valves, heart valve stents, pericardial patches, surgical patches, coronary stents, vascular grafts, vascular and structural stents, vascular or cardiovascular shunts, biological conduits, pledges, sutures, annuloplasty rings, staples, valved grafts, dermal grafts for wound healing, orthopedic spinal implants, ophthalmic implants, intrauterine devices, maxial facial reconstruction plating, intraocular lenses, clips, and sternal wires. In particular embodiments, the implantable device is an orthopedic device selected from a wire, pin, rod, nail, screw, disk, plate, bracket, or splint. In other embodiments, the article body or implantable device is selected from the group consisting of dental devices, drug delivery devices, grafts, stents, implantable cardioverter-defibrillators, heart valves, vena cava filters, endovascular coils, catheters, shunts, wound drains, drainage catheters, infusion ports, cochlear implants, endotracheal tubes, tracheostomy tubes, ventilator breathing tubes, implantable sensors, ophthalmic devices, orthopedic devices, dental implants, periodontal implants, breast implants, penile implants, maxillofacial implants, cosmetic implants, valves, appliances, scaffolding, suturing material, needles, hernia repair meshes, tension-free vaginal tape and vaginal slings, prosthetic neurological devices, ear tubes, a wound dressing, a bandage, a gauze, a tape, a pad, a sponge, a contraceptive device, and feminine hygiene products.

In some embodiments, the surface coating resides on a contact lens, a microshunt device, a microinvasive glaucoma surgery (MIGS) device, an intraocular lenses, or the like.

In some embodiments, the pharmaceutical composition is suitable for ophthalmic administration. In some embodiments, the pharmaceutical composition is suitable for intraocular ophthalmic administration. In some embodiments, intraocular ophthalmic administration is administration in the eye, such as intraocular, intracameral, intravitreal, suprachoroidal, punctal, retrobulbar, or subconjunctival.

In some embodiments, the pharmaceutical composition is suitable for subcutaneous administration.

In some embodiments, the pharmaceutical composition is suitable for intramuscular administration.

In some embodiments, the pharmaceutical composition is suitable for ocular administration. In some embodiments, the pharmaceutical composition is suitable for intraocular administration.

In certain instances, any compound and/or composition (e.g., article, implant, coating) provided herein is substantially pure, in that it contains less than about 5%, or less than about 1%, or less than about 0.1%, of other organic small molecules, such as impurities, unreacted intermediates or (e.g., processing and/or synthesis) by-products that are created, for example, in one or more of the steps of a synthesis method and/or processing step (such as heat processing, solvent processing, and/or sterilization).

In some embodiments, an implant, article, or coating provided herein comprises more than or equal to 25 wt. % (50 wt. % or more, 75 wt. % or more, 90 wt. % or more, 95 wt. % or more, or 99 wt. % or more) of a compound, and/or pharmaceutically acceptable salt thereof, provided herein. In some embodiments, an implant, article, or coating provided herein comprises less than or equal to 25 wt. % of a compound, and/or pharmaceutically acceptable salt thereof, provided herein.

In some embodiments, an implant, article, or coating provided herein comprises more than or equal to 50 wt. % (at least 60 wt. %, at least 70 wt. %, at least 80 wt. %, at least 90 wt. %, at least 95 wt. %, at least 98 wt. %, or more) of a compound, and/or pharmaceutically acceptable salt thereof, provided herein. In some embodiments, an implant, article, or coating provided herein comprises less than or equal to 50 wt. % (at most 40 wt. %, at most 30 wt. %, at most 20 wt. %, at most 10 wt. %, at most 5 wt. %, at most 1 wt. %, or less) of a compound, and/or pharmaceutically acceptable salt thereof, provided herein.

In some embodiments, the implant or article comprises at least 50 wt. % (at least 60 wt. %, at least 70 wt. %, at least 80 wt. %, at least 90 wt. %, at least 95 wt. %, at least 98 wt. %, or the like) of the compound and/or pharmaceutically acceptable salt thereof. In some instances, an article or implant provided herein comprises at least 50 wt. % of a compound as described herein. In some instances, an article or implant provided herein comprises at least 70 wt. % of a compound as described herein. In some instances, an article or implant provided herein comprises at least 90 wt. % of a compound as described herein. In some instances, an article or implant provided herein comprises at least 95 wt. % of a compound as described herein. In some instances, an article or implant provided herein comprises at least 99 wt. % of a compound as described herein. In some instances, an article or implant provided herein comprises an additional component, such as up to 20 wt. %, 15 wt. %, 10 wt. %, 5 wt. %, 1 wt. %, 0.1 wt. %, 0.01 wt. %, or less of the additional component. In some embodiments, an article or implant provided herein comprises up to 5 wt. % (e.g., up to 1 wt. %, up to 0.1 wt. %, or less) a first radical in its free form (e.g., a steroid (such as described herein)), a second radical in its free form (e.g., a prostaglandin (such as described herein), or a combination thereof (e.g., as an impurity, such as residual from a manufacturing process, such as provided herein). In some embodiments, an article or implant provided herein comprises up to 5 wt. % (e.g., up to 1 wt. %, up to 0.1 wt. %, or less) of an impurity, such as residual from a manufacturing process, such as provided herein.

In some embodiments, an article or implant provided herein comprises up to 5 wt. % (e.g., up to 1 wt. %, up to 0.1 wt. %, or less) of a steroid (as described herein).

In some embodiments, an implant, article, or coating provided herein comprises about 70 wt. % or more of a compound provided herein.

In some embodiments, an implant, article, or coating provided herein comprises about 80 wt. % or more of a compound provided herein.

In some embodiments, an implant, article, or coating provided herein comprises about 90 wt. % or more of a compound provided herein.

In some embodiments, an implant, article, or coating provided herein undergoes surface erosion to release a compound described herein. In some embodiments, a compound provided herein is released from the pharmaceutical implant, article, or coating at near zero-order in solution (e.g., buffer solution, serum, biological environment, in vivo, or the like). In some embodiments, a compound provided herein is released from the pharmaceutical implant, article, or coating at 37° C. in 100% bovine serum or at 37° C. in phosphate buffered saline (PBS) at a rate such that t₁₀ is greater than or equal to 1/10 of t₅₀.

In certain embodiments, the implant or article releases a (e.g., active) group therefrom, such as when implanted into or otherwise administered to an individual (or when placed into an aqueous medium (e.g., aqueous buffer solution), serum, or other physiological medium, such as at a physiological temperature, such as 37° C.). In some instances, a (e.g., active) group released is the free form of the first radical and/or the second radical. In certain instances, a (e.g., active) group released from the compound is an active fragment or metabolite of the first and/or second radical. In some embodiments, the implant or article undergoes surface erosion to release the compound, the first radical, and/or the second radical (or an (e.g., active) fragment or radical thereof). In some embodiments, first radical and the second radical are released from the pharmaceutical implant or article at near zero-order in solution (e.g., buffer solution, serum, biological environment, in vivo, or the like). In some embodiments, the first radical and the second radical (or an (e.g., active) fragment or metabolite thereof) are released from the pharmaceutical implant or article at 37° C. in 100% bovine serum or at 37° C. in phosphate buffered saline (PBS) at a rate such that t₁₀ is greater than or equal to 1/10 of t₅₀.

In certain aspects, a pharmaceutical composition provided herein includes an article or implant in the form of fibers, fiber meshes, woven fabrics, non-woven fabrics, pellets, cylinders, rods, hollow tubes, microparticles, nanoparticles, or other shaped articles. In some embodiments, a pellet is rounded, spherical, cylindrical, or a combination thereof. In some embodiments, the pellet has a mean diameter of 0.01 mm or more (e.g., 0.1 mm or more or 1 mm or more). In some embodiments, the pellet has a mean diameter of 5 mm or less (e.g., 5 mm or less, 0.5 mm or less, or 0.05 or less). In some embodiments, the pellet has a mean diameter from about 0.2 to 5 mm, e.g., from about 0.2 to 1 mm, from about 0.2 to 2 mm, from about 0.3 to 3 mm, from about 1.5 to 5 mm, from about 2 to 5 mm, from about 2.5 to 5 mm, from about 3 to 5 mm, from about 3.5 to 5 mm, from about 4 to 5 mm, or from about 4.5 to 5 mm. In some embodiments, a pharmaceutical composition provided herein has a non-circular shape that affects, e.g., increases, the surface area (e.g., extruded through star-shaped dye or any other form shaping process with or without a dye mold). In some embodiments, suitable pharmaceutical compositions for use with this disclosure are small regularly or irregularly shaped particles, which can be solid, porous, or hollow.

In some embodiments, certain forms of pharmaceutical compositions described herein (e.g., fibers, fiber meshes, woven fabrics, non-woven fabrics, pellets, cylinders, rods, hollow tubes, microparticles (e.g., microbeads), nanoparticles (e.g., nanobeads), or other shaped articles) provide a controllable surface area. In some embodiments, a pharmaceutical composition provided herein is injected into an individual (e.g., in need thereof) and does not require removal after completion of drug release. In certain instances, methods provided herein do not require (or comprise) removal of an article or implant, or residual materials or components thereof (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)).

In some embodiments, a composition provided herein is used as a drug delivery device containing little to no additives. This may achieve a local, sustained release and a local biological effect, while minimizing a systemic response. In some instances, a composition provided herein provides (e.g., sustained) local release (e.g., drug delivery), such as providing a local and/or a systemic effect in an individual (e.g., in need thereof). In some instances, a composition provided herein provides (e.g., sustained) systemic release (e.g., drug delivery), such as providing a systemic effect in an individual (e.g., in need thereof). In some embodiments, when present, the additives are in small amounts and do not affect the physical or bulk properties. In some embodiments, when present, the additives do not alter the drug release properties from the pharmaceutical composition but rather act to improve processing of the prodrug dimer into the shaped article. In some embodiments, the pharmaceutical compositions contain additives such as a plasticizer (e.g., to reduce thermal transition temperatures), an antioxidant (e.g., to increase stability during heat processing), a binder (e.g., to add flexibility to the fibers), a bulking agent (e.g., to reduce total drug content), a lubricant, a radio-opaque agent, a solubilizing excipient (e.g., PEG(s)), or mixtures thereof. In some embodiments, the additives are present at 30% (w/w), e.g., 20% (w/w), 10% (w/w), 7% (w/w), 5% (w/w), 3% (w/w), 1% (w/w), 0.5% (w/w), or 0.1% (w/w). Non-limiting examples of plasticizers are polyols, e.g., glycerol, ethylene glycol, diethylene glycol, triethylene glycol, tetraethylene glycol, polyethylene glycol, propylene glycol, triacetin, sorbitol, mannitol, xylitol, fatty acids, monosaccharides (e.g., glucose, mannose, fructose, sucrose), ethanolamine, urea, triethanolamine, vegetable oils, lecithin, orwaxes. Non-limiting examples of antioxidants are glutathione, ascorbic acid, cysteine, or tocopherol. The binders and bulking agents can be, e.g., polyvvinylpyrrolidone (PVP), starch paste, pregelatinized starch, hydroxypropyl methyl cellulose (HPMC), carboxymethyl cellulose (CMC), or polyethylene glycol (PEG) 6000.

In some instances, a solubilizing agent is added to increase the release rate of a component from the composition, such as, for example, a compound (e.g., or a radical thereof in its free form).

In some embodiments, the implants, articles, or compositions described herein are amorphous. In some embodiments, the implants, articles, or compositions described herein are formed by heat-based and solvent based processing methods. Non-limiting examples of heat processing methods include heat molding, injection molding, extrusion, 3D printing, melt electrospinning, fiber spinning, fiber extrusion, and/or blow molding. Non-limiting examples of solvent processing include coating, micro printing, dot printing, micropatterning, fiber spinning, solvent blow molding, emulsion-based, electrospraying, and electrospinning. In some embodiments, processing methods to form an intermediate glassy state of any of the above heat and solvent based methods as well as heat and solvent based methods that lead to glassy state material with no defined shape (e.g. spray drying, lyophilization, powder melting, etc.).

In some embodiments, a glassy state is an amorphous solid including greater than 70%, 80%, 90%, 95%, 98%, or 99% (w/w) of compositions, articles, or implants described herein. In some embodiments, the compositions, articles, or implants described herein exhibit a glass transition temperature above 38° C. In the glassy state, as measured by differential scanning calorimetry (DSC), X-ray powder diffraction (XRD), or polarized light microscopy (PLM), the level of crystallinity is, for example, from 0-15% (e.g., less than 1%, 0-1%, 0-3%, 0-5%, 0-7%, 0-9%, 0-10%, or 0-13%). In some embodiments, glass formulations are formed using heat processing or solvent processing methods described herein (e.g., in the examples).

In some embodiments, the pharmaceutical compositions described herein are prepared by electrospinning. In some embodiments, the pharmaceutical compositions of the disclosure are dissolved in a solvent (e.g., acetone) at concentrations ranging from, e.g., 10-30% w/v, and are electrosprayed to form micro- and nanobeads. In some embodiments, the solution is loaded into a syringe and injected at a rate (e.g., 0.5 mL/h) onto a stationary collection plate. In some embodiments, a potential difference (e.g., 18 kV) is maintained between the needle and collecting surface. For example, in certain instances, a concentration of 10% w/v is used to obtain nanoparticles. In other embodiments, a concentration of 30% w/v is used to obtain microbeads.

The pharmaceutical compositions of the disclosure are dissolved in a solvent (e.g., THF, or 1:1 ratio of DCM/THF). In some embodiments, the solution is loaded into a syringe and injected at a rate (e.g., 0.5 mL/h) onto a cylindrical mandrel rotating at a particular rotational speed, e.g., 1150 rpm, to obtain aligned fibers, or onto a stationary collector surface to obtain unaligned fibers. In some embodiments, a potential difference (e.g., 18 kV or 17 kV) is maintained between the needle and collecting surface for aligned and random fibers.

In some embodiments, provided herein is a method of forming an article or implant provided herein comprising producing a glassy state (e.g., an intermediate glassy state or a melt) of a compound (e.g., a crystalline form (e.g., a solid or a powder)) provided herein using heat or solvent. In other embodiments, fibers are prepared from the melt or the glass at elevated temperatures, the glassy state intermediate, or from the solution by dissolving the pharmaceutical compositions described herein in a solvent (e.g., DCM, THF, or chloroform). In some instances, compositions provided herein are heat processed from the melt state. In some instances, compositions provided herein are heat processed by heat spinning from the glassy state.

In some embodiments, the viscous melt, intermediate, or solution is fed through a spinneret and fibers are formed upon cooling (melt or heat spinning) or following solvent evaporation with warm air as the compound exits the spinneret (dry spinning). In some embodiments, wet spinning and gel spinning are used to produce the fibers disclosed herein. In some embodiments, heat spinning is performed with a glassy state intermediate and heated above the glass transition temperature (T_{g}), obtaining the viscous fluid to extrude/spin instead of the melt. Alternatively, tweezers may be dipped into melted material or concentrated solutions and retracted slowly in order to pull fibers. The rate of pulling and distance pulled may be varied to yield fibers and columnar structures of different thickness.

In some embodiments, micro-particles or nano-particles made from the pharmaceutical composition are formed using an emulsion process. In some embodiments, the micro or nano-particles are made by recrystallization. In some embodiments, the pharmaceutical composition is dissolved in an organic solvent (e.g. DCM, THF, etc.). In some embodiments, a surfactant (e.g. SDS, PVA, etc.) is added (e.g. 1%) to the solution/mixture. In some embodiments, the resulting mixture is stirred for the appropriate time at room temperature to form an emulsion. In some embodiments, the emulsion is subsequently added to Milli-Q water under stirring for an appropriate time (e.g., 1 h) to remove residual solvent. The resulting micro- or nano-particles may be collected by centrifugation and dried.

In some embodiments, injectable cylinders made from a pharmaceutical composition described herein is formed by heat extrusion. In some embodiments, the pharmaceutical composition is loaded into a hot melt extruder, heated to a temperature above the melting point (e.g., for crystalline compositions) or glass transition temperature (e.g., for pre-melted or amorphous compositions), and extruded using (i) a compressive force to push the material through the nozzle and (ii) a tensile force (or gravity) to pull the material out of the extruder. The extrudate may be cut to the desired length for suitable drug dosing for a medical indication.

In some embodiments, a milling process is used to reduce the size of an article described to form sized particles, e.g., beads, in the micrometer (microbeads) to nanometer size range (nanobeads). The milling process may be performed using a mill or other suitable apparatus. In some embodiments, dry and wet milling processes, such as, for example, jet milling, cryo-milling, ball milling, media milling, sonication, and homogenization are used in methods described herein. In some embodiments, heating of the milled microparticle above the T_{g} is performed to achieve a spherical shape. In some embodiments, particles with non-spherical shapes are used as milled.

In certain instances, a composition described herein has a limited window (e.g., short timeframe of seconds to minutes) of thermal stability, whereby the purity of the dimer is affected (e.g., minimally) at elevated temperatures. In some embodiments, an intermediate glassy state form (e.g., film, surface coating, pellet, micro-particles, or other shaped article) is made to avoid decomposition. In some embodiments, heat or solvent processing is used to remove or reduce the crystallinity of the material to form a glassy state composition. In some embodiments, the glassy state composition is heat processed at a lower temperature (e.g., processing just above the glass transition temperature (T_{g}), and below the melt temperature (Tₘ)). In some embodiments, the lower temperature allows for a longer timeframe for heat processing the glassy state material into the final shaped article, while reducing the impact of processing conditions on the purity of the compound in the article.

In some embodiments, an article or implant provided herein is formulated for administration by injection. In some instances, the injection formulation is a solid formulation. In some instances, the injection formulation is a non-aqueous formulation.

In certain embodiments, a pharmaceutical compositions described herein has a controlled release profile (e.g., by surface erosion). In some embodiments, the surface erosion allows the article or implant to maintain its physical form, while gradually decreasing in size as the surface erodes (e.g., at a constant rate), rather than by, for example, bulk erosion that is characteristic of some polymer-based drug release vehicles (e.g., polylactic/glycolic acid). In some embodiments, the surface erosion inhibits burst release and/or reduces the formation of inflammatory particulates (e.g., no or minimal crystalline particulates are formed or released from the articles or implants when drug is released as described herein). In some embodiments, compositions described herein are delivered over a period of time. For example, a slower and steadier rate of delivery (e.g., release of less than 10% of the first radical or the second radical in their free form (as a percentage of the total drug, the first radical or the second radical in their free form, present in the article or implant) at 37° C. in 100% bovine serum over 5 days) results in a reduction in the frequency with which the pharmaceutical composition is administered to a subject and/or improve the safety profile of the drug. In some embodiments, the drug release is tailored to avoid side effects of slower and longer release of the drug by engineering the article or implant to provide constant release over a comparatively shorter period of time. In some embodiments, the drug release is tailored for dose and duration suitable for the indication or administration method.

In some embodiments, the release rate is related to, for example, the drug configuration of the dimer. In some embodiments, the drug release rate from an article or implant described herein is modulated by the cleavage of dimer-linker bond through hydrolysis or enzymatic degradation. In some embodiments, the linking moiety (e.g., the linker) affects drug release rate. In some embodiments, the drug release rate is controlled by a functional group on the composition described herein to conjugate through to the linker, for example, a primary vs. a secondary hydroxyl group. In some embodiments, the release rate from a dimer is related to percentage of the loaded dimer compared to the final drug dimer formulation (e.g., by using a pharmaceutical excipient (e.g., bulking agent/excipient). In some embodiments, the release rate is controlled by the size of a microbead. In some embodiments, drug release is tailored based on the solubility of drug dimer (e.g., through selection of appropriate drug and/or linker) that will influence the rate of surface erosion (e.g., dissolution/degradation) from the article or implant. In other embodiments, drug release is affected by changes in surface area of the formulation, e.g., by changing the diameter of the microbeads. By adjusting the vide supra factors, dissolution, degradation, diffusion, and controlled release may be varied over wide ranges. For example, release may be designed to be initiated over minutes to hours, and may extend over the course of days, weeks, months, or years.

In some embodiments, a pharmaceutical composition containing a processable material described herein is administered to an individual by the following non-limiting examples: oral, sublingual, nasal, intradermal, subcutaneous, intramuscular, rectal, vaginal, intravenous, intraarterial, intracisternal, intraperitoneal, intravitreal, intraocular, topical (as by powders, creams, ointments, or drops), buccal and inhalational administration. In certain instances, the articles or implants described herein are administered parenterally as injections (intravenous, intramuscular, or subcutaneous), or locally as injections (into an ocular space). In some embodiments, the formulations described herein are admixed under sterile conditions with a pharmaceutically acceptable carrier, preservatives and/or buffers.

In some embodiments, an implant, article, or composition described herein is suitable for ophthalmic administration, subcutaneous administration, or intraspinal administration. In some embodiments, the ophthalmic administration is intraocular, subretinal, superciliary, forniceal, into Schlemm's canal, inside a bleb, intracameral, intravitreal, suprachoroidal, punctal, retrobulbar, or subconjunctival.

In some instances, an implant, article, or composition described herein is a coating on a device. In some instances, the device is a contact lenses, a microshunt device, microinvasive glaucoma surgery (MIGS) device, an intraocular lenses, or the like.

The dose of the composition comprising at least one compound as described herein differ, depending upon the individual's (e.g., human) condition, that is, general health status, age, and other factors.

Pharmaceutical compositions are administered in a manner appropriate to the disease to be treated (or prevented). An appropriate dose and a suitable duration and frequency of administration will be determined by such factors as the condition of the individual, the type and severity of the individual's disease, the particular form and/or potency of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the individual.

In other embodiments, the compositions described herein are combined with a pharmaceutically suitable or acceptable carrier (e.g., a pharmaceutically suitable (or acceptable) excipient, physiologically suitable (or acceptable) excipient, or physiologically suitable (or acceptable) carrier. Exemplary excipients are described, for example, in Remington: The Science and Practice of Pharmacy (Gennaro, 21^{st} Ed. Mack Pub. Co., Easton, PA (2005)).

### Methods of Treatment

Provided in some embodiments herein is a method of treating a medical indication or abnormality (e.g., an ophthalmic disease and/or disorder), the method comprising administering a therapeutically effective amount of a compound or composition provided herein.

In some embodiments, the medical indication or abnormality is a cardiac indication or abnormality, a neurological indication or abnormality, a respiratory indication or abnormality, a rheumatological indication or abnormality, a metabolic indication or abnormality, a urological indication or abnormality, or a bone indication or abnormality.

In some embodiments, the medical indication or abnormality is an inflammatory indication or abnormality.

In some embodiments, the medical indication or abnormality is an infection.

In some embodiments, the medical indication or abnormality is an indication that is treated, prevented, managed, or the like with a drug described in Table 2.

In some embodiments, the medical indication or abnormality is an indication that is treated, prevented, managed, or the like with a therapeutic agent of a drug class described in Table 2.

In some embodiments, the medical indication or abnormality is an indication that is treated, prevented, managed, or the like with a therapeutic agent provided in Table 2.

In some embodiments, a composition provided herein (e.g., used in a method provided herein) comprises a compound provided herein in a therapeutically effective amount (e.g., at a concentration effective to treat an ophthalmic disease or disorder in an individual in need thereof, the method comprising administering to the individual a compound, pharmaceutically acceptable salt, implant, article, or composition having the structure of any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8. In some embodiments, a composition provided herein (e.g., used in a method provided herein) comprises a compound provided herein in a therapeutically effective amount (e.g., at a concentration effective to treat glaucoma, inflammation, and/or lower intraocular pressure) in the eye.

In some embodiments, provided herein is a method of treating an ophthalmic or post-operative disease, disorder, or condition in an individual (e.g., in need of thereof), comprising administering to the individual any compound provided herein, or a pharmaceutically acceptable salt thereof, or a (e.g., pharmaceutical) composition comprising any compound provided herein, or a pharmaceutically acceptable salt thereof, such as a compound having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8.

In some embodiments, provided herein is a method of treating an ophthalmic disease, disorder, or condition in an individual (e.g., in need of thereof), comprising administering to the individual a (e.g., pharmaceutical) composition provided herein, such as, comprising any article or implant containing any compound provided herein, or a pharmaceutically acceptable salt thereof, such as at least one therapeutic agent and a compound having a structure represented by any one of Formula (I), Formula (I′), Formula (IA), Formula (IB), Formula (IC), Formula (ID), Formula (IE), Formula (IE-i), Formula (IE-ii), Formula (IE-iii), Formula (IF), Formula (II), Formula (II′), Formula (IIA), Formula (IIB), Formula (III), Formula (III′), Formula (III-A), Formula (III-B), Formula (IV), Formula (IV′), Formula (V′), Formula (VI), Formula (VI-A), Formula (VI-B), Formula (VI-C), Formula (VII), Formula (VII-A), Formula (VII-B), Formula (VIII), Formula (VIII-A), Formula (VIII-B), Formula (IX), Formula (X), or provided in Tables 4 or 6-8.

In some embodiment, the pharmaceutical composition is in the form of a solid suitable for intraocular ophthalmic administration (e.g., injection). In some embodiments, intraocular ophthalmic administration is intraocular, subretinal, superciliary, forniceal, into Schlemm's canal, inside a bleb, intracameral, intravitreal, suprachoroidal, punctal, retrobulbar, or subconjunctival.

Methods involving treating an individual, in some instances, include preventing a disease, disorder or condition from occurring in the subject which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected (e.g., such as decreasing the ocular condition of an individual by administration of an agent even though such agent does not treat the cause of the ocular condition).

In some instances, provided herein is a method of treating one or more ophthalmic disease, disorder, or condition in an individual (e.g., in need of thereof).

In some embodiments, the ophthalmic disease, disorder or condition is selected from the group consisting of glaucoma, ocular hypertension, ocular inflammation (e.g., from a surgical procedure), a bacterial infection (e.g., from a surgical procedure), diabetic macular edema, posterior inflammation, anterior inflammation, macular degeneration (e.g., wet macular degeneration (AMD)), post-cataract surgery, and retinal vein occlusion.

In some embodiments, the ocular disease or disorder is glaucoma.

In some embodiments, the ocular disease or disorder is ocular hypertension.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Analytical Methods

### Analytical Example 1: High Performance Liquid Chromatography (HPLC)

Samples (20.0 mg) are dissolved in acetonitrile (10.0 mL) to make 2 mg/mL solution. For the system: solvent A was Water+0.05% trifluoroacetic acid (TFA); solvent B was Acetonitrile+0.05% TFA; the flow rate was 1.0 mL/min; and the detection method was UV @242 nm and UV Spectra from 190 to 400 nm. The samples were loaded onto an Agilent 1100 series HPLC with either (i) a Phenomenex Gemini-NX C18 Column (5 μm; 110 Å; 250×4.6 mm; 00G-4454-E0) or (ii) Phenomenex SecurityGuard Analytical Guard Column (KJO-4282) with Gemini C18 4×3.0 mm Guard Cartridge (AJO-7597). The solvent gradient profile is shown in Table 9:

TABLE 9

| Time (min) | % A Solvent | % B Solvent |
|---|---|---|
| 0 | 80 | 20 |
| 40 | 16 | 84 |
| 42 | 0 | 100 |
| 50 | 0 | 100 |

### Analytical Example 2: Nuclear Magnetic Resonance (NMR)

Compounds (10 mg) were dissolved in 666 μL of either CDCl₃ or DMSO-d6 and loaded in an 8-inch length, 5 mm diameter NMR tube. The instrument was a Varian Mercury 400 NMR spectrometer. Proton NMR spectra were obtained with 16 scans using the default method. FIDs were processed with MestRe-C software.

### Analytical Example 3: Mass Spectrometry (MS)

Compounds were dissolved in acetonitrile at 1 mg/ml and used directly for analysis on an Agilent 6538 QTOF, using ESI MS+ as ion source.

### Analytical Example 4: Melting Point

Compound powder was prepared neat in a glass capillary tube, and melting temperature was measured manually with standard glass capillary tube melting point apparatus.

### Analytical Example 5: Differential Scanning Calorimetry (DSC)

5-10 mg of compounds were weighed in an aluminum pan. Using a Hitachi Differential Scanning Calorimeter DSC7020 or a TA Instruments DSC250, samples were heated from room temperature to 110-175° C. at 10° C./min, cooled to −30° C. at 10° C./min, and heated again to 110-175° C. at 10° C./min.

### Example 2: Chemical Synthesis

Solvents, reagents and starting materials were purchased from commercial vendors and used as received unless otherwise described. All reactions were performed at room temperature unless otherwise stated. Starting materials were purchased from commercial sources or synthesized according to the methods described herein or using literature procedures.

### Chemical Synthesis Example 1

### (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoic acid (Travoprost acid)

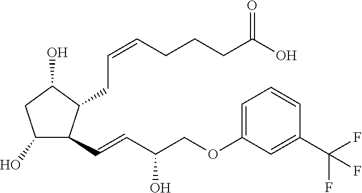 [SMILES: O=C(O)CCC/C=C\C[C@@H]1[C@@H](/C=C/[C@@H](O)COc2cccc(C(F)(F)F)c2)[C@H](O)C[C@@H]1O]

To a stirred solution of travoprost (1 g, 2.00 mmol) in MeOH (16 mL) was added 1M NaOH_{(aq)} (16 mL, 16 mmol) and the mixture stirred for 16 h. The mixture was quenched into 0.5M HCl_{(aq)} (32 mL, 16 mmol) and the aqueous extracted with DCM (2×100 mL). The DCM layers were combined dried (MgSO₄) and concentrated to give travoprost acid (916 mg, 100%) as a colorless oil. ¹H NMR (400 MHz, CDCl₃) δ 7.39 (t, J=8 Hz, 1H), 7.22 (d, J=8 Hz, 1H), 7.15 (s, 1H), 7.08 (d, J=8 Hz, 1H), 5.70 (m, 2H), 5.40 (m, 2H), 4.98 (heptet, J=6.5 Hz, 1H), 4.52 (m, 1H), 3.97 (m, 3H), 3.25 (br s, 2H), 2.60 (br s, 1H), 2.38 (m, 1H), 2.30-1.96 (m, 7H), 1.76 (dd, J=16, 4 Hz, 1H), 1.65 (quintet, J=7 Hz, 2H), 1.55 (m, 1H).

### Chemical Synthesis Example 2

### (8S,10S,13S,14S,17R)-17-hydroxy-17-(2-hydroxyacetyl)-10,13-dimethyl-1,2,6,7,8,10,12,13,14,15,16,17-dodecahydro-3H-cyclopenta[a]phenanthren-3-one (Anecortave)

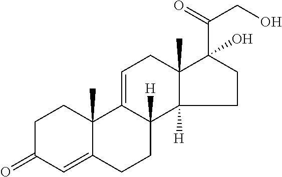

To a stirred suspension of anecortave acetate (3.0 g, 7.76 mmol) in methanol (80 mL) was added potassium hydroxide solution (30.3 mL of a 0.2M solution, 6.05 mmol) and the mixture stirred overnight. The reaction mixture was quenched into ice water (400 mL), stirred and filtered to give anecortave des acetate (2.0 g, 75%). 1H NMR (400 MHz, DMSO-d6) δ 5.66 (s, 1H); 5.52 (d, J=5 Hz, 1H), 5.28 (s, 1H, OH), 4.60 (t, J=5 Hz, 1H, OH), 4.31 (AB, J=19 Hz, Δv=82.5 Hz, further split (J=5 Hz) by OH, 2H), 2.60 (m, 3H), 2.50 (m, 1H), 2.25 (m, 3H), 2.05 (m, 3H), 1.80 (m, 2H), 1.55 (m, 2H), 1.22 (m, 1H), 1.20 (s, 3H), 1.02 (q, J=12 Hz, 1H), 0.49 (s, 3H).

### Chemical Synthesis Example 3

### 2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate (Compound 1)

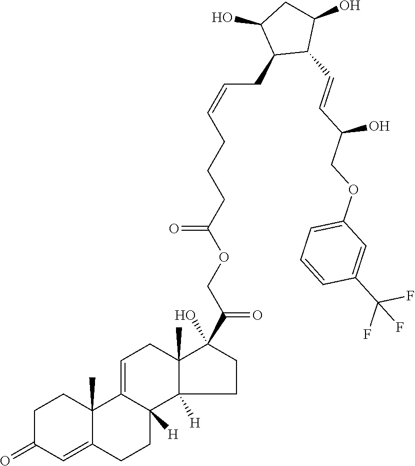 [SMILES: C[C@]12CCC(=O)C=C1CC[C@@H]1C2=CC[C@@]2(C)[C@H]1CC[C@]2(O)C(=O)COC(=O)CCC/C=C\C[C@@H]1[C@@H](/C=C/[C@@H](O)COc2cccc(C(F)(F)F)c2)[C@H](O)C[C@@H]1O]

To a stirred solution of travoprost acid (180 mg, 0.393 mmol) in dry pyridine (20 mL) under nitrogen was added anecortave (406 mg, 1.18 mmol), 4-(dimethylamino)pyridine (96 mg, 0.786 mmol) and N-(3-Dimethylaminopropyl)-N′-ethylcarbodiimide hydrochloride (150 mg, 0.786 mmol) and the mixture was stirred overnight. The mixture was concentrated and the residue dissolved in DCM (100 mL), the solution washed with 0.5 M hydrochloric acid (100 mL), water (100 mL), dried (MgSO₄) and concentrated onto 5 g reverse phase silica. Purification was performed by reverse phase Biotage automated chromatography (aqueous-MeCN gradient). Product containing fractions were combined, extracted with DCM, dried (MgSO₄) and concentrated to give the product as an off-white solid (102 mg, 33%). Melting point: 78-80° C. HPLC retention time: 31.7 min, ESI MS+ Calculated for C₄₄H₅₅F₃NaO₉⁺; 807.3696 Found: 807.3684. ¹H NMR (400 MHz, DMSO-d6) δ 7.45 (t, J=8 Hz, 1H), 7.20 (m, 3H), 5.62 (s, 1H), 5.55-5.40 (m, 6H), 5.20 (s, 1H, OH), 5.11 (br s, 1H), 4.95 (d, J=12 Hz, 1H), 4.80 (d, J=12 Hz, 1H), 4.48 (br s, 1H), 4.31 (m, 2H), 3.85 (m, 3H), 3.64 (m, 1H), 2.60 (m, 2H), 2.38 (m, 1H), 2.25 (m, 3H), 2.20 (m, 3H), 2.05 (m, 3H), 1.95 (m, 3H), 1.80 (m, 6H), 1.55 (m, 3H), 1.41 (m, 1H), 1.22 (m, 1H), 1.20 (s, 3H), 1.02 (q, J=12 Hz, 1H), 0.49 (s, 3H).

### Chemical Synthesis Example 4

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate (Compound 2)

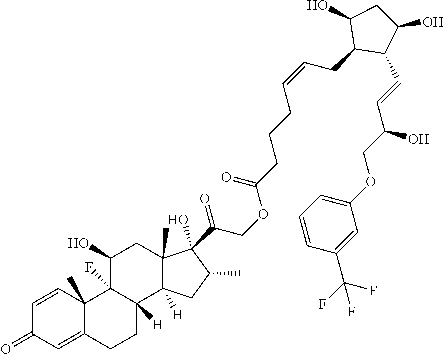 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)COC(=O)CCC/C=C\C[C@@H]1[C@@H](/C=C/[C@@H](O)COc2cccc(C(F)(F)F)c2)[C@H](O)C[C@@H]1O]

To a stirred solution of travoprost acid (180 mg, 0.393 mmol) and dexamethasone (1.54 g, 3.93 mmol) in dry THF (50 mL) under nitrogen was added 4-(dimethylamino)pyridine (96 mg, 0.786 mmol) and N-(3-Dimethylaminopropyl)-N′-ethylcarbodiimide hydrochloride (150 mg, 0.786 mmol) and the mixture was stirred overnight. The mixture was concentrated onto 10 g reverse phase silica. Purification was performed by reverse phase Biotage automated chromatography (aqueous-MeCN gradient), product containing fractions were combined, extracted with DCM, dried (MgSO₄) and concentrated to give a solid which was further purified by normal phase Biotage automated chromatography (hexane-ethyl acetate gradient). Product containing fractions were combined and concentrated to give the product as an off-white solid (120 mg, 37%). Melting point: 80° C. HPLC retention time: 30.5 min, ESI MS+ Calculated for C₄₅H₅₆F₄NaO₁₀⁺; 855.3707 Found: 855.3709. ¹H NMR (400 MHz, DMSO-d6) δ 7.45 (t, J=8 Hz, 1H), 7.20 (m, 4H), 6.20 (d, 1H), 5.99 (s, 1H), 5.60-5.35 (m, 4H), 5.20 (m, 1H), 5.11 (m, 1H), 4.95 (d, J=12 Hz, 1H), 4.76 (d, J=12 Hz, 1H), 4.50 (d, 1H), 4.31 (m, 2H), 4.05 (m, 1H), 3.89 (m, 3H), 3.64 (m, 1H), 3.59 (m, 1H), 2.83 (m, 1H), 2.60 (m, 1H), 2.38 (m, 1H), 2.25 (m, 3H), 2.20 (m, 3H), 2.05 (m, 3H), 1.95 (m, 3H), 1.78 (m, 2H), 1.60-1.40 (m, 4H), 1.30 (m, 1H), 1.21 (s, 3H), 1.02 (q, J=12 Hz, 1H), 0.82 (s, 3H), 0.78 (d, 3H).

### Chemical Synthesis Example 5

### (Z)-7-((1R,2R,3R,5S)-3,5-dihydroxy-2-((R)-3-hydroxy-5-phenylpentyl)cyclopentyl)hept-5-enoic acid (Latanoprost Acid)

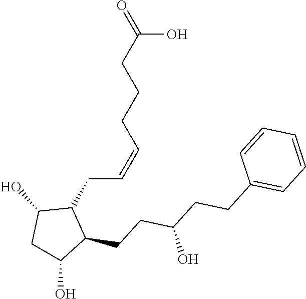

To a stirred solution of latanoprost (1 g, 2.31 mmol) in MeOH (16 mL) was added 1M NaOH_{(aq)} (18.5 mL, 18.5 mmol) and the mixture stirred for 16 h. The mixture was quenched into 0.5M HCl_{(aq)} (37 mL, 18.5 mmol) and the aqueous extracted with DCM (2×100 mL). The DCM layers were combined dried (MgSO₄) and concentrated to give latanoprost acid (902 mg, 100%) as a colorless oil. ¹H NMR (400 MHz, DMSO-d6) δ 11.98 (br s, 1H), 7.23 (m, 2H), 7.12 (m, 3H), 5.42 (m, 1H), 5.23 (m, 1H), 4.39 (m, 2H), 4.20 (m, 1H), 3.82 (m, 1H), 3.60 (m, 1H), 3.36 (m, 1H), 2.60 (m, 1H), 2.52 (m, 1H), 2.15 (m, 3H), 1.98 (m, 4H), 1.60-1.25 (m, 10H), 1.20 (m, 1H).

### Chemical Synthesis Example 6

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl (Z)-7-((1R,2R,3R,5S)-3,5-dihydroxy-2-((R)-3-hydroxy-5-phenylpentyl)cyclopentyl)hept-5-enoate (Compound 3)

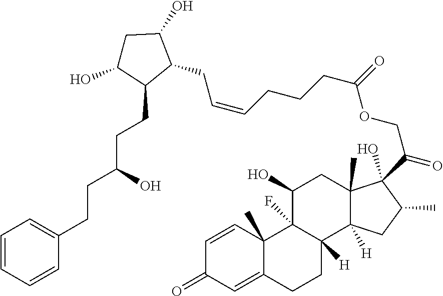 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)COC(=O)CCC/C=C\C[C@@H]1[C@@H](CC[C@@H](O)CCc2ccccc2)[C@H](O)C[C@@H]1O]

To a stirred solution of latanoprost acid (153 mg, 0.393 mmol) and dexamethasone (1.54 g, 3.93 mmol) in dry THF (50 mL) under nitrogen was added 4-(dimethylamino)pyridine (96 mg, 0.786 mmol) and N-(3-Dimethylaminopropyl)-N′-ethylcarbodiimide hydrochloride (150 mg, 0.786 mmol) and the mixture was stirred overnight. The mixture was concentrated onto 10 g reverse phase silica. Purification was performed by reverse phase Biotage automated chromatography (aqueous-MeCN gradient), product containing fractions were combined, extracted with DCM, dried (MgSO₄) and concentrated to give a solid which was further purified by normal phase Biotage automated chromatography (hexane-ethyl acetate gradient). Product containing fractions were combined and concentrated to give the product as an off-white solid (102 mg, 34%). Melting point: 78° C. HPLC retention time: 30.8 min, ESI MS+ Calculated for C₄₅H₆₂FO₉⁺; 765.4378 Found: 765.4380. ¹H NMR (400 MHz, DMSO-d6) δ 7.35-7.05 (m, 6H), 6.20 (d, 1H), 5.99 (s, 1H), 5.50-5.25 (m, 3H), 5.10 (s, 1H), 5.00 (m, 1H), 4.80 (m, 1H), 4.40 (m, 2H), 4.21 (m, 2H), 3.85 (m, 1H), 3.61 (m, 1H), 2.83 (m, 2H), 2.60 (m, 4H), 2.38 (m, 4H), 2.11 (m, 6H), 1.75 (m, 1H), 1.60-1.20 (m, 18H), 1.02 (q, J=12 Hz, 1H), 0.82 (s, 3H), 0.78 (m, 3H).

### Chemical Synthesis Example 7

### (8R,9S,13S,14S)-13-methyl-17-oxo-7,8,9,11,12,13,14,15,16,17-decahydro-6H-cyclopenta[a]phenanthren-3-yl (Z)-7-((1R,2R,3R,5S)-3,5-dihydroxy-2-((R)-3-hydroxy-5-phenylpentyl)cyclopentyl)hept-5-enoate (Compound 4)

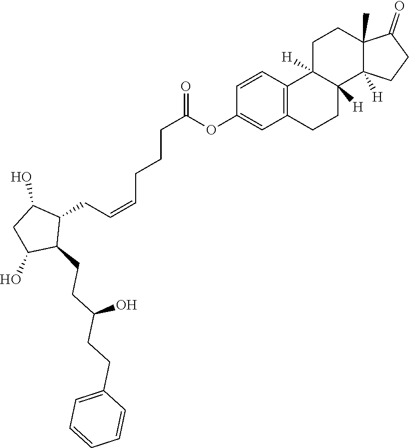 [SMILES: C[C@]12CC[C@@H]3c4ccc(OC(=O)CCC/C=C\C[C@@H]5[C@@H](CC[C@@H](O)CCc6ccccc6)[C@H](O)C[C@@H]5O)cc4CC[C@H]3[C@@H]1CCC2=O]

To a stirred solution of latanoprost acid (153 mg, 0.393 mmol) and estrone (318 mg, 1.179 mmol) in dry THF (50 mL) under nitrogen was added 4-(dimethylamino)pyridine (96 mg, 0.786 mmol) and N-(3-Dimethylaminopropyl)-N′-ethylcarbodiimide hydrochloride (150 mg, 0.786 mmol) and the mixture was stirred overnight. The mixture was concentrated onto 10 g reverse phase silica. Purification was performed by reverse phase Biotage automated chromatography (aqueous-MeCN gradient), product containing fractions were combined, extracted with DCM, dried (MgSO4) and concentrated to give a solid which was further purified by normal phase Biotage automated chromatography (hexane-ethyl acetate gradient). Product containing fractions were combined and concentrated to give the product as an off-white solid (28 mg, 11%). Melting point: 65° C. HPLC retention time: 35.9 min, ESI MS+ Calculated for C₄₁H₅₄NaO₆⁺; 665.3818 Found: 665.3820. ¹H NMR (400 MHz, DMSO-d6) δ 7.23 (m, 2H), 7.12 (m, 3H), 7.04 (m, 1H), 6.51 (m, 1H), 6.45 (m, 1H), 5.42 (m, 1H), 5.23 (m, 1H), 4.39 (m, 2H), 4.20 (m, 1H), 3.82 (m, 1H), 3.60 (m, 1H), 3.36 (m, 1H), 2.75 (m, 2H), 2.60 (m, 1H), 2.52 (m, 1H), 2.42 (m, 1H), 2.30 (m, 1H), 2.15 (m, 4H), 2.05 (m, 1H), 1.98 (m, 4H), 1.92 (m, 2H), 1.74 (m, 1H), 1.60-1.25 (m, 16H), 1.20 (m, 1H), 0.81 (s, 3H).

### Chemical Synthesis Example 8 (Compound 25)

### 1-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-1,4-dioxo-3,5,8,11-tetraoxatridecan-13-yl (Z)-7-((1R,2R,3R,5S)-3,5-dihydroxy-2-((R)-3-hydroxy-5-phenylpentyl)cyclopentyl)hept-5-enoate

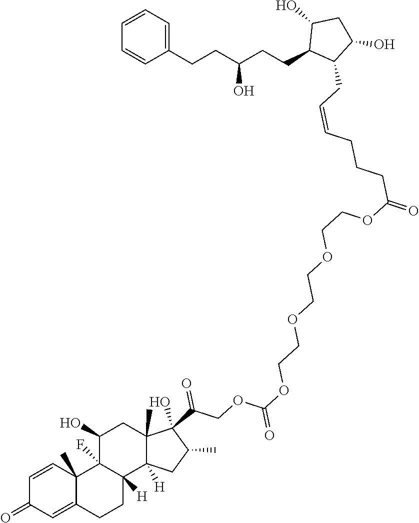 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)COC(=O)OCCOCCOCCOC(=O)CCC/C=C\C[C@@H]1[C@@H](CC[C@@H](O)CCc2ccccc2)[C@H](O)C[C@@H]1O]

Dexamethasone (314 mg, 0.80 mmol, 1.0 equiv) was dissolved in THF (20 mL) under nitrogen and phosgene solution (2.86 mL of a 1.4 M solution in toluene, 4.0 mmol, 5 equiv) was added dropwise with stirring. The mixture was stirred at room temperature overnight. Concentration of the mixture afforded the dexamethasone chloroformate as a thick oil which was dissolved in DCM (50 mL). Triethylene glycol (1.07 mL, 1.20 g, 8 mmol, 10 equiv) and pyridine (130 μL, 126 mg, 1.60 mmol, 2.0 equiv) were added and the mixture stirred for 2 h. The reaction solution was washed with water (2×50 mL) and the DCM layer concentrated onto normal phase silica (2 g) and purified by automated normal phase chromatography (ethyl acetate-hexane). The product containing fractions were combined and concentrated in vacuo to give the dexamethasone-triethyleneglycol carbonate intermediate as an off-white glassy solid (296 mg, 0.52 mmol, 65%). This was dissolved in DCM (50 mL) and latanoprost acid (202 mg, 0.52 mmol), 4-(dimethylamino)pyridine (127 mg, 1.04 mmol) and N-(3-Dimethylaminopropyl)-N′-ethylcarbodiimide hydrochloride (198 mg, 1.04 mmol) were added and the mixture was stirred overnight. The reaction solution was washed with water (2×50 mL) and the DCM layer concentrated onto reverse phase silica (2 g) and purified by automated reverse phase chromatography (acetonitrile-water). The product containing fractions were concentrated in vacuo to give the dexamethasone-triethyleneglycol-latanoprost as a colourless oil (49 mg, 0.052 mmol, 10%). HPLC retention time: 33.9 min, ESI MS+ Calculated for C₅₂H₇₄FO₁₄⁺; 941.5063 Found: 941.5065. ¹H NMR (400 MHz, DMSO-d6) δ 7.35-7.05 (m, 6H), 6.20 (m, 1H), 5.99 (s, 1H), 5.50-5.25 (m, 3H), 5.10 (s, 1H), 5.00 (m, 1H), 4.80 (m, 1H), 4.40 (m, 2H), 4.21 (m, 6H), 3.85 (m, 1H), 3.65 (m, 4H), 3.61 (m, 1H), 3.57 (s, 4H), 2.83 (m, 2H), 2.60 (m, 4H), 2.38 (m, 4H), 2.11 (m, 6H), 1.75 (m, 1H), 1.60-1.20 (m, 18H), 1.02 (q, J=12 Hz, 1H), 0.82 (s, 3H), 0.78 (m, 3H).

### Chemical Synthesis Example 9 (Compound 26)

### 4-acetamidophenyl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate

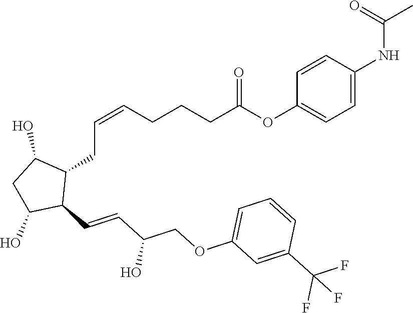 [SMILES: CC(=O)Nc1ccc(OC(=O)CCC/C=C\C[C@@H]2[C@@H](/C=C/[C@@H](O)COc3cccc(C(F)(F)F)c3)[C@H](O)C[C@@H]2O)cc1]

To a stirred solution of travoprost acid (180 mg, 0.393 mmol) and acetaminophen (297 mg, 1.97 mmol) in dry THF (50 mL) under nitrogen was added 4-(dimethylamino)pyridine (96 mg, 0.786 mmol) and N-(3-Dimethylaminopropyl)-N′-ethylcarbodiimide hydrochloride (150 mg, 0.786 mmol) and the mixture was stirred overnight. The mixture was concentrated onto 5g reverse phase silica. Purification was performed by reverse phase Biotage automated chromatography (aqueous-MeCN gradient), product containing fractions were combined, extracted with DCM, dried (MgSO₄) and concentrated to give travoprost-acetaminophen ester (139 mg, 60%) as a colourless oil. HPLC retention time: 22.6 min, ESI MS+ Calculated for C₃₁H₃₇F₃NO₇⁺; 592.2522 Found: 592.2524. ¹H NMR (400 MHz, DMSO-d6) δ 9.97 (s, 1H, NH), 7.57 (d, J=8 Hz, 2H), 7.45 (t, J=8 Hz, 1H), 7.22 (m, 2H), 7.15 (s, 1H), 6.98 (d, 2H), 5.70 (m, 2H), 5.40 (m, 2H), 4.98 (heptet, J=6.5 Hz, 1H), 4.52 (m, 1H), 3.97 (m, 3H), 3.25 (br s, 2H), 2.60 (br s, 1H), 2.38 (m, 1H), 2.30-1.96 (m, 10H), 1.76 (dd, J=16, 4 Hz, 1H), 1.65 (quintet, J=7 Hz, 2H), 1.55 (m, 1H).

### Chemical Synthesis Example 10 (Compound 27)

### ((3R,5R,8S)-tetracyclo[5.1.1.03,8.05,8]nonan-1-yl)methyl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate

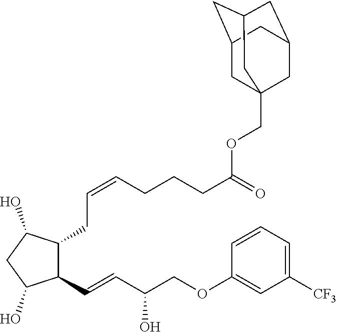 [SMILES: O=C(CCC/C=C\C[C@@H]1[C@@H](/C=C/[C@@H](O)COc2cccc(C(F)(F)F)c2)[C@H](O)C[C@@H]1O)OCC12CC3CC(CC(C3)C1)C2]

To a stirred solution of travoprost acid (180 mg, 0.393 mmol) and 1-Adamantanemethanol (327 mg, 1.97 mmol) in dry THF (50 mL) under nitrogen was added 4-(dimethylamino)pyridine (96 mg, 0.786 mmol) and N-(3-Dimethylaminopropyl)-N′-ethylcarbodiimide hydrochloride (150 mg, 0.786 mmol) and the mixture was stirred overnight. The mixture was concentrated onto 5 g reverse phase silica. Purification was performed by reverse phase Biotage automated chromatography (aqueous-MeCN gradient), product containing fractions were combined, extracted with DCM, dried (MgSO₄) and concentrated to give travoprost-acetaminophen ester (71 mg, 30%) as a colourless oil. HPLC retention time: 40.3 min, ESI MS+ Calculated for C₃₄H₄₆F₃O₆; 607.3241 Found: 607.3246. ¹H NMR (400 MHz, DMSO-d6) δ 7.60 (t, J=8 Hz, 1H), 7.22 (m, 2H), 7.15 (s, 1H), 5.60-5.40 (m, 3H), 5.20 (m, 1H), 5.10 (d, J=8.0 Hz, 1H), 4.52 (m, 1H), 4.30 (m, 2H), 3.88 (m, 3H), 3.63 (m, 1H), 3.56 (s, 2H), 2.93 (m, 1H), 2.20-1.80 (m, 10H), 1.70-1.40 (m, 15H), 1.25 (m, 1H).

### Chemical Synthesis Example 11 (Compound 28)

### (ethane-1,2-diylbis(oxy))bis(ethane-2,1-diyl) (5Z,5′Z)-bis(7-((1R,2R,3R,5S)-3,5-dihydroxy-2-((R)-3-hydroxy-5-phenylpentyl)cyclopentyl)hept-5-enoate)

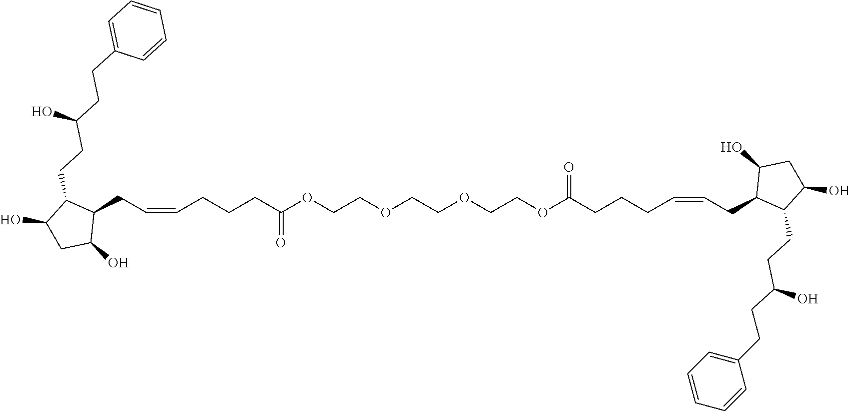 [SMILES: O=C(CCC/C=C\C[C@@H]1[C@@H](CC[C@@H](O)CCc2ccccc2)[C@H](O)C[C@@H]1O)OCCOCCOCCOC(=O)CCC/C=C\C[C@@H]1[C@@H](CC[C@@H](O)CCc2ccccc2)[C@H](O)C[C@@H]1O]

To a solution of latanoprost acid (202 mg, 0.52 mmol) and triethylene glycol (42 uL, 39 mg, 0.26 mmol) in DCM (50 mL) was added 4-(dimethylamino)pyridine (127 mg, 1.04 mmol) and N-(3-Dimethylaminopropyl)-N′-ethylcarbodiimide hydrochloride (198 mg, 1.04 mmol) were added and the mixture was stirred overnight. The reaction solution was washed with water (2×50 mL) and the DCM layer concentrated onto reverse phase silica (2 g) and purified by automated reverse phase chromatography (acetonitrile-water). The product containing fractions were concentrated in vacuo to give the latanoprost-triethyleneglycol-latanoprost as a colourless oil (37 mg, 0.042 mmol, 8%). HPLC retention time: 30.2 min, ESI MS+ Calculated for C₅₂H₇₉O₁₂; 895.5566, Found: 895.5572. ¹H NMR (400 MHz, DMSO-d6) δ 7.23 (m, 4H), 7.12 (m, 6H), 5.42 (m, 2H), 5.23 (m, 2H), 4.39 (m, 4H), 4.25-4.20 (m, 6H), 3.82 (m, 2H), 3.65 (m, 4H), 3.60-3.55 (m, 6H), 3.36 (m, 2H), 2.60 (m, 2H), 2.52 (m, 2H), 2.15 (m, 6H), 1.98 (m, 8H), 1.60-1.25 (m, 20H), 1.20 (m, 2H).

### Chemical Synthesis Example 12 (Compound 29)

### diisopropyl 7,7′-((1R,1′R,2R,2′R,3R,3′R,5S,5'S)-((3R,18R)-5,16-dioxo-3,18-diphenethyl-4,6,9,12,15,17-hexaoxaicosane-1,20-diyl)bis(3,5-dihydroxycyclopentane-2,1-diyl))(5Z,5′Z)-bis(hept-5-enoate)

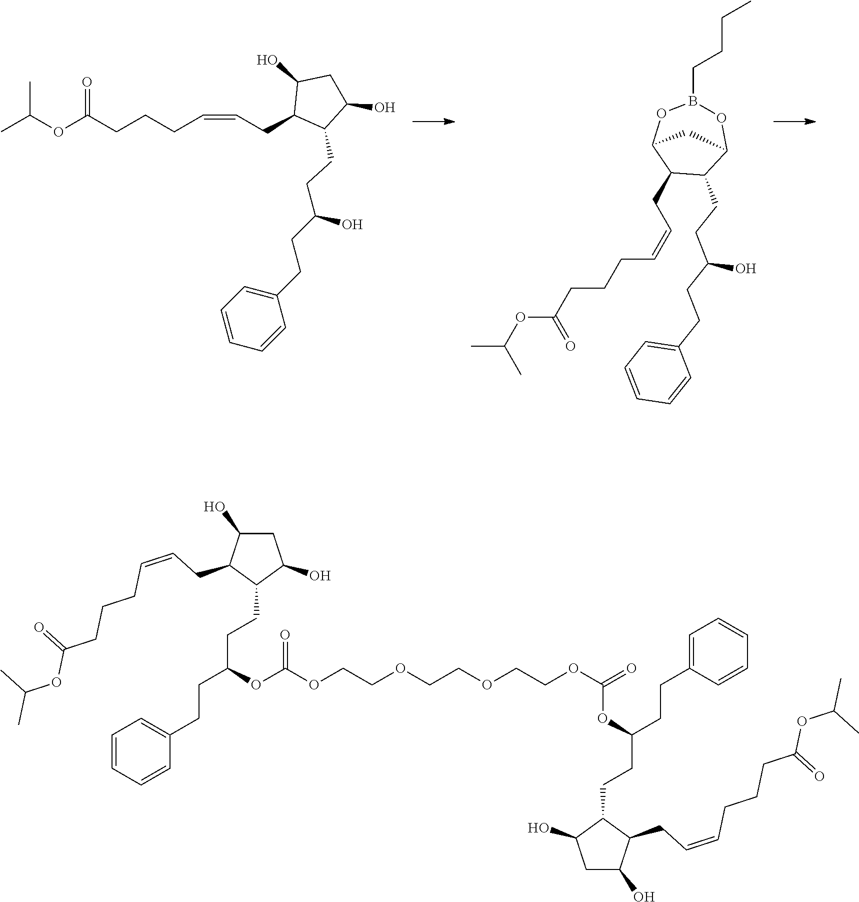 [SMILES: CC(C)OC(=O)CCC/C=C\C[C@@H]1[C@@H](CC[C@@H](O)CCc2ccccc2)[C@H](O)C[C@@H]1O ; CCCCB1O[C@H]2C[C@@H](O1)[C@H](CC[C@@H](O)CCc1ccccc1)[C@H]2C/C=C\CCCC(=O)OC(C)C ; CC(C)OC(=O)CCC/C=C\C[C@@H]1[C@@H](CC[C@H](CCc2ccccc2)OC(=O)OCCOCCOCCOC(=O)O[C@@H](CCc2ccccc2)CC[C@@H]2[C@@H](C/C=C\CCCC(=O)OC(C)C)[C@@H](O)C[C@H]2O)[C@H](O)C[C@@H]1O]

To a stirred solution of latanoprost (222.0 mg, 0.51 mmol) in dry DCM (20 mL) was added n-butylboronic acid (60.1 mg, 0.59 mmol) and the mixture stirred at reflux for 1 h under nitrogen atmosphere. The mixture was concentrated and the residue redissolved in dry DCM, the mixture heated to reflux for 3h, concentrated to give the 9, 11-boronate of latanoprost (254 mg, 100%) as a clear colourless oil which was and used directly without further purification. 1H NMR (400 MHz, CDCl3) δ (ppm): 7.28-7.17 (m, 2H), 7.17-7.03 (m, 3H), 5.49-5.27 (m, 2H), 4.93 (ddd, J=15.2, 7.6, 4.9 Hz, 1H), 4.28-4.13 (m, 1H), 4.07-3.90 (m, 1H), 3.65-3.46 (m, 1H), 2.78-2.67 (m, 1H), 2.67-2.41 (m, 1H), 2.28-2.11 (m, 4H), 2.09-1.98 (m, 2H), 1.91-1.79 (m, 1H), 1.79-1.53 (m, 7H), 1.53-1.38 (m, 3H), 1.38-1.07 (m, 12H), 0.89-0.75 (m, 3H), 0.64-0.52 (m, 2H). To a solution of 9,11-boronate of latanoprost (254 mg, 0.51 mmol) in dry DCM (25 mL) was added pyridine (164 uL, 162 mg, 2.04 mmol) and triethylene glycol bis(chloroformate) (52 uL, 70 mg, 0.255 mmol, 0.5 equiv) and the mixture stirred for 4h, methanol (5 mL) was added and the mixture stirred overnight. The mixture was concentrated onto 1g reverse phase silica. Purification was performed by reverse phase Biotage automated chromatography (aqueous-MeCN gradient), product containing fractions were combined, extracted with DCM, dried (MgSO₄) and concentrated to give latanoprost-TEG-latanoprost carbonate dimer (68 mg, 25%) as a colourless oil. HPLC retention time: 48.2 min, ESI MS+ Calculated for C₆₀H₉₁O₁₆⁺; 1067.6302, Found: 1067.6307. ¹H NMR (400 MHz, DMSO-d6) δ 7.23 (m, 4H), 7.12 (m, 6H), 5.42 (m, 2H), 5.23 (m, 2H), 4.39 (m, 4H), 4.25-4.20 (m, 6H), 3.82 (m, 2H), 3.65 (m, 4H), 3.60-3.55 (m, 6H), 3.36 (m, 2H), 2.60 (m, 2H), 2.52 (m, 2H), 2.15 (m, 6H), 1.98 (m, 8H), 1.60-1.25 (m, 20H), 1.20 (m, 2H), 1.00 (m, 12H).

### Chemical Synthesis Example 13 (Compound 30)

### isopropyl (Z)-7-((1R,2R,3R,5S)-2-((R)-3-((ethoxycarbonyl)oxy)-5-phenylpentyl)-3,5-dihydroxycyclopentyl)hept-5-enoate

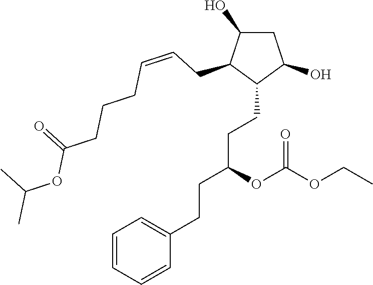 [SMILES: CCOC(=O)O[C@@H](CCc1ccccc1)CC[C@@H]1[C@@H](C/C=C\CCCC(=O)OC(C)C)[C@@H](O)C[C@H]1O]

To a solution of 9, 11-boronate of latanoprost (254 mg, 0.51 mmol) in dry DCM (25 mL) was added pyridine (82 uL, 81 mg, 1.02 mmol) and ethylchloroformate (48 uL, 55 mg, 0.51 mmol) and the mixture stirred for 4h, methanol (5 mL) was added and the mixture stirred overnight. The mixture was concentrated onto 1 g reverse phase silica. Purification was performed by reverse phase Biotage automated chromatography (aqueous-MeCN gradient), product containing fractions were combined, extracted with DCM, dried (MgSO₄) and concentrated to give latanoprost-15-ethyl carbonate (154 mg, 60%) as a colourless oil. HPLC retention time: 34.8 min. ESI MS+ Calculated for C₂₉H₄₅O₇⁺; 505.3160, Found: 505.3165. ¹H NMR (400 MHz, DMSO-d6) δ 7.23 (m, 2H), 7.12 (m, 3H), 5.42 (m, 1H), 5.23 (m, 1H), 4.80 (m, 1H), 4.60 (m, 1H), 4.39 (m, 2H), 4.20 (m, 1H), 4.03 (m, 2H), 3.82 (m, 1H), 3.60 (m, 1H), 2.60 (m, 1H), 2.52 (m, 1H), 2.15 (m, 2H), 1.98 (m, 4H), 1.80-1.25 (m, 10H), 1.20 (m, 4H), 1.16 (m, 6H).

### Chemical Synthesis Example 14: (Bimatoprost-Anecortave Ester; compound 5)

### 2-((8S,10S,13S,14S,17R)-17-hydroxy-10,13-dimethyl-3-oxo-2,3,6,7,8,10,12,13,14,15,16,17-dodecahydro-1H-cyclopenta[o]phenanthren-17-yl)-2-oxoethyl (Z)-7-((1R,2R,3R,5S)-3,5-dihydroxy-2-((S,E)-3-hydroxy-5-phenylpent-1-en-1-yl)cyclopentyl)hept-5-enoate

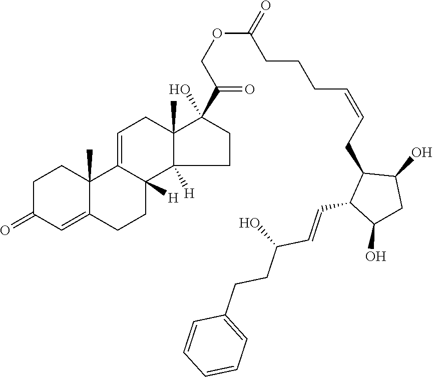 [SMILES: C[C@]12CCC(=O)C=C1CC[C@@H]1C2=CC[C@@]2(C)[C@H]1CC[C@]2(O)C(=O)COC(=O)CCC/C=C\C[C@@H]1[C@@H](/C=C/[C@@H](O)CCc2ccccc2)[C@H](O)C[C@@H]1O]

To a stirred solution of bimatoprost acid (1.0 g, 2.57 mmol) in dry pyridine (120 mL) under nitrogen was added anecortave (1.77 g, 5.14 mmol), 4-(dimethylamino)pyridine (0.62 g, 5.14 mmol) and N-(3-Dimethylaminopropyl)-N′-ethylcarbodiimide hydrochloride (0.99 g, 5.14 mmol) and the mixture was stirred for 4d at 37° C. The mixture was concentrated and the residue dissolved in DCM (150 mL), the solution washed with 0.5 M hydrochloric acid (150 mL), water (100 mL), dried (MgSO₄) and concentrated onto 5g reverse phase silica. Purification was performed by reverse phase Biotage automated chromatography (aqueous-MeCN gradient) followed by normal phase Biotage automated chromatography (hexanes-ethyl acetate). Product containing fractions were combined, concentrated, redissolved in MeCN (50 mL) and concentrated to give the product as an off-white solid (445 mg, 24%). Melting point: 110-115° C. HPLC retention time: 29.7 min, ESI MS+ Calculated for C₄₄H₅₈NaO₈⁺; 737.4024, Found: 737.4020. ¹H NMR (400 MHz, DMSO-d6) δ 7.25 (t, J=7.5 Hz, 2H), 7.20-7.10 (m, 3H), 5.66 (d, J=1.5 Hz, 1H), 5.58-5.17 (m, 6H), 4.97 (d, J=17.6 Hz, 1H), 4.84 (d, J=17.6 Hz, 1H), 4.65 (d, J=4.6 Hz, 1H), 4.49 (d, J=5.8 Hz, 1H), 4.34 (d, J=5.0 Hz, 1H), 3.92 (td, J=6.4, 3.5 Hz, 2H), 3.67 (ddd, J=13.6, 6.9, 4.0 Hz, 1H), 2.72-2.42 (m, 4H), 2.37-1.92 (m, 16H), 1.88-1.75 (m, 3H), 1.75-1.62 (m, 2H), 1.62-1.49 (m, 3H), 1.44 (ddd, J=14.1, 5.7, 2.4 Hz, 1H), 1.40-1.21 (m, 5H), 1.07-0.93 (m, 1H), 0.48 (s, 3H).

### Chemical Synthesis Example 15: (Latanoprost-Anecortave Ester; Compound 6)

### 2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl (Z)-7-((1R,2R,3R,5S)-3,5-dihydroxy-2-((R)-3-hydroxy-5-phenylpentyl)cyclopentyl)hept-5-enoate

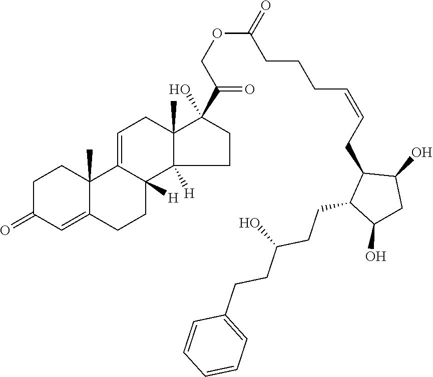 [SMILES: C[C@]12CCC(=O)C=C1CC[C@@H]1C2=CC[C@@]2(C)[C@H]1CC[C@]2(O)C(=O)COC(=O)CCC/C=C\C[C@@H]1[C@@H](CC[C@@H](O)CCc2ccccc2)[C@H](O)C[C@@H]1O]

To a stirred solution of latanoprost acid (1.0 g, 2.56 mmol) in dry pyridine (60 mL) under nitrogen was added anecortave (1.76 g, 5.12 mmol), 4-(dimethylamino)pyridine (0.62 g, 5.12 mmol) and N-(3-Dimethylaminopropyl)-N′-ethylcarbodiimide hydrochloride (0.99 g, 5.14 mmol) and the mixture was stirred for 4d at 37° C. The mixture was concentrated and the residue dissolved in DCM (150 mL), the solution washed with 0.5 M hydrochloric acid (150 mL), water (100 mL), dried (MgSO₄) and concentrated onto 4g reverse phase silica. Purification was performed by reverse phase Biotage automated chromatography (aqueous-MeCN gradient) followed by normal phase Biotage automated chromatography (hexanes-ethyl acetate). Product containing fractions were combined, concentrated, redissolved in MeCN (50 mL) and concentrated to give the product as an off-white solid (415 mg, 23%). Melting point: 122-124° C. HPLC retention time: 31.4 min, ESI MS+ Calculated for C₄₄H₆₀NaO₈⁺; 739.4186, Found: 739.4183. ¹H NMR (400 MHz, DMSO-d6) δ 7.25 (t, J=7.5 Hz, 2H), 7.21-7.10 (m, 3H), 5.65 (d, J=1.6 Hz, 1H), 5.55-5.42 (m, 3H), 5.36-5.25 (m, 1H), 4.99 (d, J=17.6 Hz, 1H), 4.85 (d, J=17.6 Hz, 1H), 4.45-4.34 (m, 2H), 4.20 (d, J=5.4 Hz, 1H), 3.94-3.84 (m, 1H), 3.68-3.57 (m, 1H), 3.38 (d, J=9.8 Hz, 1H), 2.75-2.51 (m, 5H), 2.50-2.42 (m, 4H), 2.41-1.91 (m, 10H), 1.83 (dt, J=11.7, 7.5 Hz, 3H), 1.69-1.31 (m, 12H), 1.30 (s, 3H), 1.23 (tt, J=9.3, 5.5 Hz, 1H), 1.07-0.93 (m, 1H), 0.48 (s, 3H).

### Chemical Synthesis Example 16: (Tafluprost-Anecortave Ester; Compound 7)

### 2-((85,105,135,145,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl (Z)-7-((1R,2R,3R,5S)-2-((E)-3,3-difluoro-4-phenoxybut-1-en-1-yl)-3,5-dihydroxycyclopentyl)hept-5-enoate

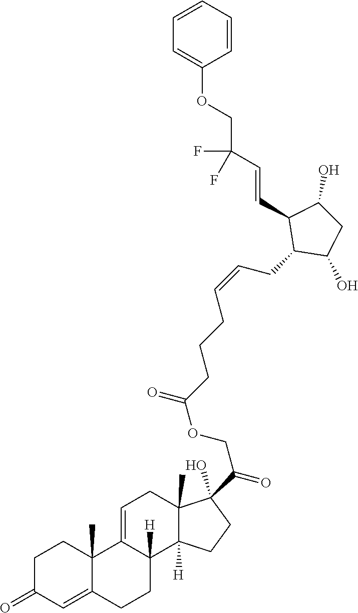

To a solution of tafluprost acid (800 mg, 1.95 mmol) and anecortave (1.34 g, 3.90 mmol) in dry pyridine (50 mL) was added DMAP (476 mg, 3.90 mmol) and EDCI (747 mg, 3.90 mmol) at 0° C., then stirred at 20° C. for 10 h. The reaction mixture was poured into DCM (50 mL), washed with 1M aqueous HCl (3×100 mL), the organic layer was dried over Na₂SO₄, filtered and concentrated to give a residue. The residue was purified by prep-HPLC (column: Phenomenex luna C18 (250*70 mm, 15 um); mobile phase: [water (0.225% FA)-ACN]; B %: 35%-80%, 23 min) to give the product (600 mg, 42%) as an off-white solid. Melting point: 107-110° C. HPLC retention time: 33.8 min, ESI MS+ Calculated for C₄₃H₅₄F₂NaO₈⁺; 759.3684, Found: 759.3690. ¹H NMR (400 MHz, DMSO-d6) δ 7.30 (t, J=7.8 Hz, 2H) 6.92-7.05 (m, 3H) 6.09 (br dd, J=15.7, 8.9 Hz, 1H) 5.77 (dt, J=15.7, 11.2 Hz, 1H) 5.65 (s, 1H) 5.49-5.55 (m, 2H) 5.39-5.48 (m, 1H) 5.22-5.31 (m, 1H) 4.93-5.02 (m, 1H) 4.79-4.89 (m, 1H) 4.73 (d, J=5.9 Hz, 1H) 4.47 (d, J=4.8 Hz, 1H) 4.33 (br t, J=12.9 Hz, 2H) 3.92 (br d, J=3.8 Hz, 1H) 3.75 (quin, J=6.9 Hz, 1H) 2.53-2.69 (m, 3H) 1.90-2.37 (m, 15H) 1.74-1.87 (m, 3H) 1.55 (quin, J=7.2 Hz, 3H) 1.39-1.49 (m, 2H) 1.25-1.37 (m, 4H) 0.92-1.07 (m, 1H) 0.48 (s, 3H)

### Chemical Synthesis Example 18: (Travoprost-Cyclohexanedimethanol-Anecortave; Compound 8)

### (4-((((2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)carbonyl)oxy)methyl)cyclohexyl)methyl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate

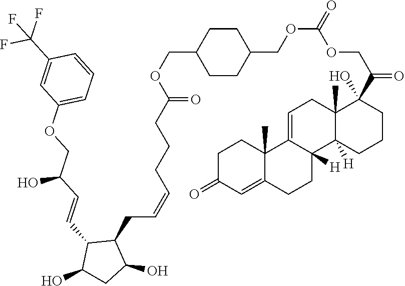 [SMILES: C[C@]12CCC(=O)C=C1CC[C@@H]1C2=CC[C@@]2(C)[C@H]1CCC[C@]2(O)C(=O)COC(=O)OCC1CCC(COC(=O)CCC/C=C\C[C@@H]2[C@@H](/C=C/[C@@H](O)COc3cccc(C(F)(F)F)c3)[C@H](O)C[C@@H]2O)CC1]

Anecortave (640 mg, 1.86 mmol) was dissolved in dry THFF (40 mL) under nitrogen and phosgene solution (6.64 mL of a 1.4 M solution in toluene, 9.29 mmol) was added dropwise with stirring. The mixture was stirred at room temperature overnight. Concentration of the mixture afforded anecortave chloroformate as a pale yellow solid (740 mg, 98%) that was used without further purification or analysis.

To a stirred suspension of 1,4-cyclohexanedimethanol (2.48 g, 1.72 mmol) in dry CH₂Cl₂ (30 mL) was added anecortave chloroformate (700 mg, 1.72 mmol) and dry pyridine (1.08 mL, 13.4 mmol). The solution was stirred at room temperature for 16 h, concentrated and the yellow residue re-dissolved in CH₂Cl₂ (50 mL). The solution was washed with 0.5M hydrochloric acid (2×50 mL), water (50 mL), dried (MgSO₄) and evaporated to give anecortave-CDM as a pale yellow solid (701 mg, 79%). HPLC retention time: 27.4 min. ESI MS+ calculated for C₃₀H₄₂NaO₇⁺; 537.2823, Found: 537.2830. ¹H NMR (400 MHz, DMSO-d6) δ 5.66 (s, 1H), 5.54-5.51 (m, 2H), 5.04 (d, J=17.8 Hz, 1H), 4.83 (d, J=17.8 Hz, 1H), 4.37-4.29 (m, 1H), 4.02 (d, J=7.2 Hz, 1H), 3.92 (d, J=6.4 Hz, 1H), 3.30-3.25 (m, 1H), 3.22-3.17 (m, 1H), 2.68-2.43 (m, 3H), 2.36-2.18 (m, 3H), 2.12-1.94 (m, 4H), 1.87-1.69 (m, 6H), 1.61-1.48 (m, 2H), 1.45-1.28 (m, 5H), 1.30 (s, 3H), 1.06-0.79 (m, 3H), 0.52-0.46 (m, 3H).

A stirred solution of travoprost acid (200 mg, 0.436 mmol) in dry MeCN (10 mL) under nitrogen was stirred at −15° C. (ice/salt bath). N-Methylmorpholine (96.8 μL, 0.872 mmol) and isobutyl chloroformate (57.8 μL, 0.436 mmol) were added and the solution stirred for 10 mins. The solution was then added dropwise to a suspension of anecortave-CDM (453 mg, 0.872 mmol) in dry MeCN (20 mL) under nitrogen at −15° C. (ice/salt bath). After 10 mins stirring, the mixture was warmed to room temperature and stirred for 3 hours. The mixture was concentrated onto reverse phase silica (3 g) and purified by automated reverse phase chromatography (aqueous-MeCN). The product containing fractions were concentrated in vacuo to give the product as a colorless solid (53 mg, 13%). For thermal property measurements, the solid was dissolved in DCM and concentrated in vacuo to give a colorless solid. Melting point: not observed (T_{g}=˜43° C.). HPLC retention time: 37.8 min. ESI MS+ calculated for Cs₃He₉F3NaO₂+; 977.4633, Found: 977.4640. ¹H NMR (400 MHz, DMSO-d6) δ 7.49 (t, J=8.0 Hz, 1H), 7.26-7.19 (m, 2H), 7.18 (s, 1H), 5.65 (s, 1H), 5.58-5.38 (m, 5H), 5.26-5.18 (m, 1H), 5.10 (d, J=4.8 Hz, 1H), 5.02 (dd, J=17.8, 1.8 Hz, 1H), 4.82 (d, J=17.8 Hz, 1H), 4.52 (d, J=5.8 Hz, 1H), 4.35-4.26 (m, 2H), 4.01 (d, J=7.2 Hz, 1H), 3.96-3.86 (m, 5H), 3.78 (d, J=6.5 Hz, 1H), 3.71-3.63 (m, 1H), 2.66-2.42 (m, 3H), 2.35-2.25 (m, 2H), 2.25-2.10 (m, 6H), 2.10-2.03 (m, 2H), 2.00-1.92 (m, 5H), 1.83-1.65 (m, 5H), 1.60-1.26 (m, 12H), 1.29 (s, 3H), 1.05-0.81 (m, 3H), 0.47 (s, 31H).

### Chemical Synthesis Example 19: (Travoprost-Naltrexone; Compound 9)

### (4R,4aS,7aR,12bS)-3-(cyclopropylmethyl)-4α-hydroxy-7-oxo-2,3,4,4a,5,6,7,7a-octahydro-1H-4,12-methanobenzofuro[3,2-e]isoquinolin-9-yl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate

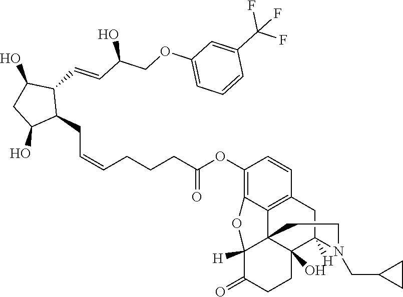 [SMILES: O=C(CCC/C=C\C[C@@H]1[C@@H](/C=C/[C@@H](O)COc2cccc(C(F)(F)F)c2)[C@H](O)C[C@@H]1O)Oc1ccc2c3c1O[C@H]1C(=O)CC[C@@]4(O)[C@@H](C2)N(CC2CC2)CC[C@]314]

To a stirred solution of travoprost acid (122 mg, 0.27 mmol) and naltrexone.HCl (100 mg, 0.27 mmol) in dry DCM (20 mL) under nitrogen was added 4-(dimethylamino)pyridine (132 mg, 1.08 mmol) and N-(3-Dimethylaminopropyl)-N′-ethylcarbodiimide hydrochloride (104 mg, 0.54 mmol) and the mixture was stirred overnight. The mixture was concentrated onto 2g reverse phase silica. Purification was performed by reverse phase Biotage automated chromatography (aqueous-MeCN gradient), product containing fractions were combined and concentrated, the residue dissolved in MTBE (20 mL) and concentrated to give travoprost-naltrexone ester (84 mg, 40%) as a glassy white solid. Melting point: 55-57° C. HPLC retention time: 17.6 min, ESI MS+ Calculated for C₄₃H₅₁F₃NO₉⁺; 782.3516, Found: 782.3512. ¹H NMR (400 MHz, DMSO-d6) δ 7.50 (t, J=7.9 Hz, 1H), 7.32-7.15 (m, 3H), 6.80 (d, J=8.2 Hz, 1H), 6.70 (d, J=8.3 Hz, 1H), 5.64-5.39 (m, 3H), 5.34-5.23 (m, 1H), 5.11 (d, J=4.8 Hz, 2H), 4.90 (s, 1H), 4.53 (d, J=5.8 Hz, 1H), 4.36 (d, J=4.9 Hz, 1H), 4.31 (p, J=5.3 Hz, 1H), 3.93 (dq, J=8.9, 4.8 Hz, 2H), 3.69 (p, J=7.4 Hz, 1H), 3.16 (d, J=5.6 Hz, 1H), 3.09-3.06 (m, 1H), 2.97-2.84 (m, 2H), 2.70-2.53 (m, 2H), 2.43-2.30 (m, 4H), 2.26-1.89 (m, 8H), 1.77 (td, J=9.7, 5.8 Hz, 1H), 1.62 (p, J=7.3 Hz, 2H), 1.49-1.20 (m, 4H), 1.10 (s, 1H), 0.87 (dtd, J=14.1, 6.9, 4.0 Hz, 1H), 0.58-0.42 (m, 2H), 0.19-0.09 (m, 2H).

### Chemical Synthesis Example 20: (Timolol-Deoxycholic Acid; Compound 10)

### (S)-1-(tert-butylamino)-3-((4-morpholino-1,2,5-thiadiazol-3-yl)oxy)propan-2-yl (R)-4-((3R,5R,8R,95,10S,12S,13R,14S,17R)-3,12-dihydroxy-10,13-dimethylhexadecahydro-1H-cyclopenta[a]phenanthren-17-yl)pentanoate

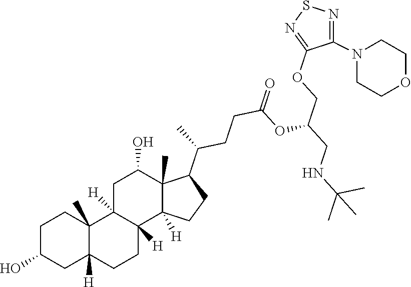 [SMILES: C[C@H](CCC(=O)O[C@@H](CNC(C)(C)C)COc1nsnc1N1CCOCC1)[C@H]1CC[C@H]2[C@@H]3CC[C@@H]4C[C@H](O)CC[C@]4(C)[C@H]3C[C@H](O)[C@]12C]

To a stirred solution of timolol free base (316 mg, 1.0 mmol) and deoxycholic acid (393 mg, 1.0 mmol) in dry DCM (20 mL) under nitrogen was added 4-(dimethylamino)pyridine (244 mg, 2.0 mmol) and N-(3-Dimethylaminopropyl)-N′-ethylcarbodiimide hydrochloride (384 mg, 2.0 mmol) and the mixture was stirred for 2d. The mixture was concentrated onto 1 g reverse phase silica. Purification was performed by reverse phase Biotage automated chromatography (aqueous-MeCN gradient), product containing fractions were combined and concentrated, the residue dissolved in MTBE (20 mL) and concentrated to give timolol-deoxycholic ester (242 mg, 35%) as a glassy white solid. Melting point: 73° C. HPLC retention time: 36.2 min, ESI MS+ Calculated for C₃₇H₆₃N₄O₆⁺; 691.4463, Found: 691.4464. ¹H NMR (400 MHz, DMSO-d6) δ 5.11 (qd, J=6.3, 2.8 Hz, 1H), 4.61 (dd, J=11.4, 2.8 Hz, 1H), 4.51-4.41 (m, 2H), 4.19 (d, J=4.1 Hz, 1H), 3.77 (d, J=3.8 Hz, 1H), 3.68 (t, J=4.8 Hz, 4H), 3.48-3.34 (m, 5H), 2.70 (d, J=6.3 Hz, 2H), 2.33 (ddd, J=14.4, 9.0, 4.9 Hz, 1H), 2.16 (dq, J=15.5, 8.1 Hz, 1H), 1.86-1.41 (m, 11H), 1.38-1.11 (m, 11H), 1.11 (s, 1H), 1.08-0.93 (m, 11H), 0.90 (d, J=6.2 Hz, 3H), 0.84 (s, 3H), 0.55 (s, 3H).

### Chemical Synthesis Example 21: (Bimatoprost(C₁₅)-Anecortave Carbonate; Compound 11)

### (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl (2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl) carbonate

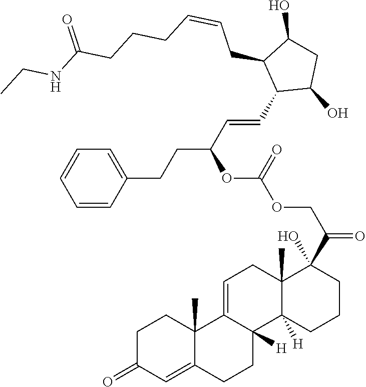 [SMILES: CCNC(=O)CCC/C=C\C[C@@H]1[C@@H](/C=C/[C@H](CCc2ccccc2)OC(=O)OCC(=O)[C@@]2(O)CCC[C@H]3[C@@H]4CCC5=CC(=O)CC[C@]5(C)C4=CC[C@@]32C)[C@H](O)C[C@@H]1O]

To a stirred solution of bimatoprost amide (100 mg, 0.241 mmol) in dry pyridine (3 mL) under nitrogen was added anecortave chloroformate (196 mg, 0.482 mmol). The yellow solution was stirred for 4 days, concentrated and re-dissolved in CH₂Cl₂ (5 mL). The mixture was concentrated onto reverse phase silica (2 g) and purified by automated reverse phase chromatography (aqueous-MeCN). The product containing fractions were concentrated to give a solid that was further purified by normal phase Biotage automated chromatography (hexane-ethyl acetate gradient). The product containing fractions were concentrated in vacuo to give the product as a colorless solid (23 mg, 12%). Melting point: 110° C. HPLC retention time: 30.5 min. ESI MS+ calculated for C₄₇H₆₃NNaO₉⁺; 808.4395, Found: 808.4397. ¹H NMR (400 MHz, DMSO-d6) 7.68 (t, J=5.7 Hz, 1H, NH), 7.30-7.22 (m, 2H), 7.19-7.12 (m, 3H), 5.65 (s, 1H), 5.55-5.38 (m, 5H), 5.33-5.26 (m, 1H), 5.03 (d, J=17.8 Hz, 1H), 4.82-4.76 (m, 2H), 4.70-4.64 (m, 2H), 3.98-3.89 (m, 2H), 3.07-2.99 (qd, J=7.2, 5.7 Hz, 2H), 2.67-2.43 (m, 6H), 2.38-2.15 (m, 5H), 2.15-1.89 (m, 10H), 1.86-1.63 (m, 3H), 1.59-1.21 (m, 8H), 1.29 (s, 3H), 0.98 (t, J=7.2 Hz, 3H), 0.47 (s, 3H).

### Chemical Synthesis Example 22: (Bimatoprost(C₁₅)-Succ-Anecortave ester; Compound 31)

### 4-(2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)-4-oxobutanoic acid

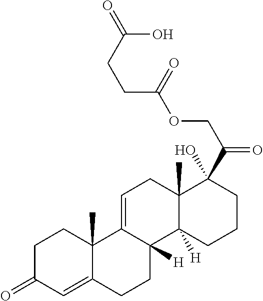 [SMILES: C[C@]12CCC(=O)C=C1CC[C@@H]1C2=CC[C@@]2(C)[C@H]1CCC[C@]2(O)C(=O)COC(=O)CCC(=O)O]

To a stirred soln of anecortave desacetate (1.0 g, 2.90 mmol) and DMAP (354 mg, 2.90 mmol) in dry THF (60 mL) was added succinic anhydride (2.9 g, 29.0 mmol) and the mixture stirred for 2 days at 50° C. The mixture was quenched into 0.5M HCl_{(aq)} soln and the aqueous extracted with DCM (2×60 mL). The combined DCM extracts were concentrated onto reverse phase silica (6 g) and purified by reverse phase automated chromatography to give anecortave-succinate (0.73 g, 57%) as an off-white solid. HPLC retention time: 20.5 min.

### (Z)-7-((1R,5S,6R,7R)-3-butyl-7-((S,E)-3-hydroxy-5-phenylpent-1-en-1-yl)-2,4-dioxa-3-borabicyclo[3.2.1]octan-6-yl)-N-ethylhept-5-enamide

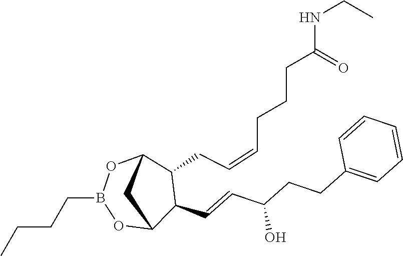 [SMILES: CCCCB1O[C@H]2C[C@@H](O1)[C@H](/C=C/[C@@H](O)CCc1ccccc1)[C@H]2C/C=C\CCCC(=O)NCC]

To a stirred solution of bimatoprost (830 mg, 2.0 mmol) in dry DCM (100 mL) was added n-butylboronic acid (306 mg, 3.0 mmol) and the mixture stirred at reflux for 1 h under nitrogen atmosphere. The mixture was concentrated and the residue redissolved in dry DCM, the mixture heated to reflux for 3h, concentrated to give the 9,11-boronate of bimatoprost (254 mg, 100%) as a clear colourless oil which was and used directly without further purification. ¹H NMR (400 MHz, DMSO-d6) δ 7.70 (s, 1H), 7.35-7.23 (m, 2H), 7.23-7.11 (m, 3H), 5.53-5.29 (m, 3H), 4.73 (d, J=4.7 Hz, 1H), 4.26 (s, 1H), 4.02 (dd, J=2.7, 1.4 Hz, 1H), 3.87 (p, J=6.0 Hz, 1H), 3.03 (qd, J=7.2, 5.5 Hz, 2H), 2.65-2.50 (m, 2H), 2.27 (dd, J=7.7, 5.7 Hz, 1H), 2.17 (h, J=6.4 Hz, 2H), 2.06-1.96 (m, 4H), 1.92 (dt, J=12.9, 2.6 Hz, 1H), 1.86-1.71 (m, 2H), 1.71-1.57 (m, 2H), 1.57-1.46 (m, 2H), 1.36-1.18 (m, 4H), 0.98 (t, J=7.2 Hz, 3H), 0.91-0.80 (m, 4H), 0.57 (dq, J=7.9, 3.6 Hz, 2H)

### (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl (2-((8S,10S,13S,14S,17R)-17-hydroxy-10,13-dimethyl-3-oxo-2,3,6,7,8,10,12,13,14,15,16,17-dodecahydro-1H-cyclopento[a]phenanthren-17-yl)-2-oxoethyl) succinate

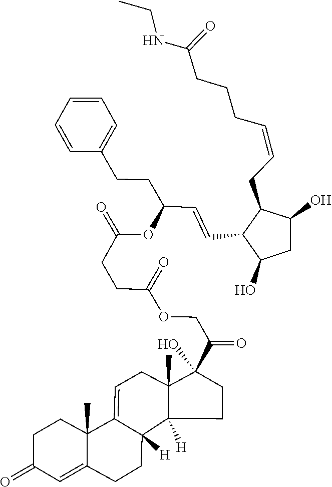 [SMILES: CCNC(=O)CCC/C=C\C[C@@H]1[C@@H](/C=C/[C@H](CCc2ccccc2)OC(=O)CCC(=O)OCC(=O)[C@@]2(O)CC[C@H]3[C@@H]4CCC5=CC(=O)CC[C@]5(C)C4=CC[C@@]32C)[C@H](O)C[C@@H]1O]

To a stirred solution of 9,11-boronate of bimatoprost (963 mg, 2.0 mmol), anecortave-succinate (1.29 g, 2.9 mmol) and DMAP (488 mg, 4.0 mmol) in DCM (100 mL) was added EDC. HCl (767 mg, 4.0 mmol) and the mixture stirred for 11 days. The mixture was concentrated onto reverse phase silica (10 g) and purified by reverse phase automated chromatography and recrystallisation from MeCN to give bimatoprost(C₁₅)-succ-anecortave ester (210 mg, 15%) as an off white solid. Melting point: 105-107° C. HPLC retention time: 30.0 min. ESI MS+ calculated for C₅₀H₆₇NNaO₁₀⁺; 864.4657, Found: 864.4666. ¹H NMR (400 MHz, DMSO-d6) δ 7.67 (s, 1H), 7.30-7.21 (m, 2H), 7.17 (ddd, J=9.6, 3.8, 2.6 Hz, 3H), 5.65 (s, 1H), 5.56-5.35 (m, 5H), 5.31-5.21 (m, 1H), 5.16 (q, J=6.2 Hz, 1H), 5.01 (d, J=17.6 Hz, 1H), 4.86 (d, J=17.6 Hz, 1H), 4.56 (d, J=5.9 Hz, 1H), 4.38 (d, J=4.8 Hz, 1H), 3.90 (s, 1H), 3.67 (p, J=6.5 Hz, 1H), 3.03 (qd, J=7.2, 5.5 Hz, 2H), 2.77-2.52 (m, 8H), 2.48-2.38 (m, 1H), 2.34 (s, 1H), 2.31-2.26 (m, 1H), 2.26-2.01 (m, 6H), 2.01-1.90 (m, 7H), 1.82 (qd, J=16.8, 6.7 Hz, 5H), 1.60-1.37 (m, 4H), 1.28 (s, 5H), 0.98 (t, J=7.2 Hz, 4H), 0.45 (s, 3H).

### Chemical Synthesis Example 23: (Bimatoprost(C₁₅)-Terephthalate-Anecortave; Compound 32)

### 2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl 4-formylbenzoate

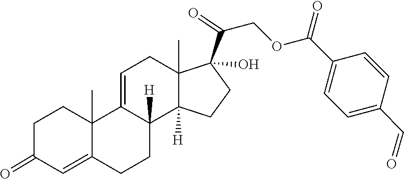

Anecortave (1.17 g, 3.40 mmol), 4-formylbenzoic acid (561 mg, 3.74 mmol) and DMAP (831 mg, 6.80 mmol) were added to a flask and the flask purged with Ar. Dry THF (60 mL) was added, followed by EDC.HCl (1.30 g, 6.80 mmol) in one portion. The suspension was stirred at 37° C. for 20 h. The mixture was concentrated in vacuo and the residue dissolved in CH₂Cl₂ (200 mL). The solution was washed with 0.5M HCl_{(aq.)} (200 mL), sat. NaHCO_{3(aq.)} (100 mL), dried over MgSO₄, filtered and evaporated to give the product as an off-white solid (1.52 g, 94%). HPLC retention time: 30.4 min. ESI MS+ calculated for C₂₉H₃₃O₆⁺; 477.2272, Found: 477.2274. ¹H NMR (400 MHz, DMSO-d6) δ 10.12 (s, 1H), 8.19-8.16 (m, 2H), 8.08-8.04 (m, 2H), 5.66-5.63 (m, 1H), 5.62 (s, 1H), 5.55 (d, J=5.7 Hz, 1H), 5.31 (d, J=17.6 Hz, 1H), 5.15 (d, J=17.6 Hz, 1H), 2.71 (d, J=17.0 Hz, 1H), 2.63-2.42 (m, 2H), 2.36-2.21 (m, 4H), 2.13-1.79 (m, 6H), 1.64-1.57 (m, 1H), 1.37-1.28 (m, 1H), 1.30 (s, 3H), 1.08-0.94 (m, 1H), 0.53 (s, 3H).

### 4-((2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)carbonyl)benzoic acid

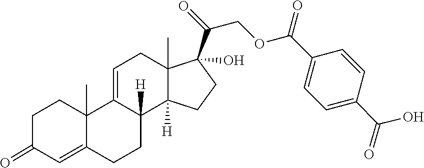

To a stirred solution of anecortave-4-formylbenzoate (340 mg, 0.71 mmol) in t-BuOH (10 mL) was added 2-Me-2-butene (2 mL), a solution of NaH₂PO₄ (857 mg, 7.14 mmol) in H₂O (5 mL) and NaOCl₂ (452 mg). The yellow solution was stirred at room temperature for 20 h. The mixture was partitioned between 0.5M HCl_{(aq.)} (50 mL) and CH₂Cl₂ (50 mL) and the organics separated. The aqueous layer was extracted with CH₂Cl₂ (50 mL) and the combined organics dried over MgSO₄, filtered and evaporated to give the product as an off-white solid (330 mg, 94%). HPLC retention time: 26.6 min. ESI MS+ calculated for C₂₉H₃₂NaO₇⁺; 515.2040, Found: 515.2043. ¹H NMR (400 MHz, DMSO-d6) δ 8.10-8.07 (m, 4H), 5.67-5.63 (m, 1H), 5.60 (s, 1H), 5.55 (d, J=5.7 Hz, 1H), 5.30 (d, J=17.6 Hz, 1H), 5.14 (d, J=17.6 Hz, 1H), 2.71 (d, J=17.0 Hz, 1H), 2.63-2.42 (m, 2H), 2.37-2.20 (m, 4H), 2.13-1.78 (m, 6H), 1.64-1.56 (m, 1H), 1.37-1.28 (m, 1H), 1.30 (s, 3H), 1.08-0.94 (m, 1H), 0.53 (s, 3H).

### (R,E)-1-((1S,2S,3R,5S)-2-((Z)-7-(ethylamino)-7-oxohept-2-en-1-yl)-3,5-dihydroxycyclopentyl)-5-phenylpent-1-en-3-yl (2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl) terephthalate

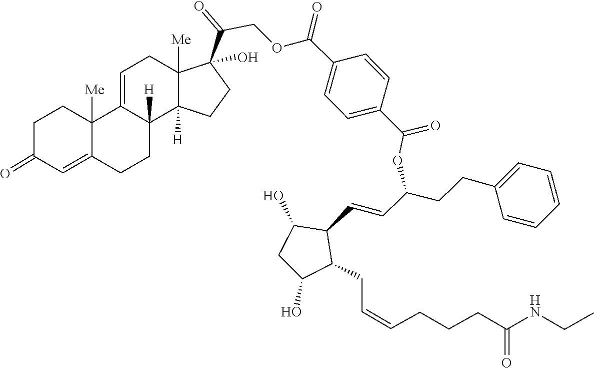

To a stirred solution of bimatoprost amide (232 mg, 0.56 mmol) in dry CH₂Cl₂ (10 mL) was added n-butylboronic acid (85.4 mg, 0.84 mmol). The solution was stirred at 37° C. for 16 h and concentrated in vacuo. The residue was twice dissolved in toluene (10 mL) and evaporated to dryness. The flask was purged with Ar and the residue dissolved in dry THF (20 mL). To the solution was added anecortave-4-carboxybenzoate (330 mg, 0.67 mmol), DMAP (81.8 mg, 0.67 mmol) and EDC.HCl (256 mg, 1.34 mmol). The mixture was stirred at room temperature for 24 h and then concentrated in vacuo to give a pale yellow residue that was partitioned between 0.5M HCl_{(aq.)} (50 mL) and CH₂Cl₂ (50 mL). The organics were separated, concentrated onto reverse phase silica (3 g) and purified by automated reverse phase chromatography (aqueous-MeCN). The product containing fractions were concentrated to give a solid that was further purified by normal phase Biotage automated chromatography (hexane-ethyl acetate gradient). The product containing fractions were concentrated in vacuo to give the product as a colorless solid (82 mg, 16%). Melting point: 92′° C. HPLC retention time: 35.6 min. ESI MS+ calculated for C₅₄H₆₇NNaO₁₀⁺; 912.4657, Found: 912.4646. ¹H NMR (400 MHz, DMSO-d6) δ 8.14-8.04 (m, 4H), 7.67-7.63 (m, 1H), 7.28-7.12 (m, 5H), 5.65 (s, 1H), 5.63-5.59 (m, 3H), 5.55 (d, J=5.9 Hz, 1H), 5.45-5.35 (m, 2H), 5.34-5.10 (m, 3H), 4.57 (d, J=5.9 Hz, 1H), 4.38 (d, J=4.8 Hz, 1H), 3.93-3.86 (m, 1H), 3.71-3.62 (m, 1H), 3.06-2.96 (m, 2H), 2.75-2.64 (m, 3H), 2.61-2.42 (m, 4H), 2.35-1.79 (m, 20H), 1.65-1.55 (m, 1H), 1.51-1.37 (m, 3H), 1.36-1.28 (m, 1H), 1.31 (s, 3H), 0.96 (t, J=7.2 Hz, 3H), 0.53 (s, 3H).

### Chemical Synthesis Example 24: (Bimatoprost-OEt(C₁₅)-Anecortave; Compound 33)

### Ethyl (Z)-7-((1S,2S,3S,5R)-3,5-dihydroxy-2-((3R,E)-3-(((2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)carbonyl)oxy)-5-phenylpent-1-en-1-yl)cyclopentyl)hept-5-enoate

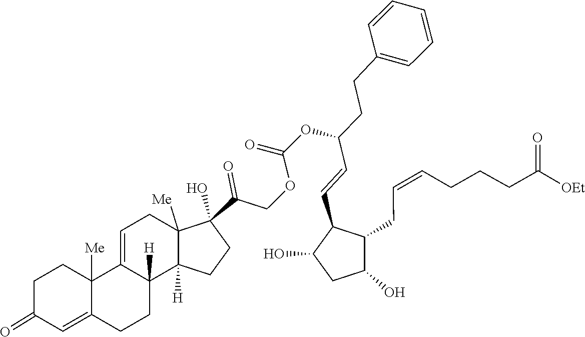

To a stirred solution of bimatoprost ethyl ester (230 mg, 0.55 mmol) in dry pyridine (3 mL) under nitrogen was added anecortave chloroformate (313 mg, 0.77 mmol) in CH₂Cl₂ (6 mL). The yellow solution was stirred for 2 h, concentrated and re-dissolved in CH₂Cl₂ (6 mL). The mixture was concentrated onto reverse phase silica (2 g) and purified by automated reverse phase chromatography (aqueous-MeCN). The product containing fractions were concentrated to give a solid that was further purified by normal phase Biotage automated chromatography (hexane-ethyl acetate gradient). The product containing fractions were concentrated in vacuo to give the product as a colorless solid (32 mg, 8%). Melting point: 62° C. HPLC retention time: 36.9 min. ESI MS+ calculated for C₄₇H₆₂NaO₁₀⁺; 809.4235, Found: 809.4241. ¹H NMR (400 MHz, DMSO-d6) 7.27-7.21 (m, 2H), 7.18-7.11 (m, 3H), 5.64 (s, 1H), 5.53-5.40 (m, 5H), 5.31-5.22 (m, 1H), 5.02 (d, J=17.8 Hz, 1H), 4.78 (d, J=17.8 Hz, 1H), 4.76 (s, 1H), 4.69-4.62 (m, 2H), 4.00 (q, J=7.1 Hz, 2H), 3.96-3.88 (m, 2H), 2.67-2.42 (m, 7H), 2.38-2.15 (m, 6H), 2.15-1.88 (m, 8H), 1.85-1.61 (m, 4H), 1.59-1.21 (m, 6H), 1.28 (s, 3H), 1.14 (t, J=7.1 Hz, 3H), 1.05-0.92 (m, 1H), 0.46 (s, 3H).

### Chemical Synthesis Example 25: (Anecortave-Gly-Bimatoprost; Compound 34)

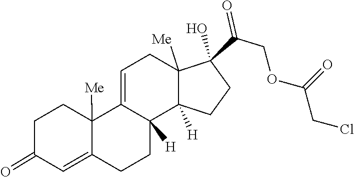 [SMILES: CC12CCC(=O)C=C1CC[C@@H]1C2=CCC2(C)[C@H]1CC[C@]2(O)C(=O)COC(=O)CCl]

### 2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl 2-chloroacetate (Anecortave chloroacetate)

To a stirred suspension of anecortave desacetate (1 g, 2.90 mmol) in dry CH₂Cl₂ (20 mL) was added Et₃N (1.21 mL, 8.71 mmol). Chloroacetyl chloride (693 μL, 8.71 mmol) was added dropwise over 10 min and the brown suspension stirred for 16 h. The reaction mixture was diluted with CH₂Cl₂ (50 mL) and the organics washed with 0.5M HCl_{(aq)} (70 mL). Organics were dried over anhydrous MgSO₄, filtered and evaporated to give the product (anecortave chloroacetate) as a brown solid (1.21 g, 99%). HPLC retention time: 27.3 min (Non-polar method). ESI MS+ calculated for C₂₃H₃₀ClO₅⁺; 421.1776, Found: 421.1778.

### 2-(2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)-2-oxoethyl (Z)-7-((1R,2R,3R,5S)-3,5-dihydroxy-2-((S,E)-3-hydroxy-5-phenylpent-1-en-1-yl)cyclopentyl)hept-5-enoate

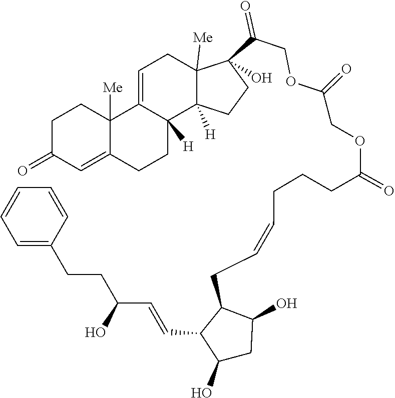 [SMILES: CC12CCC(=O)C=C1CC[C@@H]1C2=CCC2(C)[C@H]1CC[C@]2(O)C(=O)COC(=O)COC(=O)CCC/C=C\C[C@@H]1[C@@H](/C=C/[C@@H](O)CCc2ccccc2)[C@H](O)C[C@@H]1O]

A stirred suspension of anecortave chloroacetate (150 mg, 0.36 mmol), bimatoprost acid (173 mg, 0.45 mmol), Cs₂CO₃ (145 mg, 0.45 mmol) and NaI (107 mg, 0.71 mmol) in MeCN (5 mL) was placed in a pre-heated oil-bath at 70° C. and the mixture stirred for 20 h. The reaction mixture was cooled to room temperature, filtered and the solution concentrated onto C18 silica gel. The crude material was twice purified by automated reverse phase chromatography (aqueous-MeCN) to give the product as a colorless solid (48 mg, 17%). HPLC retention time: 29.4 min (Latanoprost method). ESI MS+ calculated for C₄₆H₆₀NaO₁₀⁺; 795.4079, Found: 795.4083. 1H NMR (400 MHz, DMSO) δ 7.26-7.21 (m, 2H), 7.20-7.10 (m, 3H), 5.65-5.62 (m, 1H), 5.55-5.44 (m, 3H), 5.42-5.31 (m, 2H), 5.28-5.20 (m, 1H), 5.13-5.06 (m, 1H), 4.94-4.84 (m, 1H), 4.78-4.68 (m, 2H), 4.63 (d, J=4.6 Hz, 1H), 4.48 (d, J=5.8 Hz, 1H), 4.33 (d, J=5.0 Hz, 1H), 3.88 (qd, J=6.2, 3.7 Hz, 2H), 3.69-3.61 (m, 1H), 3.27 (s, 1H), 2.66-2.60 (m, 1H), 2.60-2.50 (m, 3H), 2.48-2.42 (m, 1H), 2.34-2.08 (m, 7H), 2.07 (s, 2H), 2.08-2.02 (m, 1H), 1.97 (dt, J=13.8, 8.8 Hz, 5H), 1.85-1.79 (m, 1H), 1.81-1.73 (m, 2H), 1.73-1.58 (m, 2H), 1.58-1.51 (m, 1H), 1.53-1.48 (m, 1H), 1.41 (ddd, J=14.1, 5.7, 2.5 Hz, 1H), 1.37-1.24 (m, 2H), 1.28 (s, 3H), 1.03-0.93 (m, 1H), 0.46 (d, J=3.1 Hz, 3H).

### Chemical Synthesis Example 26: (Anecortave-Gly-Travoprost; Compound 35)

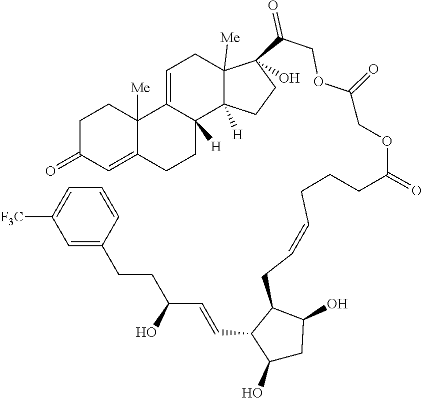 [SMILES: CC12CCC(=O)C=C1CC[C@@H]1C2=CCC2(C)[C@H]1CC[C@]2(O)C(=O)COC(=O)COC(=O)CCC/C=C\C[C@@H]1[C@@H](/C=C/[C@@H](O)CCc2cccc(C(F)(F)F)c2)[C@H](O)C[C@@H]1O]

### 2-(2-((8S,14S,17R)-17-hydroxy-10,13-dimethyl-3-oxo-2,3,6,7,8,10,12,13,14,15,16,17-dodecahydro-1H-cyclopentafalphenanthren-17-yl)-2-oxoethoxy)-2-oxoethyl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate

A stirred suspension of anecortave chloroacetate (150 mg, 0.36 mmol), travoprost acid (180 mg, 0.36 mmol), Cs₂CO₃ (117 mg, 0.36 mmol) and NaI (54 mg, 0.36 mmol) in MeCN (5 mL) was placed in a pre-heated oil-bath at 50° C. and the mixture stirred for 72 h. The reaction mixture was cooled to room temperature, filtered and the solution concentrated onto C18 silica gel. The crude material was purified by automated reverse phase chromatography (aqueous-MeCN) to give the product as a colorless solid (125 mg, 41%). HPLC retention time: 31.3 min (Latanoprost method). ESI MS+ calculated for C₄₆H₅₇F₃NaO₁₁⁺; 865.3732, Found: 865.3734. 1H NMR (400 MHz, DMSO) δ 7.48 (t, J=8.0 Hz, 1H), 7.28-7.15 (m, 3H), 5.63 (d, J=1.5 Hz, 1H), 5.58-5.38 (m, 5H), 5.27-5.16 (m, 1H), 5.13-5.04 (m, 2H), 4.94-4.82 (m, 1H), 4.79-4.66 (m, 2H), 4.50 (d, J=5.8 Hz, 1H), 4.35-4.24 (m, 2H), 3.97-3.84 (m, 3H), 3.72-3.61 (m, 1H), 3.29 (s, 2H), 3.26 (s, 1H), 2.67-2.54 (m, 2H), 2.53 (s, 1H), 2.45-2.40 (m, 1H), 2.33-2.24 (m, 4H), 2.24-2.06 (m, 4H), 2.00-1.91 (m, 4H), 1.84-1.70 (m, 3H), 1.58-1.46 (m, J=7.5 Hz, 3H), 1.41 (ddd, J=14.1, 5.7, 2.5 Hz, 1H), 1.29 (s, 1H), 1.27 (s, 3H), 1.05-0.94 (m, 1H), 0.45 (d, J=2.6 Hz, 3H).

### Chemical Synthesis Example 27: (Anecortave-Lact-Travoprost; Compound 36)

### 2-((8S,14S,17R)-17-hydroxy-10,13-dimethyl-3-oxo-2,3,6,7,8,10,12,13,14,15,16,17-dodecahydro-1H-cyclopentafalphenanthren-17-yl)-2-oxoethyl 2-bromopropanoate (Anecortave 2-bromopropanoate)

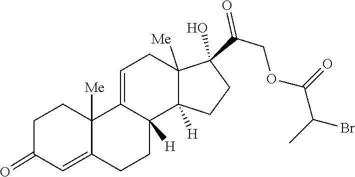 [SMILES: CC(Br)C(=O)OCC(=O)[C@@]1(O)CC[C@H]2[C@@H]3CCC4=CC(=O)CCC4(C)C3=CCC21C]

To a stirred suspension of anecortave desacetate (250 mg, 0.73 mmol) in dry 2-MeTHF (10 mL) was added Et₃N (202 μL, 1.45 mmol) and pyridine (50 μL). 2-Bromopropionyl bromide (91 μL, 0.87 mmol) was added dropwise over 10 min and the suspension stirred at room temperature for 30 mins. The reaction mixture was diluted with CH₂Cl₂ (10 mL) and the organics washed with 0.5M HCl_{(aq)} (20 mL). Organics were dried over anhydrous MgSO₄, filtered and evaporated to give the product (Anecortave 2-bromopropionate) as a pale yellow solid (347 mg, 99%). HPLC retention time: 30.6, 30.9 min (Non-polar method). ESI MS+ calculated for C₂₄H₃₂BrO₅⁺; 479.1428, Found: 479.1425.

### 1-(2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)-1-oxopropan-2-yl (Z)-7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate

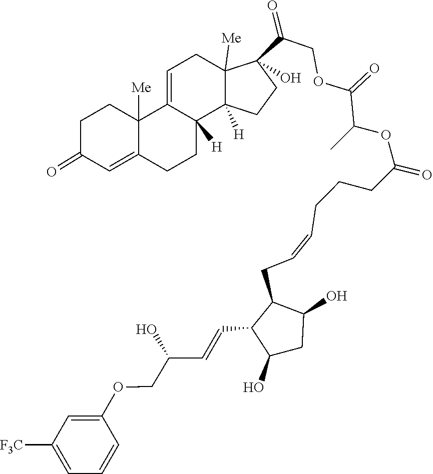 [SMILES: CC(OC(=O)CCC/C=C\C[C@@H]1[C@@H](/C=C/[C@@H](O)COc2cccc(C(F)(F)F)c2)[C@H](O)C[C@@H]1O)C(=O)OCC(=O)[C@@]1(O)CC[C@H]2[C@@H]3CCC4=CC(=O)CCC4(C)C3=CCC21C]

A stirred suspension of anecortave 2-bromopropionate (175 mg, 0.36 mmol), travoprost acid (180 mg, 0.36 mmol), Cs₂CO₃ (117 mg, 0.36 mmol) and NaI (54 mg, 0.36 mmol) in MeCN (5 mL) was placed in a pre-heated oil-bath at 70° C. and the mixture stirred for 16 h. The reaction mixture was cooled to room temperature, filtered and the solution concentrated onto C18 silica gel. The crude material was purified by automated reverse phase chromatography (aqueous-MeCN) and normal phase chromatography (EtOAc-Hexanes) to give the product as a colorless waxy solid (57 mg, 19%). HPLC retention time: 33.0, 33.2 min (Latanoprost method). ESI MS+ calculated for C₄₇H₅₉F₃NaO₁₁⁺; 879.3902, Found: 879.3902. 1H NMR (400 MHz, DMSO) δ 7.48 (t, J=8.0 Hz, 1H), 7.27-7.15 (m, 3H), 5.63 (d, J=1.4 Hz, 1H), 5.60-5.35 (m, 4H), 5.27-5.16 (m, 1H), 5.16-4.76 (m, 4H), 4.50 (d, J=5.8 Hz, 1H), 4.35-4.26 (m, 2H), 3.95-3.82 (m, 3H), 3.70-3.62 (m, 1H), 2.67-2.49 (m, 2H), 2.48-2.39 (m, 1H), 2.34-2.12 (m, 6H), 2.14-1.96 (m, 3H), 1.99-1.91 (m, 5H), 1.88 (s, 2H), 1.82-1.71 (m, 3H), 1.58-1.35 (m, 3H), 1.29 (s, 2H), 1.27 (d, J=2.0 Hz, 4H), 1.22 (s, 2H), 1.05-0.94 (m, 1H), 0.84 (q, J=7.5 Hz, 1H), 0.45 (d, J=3.3 Hz, 3H).

### Chemical Synthesis Example 28: (Bimatoprost(C₁)Anecortave(C₁₅) acetate; Compound 51)

### 2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl (Z)-7-((1R,2R,3R,5S)-2-((S,E)-3-acetoxy-5-phenylpent-1-en-1-yl)-3,5-dihydroxycyclopentyl)hept-5-enoate

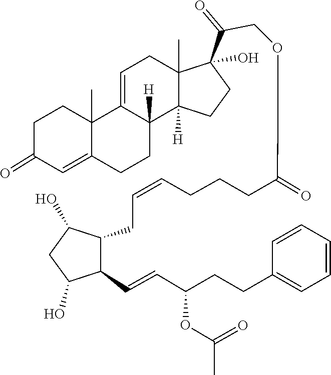

To a stirred solution of Bim-Anec (200 mg, 0.28 mmol) in dry CH₂Cl₂ (10 mL) was added n-butylboronic acid (42.8 mg, 0.42 mmol). The solution was stirred at 37° C. for 16 h and concentrated in vacuo. The residue was twice dissolved in toluene (10 mL) and evaporated to dryness. The residue was then dissolved in CH₂Cl₂ (8 mL) and the mixture stirred at 0° C., followed by addition of acetic anhydride (8 mL) and pyridine (68 μL, 0.84 mmol). The mixture was stirred at room temperature for 16 h and MeOH (20 mL) added. After 16 h the mixture was concentrated onto reverse phase silica (2 g) and purified by automated reverse phase chromatography (aqueous-MeCN). The product containing fractions were concentrated in vacuo to give the product as a colorless solid (62 mg, 29%). Melting point: 125° C. HPLC retention time: 34.4 min. ESI MS+ calculated for C₄₆H₆₀NaO₉⁺; 779.4130, Found: 779.4132. 1H NMR (400 MHz, DMSO-d6) δ 7.30-7.24 (m, 2H), 7.20-7.14 (m, 3H), 5.66 (s, 1H), 5.55-5.41 (m, 5H), 5.31-5.22 (m, 1H), 5.14 (q, J=6.5 Hz, 1H), 4.97 (d, J=17.6 Hz, 1H), 4.84 (d, J=17.6 Hz, 1H), 4.57 (d, J=5.9 Hz, 1H), 4.38 (d, J=4.8 Hz, 1H), 3.94-3.88 (m, 1H), 3.72-3.62 (m, 1H), 2.70-2.53 (m, 5H), 2.36-1.88 (m, 21H), 2.00 (s, 3H), 1.60-1.50 (m, 3H), 1.47-1.40 (m, 1H), 1.36-1.28 (m, 1H), 1.30 (s, 3H), 1.07-0.94 (m, 1H), 0.48 (s, 3H).

### Chemical Synthesis Example 29: (Bimatoprost(C₁)Anecortave(C₁₅) benzoyl; Compound 52)

### (3S,E)-1-((1R,2R,3S,5R)-3,5-dihydroxy-2-((Z)-7-(2-((8S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)-7-oxohept-2-en-1-yl)cyclopentyl)-5-phenylpent-1-en-3-yl benzoate

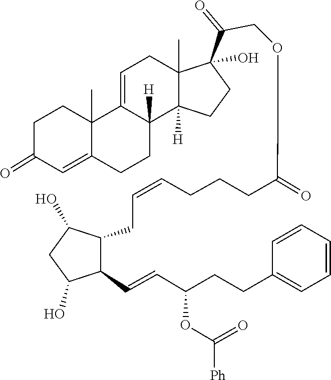

To a stirred solution of Bim-Anec (100 mg, 0.14 mmol) in dry CH₂Cl₂ (3 mL) was added n-butylboronic acid (21.4 mg, 0.21 mmol). The solution was stirred at 37° C. for 16 h and concentrated in vacuo. The residue was twice dissolved in toluene (5 mL) and evaporated to dryness. The residue was then dissolved in CH₂Cl₂ (3 mL) and the mixture stirred at 0° C., followed by addition of pyridine (1 mL) and benzoyl chloride (23.4 μL, 0.21 mmol). The mixture was stirred at room temperature for 24 h, concentrated onto reverse phase silica (2g) and purified by automated reverse phase chromatography (aqueous-MeCN). The product containing fractions were concentrated in vacuo to give the product as a colorless solid (36 mg, 32%). Melting point: 127° C. HPLC retention time: 39.6 min. ESI MS+ calculated for C₅₂H₆₂NaO₉⁺; 814.4286, Found: 814.4285. ¹H NMR (400 MHz, DMSO-d6) 1H NMR (400 MHz, DMSO-d6) δ 7.96-7.92 (m, 2H), 7.66 -7.60 (m, 1H), 7.50 (t, J=8.0 Hz, 2H), 7.28-7.22 (m, 2H), 7.20-7.12 (m, 3H), 5.64 (s, 1H), 5.61-5.58 (m, 2H), 5.51-5.37 (m, 4H), 5.22 (q, J=7.2 Hz, 1H), 4.96 (dd, J=17.7 Hz, 1H), 4.82 (d, J=17.7 Hz, 1H), 4.56 (d, J=5.9 Hz, 1H), 4.37 (d, J=4.8 Hz, 1H), 3.94-3.85 (m, 1H), 3.70-3.63 (m, 1H), 2.69-2.58 (m, 3H), 2.57-2.40 (m, 2H), 2.34-1.90 (m, 18H), 1.86-1.74 (m, 3H), 1.60-1.45 (m, 3H), 1.45-1.38 (m, 1H), 1.36-1.24 (m, 1H), 1.27 (s, 3H), 1.04-0.91 (m, 1H), 0.47 (s, 3H).

### Chemical Synthesis Example 30: (Dexamethasone-TEG-Axitinib; Compound 54)

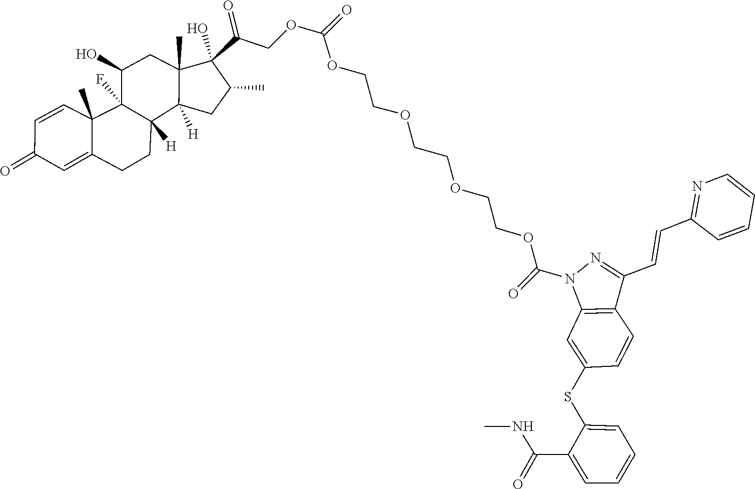 [SMILES: CNC(=O)c1ccccc1Sc1ccc2c(/C=C/c3ccccn3)nn(C(=O)OCCOCCOCCOC(=O)OCC(=O)[C@@]3(O)[C@H](C)C[C@H]4[C@@H]5CCC6=CC(=O)C=C[C@]6(C)[C@@]5(F)[C@@H](O)C[C@@]43C)c2c1]

### 1-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-1,4-dioxo-3,5,8,11-tetraoxatridecan-13-yl 6-((2-(methylcarbamoyl)phenyl)thio)-3-((E)-2-(pyridin-2-yl)vinyl)-1H-indazole-1-carboxylate

To a stirred solution of dexamethasone (392 mg, 1 mmol) in dry THF (50 mL) under nitrogen was added phosgene solution (3.57 mL of a 1.4M soln in toluene, 5 mmol). The mixture was stirred overnight at RT then concentrated to give the dexamethasone chloroformate (455 mg, 100%) as an orange solid. This was dissolved in dry DCM (50 mL) and triethylene glycol (1.07 mL, 1.20 g, 8 mmol) and pyridine (161 μL, 158 mg, 2.0 mmol) were added and the mixture stirred for 2 h under nitrogen. The reaction solution was concentrated onto normal phase silica (2 g) and purified twice by automated normal phase chromatography (ethyl acetate-hexane). The product containing fractions were combined and concentrated in vacuo to give the Dex-TEG intermediate as an off-white glassy solid (369 mg, 65%). HPLC retention time 19.0 min (Non-polar method). ESI MS+ calculated for Chemical Formula: C29H42FO10⁺; 569.2757 Found: 569.2747. ¹H NMR (400 MHz, DMSO) δ 7.29 (d, J=10.1 Hz, 1H), 6.23 (dd, J=10.2, 1.9 Hz, 1H), 6.01 (t, J=1.7 Hz, 1H), 5.40 (dd, J=5.0, 1.4 Hz, 1H), 5.18 (s, 1H), 5.08 (d, J=17.7 Hz, 1H), 4.79 (d, J=17.7 Hz, 1H), 4.57 (s, 1H), 4.25-4.17 (m, 2H), 4.17-4.05 (m, 1H), 3.69-3.59 (m, 2H), 3.59-3.51 (m, 4H), 3.49 (dd, J=5.4, 4.2 Hz, 2H), 3.46-3.38 (m, 2H), 2.88 (ddd, J=11.1, 7.2, 4.1 Hz, 1H), 2.62 (td, J=13.6, 6.2 Hz, 1H), 2.35 (ddd, J=25.3, 12.4, 5.0 Hz, 2H), 2.22-2.05 (m, 2H), 1.85-1.72 (m, 1H), 1.64 (q, J=11.7 Hz, 1H), 1.59-1.51 (m, 1H), 1.49 (s, 3H), 1.36 (tt, J=12.7, 6.3 Hz, 1H), 1.08 (ddd, J=12.2, 8.1, 4.1 Hz, 1H), 0.89 (s, 3H), 0.79 (d, J=7.2 Hz, 3H).

To a stirred soln of Dex-TEG (200 mg, 0.35 mmol) in dryTHF (10 mL) under nitrogen was added phosgene solution (1.25 mL of a 1.4M soln in toluene, 1.75 mmol) and the mixture stirred for 5 h. The mixture was concentrated to give the Dex-TEG chloroformate intermediate (221 mg, 0.35 mmoL) which was dissolved in DCM (20 mL). Axitinib (135 mg, 0.35 mmol) and triethylamine (49 uL, 35 mg, 0.35 mmol) were added with stirring under nitrogen. The mixture was stirred for 2h, part concentrated and purified by automated normal phase chromatography (DCM-5% MeOH/DCM). The product containing fractions were combined and concentrated in vacuo to give the Dex-TEG-Axit (85 mg, 25%) as an off-white glassy solid. HPLC retention time 20.8 min (Non-polar method). ESI MS+ calculated for C22H58FN4O12S+; 981.3750 Found: 981.3742 ¹H NMR (400 MHz, DMSO) δ 8.69-8.62 (m, 1H), 8.38 (d, J=4.7 Hz, 1H), 8.32 (dd, J=8.5, 0.7 Hz, 1H), 8.20 (dd, J=1.6, 0.6 Hz, 1H), 7.94 (d, J=16.4 Hz, 1H), 7.86 (td, J=7.7, 1.8 Hz, 1H), 7.84-7.71 (m, 2H), 7.57-7.49 (m, 1H), 7.44-7.37 (m, 1H), 7.37-7.32 (m, 3H), 7.28 (d, J=10.2 Hz, 1H), 7.21-7.12 (m, 1H), 6.22 (dd, J=10.1, 1.9 Hz, 1H), 6.00 (t, J=1.7 Hz, 1H), 5.42-5.34 (m, 1H), 5.15 (s, 1H), 5.05 (d, J=17.7 Hz, 1H), 4.75 (d, J=17.7 Hz, 1H), 4.63-4.52 (m, 2H), 4.25-4.07 (m, 3H), 3.88-3.75 (m, 2H), 3.67-3.50 (m, 6H), 2.91-2.79 (m, 1H), 2.76 (d, J=4.6 Hz, 3H), 2.59 (td, J=13.5, 5.9 Hz, 1H), 2.42-2.20 (m, 2H), 2.20-2.01 (m, 2H), 1.81-1.70 (m, 1H), 1.55 (dd, J=31.5, 13.0 Hz, 2H), 1.47 (s, 3H), 1.34 (dt, J=12.8, 7.2 Hz, 1H), 1.10-0.97 (m, 1H), 0.86 (s, 3H), 0.76 (d, J=7.2 Hz, 3H).

### Chemical Synthesis Example 31: (Naltrexone-CDM-Indomethacin; Compound 55)

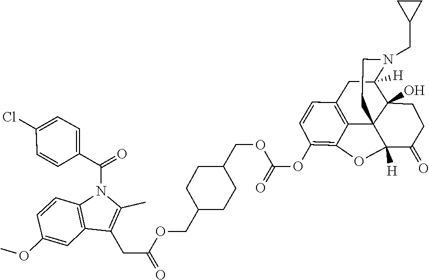 [SMILES: COc1ccc2c(c1)c(CC(=O)OCC1CCC(COC(=O)Oc3ccc4c5c3O[C@H]3C(=O)CC[C@@]6(O)[C@@H](C4)N(CC4CC4)CC[C@]536)CC1)c(C)n2C(=O)c1ccc(Cl)cc1]

### (4-((((((4R,4a5,7aR,12bS)-3-(cyclopropylmethyl)-4α-hydroxy-7-oxo-2,3,4,4a,5,6,7,7a-octahydro-1H-4,12-methanobenzofuro[3,2-e]isoquinolin-9-yl)oxy)carbonyl)oxy)methyl)cyclohexyl)methyl 2-(1-(4-chlorobenzoyl)-5-methoxy-2-methyl-1H-indol-3-yl)acetate

To a stirred soln of Indomethacin (1.0 g, 2.79 mmol), 1,4-cyclohexanedimethanol (2.01 g, 13.95 mmol) and DMAP (0.34 g, 2.79 mmol) in DCM (100 mL) was added EDC.HCl and the mixture stirred for 4 h at RT. The mixture was part concentrated and purified by automated normal phase chromatography (DCM-5% MeOH/DCM). The product containing fractions were combined and concentrated in vacuo to give the Indomethacin-CDM (1.35 g, 99%) as a yellow solid. To a solution of indomethacin-CDM (675 mg, 1.40 mmol) in dry THF under nitrogen was added phosgene solution (5.0 mL of a 1.4M soln in toluene, 6.95 mmol). The mixture was stirred for 6 h and concentrated to give the Indom-CDM chloroformate intermediate (762 mg, 100%) which was dissolved in DCM (25 mL). Naltrexone hydrochloride (527 mg, 1.40 mmol) and DMAP (171 mg, 1.40 mmol) were added and the mixture stirred for 2d. The mixture was concentrated onto reverse phase silica (2 g) and purified by automated reverse phase chromatography (80% Water/20% MeCN-100% MeCN). The product containing fractions were combined and concentrated in vacuo to give the Naltrexone-CDM-Indomethacin (353 mg, 30%) as a light yellow solid. HPLC retention time: 26.9 min as a double peak (Non-polar method). ESI MS+ calculated for C48H52ClN2O10+; 851.3305, Found 851.3301 ¹H NMR (400 MHz, DMSO) δ 7.70-7.60 (m, 4H), 7.04 (t, J=2.9 Hz, 1H), 6.98-6.89 (m, 2H), 6.76-6.68 (m, 2H), 5.14 (s, 1H), 4.94 (d, J=3.2 Hz, 1H), 4.09 (dd, J=7.3, 1.8 Hz, 1H), 4.05-3.95 (m, 2H), 3.88 (d, J=6.2 Hz, 1H), 3.80-3.73 (m, 5H), 3.17 (d, J=5.5 Hz, 1H), 3.05 (s, 1H), 2.98-2.89 (m, 1H), 2.62 (ddd, J=24.6, 15.1, 6.3 Hz, 2H), 2.39 (q, J=10.1 Hz, 3H), 2.23 (d, J=3.3 Hz, 3H), 2.11 (dd, J=13.9, 2.9 Hz, 1H), 2.00-1.90 (m, 1H), 1.87-1.75 (m, 2H), 1.69 (s, 2H), 1.63-1.20 (m, 8H), 1.06-0.80 (m, 4H), 0.54-0.44 (m, 2H).

### Chemical Synthesis Example 32: (Hydrocortisone-Succ-Axitinib; Compound 56)

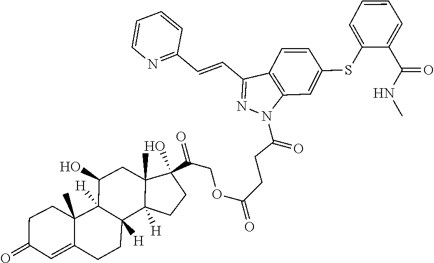 [SMILES: CNC(=O)c1ccccc1Sc1ccc2c(/C=C/c3ccccn3)nn(C(=O)CCC(=O)OCC(=O)[C@@]3(O)CC[C@H]4[C@@H]5CCC6=CC(=O)CC[C@]6(C)[C@H]5[C@@H](O)C[C@@]43C)c2c1]

### 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl 4-(6-((2-(methylcarbamoyl)phenyl)thio)-3-((E)-2-(pyridin-2-yl)vinyl)-1H-indazol-1-yl)-4-oxobutanoate

To a stirred suspension of HC-hemisuccinate (200 mg, 0.43 mmol) and axitinib (167 mg, 0.43 mmol) in anhydrous DMF (4 mL) was added DIPEA (151 μL, 0.86 mmol) followed by HBTU (164 mg, 0.43 mmol). The resulting solution was then stirred at room temperature. After 16 h, the colorless precipitate was isolated by filtration on a glass fritted funnel and the filter cake washed with TBME (10 mL). The crude solid was recrystallized from hot acetone (5 mL) and dried in a vacuum oven at 40° C. for 24 h to give the pure product as a colorless solid (196 mg, 55%). HPLC retention time: 21.0 min (Non-polar method). ESI MS+ calculated for C₄₇H₅₁N₄O₈S⁺; 831.3422, Found: 831.3417. 1H NMR (400 MHz, DMSO) δ 8.71-8.64 (m, 1H), 8.43 (q, J=4.6 Hz, 1H), 8.39-8.32 (m, 2H), 7.97 (d, J=16.4 Hz, 1H), 7.93-7.81 (m, 2H), 7.80 (d, J=7.8 Hz, 1H), 7.58-7.50 (m, 1H), 7.47 (dd, J=8.4, 1.5 Hz, 1H), 7.43-7.32 (m, 3H), 7.23-7.14 (m, 1H), 5.57 (d, J=1.5 Hz, 1H), 5.42 (s, 1H), 5.15 (d, J=17.6 Hz, 1H), 4.82 (d, J=17.6 Hz, 1H), 4.33 (d, J=3.9 Hz, 1H), 4.27 (s, 1H), 3.58-3.45 (m, 2H), 2.94-2.86 (m, 3H), 2.78 (d, J=4.6 Hz, 3H), 2.46-2.32 (m, 2H), 2.24-2.15 (m, 2H), 2.13-2.06 (m, 1H), 1.93 (d, J=11.8 Hz, 3H), 1.84-1.74 (m, 1H), 1.70-1.62 (m, 3H), 1.52-1.42 (m, 1H), 1.37 (s, 3H), 1.33-1.24 (m, 1H), 1.04-0.96 (m, 1H), 0.91-0.84 (m, 1H), 0.78 (s, 3H).

### Chemical Synthesis Example 33: (Anecortave-TEG-Axitinib; Compound 57)

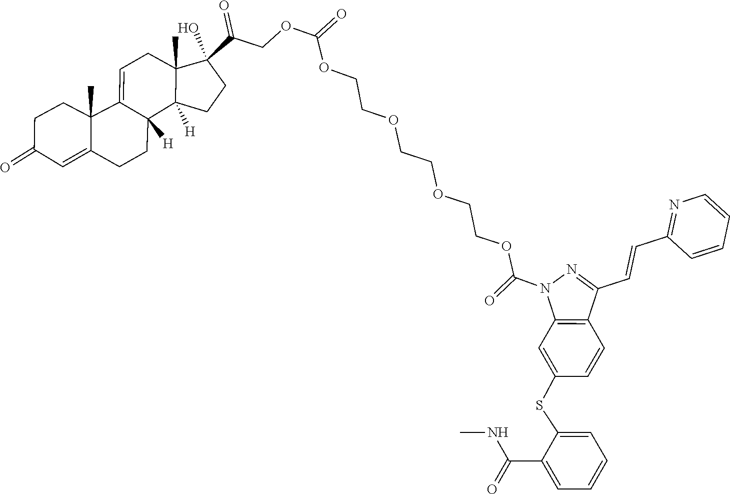 [SMILES: CNC(=O)c1ccccc1Sc1ccc2c(/C=C/c3ccccn3)nn(C(=O)OCCOCCOCCOC(=O)OCC(=O)[C@@]3(O)CC[C@H]4[C@@H]5CCC6=CC(=O)CC[C@]6(C)C5=CC[C@@]43C)c2c1]

### 1-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-1,4-dioxo-3,5,8,11-tetraoxatridecan-13-yl 6-((2-(methylcarbamoyl)phenyl)thio)-3-((E)-2-(pyridin-2-yl)vinyl)-1H-indazole-1-carboxylate

To a stirred solution of anecortave des acetate (344 mg, 1 mmol) in dry THF (50 mL) under nitrogen was added phosgene solution (3.57 mL of a 1.4M soln in toluene, 5 mmol). The mixture was stirred overnight at RT then concentrated to give the anecortave des acetate chloroformate (407 mg, 100%) as an orange solid. This was dissolved in dry DCM (50 mL) and triethylene glycol (1.07 mL, 1.20 g, 8 mmol) and pyridine (161 μL, 158 mg, 2.0 mmol) were added and the mixture stirred for 2 h under nitrogen. The reaction solution was concentrated onto normal phase silica (2 g) and purified twice by automated normal phase chromatography (ethyl acetate-hexane). The product containing fractions were combined and concentrated in vacuo to give the Anec-TEG intermediate as an off-white glassy solid (120 mg, 23%). HPLC retention time: 18.9 min (Non-polar method). ESI MS+ calculated for C₂₈H₄₁O₉⁺; 521.2745, Found 521.2746 ¹H NMR (400 MHz, DMSO) δ 5.66 (s, 1H), 5.54 (s, 2H), 5.06 (d, J=17.8 Hz, 1H), 4.85 (d, J=17.7 Hz, 1H), 4.56 (t, J=5.5 Hz, 1H), 4.26-4.14 (m, 2H), 3.66-3.59 (m, 2H), 3.59-3.38 (m, 10H), 2.63 (s, 3H), 2.30 (m, 4H), 2.04 (d, J=39.1 Hz, 2H), 1.91-1.73 (m, 3H), 1.54 (s, 1H), 1.30 (s, 4H), 0.50 (s, 3H).

To a stirred soln of Anec-TEG (120 mg, 0.23 mmol) in dry THF (10 mL) under nitrogen was added phosgene solution (0.82 mL of a 1.4M soln in toluene, 1.15 mmol) and the mixture stirred for 19 h. The mixture was concentrated to give the Anec-TEG chloroformate intermediate (134 mg, 100%) which was dissolved in DCM (10 mL). Axitinib (89 mg, 0.23 mmol) and triethylamine (32 uL, 23 mg, 0.23 mmol) were added with stirring under nitrogen. The mixture was stirred for 2h, part concentrated and purified by automated normal phase chromatography (DCM-5% MeOH/DCM). The product containing fractions were combined and concentrated in vacuo to give the Anec-TEG-Axit (52 mg, 24%) as an off-white glassy solid. HPLC retention time: 21.6 min (Non-polar method). ESI MS+ calculated for C51H57N4O11S+; 933.3739, Found 933.3748 ¹H NMR (400 MHz, DMSO) δ 8.65 (dt, J=4.7, 1.6 Hz, 1H), 8.38 (q, J=4.6 Hz, 1H), 8.34-8.26 (m, 1H), 8.20 (d, J=1.5 Hz, 1H), 8.04-7.72 (m, 4H), 7.60-7.48 (m, 1H), 7.48-7.27 (m, 4H), 7.27-7.06 (m, 1H), 5.64 (d, J=1.5 Hz, 1H), 5.49 (d, J=8.7 Hz, 2H), 5.00 (d, J=17.8 Hz, 1H), 4.78 (d, J=17.8 Hz, 1H), 4.66-4.48 (m, 2H), 4.26-4.07 (m, 2H), 3.90-3.74 (m, 2H), 3.72-3.48 (m, 6H), 2.76 (d, J=4.6 Hz, 3H), 2.60 (d, J=18.1 Hz, 2H), 2.47-2.38 (m, 2H), 2.38-2.11 (m, 3H), 2.11-1.89 (m, 3H), 1.77 (tdd, J=17.2, 12.9, 7.0 Hz, 3H), 1.67-1.44 (m, 1H), 1.26 (s, 4H), 1.11-0.82 (m, 1H), 0.45 (s, 3H).

### Chemical Synthesis Example 34: (Hydrocortisone-Succ-Simvastatin; Compound 58)

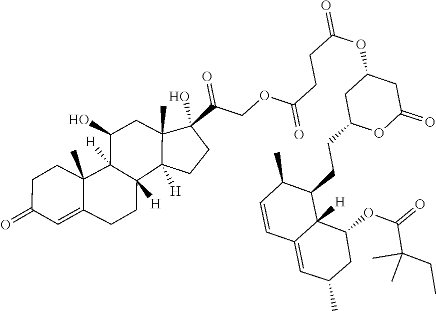 [SMILES: CCC(C)(C)C(=O)O[C@@H]1C[C@H](C)C=C2C=C[C@@H](C)[C@@H](CC[C@@H]3C[C@H](OC(=O)CCC(=O)OCC(=O)[C@@]4(O)CC[C@H]5[C@@H]6CCC7=CC(=O)CC[C@]7(C)[C@H]6[C@@H](O)C[C@@]54C)CC(=O)O3)[C@@H]21]

### 2-((8S,9S,10R,11S,13S,14S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl ((2R,4S)-2-(2-((1R,2R,6S,8R,8aS)-8-((2,2-dimethylbutanoyl)oxy)-2,6-dimethyl-1,2,6,7,8,8a-hexahydronaphthalen-1-yl)ethyl)-6-oxotetrahydro-2H-pyran-4-yl) succinate

To a stirred solution of HC-hemisuccinate (231 mg, 0.50 mmol), simvastatin (209 mg, 0.5 mmol) and DMAP (61 mg, 0.5 mmol) in DCM (25 mL) was added EDC.HCl (96 mg, 0.5 mmol) and the mixture stirred for 4 h. The mixture was concentrated onto reverse phase silica (2 g) and purified by automated reverse phase chromatography (80% Water/20% MeCN-100% MeCN). The product containing fractions were combined and concentrated in vacuo to give the HC-Succ-Simvastatin (176 mg, 41%) as an off-white solid. HPLC retention time: 44.3 min (Non-polar method). ESI MS+ calculated for C50H71O12+; 863.4940, Found 863.4937 ¹H NMR (400 MHz, DMSO) δ 6.04-5.86 (m, 1H), 5.86-5.72 (m, 1H), 5.56 (d, J=1.5 Hz, 1H), 5.50 (t, J=3.4 Hz, 1H), 5.39 (s, 1H), 5.26-5.00 (m, 3H), 4.76 (d, J=17.5 Hz, 1H), 4.43 (dt, J=7.1, 3.8 Hz, 1H), 4.33 (d, J=3.9 Hz, 1H), 4.32-4.19 (m, 1H), 2.86 (dd, J=17.8, 5.4 Hz, 1H), 2.73-2.53 (m, 5H), 2.47-2.23 (m, 6H), 2.24-2.04 (m, 3H), 1.95 (tdd, J=13.3, 8.6, 2.9 Hz, 5H), 1.89-1.54 (m, 8H), 1.55-1.38 (m, 3H), 1.40-1.14 (m, 7H), 1.14-0.89 (m, 10H), 0.84 (d, J=7.0 Hz, 4H), 0.81-0.65 (m, 6H).

### Chemical Synthesis Example 35: (Dexamethasone-Hex-Naproxen; Compound 59)

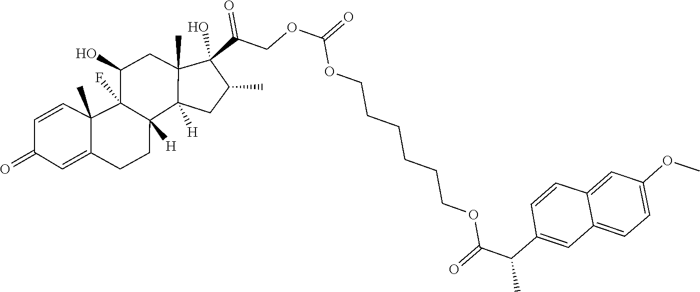 [SMILES: COc1ccc2cc([C@H](C)C(=O)OCCCCCCOC(=O)OCC(=O)[C@@]3(O)[C@H](C)C[C@H]4[C@@H]5CCC6=CC(=O)C=C[C@]6(C)[C@@]5(F)[C@@H](O)C[C@@]43C)ccc2c1]

### 6-(((2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)oxy)hexyl (S)-2-(6-methoxynaphthalen-2-yl)propanoate

To a stirred solution of dexamethasone (784 mg, 2 mmol) in dry THF (100 mL) under nitrogen was added phosgene solution (7.14 mL of a 1.4M solution in toluene, 10 mmol). The mixture was stirred overnight at RT then concentrated to give dexamethasone chloroformate (910 mg, 100%) as an orange solid. Dexamethasone chloroformate (455 mg, 1 mmol) was dissolved in dry DCM (50 mL) and 1,6-hexanediol (0.98 mL, 8 mmol) and pyridine (161 μL, 2.0 mmol) were added and the mixture stirred for 16 h under nitrogen. The reaction solution was concentrated onto normal phase silica (2 g) and purified twice by automated normal phase chromatography (ethyl acetate-hexane). The product containing fractions were combined and concentrated in vacuo to give 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl (6-hydroxyhexyl) carbonate (Dex-Hex) as an off-white glassy solid (120 mg, 22%). HPLC retention time: 24.8 min (Non-polar method). ESI MS+ calculated for C₂₉H₄₂FO₈⁺; 537.2847, Found: 537.2848. ¹H NMR (400 MHz, DMSO) δ 7.31 (d, J=10.2 Hz, 1H), 6.24 (dd, J=10.1, 2.0 Hz, 1H), 6.02 (s, 1H), 5.42 (d, J=4.6 Hz, 1H), 5.19 (s, 1H), 5.08 (d, J=17.7 Hz, 1H), 4.79 (d, J=17.7 Hz, 1H), 4.35 (t, J=5.1 Hz, 1H), 4.18-4.14 (m, 1H), 4.11 (t, J=6.6 Hz, 2H), 3.45-3.36 (m, 2H), 2.96-2.86 (m, 1H), 2.70-2.66 (m, 1H), 2.38-2.29 (m, 2H), 2.20-2.10 (m, 2H), 1.79 (s, 1H), 1.68-1.50 (m, 4H), 1.51 (s, 3H), 1.49-1.40 (m, 2H), 1.38-1.28 (m, 5H), 1.11-1.06 (m, 1H), 0.91 (s, 3H), 0.81 (d, J=7.2 Hz, 3H).

To a stirred suspension of Dex-Hex (120 mg, 0.22 mmol) in DCM (20 mL) was added Naproxen (51.4 mg, 0.22 mmol), DMAP (27.3 mg, 0.22 mmol) and EDC.HCl (42.9 mg, 0.22 mmol). The resulting suspension was then stirred at room temperature for 16 h. The mixture was concentrated onto reverse phase silica (3 g) and purified by automated reverse phase chromatography (aqueous-MeCN). The product containing fractions were concentrated in vacuo to give the product as a colorless solid (43 mg, 26%). HPLC retention time: 40.3 min (Non-polar method). ESI MS+ calculated for C₄₃H₅₄FO₁₀⁺; 749.3696, Found: 749.3692. 1H NMR (400 MHz, DMSO) δ 7.79 (t, J=8.3 Hz, 2H), 7.71 (d, J=1.8 Hz, 1H), 7.39 (dd, J=8.5, 1.9 Hz, 1H), 7.34-7.26 (m, 2H), 7.15 (dd, J=9.0, 2.5 Hz, 1H), 6.22 (dd, J=10.1, 1.9 Hz, 1H), 6.01 (t, J=1.7 Hz, 1H), 5.42 (dd, J=5.1, 1.3 Hz, 1H), 5.18 (s, 1H), 5.07 (d, J=17.7 Hz, 1H), 4.77 (d, J=17.7 Hz, 1H), 4.18-4.11 (m, 1H), 4.09-3.91 (m, 4H), 3.94-3.85 (m, 1H), 3.86 (s, 3H), 2.93-2.83 (m, 1H), 2.61 (td, J=13.8, 6.4 Hz, 1H), 2.35-2.25 (m, 2H), 2.20-2.06 (m, 2H), 1.80-1.73 (m, 1H), 1.69-1.60 (m, 1H), 1.60-1.38 (m, 11H), 1.41-1.30 (m, 1H), 1.27-1.12 (m, 4H), 1.07 (ddd, J=12.1, 8.0, 4.0 Hz, 1H), 0.89 (s, 3H), 0.79 (d, J=7.3 Hz, 3H).

### Example 3: Formation and Evaluation of Processable Conjugates

### Process Example 1: Heat Processing Pellets

Method A: A compound provided herein is formed into a pellet in the glassy state by heat molding. Crystalline powder of the compound is melted at a temperature between 85° C. to 130° C. and pressed into a cylindrical mold of ^{˜}1 mm height×1 mm diameter.

Method B: A compound provided herein (e.g., Compound 54, Compound 55, or Compound 56) is formed into a pellet in the glassy state by heat molding. Powder of the compound is melted at a temperature above 110° C. (e.g., 110° C. to 190° C.) and pressed into a cylindrical mold of ^{˜}1 mm height×1 mm diameter.

### Process Example 2: Solvent Processing

A compound provided herein (e.g., Compound 4) was formed into a thin film coating on a polymer surface by solvent casting. The compound (e.g., Compound 4) was dissolved in acetone at 50 mg/ml. 20 μl was cast onto a Dacron coupon and left to air dry at room temperature overnight followed by 2 h under vacuum at 50° C.

### Process Example 3: Heat Processing Rods

A compound of the disclosure was formed into a rod in the glassy state by heat extrusion. The conjugate compound was initially melted at a temperature up to 140° C. The resulting material was then loaded into a heat extruder with a 30G die head, heated between 70° C. to 125° C., and pressure was applied to a piston to form extrudate from the extruder. The extrudate was cut to different lengths.

### Example 4: Drug Release Evaluation from Pellets or Extruded Rods

Drug release from heat-molded pellets or extruded rods of Compounds of the disclosure were assessed in either fetal bovine serum (FBS), phosphate buffered saline (PBS), or 1% FBS in PBS (v/v). Heat-molded pellets or extruded rods were placed in 20 ml glass vials, to which was added 2 ml of release buffer. Samples were incubated at 37° C. with constant agitation at 115 rpm. At intervals up to 14 days in length, release buffer was assessed for released drug and then fully replaced with 2 ml of fresh buffer. For FBS release conditions, acetonitrile was added to precipitate proteins and extract drug release products. Samples were analyzed by high performance liquid chromatography (HPLC) to quantify drug products.

### Example 5. Sterilization of a Heat Processed Rod

A compound provided herein (e.g., Compound 5) was formed into rods by melt extrusion and cut to length. The resulting implants were loaded into the lumen of needles and terminally sterilized by ethylene oxide, gamma irradiation, and E-beam. Following sterilization, samples were dissolved in a suitable solvent and assessed for changes in purity due to sterilization by HPLC.

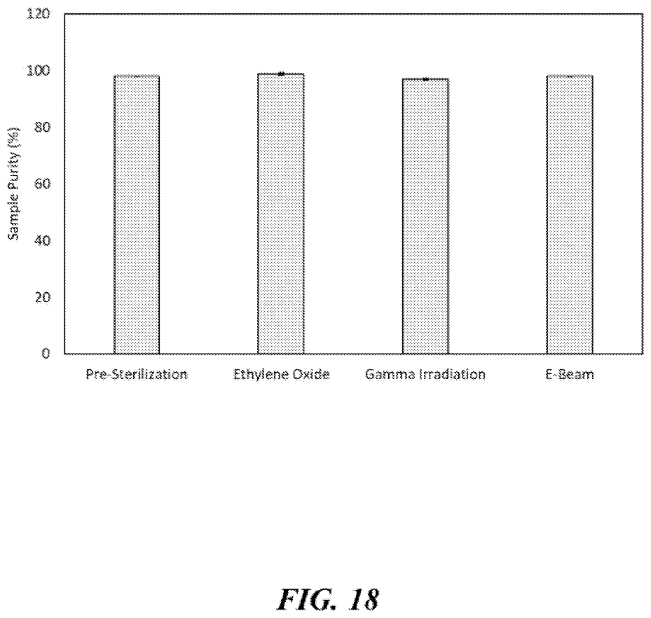

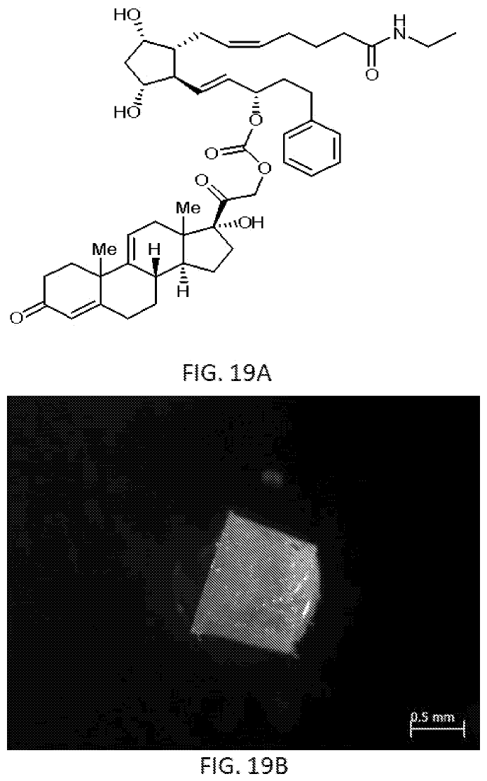

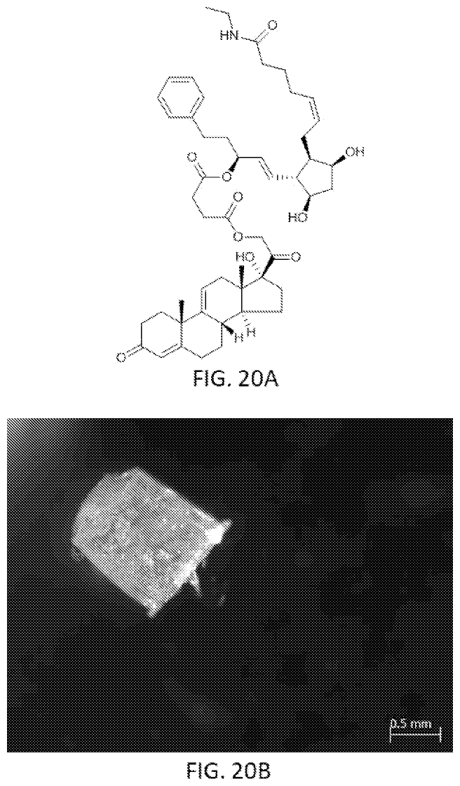

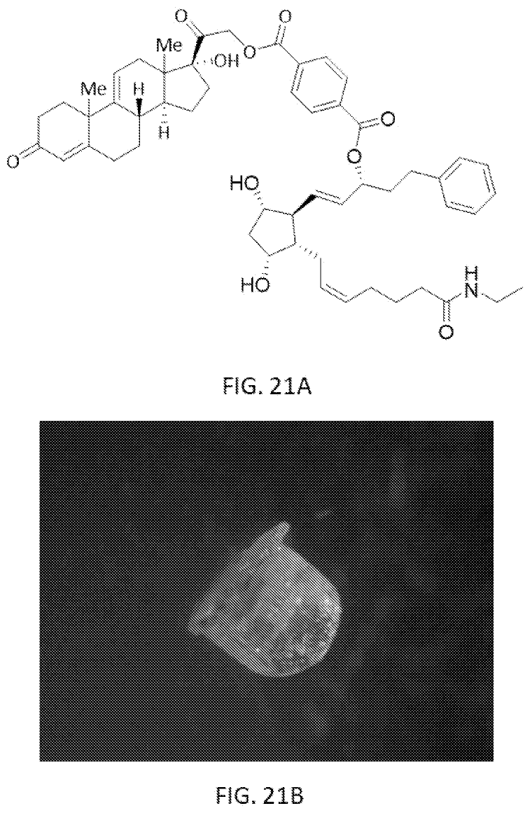

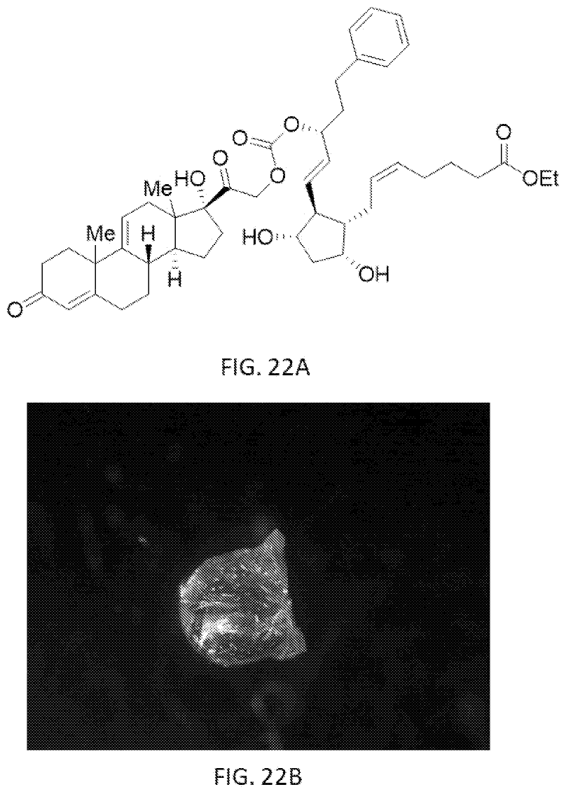

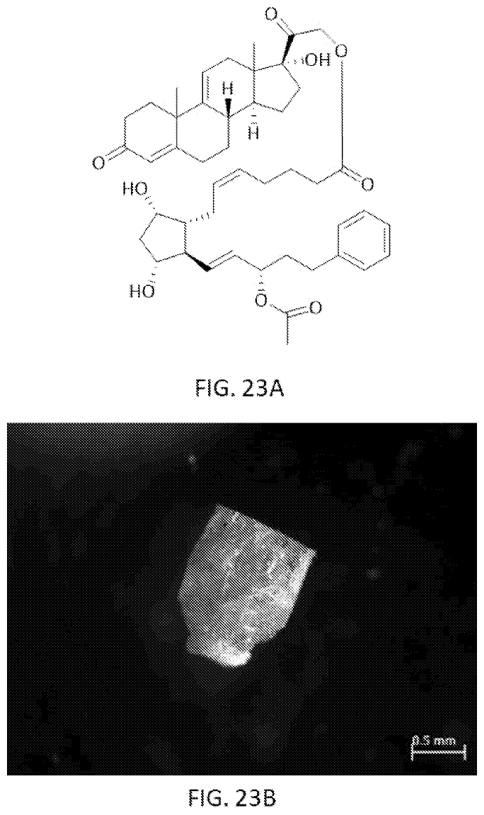

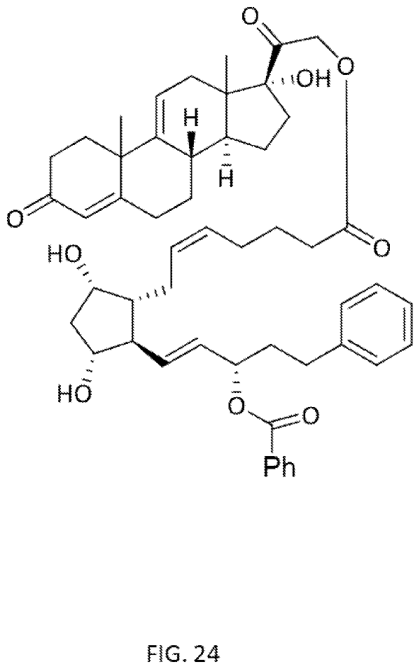

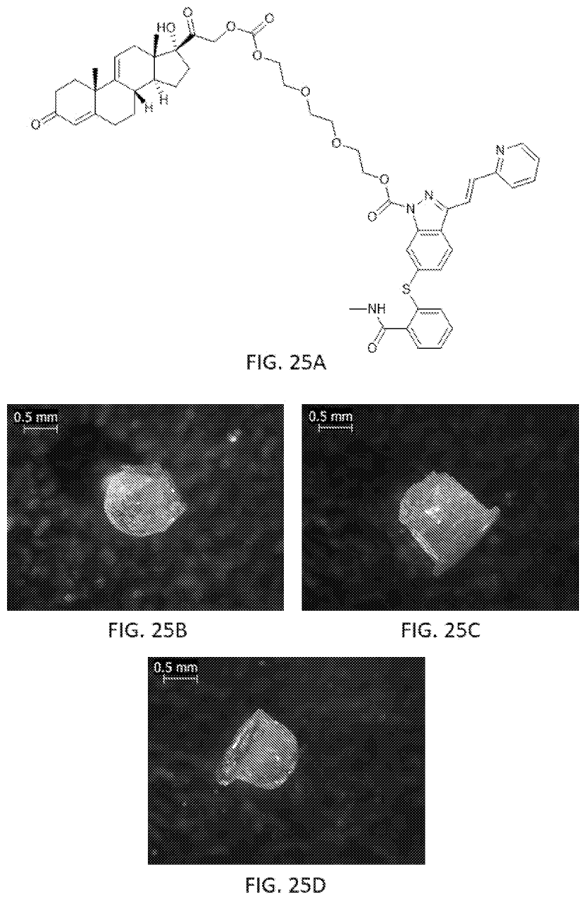

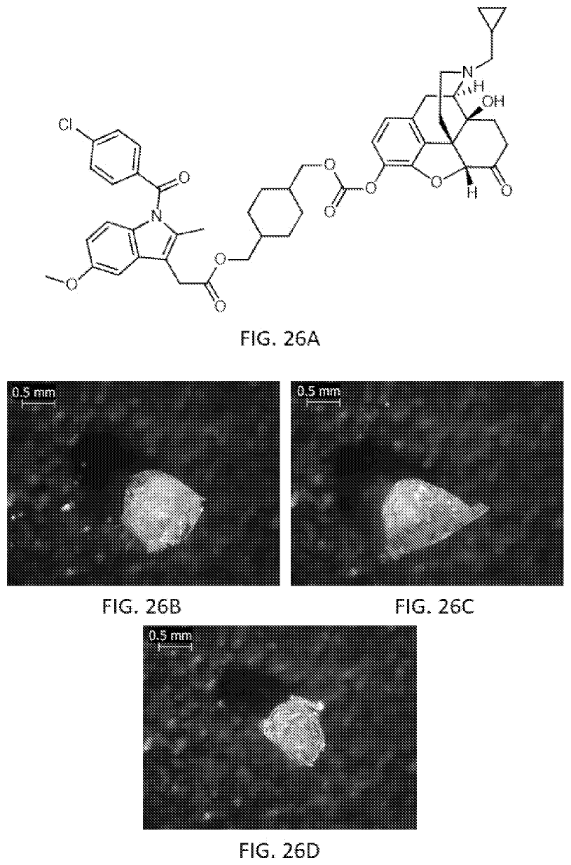

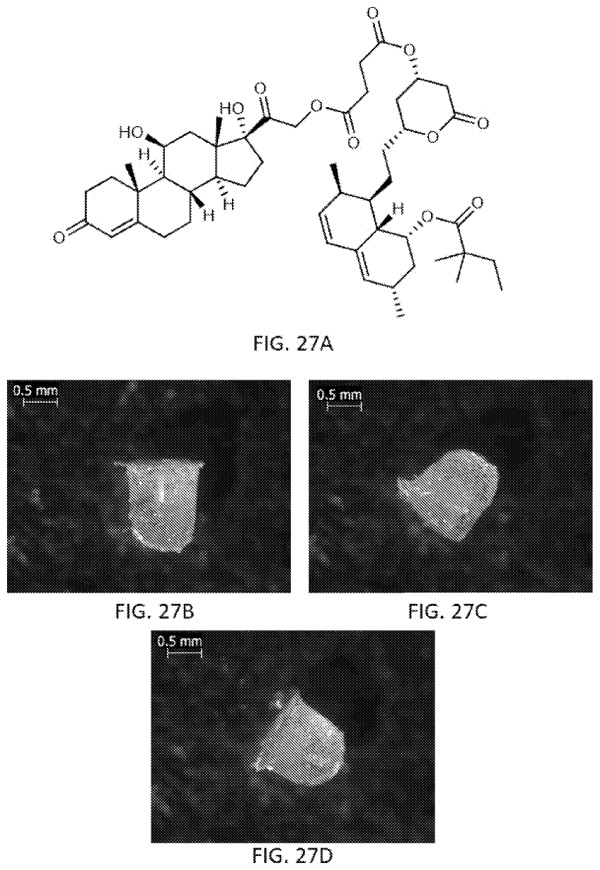

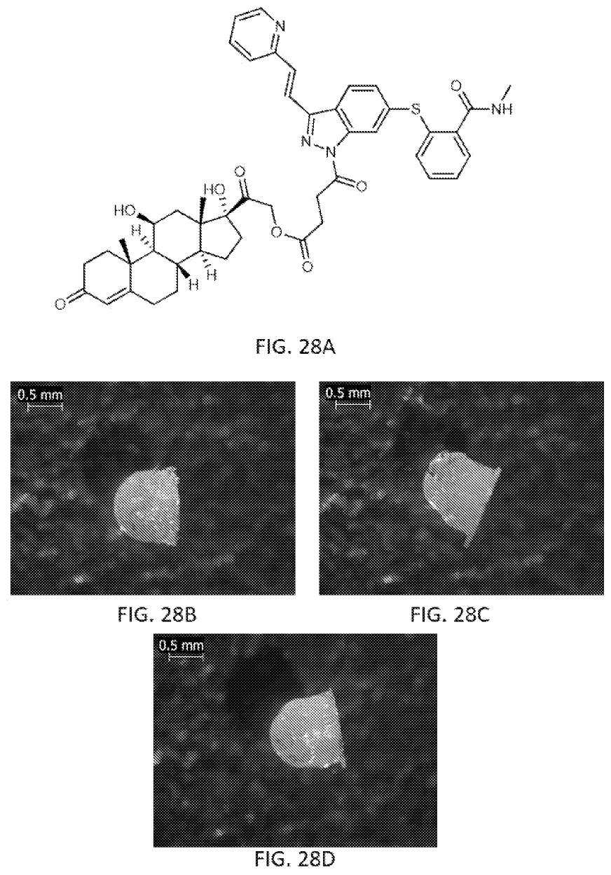

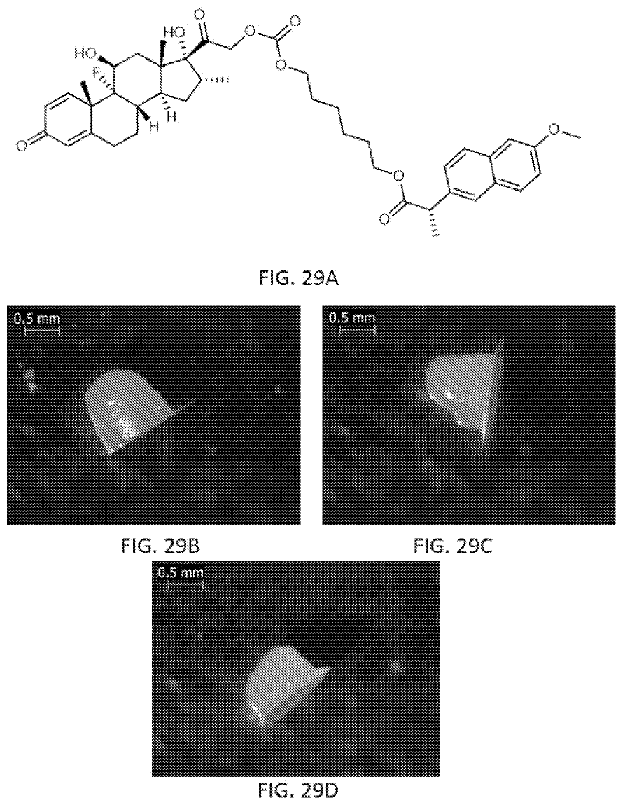

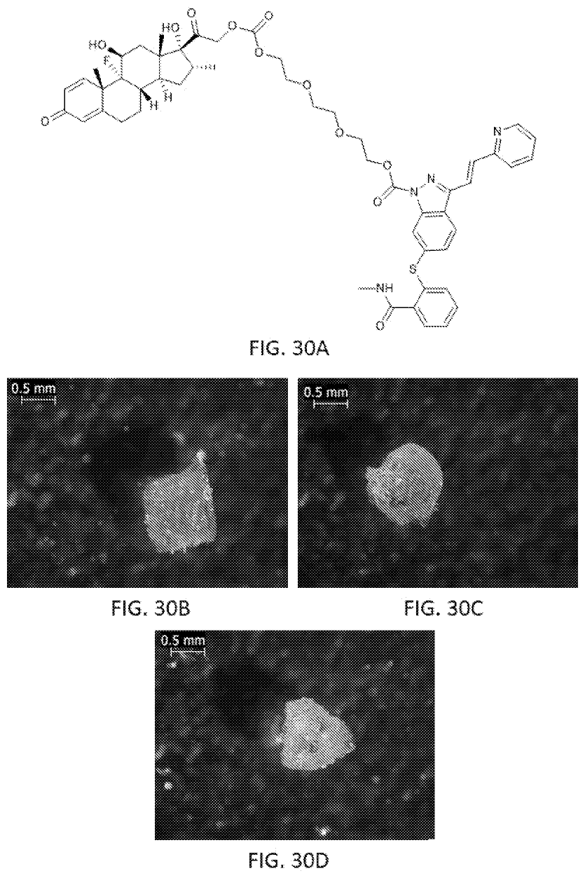

For example, FIG. 18 shows purity of compound 5 before (pre-sterilization) or post sterilization of Compound 5 in ethylene oxide, or by gamma-radiation or E-beam.

### Example 6: In Vivo Evaluation

### Biological Example 1: Implantation of Extruded Rod in Rabbit Eye

A compound provided herein (e.g., Compound 5) was formed into rods by melt extrusion and were cut to 1, 1.5 or 2 mm length. The resulting implants were loaded in the lumen of needles, terminally sterilized, and injected into the anterior chamber of rabbits. Implants settled into the inferior iridocorneal angle and were visualized by anterior chamber optical coherence tomography.

### Biological Example 2: Implantation of Extruded Rod in Rabbit Eye

A compound provided herein (e.g., Compound 6) was formed into rods by melt extrusion and were cut to 1.5 mm length. The resulting implants were loaded in the lumen of needles, terminally sterilized, and injected into the anterior chamber of rabbits. Implants settled into the inferior iridocorneal angle and were visualized by anterior chamber optical coherence tomography.

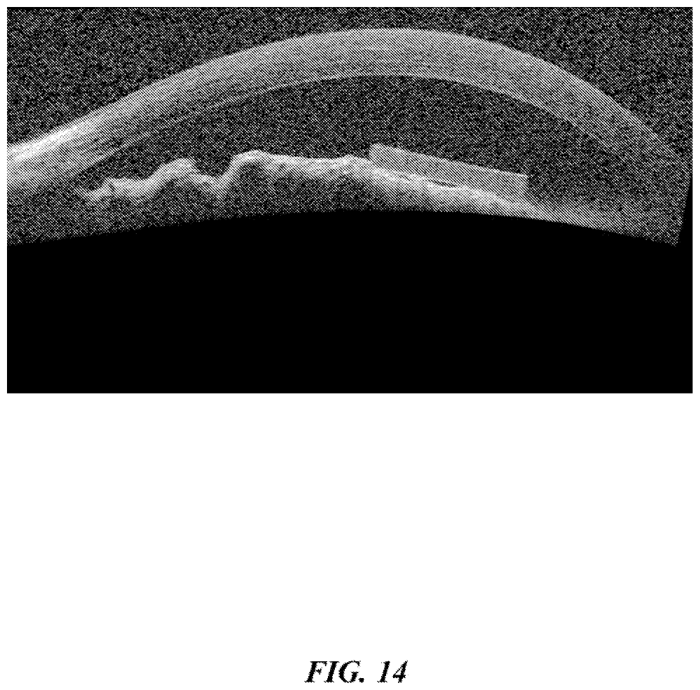

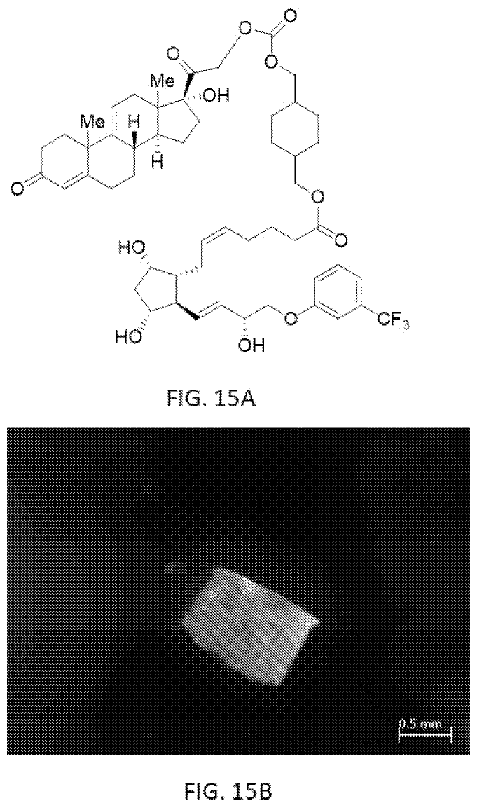

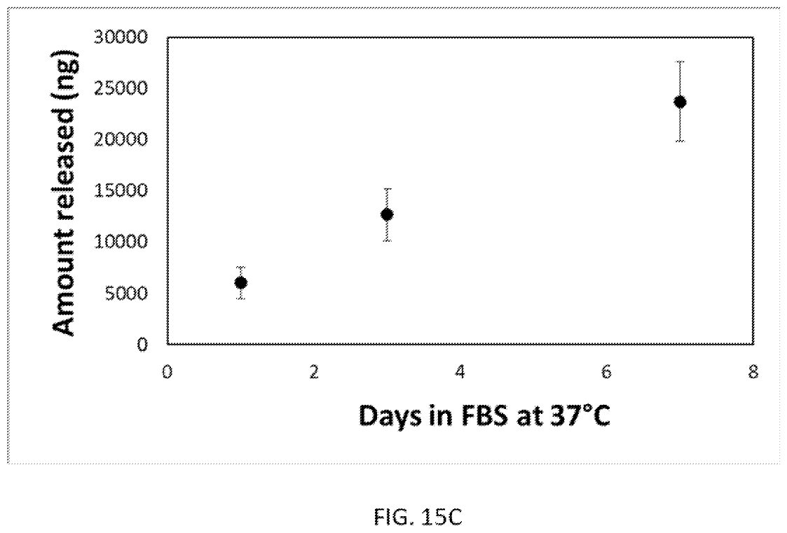

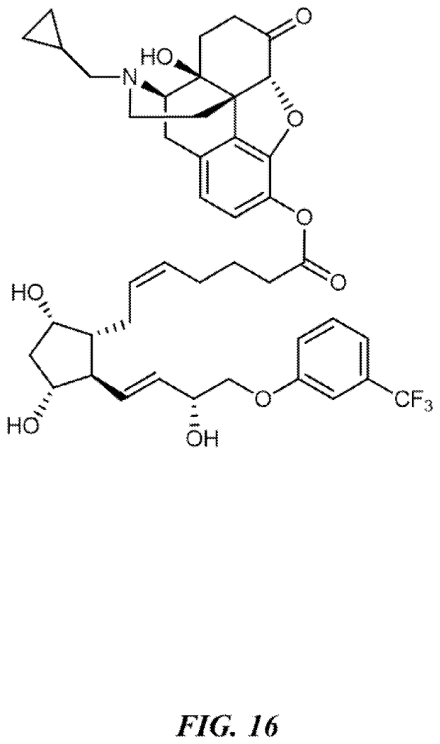

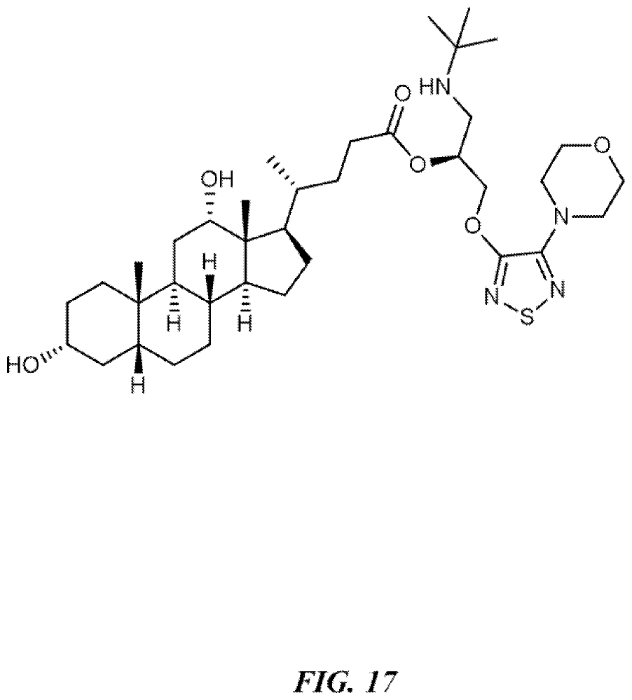

For example, FIG. 14 shows an extruded rod steroid-prostaglandin heterodimer (bimatoprost-anecortave, Compound 5) exemplified herein in a rabbit eye.

We claim:

1. A compound comprising a first radical and a second radical, the first radical has a structure represented by Formula (I′): 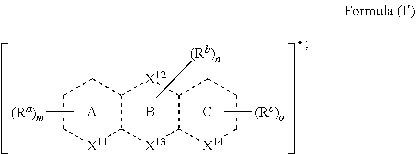
wherein: [symbol]
is a single bond or a double bond;
each R^{a}, R^{b}, and R^{c} are independently selected the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted;
or any one of R^{a}, R^{b}, or R^{c} are taken together with another of R^{a}, R^{b}, or R^{c} to form a substituted or an unsubstituted cycloalkyl or heterocycloalkyl;
X¹¹, X¹², X¹³, and X¹⁴ are each independently selected from the group consisting of a bond and Q_{y}, wherein each Q is independently selected from the group consisting of —O—, —NR—, —S(R)ₓ—, and —C(R)_{z}—;
each x is independently 0-5;
each y is independently 1-3;
each z is independently 1 or 2;
each R is independently selected from the group consisting of H, halogen, alkyl, heteroalkyl, cycloalkyl, heterocycloalkyl, alkoxy, amino, hydroxy, and thiol, or is taken together with another R to form an oxo; and
each of m, n, and o are independently 0-6;
the second radical being a therapeutically active agent (or drug) (e.g., in its free form) and the first radical (e.g., a radical of a carrier) being different than the second radical;
the first radical and the second radical being attached to a linker (e.g., that links the first radical and the second radical);
wherein, either the first radical, the second radical, or both the first radical and the second radical is not a steroid,
or a pharmaceutically-acceptable salt or solvate thereof.

2. The compound of claim 1, wherein the linker (e.g., L) is not a bond.

3. A compound having a structure represented by Formula (I):
D1-L-D2 Formula (I);
wherein:
D1 is a radical of a carrier (e.g., a radical of an inactive agent (e.g., a non-medicinal agent (e.g., an agent that (in its free form) does not provide a (e.g., significant, measurable, and/or direct) therapeutic effect and/or benefit (e.g., to an individual (e.g., in need thereof)));
D2 is a radical of an active agent (e.g., a therapeutically active agent) (or drug);
L is -(Q¹-M-Q²)-;
Q¹ and Q² are each independently absent or (C═X¹)X²;
X¹ is O or S;
X² is O, S, or NR¹;
R¹ is hydrogen or C₁-C₆ alkyl; and
M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl),
wherein, either the first radical, the second radical, or both the first radical and the second radical is not a steroid,
or a pharmaceutically-acceptable salt or solvate thereof.

4. The compound of any one of the preceding claims, wherein the first radical or D1 is a radical of a natural compound (e.g., naturally occurring in nature or in the body).

5. The compound of any one of the preceding claims, wherein the first radical or D1 comprises a three-ring core structure (e.g., having a structure represented by Formula (I′)), wherein the three-ring core structure is a processable carrier group).

6. The compound of any one of the preceding claims, wherein the first radical or D1 and the second radical or D2 comprises a three-ring core structure (e.g., having a structure represented by Formula (I′))).

7. The compound of any one of the preceding claims, wherein the first radical or D1 is a radical of any compound provided in Table 1 or Table 2.

8. The compound of any one of the preceding claims, wherein the first radical or D1 is a radical of a steroid (e.g., an anti-inflammatory steroid (e.g., dexamethasone, hydrocortisone, or triamcinolone), an angiostatic steroid (e.g., anecortave), or a benign steroid (e.g., cholesterol, cholic acid, or deoxycholic acid)).

9. The compound of any one of the preceding claims, wherein the first radical or D1 is a radical of a compound selected from the group consisting of ursolic acid, amyrin, isoarborinol, boswellic acid, chamaecydin, cucurbalsaminol A, cycloartenol, lanosterol, dichapetalin, oleanolic acid, lepidolide, panaxatriol, riboflavin (vitamin B2), santonic acid, tetrahydrocannabiorcol, plicadin, annonamine, boldine, pukatiene, and (−)-stepholidine.

10. The compound of any one of the preceding claims, wherein the second radical or D2 is a radical of: an angiotensin-converting-enzyme (ACE) inhibitor (e.g., enalapril, captopril, cilazapril, fosinopril, lisinopril, moexipril, perindopril, quinapril, ramipril, trandolapril, zofenopril, or the like), an immunosuppressant (e.g., everolimus, tacrolimus, or the like), an angiotensin II receptor blocker (e.g., candesartan, eprosartan, irbesartan, losartan, olmesartan, telmisartan, valsartan, azilsartan, or the like), an atypical antipsychotic (e.g., paliperidone or the like), a human immunodeficiency virus (HIV) integrase inhibitor (e.g., dolutegravir or the like), a tyrosine kinase inhibitor (e.g., axitinib or the like), a beta-3-adrenergic agonist (e.g., mirabegron or the like), a 1,3-benzoxazole (e.g., tafamidis or the like), a statin (rosuvastatin, simvastatin, or the like), a vasopressin receptor antagonist (e.g., tolvaptan or the like), an antineoplastic (e.g., belzutifan, dichapetalin, or the like), a dopamine agonist (e.g., rotigotine or the like), an acetyl cholinesterase inhibitor (e.g., donepezil, galantamine, or the like), an anti-epileptic (e.g., valproic acid or the like), an opioid agonist (e.g., oxycodone, hydrocodone, or the like), an opioid antagonist (e.g., naltrexone, naloxone, nalmefene, or the like), an antimuscarinic (e.g., fesoterodine, tolterodine, or the like), an anticholinergic (e.g., darifenacin or the like), a vasodilator (e.g., treprostinil or the like), an anti-arrhythmic (e.g., propafenone or the like), an nonsteroidal anti-inflammatory drug (NSAID) (e.g., naproxen, bromfenac, celecoxib, indomethacin, or the like), an antidepressant (e.g., paroxetine, bupropion, noritriptyline, or the like), a vitamin (e.g., a vitamin D₃ (e.g., calcifediol or the like)), an antihistamine (e.g., cetirizine, desloratadine, or the like), a triterpenoid topoisomerase inhibitor (e.g., betulinic acid or the like), a triterpenoid (e.g., acetoxolone or the like), a mineralocorticoid (e.g., fludrocortisone or the like), nicotinic acetylcholine receptor agonist (e.g., varenicline or the like), a fatty acid synthase (FAS) inhibitor (e.g., bicycol or the like), a carbonic anhydrase inhibitor (e.g., dorzolamide or the like), an alpha-adrenergic agonist (e.g., brimonidine), a sympathomimetic (e.g., epinephrine), a Rho-associated kinase (ROCK) inhibitor (e.g., fasudil, hydroxyfasudil, ripasudil, netarsudil, belumosudil, verosudil, or thiazovivin), a prostaglandin (e.g., latanoprost, latanoprost acid, travoprost, travoprost acid, tafluprost, tafluprost acid, bimatoprost, or bimatoprost acid), or any combination thereof.

11. The compound of any one of the preceding claims, wherein the second radical or D2 is a radical of: a nonsteroidal anti-inflammatory drug (NSAID) (e.g., naproxen, bromfenac, celecoxib, indomethacin, or the like), a tyrosine kinase inhibitor (e.g., axitinib or the like), a statin (e.g., rosuvastatin, simvastatin, or the like), an antineoplastic (e.g., belzutifan, dichapetalin, fluorouracil, or the like), or any combination thereof.

12. The compound of any one of the preceding claims, wherein the second radical or D2 is a radical of a steroid (e.g., an anti-inflammatory steroid (e.g., dexamethasone, hydrocortisone, or triamcinolone) or an angiostatic steroid (e.g., anecortave)).

13. The compound of any one of the preceding claims, wherein the second radical or D2 is a radical of a prostaglandin (e.g., latanoprost, latanoprost acid, travoprost, travoprost acid, tafluprost, tafluprost acid, bimatoprost, or bimatoprost acid).

14. The compound of any one of the preceding claims, wherein the second radical or D2 is a radical of any therapeutic agent (or drug) provided herein (e.g., any therapeutic agent (or drug) provided in Table 2).

15. The compound of any one of the preceding claims, wherein the linker (e.g., L) is or comprises (e.g., a diradical (e.g., a molecular species (e.g., an organic compound) with two electrons occupying degenerate molecular orbitals) of) one or more linker groups, each linker group being independently selected from any linker or linker group provided herein (e.g., any linker or linker group provided in Table 3).

16. The compound of any one of the preceding claims, wherein the first radical or D1 is a radical of any carrier provided herein (e.g., any carrier provided in Table 1 or Table 2), the second radical or D2 is a radical of any therapeutic agent (or drug) provided herein (e.g., any therapeutic agent (or drug) provided in Table 2), and the linker (e.g., L) is or comprises (e.g., a diradical (e.g., a molecular species (e.g., an organic compound) with two electrons occupying degenerate molecular orbitals) of) one or more linker groups, each linker group being independently selected from any linker or linker group provided herein (e.g., any linker or linker group provided in Table 3).

17. A compound comprising a structure of Formula (IE): 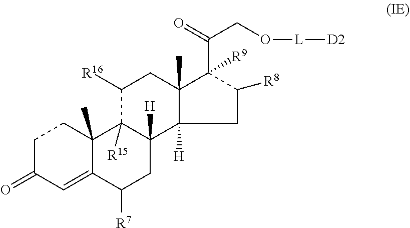
wherein: [symbol]
is a single bond or a double bond;
R⁷ is hydrogen or halogen;
R⁸ is hydrogen or C₁-C₄ alkyl;
R⁹ is absent, hydrogen, or hydroxyl;
R¹⁵ is absent, hydrogen, or halogen;
R¹⁶ is hydrogen or hydroxyl;
D2 is a radical of an active agent (e.g., a therapeutically active agent) (or drug);
L is -(Q¹-M-Q²)-;
Q¹ and Q² are each independently absent or (C═X¹)X²;
X¹ is O or S;
X² is O, S, or NR¹;
R¹ is hydrogen or C₁-C₆ alkyl; and
M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl);
wherein D2 is not a steroid;
or a pharmaceutically acceptable salt or solvate thereof.

18. The compound of any one of the preceding claims, wherein R⁷ is hydrogen.

19. The compound of any one of the preceding claims, wherein R⁸ is methyl.

20. The compound of any one of the preceding claims, wherein R⁹ is hydroxyl.

21. The compound of any one of the preceding claims, wherein R¹⁵ is fluoro.

22. The compound of any one of the preceding claims, wherein R¹⁶ is hydroxyl.

23. The compound of any one of claims 18-22, wherein the compound has a structure of Formula (IE-i):

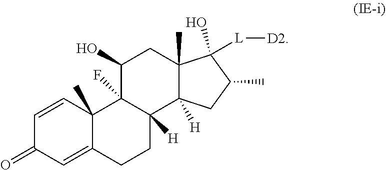

24. The compound of any one of claims 17, 18, or 20-22, wherein R⁸ is hydrogen.

25. The compound of any one of claims 17-20 or 22, wherein R¹⁵ is absent.

26. The compound of any one of claims 18-21, wherein R¹⁶ is hydrogen.

27. The compound of any one of claims 24-26, wherein the compound has a structure of Formula (IE-ii):

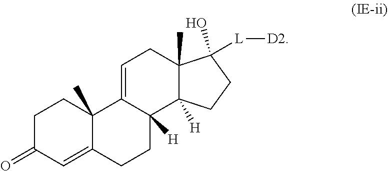

28. The compound of any one of claims 17-20, 22, 24, and 26, wherein R¹⁵ is hydrogen.

29. The compound of claim 28, wherein the compound has a structure of Formula (IE-iii):

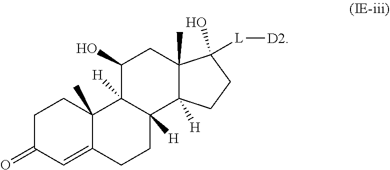

30. A compound comprising a first radical and a second radical, the first radical comprising a structure of Formula (IF): 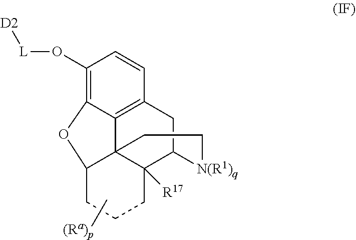
wherein: [symbol]
is a single bond or a double bond;
each R^{a} is independently selected the group consisting of hydrogen, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted;
or a first R^{a} is taken together with another R^{a} to form oxo, a substituted or an unsubstituted cycloalkyl or heterocycloalkyl;
each R¹ is independently H, alkyl, heteroalkyl, cycloalkyl, or heterocycloalkyl, wherein the alkyl, heteroalkyl, cycloalkyl, or heterocycloalkyl are optionally substituted;
R¹⁷ is H or —OH;
p is 0-8; and
q is 1 or 2;
D2 is a radical of an active agent (e.g., a therapeutically active agent) (or drug);
L is -(Q¹-M-Q²)-;
Q¹ and Q² are each independently absent or (C═X¹)X²;
X¹ is O or S;
X² is O, S, or NR¹;
R¹ is hydrogen or C₁-C₆ alkyl; and
M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl);
wherein D2 is not an opioid;
or a pharmaceutically acceptable salt or solvate thereof.

31. The compound of claim 30, wherein R¹ is substituted alkyl.

32. The compound according to claim 30 or 31, wherein R¹⁷ is hydroxyl.

33. The compound of any one of claims 30-32, wherein R^{a} is hydrogen.

34. The compound of any one of the preceding claims, wherein Q¹ and Q² are each independently absent, C═O, C=S, (C═O)O, (C═O)S, (C═O)N, or (C═S)S.

35. The compound of any one of the preceding claims, wherein Q¹ and Q² are each independently absent, C═O, or (C═O)O.

36. The compound of any one of the preceding claims, wherein M is substituted or unsubstituted alkyl (e.g., C₁-C₆ alkyl), substituted or unsubstituted heteroalkyl (e.g., C₁-C₆ heteroalkyl), or substituted or unsubstituted aryl.

37. The compound of any one of the preceding claims, wherein M is substituted or unsubstituted alkyl (e.g., C₁-C₆ alkyl (e.g., alkyl-carbocyclyl-alkyl)) or substituted or unsubstituted heteroalkyl (e.g., C₁-C₆ heteroalkyl).

38. The compound of any one of the preceding claims, wherein L is —(CH₂CH₂O)—, —(CH₂CH₂O)₂—(CH₂CH₂O)₃—, -methyl-cyclohexyl-methyl-, —CH₂—, —CH₂CH₂—, —CH₂CH₂CH₂—, —CH₂CH₂CH₂CH₂—, —CH₂CH₂CH₂CH₂CH₂—, or —CH₂CH₂CH₂CH₂CH₂CH₂—.

39. The compound of any one of the preceding claims, wherein the compound does not have the structure:

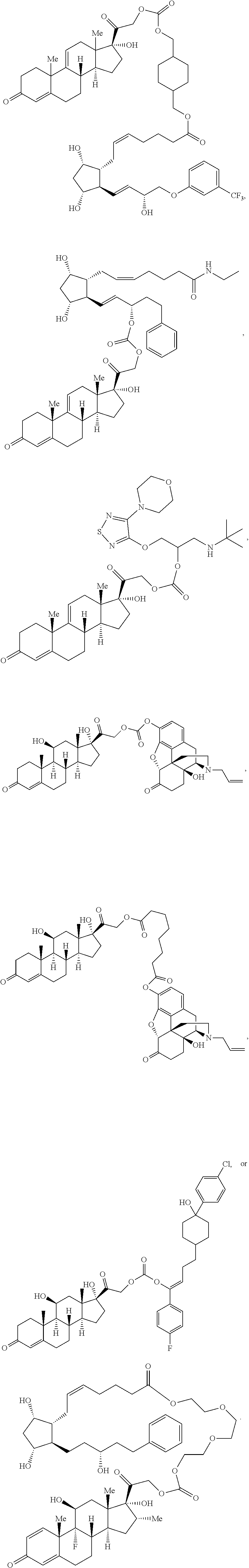

40. The compound of any one of claims 1-34, wherein the compound is:

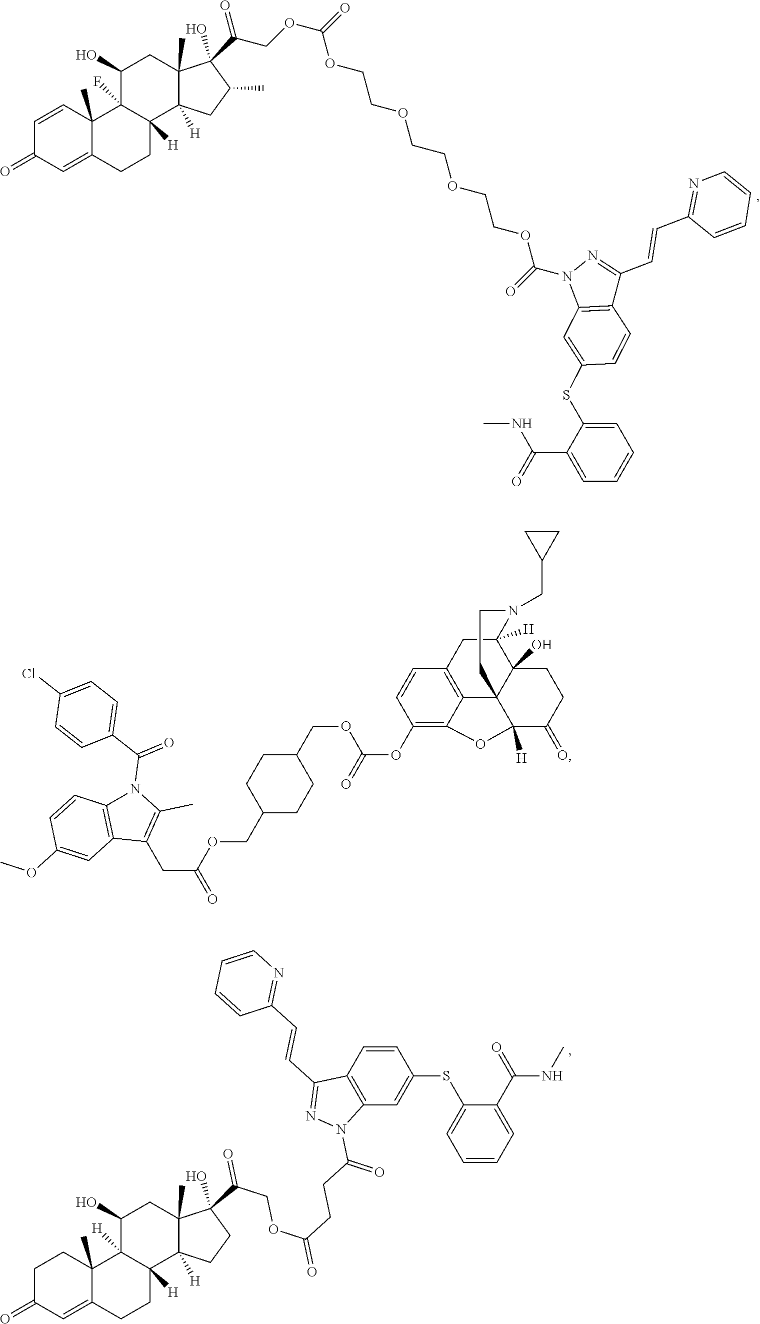 [SMILES: CNC(=O)c1ccccc1Sc1ccc2c(/C=C/c3ccccn3)nn(C(=O)OCCOCCOCCOC(=O)OCC(=O)[C@@]3(O)[C@H](C)C[C@H]4[C@@H]5CCC6=CC(=O)C=C[C@]6(C)[C@@]5(F)[C@@H](O)C[C@@]43C)c2c1 ; COc1ccc2c(c1)c(CC(=O)OCC1CCC(COC(=O)Oc3ccc4c5c3O[C@H]3C(=O)CC[C@@]6(O)[C@@H](C4)N(CC4CC4)CC[C@]536)CC1)c(C)n2C(=O)c1ccc(Cl)cc1 ; CNC(=O)c1ccccc1Sc1ccc2c(/C=C/c3ccccn3)nn(C(=O)CCC(=O)OCC(=O)[C@@]3(O)CC[C@H]4[C@@H]5CCC6=CC(=O)CC[C@]6(C)[C@H]5[C@@H](O)C[C@@]43C)c2c1]

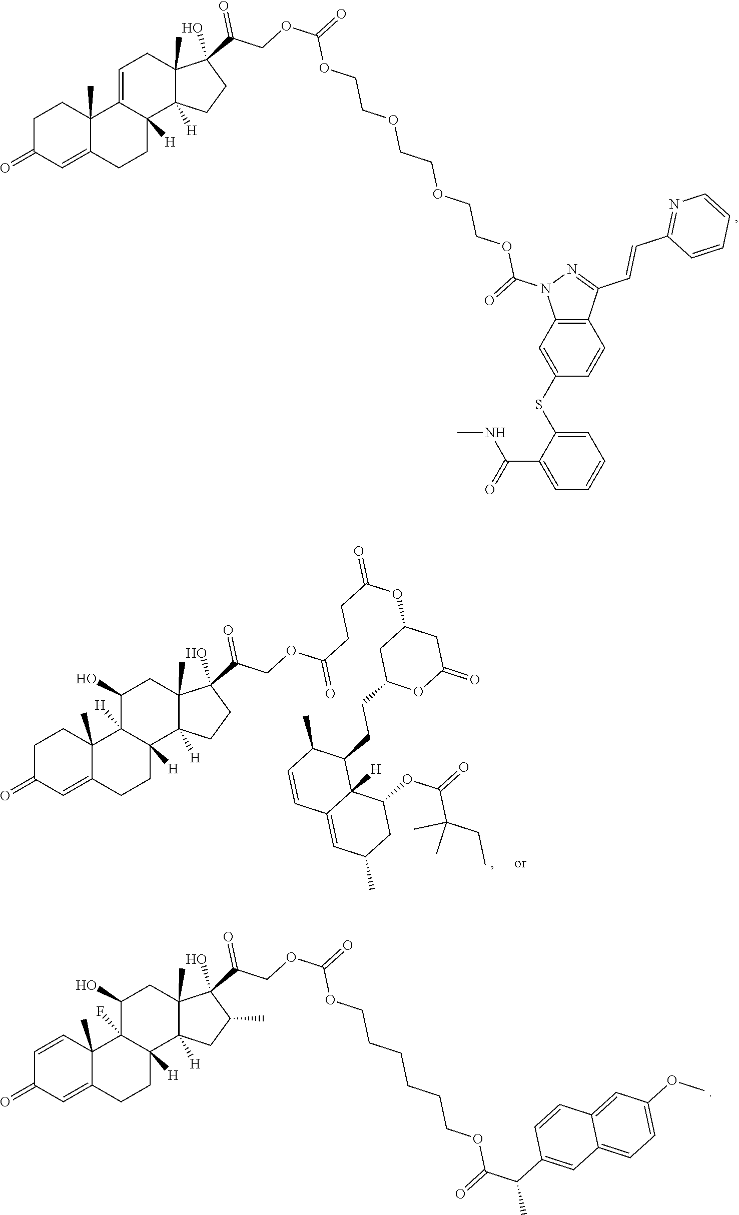 [SMILES: CNC(=O)c1ccccc1Sc1ccc2c(/C=C/c3ccccn3)nn(C(=O)OCCOCCOCCOC(=O)OCC(=O)[C@@]3(O)CC[C@H]4[C@@H]5CCC6=CC(=O)CC[C@]6(C)C5=CC[C@@]43C)c2c1 ; CCC(C)(C)C(=O)O[C@@H]1C[C@H](C)C=C2C=C[C@@H](C)[C@@H](CC[C@@H]3C[C@H](OC(=O)CCC(=O)OCC(=O)[C@@]4(O)CC[C@H]5[C@@H]6CCC7=CC(=O)CC[C@]7(C)[C@H]6[C@@H](O)C[C@@]54C)CC(=O)O3)[C@@H]21 ; COc1ccc2cc([C@H](C)C(=O)OCCCCCCOC(=O)OCC(=O)[C@@]3(O)[C@H](C)C[C@H]4[C@@H]5CCC6=CC(=O)C=C[C@]6(C)[C@@]5(F)[C@@H](O)C[C@@]43C)ccc2c1]

41. A compound having a structure represented by Formula (ID):
D1-L-D2 Formula (ID);
wherein:
D1 is a steroid radical;
D2 is a prostaglandin radical;
L is -(Q¹-M-Q²)-;
Q¹ and Q² are each independently absent or (C═X¹)X²;
X¹ is O or S;
X² is O, S, or NR¹;
R¹ is hydrogen or C₁-C₆ alkyl; and
M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl),
or a pharmaceutically-acceptable salt or solvate thereof.

42. The compound of claim 41, wherein D1 is angiostatic steroid (e.g., anecortave) or a benign steroid (e.g., cholesterol).

43. The compound according to claim 41 or 42, wherein D1 is a corticosteroid (e.g., glucocorticoid or mineralcorticoid), a sex steroid, a neurosteroid, an aminosteroid, or a secosteroid.

44. The compound of any one of the preceding claims, wherein D1 is anecortave (e.g., anecortave desacetate).

45. A compound having a structure represented by Formula (II): 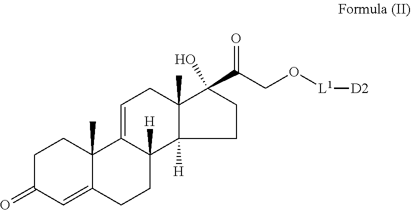
or a pharmaceutically acceptable salt or solvate thereof,
wherein,
D2 is a prostaglandin radical;
L¹ is -(Q¹-M-Q²)-;
Q¹ and Q² are each independently absent or (C═X¹)X²;
X¹ is O or S;
X² is O, S, or NR¹;
R¹ is hydrogen or C₁-C₆ alkyl; and
M comprises one or more linker group, each linker group being independently selected from the group consisting of substituted or unsubstituted alkyl (e.g., C₁—C₆ alkyl), substituted or unsubstituted aryl, substituted or unsubstituted heteroaryl, and substituted or unsubstituted heteroalkyl (e.g., C₁-C₆ heteroalkyl).

46. The compound of any one of the preceding claims, wherein D2 is selected from the group consisting of latanoprost, latanoprost acid, travoprost, travoprost acid, tafluprost, tafluprost acid, bimatoprost, bimatoprost acid, sepetaprost, sepetaprost acid, 7-[(1R,2R,3R,5S)-3,5-dihydroxy-2-[(3R)-3-hydroxy-5-phenylpentyl]cyclopentyl]-4-(3-thioxo-3H-1,2-dithiol-5-yl)phenyl ester, 5Z-heptenoic acid, latanoprostene bunod, and (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl 6-(nitrooxy)hexanoate, or a fragment or radical of any of the foregoing.

47. The compound of any one of the preceding claims, wherein D2 is a radical represented by a structure of Formula (III): 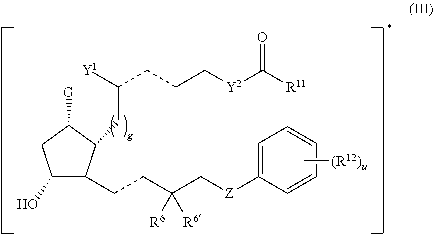
wherein:
each [symbol] is independently a single bond or a double bond;
G is OH;
Y¹ is hydrogen;
or G is taken together with Y¹ to form —O—CH₂—;
Y² is a bond or alkylene (e.g., —CH₂—);
g is 1 or 2;
Z is —O— or alkylene (e.g., —CH₂—);
R⁶ and R^{6′} are each independently hydrogen, halogen, alkyl, —OR¹⁴, or R⁶ and R^{6′} are taken together to form an oxo (e.g., R⁶ and R^{6′} are each independently hydrogen, halogen, or OH);
R¹⁴ is hydrogen, unsubstituted or substituted alkyl (e.g., substituted or unsubstituted C₁-C₆ alkyl (e.g., alkyl substituted with NO₂ or alkyl substituted with oxo and ONO₂)), or a point of attachment to the linker (e.g., L¹);
R¹¹ is —OR¹³ or —NR¹³R^{13b};
R¹³, R^{13a}, and R^{13b} are each independently selected from the group consisting of hydrogen, unsubstituted or substituted alkyl (e.g., substituted or unsubstituted C₁-C₆ alkyl (e.g., alkyl substituted with NO₂ or alkyl substituted with oxo and ONO₂)), substituted or unsubstituted aryl (e.g., aryl substituted with (cyclic) heteroalkyl (e.g., aryl substituted with 3H-1,2-dithiole-3-thione)), or a point of attachment to the linker (e.g., L¹);
each R¹² is independently halogen (e.g., fluoro or chloro) or alkyl (e.g., haloalkyl (e.g., CF₃)); and
u is 0-5.

48. The compound of claim 47, wherein G is OH.

49. The compound according to claim 47 or 48, wherein Y¹ is hydrogen.

50. The compound of any one of claims 47-49, wherein Y² is alkylene (e.g., —CH₂—).

51. The compound of any one of claims 47-50, wherein g is 1.

52. The compound of any one of claims 47-51, wherein G is OH, Y¹ is hydrogen, Y² is —CH₂—, and g is 1.

53. The compound of any one of claims 47-52, wherein R¹³ and R¹⁴ are each independently selected from the group consisting of hydrogen, unsubstituted or substituted alkyl (e.g., substituted or unsubstituted C₁-C₆ alkyl (e.g., alkyl substituted with NO₂ or alkyl substituted with oxo and ONO₂)), substituted or unsubstituted aryl (e.g., aryl substituted with (cyclic) heteroalkyl (e.g., aryl substituted with 3H-1,2-dithiole-3-thione)), or a point of attachment to the linker (e.g., L¹).

54. The compound of any one of claims 47-53, wherein R¹⁴, R¹³, R^{13a}, and R^{13b} are each independently selected from the group consisting of hydrogen, substituted or unsubstituted C₁-C₆ alkyl, or a point of attachment to the linker (e.g., L¹).

55. The compound of any one of the preceding claims, wherein D2 is a radical represented by a structure of Formula (III-A): 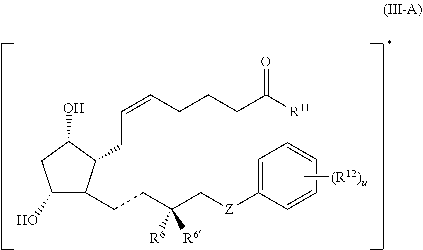
wherein: [symbol]
a single bond or a double bond;
Z is —O— or —CH₂—;
R⁶ and R^{6′} are each independently hydrogen, halogen, or —OR¹⁴;
R¹⁴ is hydrogen or a point of attachment to the linker (e.g., L¹);
R¹¹ is —OR¹³ or —NR^{13a}R^{13b};
R¹³, R^{13a}, and R^{13b} are each independently hydrogen, C₁-C₃ alkyl, or a point of attachment to the linker (e.g., L¹);
each R¹² is independently halogen (e.g., fluoro or chloro) or alkyl (e.g., haloalkyl (e.g., CF₃)); and
u is 0-5.

56. The compound of claim 47, wherein G together with Y¹ forms —O—CH₂—, and g is 2, and D2 is a radical represented by a structure of formula (III-B):

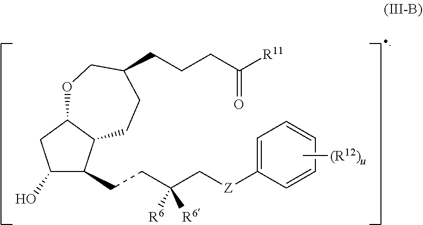

57. The compound of any one of claims 47-56, wherein Z is —O—.

58. The compound of any one of claims 47-56, wherein Z is —CH₂—.

59. The compound of any one of claims 47-58, wherein R¹² is CF₃ and u is 1.

60. The compound of any one of claims 47-58, wherein R¹² is F and u is 2.

61. The compound of any one of claims 47-58, wherein u is 0.

62. The compound of any one of claims 47-58, wherein u is 1.

63. The compound of any one of claims 47-58, wherein u is 2.

64. The compound of any one of claims 47-63, wherein R⁶ and R^{6′} are each independently fluoro.

65. The compound of any one of claims 47-63, wherein R⁶ is OH and R^{6′} is hydrogen.

66. The compound of any one of claims 47-65, wherein R¹ is OH or NH(C₁-C₃ alkyl).

67. The compound of any one of claims 47-65, wherein R¹¹ is —NHCH₂CH₃.

68. The compound of any one of claims 47-65, wherein R¹¹ is OH.

69. The compound of any one of claims 47-68, wherein R⁶ is —OR¹⁴ and R¹⁴ is a point of attachment to the linker (e.g., L¹), or R¹¹ is —OR¹³ and R¹³ is a point of attachment to the linker (e.g., L¹).

70. The compound of any one of claims 47-69, wherein R¹¹ is —OR¹³, R¹³ is a point of attachment to the linker (e.g., L¹), R⁶ is OH, and R^{6′} is hydrogen.

71. The compound of any one of claims 47-69, wherein R¹¹ is OH, R⁶ is —OR¹⁴, R¹⁴ is a point of attachment to the linker (e.g., L¹), and R^{6′} is hydrogen.

72. The compound of any one of the preceding claims, wherein D2 is latanoprost, bimatoprost, travoprost, or an acid or radical thereof.

73. The compound of any one of the preceding claims, wherein D2 is C1-latanoprost (radical), C15-latanoprost (radical), C1-bimatoprost (radical), C15-bimatoprost (radical), C1-travoprost (radical), or C15-travoprost (radical) (e.g., the carbon atom (e.g., C1 or C15) indicating which carbon atom the hydroxy radical (e.g., R⁶, R^{6′}, or R¹¹) is attached)).

74. The compound of any one of the preceding claims, wherein Q¹ and Q² are each independently absent, C═O, C=S, (C═O)O, (C═O)S, (C═O)N, or (C═S)S.

75. The compound of any one of the preceding claims, wherein Q¹ and Q² are each independently C═O or (C═O)O.

76. The compound of any one of the preceding claims, wherein Q¹ and Q² are each independently is C═O.

77. The compound of any one of the preceding claims, wherein Q¹ and Q² are C═O.

78. The compound of any one of the preceding claims, wherein Q¹ is C═O and Q² is absent.

79. The compound of any one of the preceding claims, wherein Q¹ is absent and Q² is C═O.

80. The compound of any one of the preceding claims, wherein Q¹ and Q² are (C═O)O.

81. The compound of any one of the preceding claims, wherein Q¹ is (C═O)O and Q² is absent.

82. The compound of any one of the preceding claims, wherein Q¹ is absent and Q² is (C═O)O.

83. The compound of any one of the preceding claims, wherein Q¹ is C═O and Q² is (C═O)S.

84. The compound of any one of the preceding claims, wherein Q¹ is (C═O)S and Q² is C═O.

85. The compound of any one of the preceding claims, wherein Q¹ is C═O and Q² is (C═S)S.

86. The compound of any one of the preceding claims, wherein Q¹ is (C═S)S and Q² is C═O.

87. The compound of any one of the preceding claims, wherein Q¹ is C═O and Q² is (C═O)N.

88. The compound of any one of the preceding claims, wherein Q¹ is (C═O)N and Q² is C═O.

89. The compound of any one of the preceding claims, wherein M is substituted or unsubstituted alkyl (e.g., C₁-C₆ alkyl), substituted or unsubstituted heteroalkyl (e.g., C₁-C₆ heteroalkyl), or substituted or unsubstituted aryl.

90. The compound of any one of the preceding claims, wherein M is substituted or unsubstituted alkyl (e.g., C₁-C₆ alkyl).

91. The compound of any one of the preceding claims, wherein M is substituted alkyl (e.g., C₁-C₆ alkyl), the alkyl being substituted with one or more substituent, each substituent being independently selected from the group consisting of oxo, halo, alkyl, and heteroalkyl (e.g., —NHCOCH₃).

92. The compound of any one of the preceding claims, wherein M is alkyl (e.g., C₁-C₆ alkyl) substituted with oxo.

93. The compound of any one of the preceding claims, wherein M is alkyl (e.g., C₁-C₆ alkyl) substituted with one or more —NHCOCH₃.

94. The compound of any one of the preceding claims, wherein M is —CH(NHCOCH₃)CH₂—.

95. The compound of any one of the preceding claims, wherein M is unsubstituted alkyl (e.g., C₁-C₆ alkyl).

96. The compound of any one of the preceding claims, wherein M is —(CH₂)ₘ—, m being 1-10.

97. The compound of any one of the preceding claims, wherein M is —CH₂—, —CH₂CH₂—, or —CH₂CH₂CH₂—.

98. The compound of any one of the preceding claims, wherein M is substituted or unsubstituted heteroalkyl (e.g., C₁-C₆ heteroalkyl).

99. The compound of any one of the preceding claims, wherein M is unsubstituted heteroalkyl (e.g., C₁-C₆ heteroalkyl).

100. The compound of any one of the preceding claims, wherein M is —SCH₂CH₂—.

101. The compound of any one of the preceding claims, wherein M is substituted or unsubstituted aryl.

102. The compound of any one of the preceding claims, wherein M is unsubstituted aryl (e.g., unsubstituted phenyl).

103. The compound of any one of the preceding claims, wherein M is alkyl (e.g., methyl) substituted with oxo and Q¹ and Q² are absent.

104. The compound of any one of the preceding claims, wherein:
Q¹ and Q² are (C═O)O; and
M is —CH₂CH₂CH₂—.

105. The compound of any one of the preceding claims, wherein:
Q¹ and Q² are C═O; and
M is —CH₂CH₂— or phenyl.

106. The compound of any one of the preceding claims, wherein:
Q¹ is C═O and Q² is absent; and
M is —CH₂—, —CH₂CH₂—, —CHCH₃—, or —CH(NHCOCH₃)CH₂—.

107. The compound of any one of the preceding claims, wherein:
Q¹ is C═O and Q² is absent; and
M is —SCH₂CH₂—.

108. The compound of any one of the preceding claims, wherein:
Q¹ is C═O and Q² is S(C═O); and
M is —CH₂CH₂—.

109. The compound of any one of the preceding claims, wherein:
Q¹ is C═O and Q² is S(C═S); and
M is —CH₂CH₂—.

110. The compound of any one of the preceding claims, wherein:
Q¹ is C═O and Q² is N(C═O); and
M is —CH₂CH₂—.

111. The compound of any one of the preceding claims, wherein the linker (e.g., L or L¹) is C═O, —C═OCH₂CH₂C═O—, —C═OphenylC═O—, —C═OCH₂CH₂—, —(C═O)OCH₂CH₂CH₂O(C═O)—, —C═OCH(NHCOCH₃)CH₂—, —C═OCH₂O(C═O)—, —(C═O)OCH₂C═O—, —C═OCH(CH₃)O(C═O)—, —(C═O)OCH(CH₃)C═O—, —C═OCH₂CH₂S—, —C═OCH₂CH₂SC═O—, —C═OCH₂CH₂SC═S—, or —C═OCH₂CH₂NHC═O—.

112. The compound of any one of the preceding claims, wherein the linker (e.g., L or L¹) is —C═OCH₂CH₂C═O—, —C═OphenylC═O—, —C═OCH₂CH₂—, —(C═O)OCH₂CH₂CH₂O(C═O)—, —C═OCH(NHCOCH₃)CH₂—, —C═OCH₂O(C═O)—, —(C═O)OCH₂C═O—, —C═OCH(CH₃)O(C═O)—, —(C═O)OCH(CH₃)C═O—, —C═OCH₂CH₂S—, —C═OCH₂CH₂SC═O—, —C═OCH₂CH₂SC═S—, or —C═OCH₂CH₂NHC═O—.

113. The compound of claim 112, wherein the linker (e.g., L or L¹) is C═O.

114. The compound of any one of the preceding claims, wherein the compound does not have the structure:

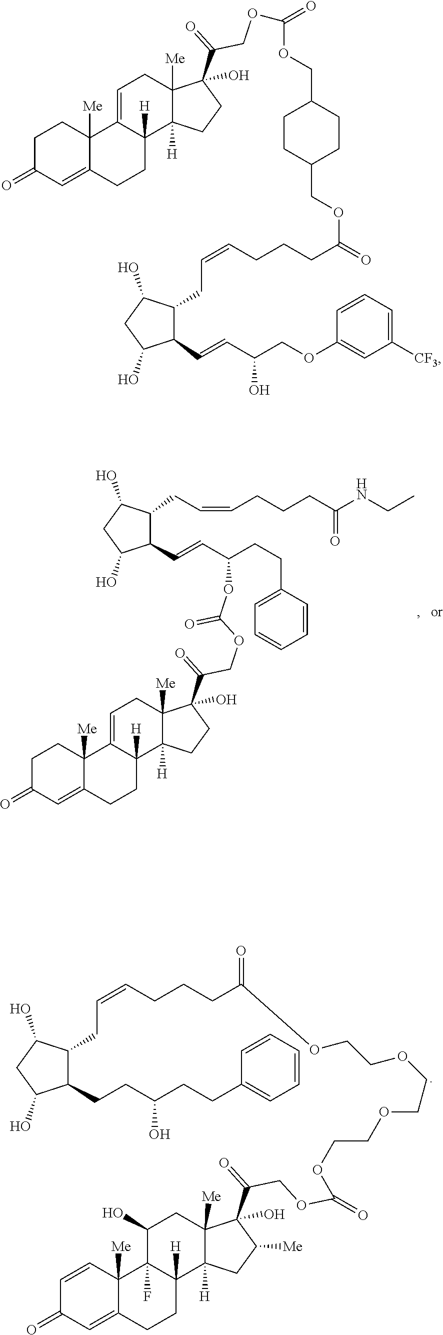

115. The compound of any one of the preceding claims, wherein the compound is represented by the structure: 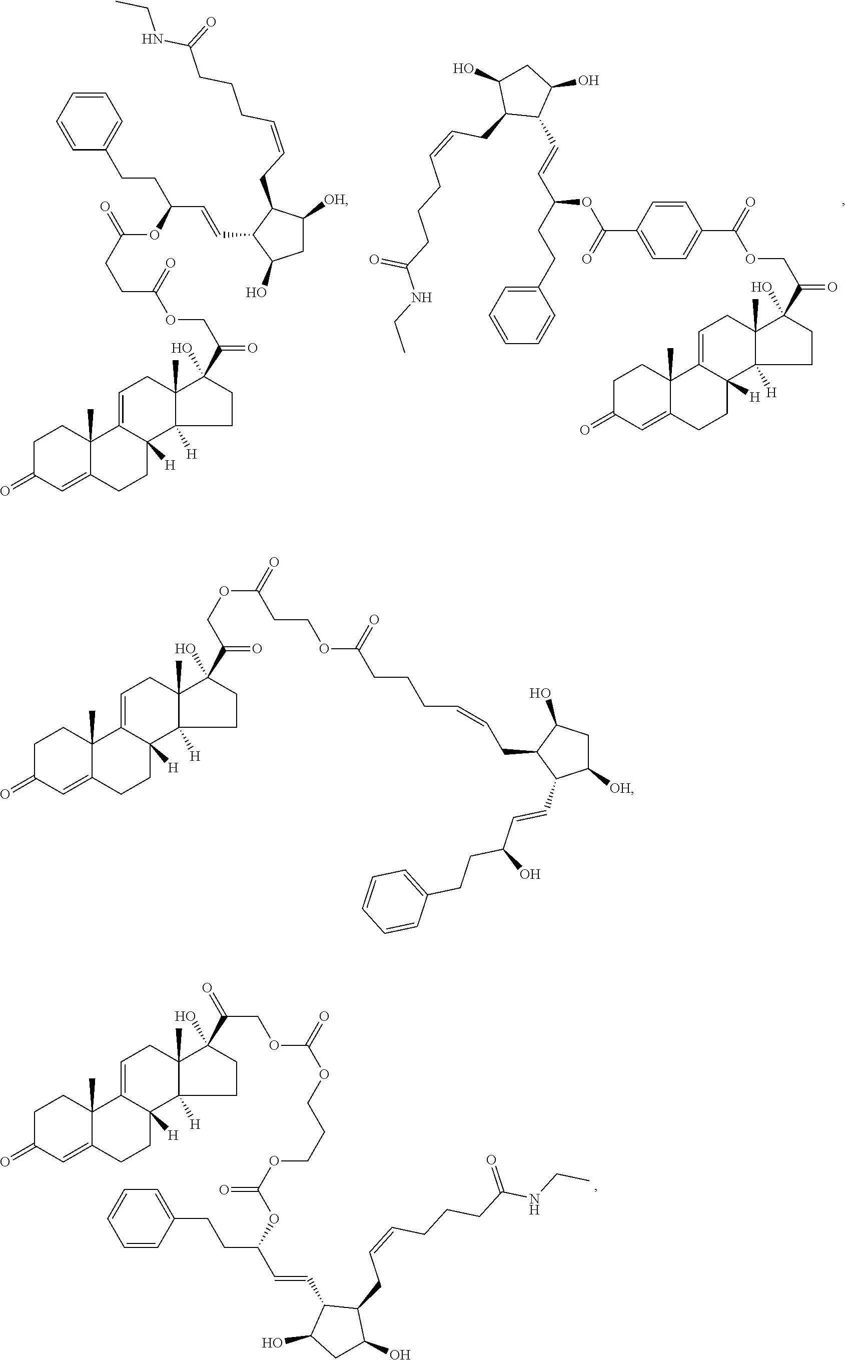 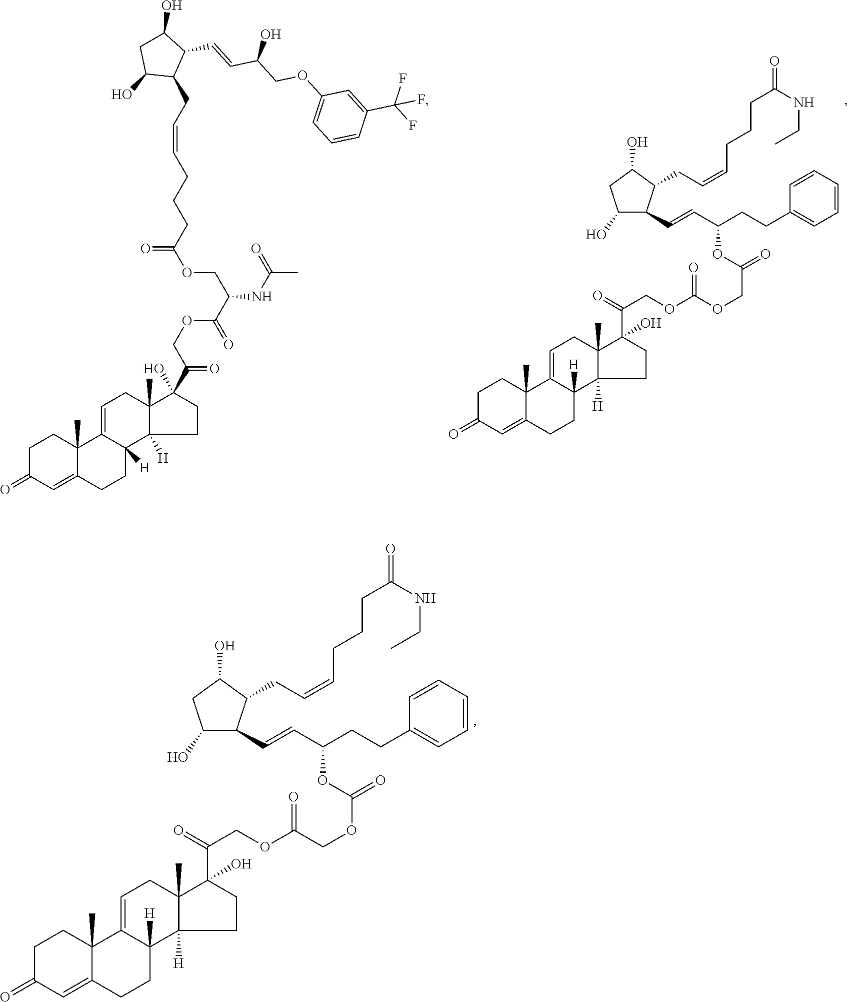 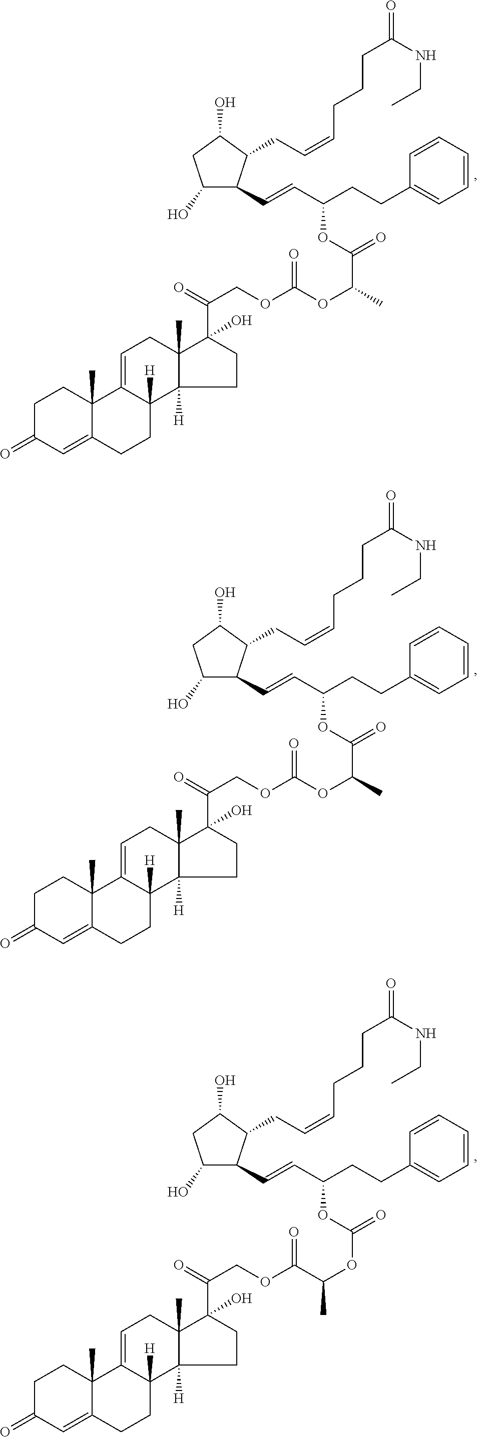 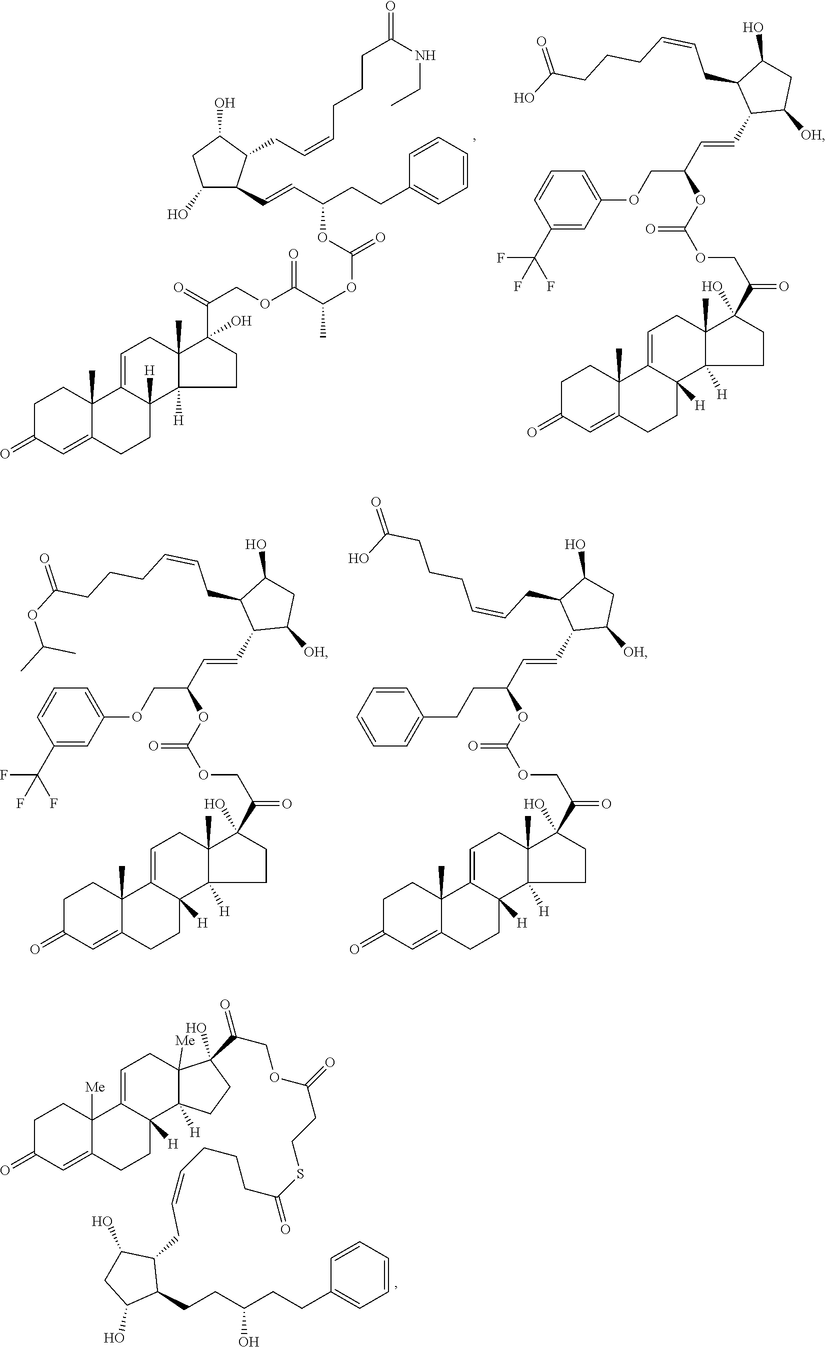 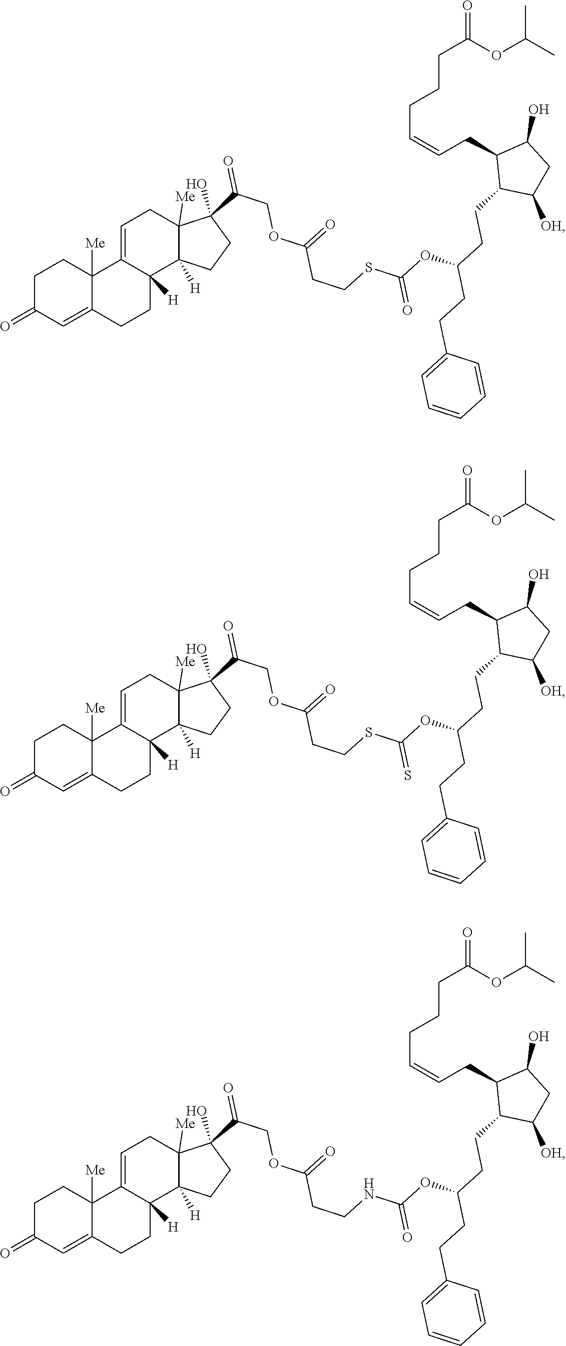 [SMILES: CC(C)OC(=O)CCC/C=C\C[C@@H]1[C@@H](CC[C@H](CCc2ccccc2)OC(=O)SCCC(=O)OCC(=O)[C@@]2(O)CC[C@H]3[C@@H]4CCC5=CC(=O)CCC5(C)C4=CCC32C)[C@H](O)C[C@@H]1O ; CC(C)OC(=O)CCC/C=C\C[C@@H]1[C@@H](CC[C@H](CCc2ccccc2)OC(=S)SCCC(=O)OCC(=O)[C@@]2(O)CC[C@H]3[C@@H]4CCC5=CC(=O)CCC5(C)C4=CCC32C)[C@H](O)C[C@@H]1O ; CC(C)OC(=O)CCC/C=C\C[C@@H]1[C@@H](CC[C@H](CCc2ccccc2)OC(=O)NCCC(=O)OCC(=O)[C@@]2(O)CC[C@H]3[C@@H]4CCC5=CC(=O)CCC5(C)C4=CCC32C)[C@H](O)C[C@@H]1O]
or a pharmaceutically acceptable salt or solvate thereof.

116. A compound having a structure represented by Formula (IV):
D1-L²-D2 Formula (IV);
wherein:
D1 is a steroid radical;
D2 is a substituted prostaglandin radical; and
L² is a linker,
or a pharmaceutically-acceptable salt or solvate thereof.

117. The compound of claim 116, wherein the L² is a bond.

118. The compound of any one of the preceding claims, wherein D1 is anecortave (e.g., anecortave desacetate).

119. The compound of any one of the preceding claims, wherein D1 is a corticosteroid (e.g., glucocorticoid or mineralcorticoid), a sex steroid, a neurosteroid, an aminosteroid, or a secosteroid.

120. The compound of any one of the preceding claims, wherein D2 is selected from the group consisting of substituted latanoprost, substituted latanoprost acid, substituted travoprost, substituted travoprost acid, substituted tafluprost, substituted tafluprost acid, substituted bimatoprost, substituted bimatoprost acid, substituted sepetaprost, substituted sepetaprost acid, substituted 7-[(1R,2R,3R,5S)-3,5-dihydroxy-2-[(3R)-3-hydroxy-5-phenylpentyl]cyclopentyl]-4-(3-thioxo-3H-1,2-dithiol-5-yl)phenyl ester, 5Z-heptenoic acid, substituted latanoprostene bunod, and substituted (S,E)-1-((1R,2R,3S,5R)-2-((Z)-7-(ethylamino)-7-oxohept-2-en-1-yl)-3,5-dihydroxycyclopentyl)-5-phenylpent-1-en-3-yl 6-(nitrooxy)hexanoate, or a fragment or radical of any of the foregoing.

121. The compound of any one of the preceding claims, wherein D2 is substituted latanoprost (e.g., latanoprost substituted with substituted alkyl (e.g., alkyl substituted with one or more alkyl substituent, each alkyl substituent being independently selected from the group consisting of oxo and aryl)), substituted bimatoprost (e.g., bimatoprost substituted with substituted alkyl (e.g., alkyl substituted with one or more alkyl substituent, each alkyl substituent being independently selected from the group consisting of oxo and aryl)), substituted travoprost (e.g., travoprost substituted with substituted alkyl (e.g., alkyl substituted with one or more alkyl substituent, each alkyl substituent being independently selected from the group consisting of oxo and aryl)), or an acid or radical thereof.

122. The compound of any one of the preceding claims, wherein D2 is substituted C1-latanoprost (radical), substituted C15-latanoprost (radical), substituted C1-bimatoprost (radical), substituted C15-bimatoprost (radical), substituted C1-travoprost (radical), or substituted C15-travoprost (radical) (e.g., the carbon atom (e.g., C1 or C15) indicating which carbon atom the hydroxy radical (e.g., R⁶, R^{6′}, or R¹¹) is attached)).

123. The compound of any one of the preceding claims, wherein D2 is C1-bimatoprost (radical) substituted at C15 with substituted alkyl (e.g., alkyl substituted with one or more alkyl substituent, each alkyl substituent being independently selected from the group consisting of oxo and aryl).

124. The compound of any one of the preceding claims, wherein D2 is C1-bimatoprost (radical) substituted at C15 with alkyl substituted with oxo.

125. The compound of any one of the preceding claims, wherein D2 is C1-bimatoprost (radical) substituted at C15 with alkyl substituted with oxo and aryl.

126. The compound of any one of the preceding claims, wherein D2 is C1-bimatoprost (radical) substituted at C9 and C11 with substituted alkyl (e.g., alkyl substituted with oxo), the C9 and C11 being taken together with the substituted alkyl (e.g., to form a substituted heterocyclyl).

127. The compound of any one of the preceding claims, wherein D2 is C1-bimatoprost (radical) substituted at C9 and C11 with alkyl substituted with oxo, the C9 and C11 being taken together with the alkyl substituted with oxo (e.g., to form a substituted heterocyclyl).

128. The compound of any one of the preceding claims, wherein D2 is C1-latanoprost (radical) substituted at C15 with substituted alkyl (e.g., alkyl substituted with one or more alkyl substituent, each alkyl substituent being independently selected from the group consisting of oxo and aryl).

129. The compound of any one of the preceding claims, wherein D2 is C1-latanoprost (radical) substituted at C15 with alkyl substituted with oxo.

130. The compound of any one of the preceding claims, wherein D2 is C1-latanoprost (radical) substituted at C15 with alkyl substituted with oxo and aryl.

131. The compound of any one of the preceding claims, wherein D2 is C1-latanoprost (radical) substituted at C9 and C11 with substituted alkyl (e.g., alkyl substituted with oxo), the C9 and C11 being taken together with the substituted alkyl (e.g., to form a substituted heterocyclyl).

132. The compound of any one of the preceding claims, wherein D2 is C1-latanoprost (radical) substituted at C9 and C11 with alkyl substituted with oxo, the C9 and C11 being taken together with the alkyl substituted with oxo (e.g., to form a substituted heterocyclyl).

133. A compound represented by the structure: 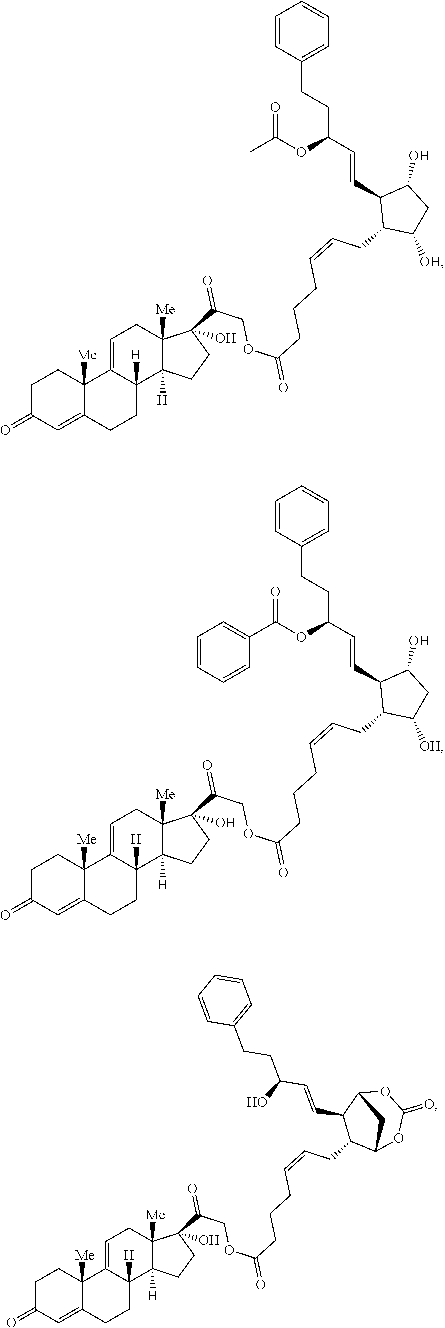
or a pharmaceutically acceptable salt or solvate thereof.

134. A pharmaceutical implant or article comprising a compound of any one of the preceding claims, or a pharmaceutically-acceptable salt thereof.

135. The pharmaceutical implant or article of any one of the preceding claims, wherein the implant or article comprises at least 50 wt. % (at least 60 wt. %, at least 70 wt. %, at least 80 wt. %, at least 90 wt. %, at least 95 wt. %, at least 98 wt. %, or the like) of the compound or pharmaceutically acceptable salt thereof.

136. The pharmaceutical implant or article of claim 134 or 135, wherein the implant or article comprises at least 70 wt. % of the compound or pharmaceutically acceptable salt thereof.

137. The pharmaceutical implant or article of any one of claims 134-136, wherein the implant or article comprises at least 90 wt. % (e.g., about 90 wt. % or more, about 95 wt. % or more, or about 99 wt. % or more) of the compound or pharmaceutically acceptable salt thereof.

138. The pharmaceutical implant or article of any one of the preceding claims, wherein the implant or article undergoes surface erosion to release the compound, the first radical (e.g., a steroid radical) (in its free form), D1 (in its free form), D2 (in its free form), and/or the second radical (e.g., a prostaglandin radical) (in its free form).

139. The pharmaceutical implant or article of any one of the preceding claims, wherein the first radical (D1) (e.g., a steroid radical) and the second radical (D2) (e.g., a prostaglandin radical) are released (in their free form) from the pharmaceutical implant or article at near zero-order in a buffered solution or in vivo.

140. The pharmaceutical implant or article of any one of the preceding claims, wherein the first radical (D1) (e.g., a steroid radical) and the second radical (D2) (e.g., a prostaglandin radical) are released from the pharmaceutical implant or article (in their free form) at 37° C. in 100% bovine serum or at 37° C. in phosphate buffered saline (PBS) at a rate such that t₁₀ is greater than or equal to 1/10 of t₅₀.

141. The pharmaceutical implant or article of any one of the preceding claims, wherein the first radical (D1) (e.g., a steroid radical) and the second radical (D2) (e.g., a prostaglandin radical) are released from the pharmaceutical implant or article (in their free form) at 37° C. in 1% fetal bovine serum (FBS) in phosphate buffered saline (PBS) at a rate such that t₁₀ is greater than or equal to 1/10 of t₅₀.

142. A pharmaceutical composition comprising a compound of any one of claims 1-141, or a pharmaceutically-acceptable salt thereof, and at least one pharmaceutically-acceptable excipient.

143. The implant, article, or composition of any one of the preceding claims, wherein the pharmaceutical composition is suitable for ophthalmic administration, subcutaneous administration, intramuscular, or intraspinal administration.

144. The implant, article, coatings, or composition of any one of the preceding claims, in a form suitable for ophthalmic administration.

145. The implant, article, or composition of claim 144, wherein the ophthalmic administration is intraocular, intracameral, intravitreal, suprachoroidal, punctal, retrobulbar, or subconjunctival.

146. A method of treating a medical indication orabnormality in an individual in need thereof, the method comprising administering to the individual a compound, pharmaceutically acceptable salt, implant, article, coating, or composition of any one of the preceding claims.

147. A method of treating an ophthalmic disease or disorder in an individual in need thereof, the method comprising administering to the individual a compound, pharmaceutically acceptable salt, implant, article, or composition of any one of the preceding claims.

148. The method of claim 147, wherein the ophthalmic disease or disorder is glaucoma.

149. The method of claim 147, wherein the ophthalmic disease or disorder is ocular hypertension.

150. The method of claim 147, wherein the ophthalmic disease or disorder is selected from the group consisting of ocular inflammation, diabetic macular edema, posterior inflammation, anterior inflammation, macular degeneration (e.g., wet macular degeneration (AMD) or dry AMD), post-cataract surgery, and retinal vein occlusion.

151. The method of any one of claims 145-150, wherein the article or implant is at least partially biodegradable.

152. The method of any one of claims 145-150, wherein the article or implant is non-biodegradable.

153. The method of any one of claims 145-152, wherein removal of the article or implant is not required (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)).

154. The method of any one of claims 145-153, wherein the article or implant is not removed (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)).
